SEQUENCE LISTING (99 sequences)

SEQ ID NO: 1 - DNA, 188641 bp, Homo sapiens
  source 1..188641: mol_type = genomic DNA
gtagtaggtctttggcattaggagcttgagcccagacggccctagcagggaccccagcgcccgagagaccatgcagaggtcgcctctggaaaaggccagcgttgtctccaaactttttttcaggtgagaaggtggccaaccgagcttcggaaagacacgtgcccacgaaagaggagggcgtgtgtatgggttgggtttggggtaaaggaataagcagtttttaaaaagatgcgctatcattcattgttttgaaagaaaatgtgggtattgtagaataaaacagaaagcattaagaagagatggaagaatgaactgaagctgattgaatagagagccacatctacttgcaactgaaaagttagaatctcaagactcaagtacgctactatgcacttgttttatttcatttttctaagaaactaaaaatacttgttaataagtacctaagtatggtttattggttttcccccttcatgccttggacacttgattgtcttcttggcacatacaggtgccatgcctgcatatagtaagtgctcagaaaacatttcttgactgaattcagccaacaaaaattttggggtaggtagaaaatatatgcttaaagtatttattgttatgagactggatatatctagtatttgtcacaggtaaatgattcttcaaaaattgaaagcaaatttgttgaaatatttattttgaaaaaagttacttcacaagctataaattttaaaagccataggaatagataccgaagttatatccaactgacatttaataaattgtattcatagcctaatgtgatgagccacagaagcttgcaaactttaatgagattttttaaaatagcatctaagttcggaatcttaggcaaagtgttgttagatgtagcacttcatatttgaagtgttctttggatattgcatctactttgttcctgttattatactggtgtgaatgaatgaataggtactgctctctcttgggacattacttgacacataattacccaatgaataagcatactgaggtatcaaaaaagtcaaatatgttataaatagctcatatatgtgtgtaggggggaaggaatttagctttcacatctctcttatgtttagttctctgcatgtgcagttaatcctggaactccggtgctaaggagagactgttggcccttgaaggagagctcctccctgtggatgagagagaaggactttactctttggaattatctttttgtgttgatgttatccaccttttgttactccacctataaaatcggcttatctattgatctgttttcctagtccttataaagtcaaaatgttaattggcataaattatagactttttttagcagagaactttgaggaacctaaatgccaaccagtctaaaaatgcagttttcagaagaatgaatatttcatggatagttctaaatactaatgaactttaaaatagcttactattgatctgtcaaagtgggtttttatataattttctttttacaaatcacctgacacatttaatataggttaaaaaatgctatcaggctggtttgcaaagaaaatgtattacaaaggctgctaagtgtgttaagagcatactcatttctgttctccaaaatatttcataaggtgctttaagaataggtatgtttttaaaagttaagttcctactatttataggaactgacaatcacctaaaataccaatgattacaaacttccttctggccttctggactgcaattctaaaagtgtaaaaaacatattttctgcattaagttaggcagtattgcttagttttcaaagtggtaggctttggagtcagattattttgattcagatcctacatctactgtttagtagctctgttgcctgaggcaggtcccttaacatctctgtgtgtgacttgacctttaaaatttggagactgtcataggggttaatcccttgagaaaatgaatgtgaaaagttagcctaatgttaactgctattattatggattaccatattttcacattcatcacagtacatgcaccttgttaatataagatgctcaattcatctttgagtataattttgtgactctcaatctggatatgcaatgagtgggcctgtatgagaatttaatttatgaaaaattgtgtttcacatggccttaccagatatacaggaaacacgtcacatgtttctattgtatgttgttaaatgccttagaatttaactttctgaataggatcccttcagtttgagagtcataaaagagtaaaattattatggtatgagttatagattgtattgaatatctctttatatgtctaggttttgtcattggaaaaccaaaaagtttggaaaaaaaatctaagttatttcttactttcttaattttgtgtggatttcacatcaagtataaaatttgaagaacatctgaactatcataatccatatatatatataaaataaacataatctaagagagaatttcaccatgaaaaattcaggtagttcatgactatcagagcaaacaagtacattaaattgaaacttttatgaaaataacatttatgaaataggaagctatttttaaactagaagtgatatattagcatataatttataattcatatacaagtgggattgatttataaatggtcaccaacagagattgtgctatttaatttgggaaaattttttaaatttacattttctcacaacttttaaggtagttattcagtttgttcctctctgtctcttctctcatgccctgaatttttcatatttcgtttagttgtaagagtgtatatcaaaccgtgtgtcacatgacataacttgaattttcgtcgtgatatctgtgctatgtctaggtctatactgaggaactgtgggaaccccacagaatccaagtatacagtgccactgatttcttacaagggatgtggggtctcctgtaaactctgcagttagtctcaagtaagaccaaagagtaaaatattgttaggatctaaggtggaaattcagcaaagaatcacatagtctaagtctcgagtttaacagtaagataatttgagatacttttgtaattattaaacacaaagtaatgagagattttaaaacaaacaaatacacctgaatttatatatcagaataggtatggtggttcaaaatagctatctaataaaaaccacactcctattctaaacatttgcctttgatcaaaataattttgggtctcttattatgaaattgcctttctaaataatacataaatttcttctcataagtatatattagccacattattttattgttattgttttatattcatagcttgctttagattaaaaattatattacccagactggtctcttggacttgcttccaagtgacttttgactgtatcacaaaatcaaattcactctgaaaatataaagatttttcatcataatttcctttgttaacagccaagtgctacctaattttaggtgttttcattaaaaaaaaatgcattgcaaactttaaagacaattcttttgtttgtttgtttttaaaagacagagtctcactctgttgcccaggctagagtgcagtgacacaatcataactcactgcaacctccacctcctgggctcaagtgagccttccatcttgcctcacgagtagctgggtcttcaggtgtacaggtgtgtaccaccatgcctggctaactttttttttttttaagttatatagagacagtatctcactatgttgcccaggctgctcttggagctcctggcctcaagttatcctcccactcagtctcccaaagtgctgggattacaggcgtaagccacctcaccctgtcagcctaaagacagtgcttaatgaagagaaatataagtgctttgagcaatggaagtataattaaaattatactatgaaagatttataaagatgaccattttgaatgggaccacacttatttggttatataaattatgatacactattaaaaattcatcatgatgattttgtatttacattttatttacatgtttgcaatttgtgaggaaagctaaaattatggctaagccataaatatttttgcagtttgttgagggtgtttgtaaaagtgttgccaaggaagaccagttggctacccaaacaagggtttagtctaggtctgatcaatacatacacattatctcaggtttgtctatcagaaaaaccttaggttatccaaatcaaaataaaatagatgcataaaacaaaggccaatatgtgttgaacaattatattgtgatatacaactgccaagcattcccgattaccatgactccatttagtcagtccatgggcaaatgccatcaatgaggacagcccagggtttccatattctctcttggctttacatcctataggaattggaggggcccacctctgggataggagcccttctgtcttgaacaatgttgtctgaacactaacaaatgttgactttctacaccagtccctcaatagtcttttctatttatccttttgctgaccatgttttgttattacacagttgagatttttcagctgggaatctgtgttaattttgtattaattttgattagcttaactctcagagttctaaaagtacctcctgtacctgatatatgacaaaaattataattacatttatttatatataaaatatctttgtatatgtaaaatatctttgtatatataattatataattgtttcttttaattttgcaaattttaaaaagttctcctttgttttgaagtttattcctatagttttttatatgctagttaaattattaatcacttgattcaagtaatattcttatatacttataaggaatagtgtagttttaatatttaattccttgctaaagagagaagtggaatctatttttcttagctacttcatcaatattttatgtttgatgtgacagtcaaaatatccctcagagctaactgttacactagggaaatcacggttttccagttttccatttatgtgttatgggagggagtggaacttagtgtaataatattcaatacataaatgttaacacttgtttaaaggtccttgagtgagtactgctataaaatgcattattattgctagtgtcatttcacaagagcctataatttcagtgtgatagagctacaatataagtatagtattgcaaaaccatcaggaagggtgttaactatttagcatgcagttatgtgttggttgtcaaaacgttaaaaacatctctgactcagcagcaattttggcaattttgatcctgaggcatctgtgtagggcatcttcctggagaaaaacctctgagatgcaatgaggtcaaaaggggaaaacagactatgataaagatcaagttgtttggagatcttgtagaaagattaatttacaaatatgtcaagtgcattatcatggaggaaaacattgctatttctgttggttctcttcagagctctagaatcaatttaccacatagttgtttcagtgtgaaattagcattacagagtggctttacggctttactgtagggcattgtgtcagcaaagagcttaggcttcttttagcaagaagcttgtaaaaatttaatttactcttagattgcttgatgtagagaattacattcctacagagctctgaaaaatcttttttcagagtttttcacagctgtattcaagttgcaaggcttgtcaactttgctatttttctgtgcagctctgttaacttattattatcttttgacataaattatgattccaaattgtaaagctctggatgtcagggccttttctaatttgtttagtatgatattcagaccatttcaagactcttccgtggaacaatttaataaagatttttttgtgatgttaatgagttcatggtgatcaaccctagagacctgtgtctattgtagatcgatgacattcaacagtcctgcagtgctggcatcattttgataaaaaggggtcaaagcaagtgggactgtgggcagatttttaatgcttagaacaattattccatcgaagttttcttgtgtcccttctgccttagcctttgtaggatagcatgcttgctaatttcttgctcatggggtaaggaaatgaagatttttgctaggtccgtaggattattaggactactcaggcctgaagctatgcctggatatagccagaaaactctcccatagcttgctccaaggagctgagatacagcagtacttcctttgtaggtcatgattctgggtaacctggaagatgacctcattcatattctgtattctatgtgagacgttaagaaggtagaggtggccaagaaggaaattgttgctgcctttatggaacaaattatctgaaacccagctttctcgagggcttcattgaagtactcaactggggcacttaacccagtctaaggctggtcaaggaaggcttgctgggggaagtgtcttttgtattcacacctaaaggaggttattcaattagaattatccaaagagggtagggatgggctaggaaaaatttaaacaggtagtgtggaggactgacaggataagtaagcatggcaccttcaaaatatcctgagaagttccctatgacgggaacataaaatatgtgacagagatttgtgggagatgggtctggaaactctagcaggggccagatcgtaagggggctttgtaggctttgtaggctttgtttgggctttatcatactggaagtgaaaagccatggcttttaaacaggagagggacataatcagttcatatactgttgcagttttgtaaaagaaaagatgagctgaaagagtggccatggtggaggtgggtggggtgggggggagggggcggggagagagagagagagagagagatttgaaagacatttaggaggtaaaatcaactggtttggtaatcaattagtagttgaaggtgaaggaaagagaagagttaaggataacatctatatttgttgatttggataatagaggggacagtggtgctgcttattgaatgagaaaatttaatcggagaagaaggcatggagcaggagtgcagacctatgtgactctacttctctcaaaaccagaaacggaaatgatgtatatggctcagggttaggtaatatggttatttgaaaatgtattaaagtgatttagagcttagtcttaggtaagagatataagatgtctgaggtgacagttttataaatatgtagagtgcccacttgtttggccttattgtggcatagtgtgacctgagagtgttaggaagaagcagctgagttctagggacagtactggttaaattctacttagaaattatacttagaactctcctatataacctgctaactgatgtctgaacctcctgataacttcactcctttaggcagtgcttttcacatcacgggacacaacatatgagagatcatagaaattcaatgtggtatgaaaatctgcttgggacttcagatattgtctccagtgattgaataaaaataggagctcacctactatgatgaggtttctgtgtgtgttaaaagaaggttttcattacttttgaaaaggttatgtatccttgttttatgttaaaactttgagctttgttaaatatgcagagttctctttcttagcatggactacagaggtgcaactacctcctacctgacttcacatctactcccaaatgcctagtgaaggcttaataatttcaaaaagggactctagaatttcatttgataccagtcagacaaatgtgtgaaaattaagcataataggcagaatcccaggggtactgacagctgtattaagaggtgattcaagggctaaaccttagagtccagcattggttatgggtgtgacaagaaaatgaagcctatgttggctgggattagcaaccacagttctagaggaagcaaggtggagaaactatatagggggctccctttgtacgttttatttattttaaacatctctataaactctagaaattaaaacaacaataccaacacaaaagcatcactttttcgaccaaagaccattgctatacttttttgtgtaaagggctagatagtaaatattttcagctttgtgggccacataagtctctgcaatagacaatatgcaaacaaataagcatggctgtgtttcaattaaactttattatgaacattaaaatttgaatttcatataacttttacatgttgcaaaatattctttatttaaattctattgcaatatgctttaaaagatacagtttttagtctttcttagtttaaaataaaatctagaaaaaattttaagtcttctataactttttttcggtaactgaataattttaaaagtaagtgaaacatttagacatgcaaaatggacttttcagaagaagaaaatggtagcttaacagttattagattattgtccagaataatttttgacttataagtctctgttgaccatttcattgcctctttttttggaatatgcatcttttaatgtgtccttcaaggcaaaggctctatcttatctatcttgtgtcttgcattttcccagggcaatgtttttcacaatttttttaaaaaacaatactgtaatcaattttcaaataaaattttccatgggaccgcagtgtatacaaatagcagtgacaataaaagataataactctcccataaatacaaagaaacagttaacctagtgctctaaagtaaaggctacagtgattttgtataacatttatatgtaattttcttgatcctacatggttgtgtttttcacagtgttatgtttctgaaatcgagatgccttttataattgatgtcaaaagaaacttgtcagccacaaggcccaggaataagttgtaatatgggaacttagcaatacataaaggtatatatactcctgtgacctcagctgaattatttgcattggttgcatcccacaaggttgactcttaaataaatttagtttgttgcttgaaatttcttgggataaattactttgtgatgtagttttgaaaaaaaaacaggtaatatttagtctgaagtttgtctgacatactaagcaatgtaattaaagtagaagtcgcctaagctcagcactttattatgccttgaaattatactgcctgtcctacaggtgaaggtgttatgaatgcagtttgtcactgtaactctattcatagctctgaaaggctgagagtgactcagaagaatatttttgctctgaatatgaagaacgcttagactaaaactttaattacgatgctgaagaagaaagtggtaggtgattgcatgaataagtatgtaatattgttaatttctaaaaactgtgtatagttaatgtagtgcttctttttggaaaggctattgttaaattgatggtaaattctataaccaatatcaccttaaagcaagtacgcatgataaagtattataaaaccatgataatatcatatgtggcttattattgttccctgagtgttgtacaactctgttatgctgtgatgaaacctcatgcaaacaggtatgtcaaagatatgatgggctgttaactgagcttggcccacatatggtgtagtgacatgctcactaatgcagtgcagagataaccaataacagatcataacaggtttaaatatgtgcaaggagatgtcagcagaagctttcctacatagtgaatactaaacaagcctgacagcccaggatcatgttcggatcaatctagtgtgctaaaattaacatatagtcctacatttgagaatgtgtgattttcttggttcctgtctataaaataatattttaaaatacatacatttcaaatcagaagttggtgaattcactgaaatatttctagagaacactaggtattggggctcatagtgtgaaaaccactgacttaattcttcccccatcttggttgttcctgatcttcccttgtgtccccattccagccatttgtatccttagaaaatgatctcatattctacttcatctttatcttcattgtcaactgtcaggtagcaatatatgatggaagaagcatgtactttggaatcagacagacctggctggaatcctaactctgtcacttattaacaatgtgatcttaggcaatttacttaatctctctgaacctcagctactctcgtcagtacaatgagttatccttatctttacatggcacagtattattatgatatcaaaaattcattgagtatttactctgcatattagtcaaggttctccagagaagtagaaccaatgatacacacacacacacacacacacacacacacacacacacacacaatttattataaggaattgacttacatgattatgatggctaacaagtccaaaatctgcagtatgggtcagctggcaggaaacccaggagagtcaatgttccagtttgagtctgaaggcagtctgttggggaatttcgtccttctctgggaggccagcctttttgttctatacaggccttcaaccgattggatgaagttcacctttattagtgagggcaatctgctttaaccaaagtttactgatttaaatgttaatctcatccaaaaacacccacccagttgacacataaaattaaccatcactctctgtaagcactttctatgcattaagtgatagcaaataatgccagacatagggcgtctttaataaatggtaagcactgttatcagcaacaacaggattattataattagcaccttttcatctttctgtctgggctctgagaaagtacctctcttctctaaatttatccctcctttcctatgaattagacccagtgctttctctgaattatgaaggtcacactcctacaaatgccccttcccaattgcacatctgtcggctttctttgccattgacttttatctctagcttttaaatttacaggcatatgtcagttaacaatgggaatgcgttctgggtaatatgtccttaggcaattttatcgttgtgagaatactatagagtatacctacacaagcctagatgtcgtatagcctactacacacctaggcaatatgacatagtcttttgcttctaggctacaaacctgtacggcttgttactatactgaatactgcaggcagttgtgacacagtggtatttgcatatcggaacatgtctaaacacagaaaaggtgcactaaaaatactatgtagtgatctcatgggaccaccattgtatatgcagtctgctgtagactgaaatgtcatgcagtgcataactgtatcttaaatactcaaagtatcacctttgtttgtttgtccccttgtgtgcatcatcctaacgtggaatttctctgttgattagggccagcgtattagtttgctagggctaccataacaaaataccacaaatttggtggcttaaataacaggaatttattatcttatggttttgaagactagaagtacaagatcaaggtgttggcaggtttttcttctaagggccatgaggaagagtctattccatgcctttcccctaccttctggtggtttgctagaaatccttggcattccttgacttacagaggcatcaccctgatctctgttttcatcttcacatggcattctccctgtgagcctgtctctgtgtccaaacttctttactattaatataaggacaccagtcatattggattagggtctactttagtgacctcattggaatgttattacctctgtaaagatcctatctctaaataaggtcacatccttaggtaccgggggttaggactcaaacatacctttttttggggaaacacaattcaacctataacaattgataacactctttaggagcagaatgcgatatggaagtaatttgagaccataaagtatatacatgtagggagttaatctatgaaacctattgaaagccatatatacctcatgtatagtggtccataaatagcatggagacattgcagaggatgttaagtgatatgatacaggaacaatccaagaaggtcataagaaaaaggaccttttgctcttgagaggactgaagaatgactttccatttatgaaattttggtacatgtccactaaaaataggatgaaggccaaacttaggaagaatattttgataatggagaaggttgcatataaaaacattttattgaggacaattaaataatgttggctggaagttttaggatgatcatctttaggactcagaaaaagagaagaaacattattaaagaattgtccctgaacaagtataggcaccctcacatttgcattgcatttactatagaattgaaaaatgttttgaccttttttttttggcttttaatatatttgaccaagagtaacagctaagcaatacctatttgcaatcagtgtcatcatgtgggctccaaacatatcatgtttgtgtaattaattgattgacccattaatttgttcaatttctgctctgttccaggcactgaacaacatgatggagataaaagataaatattacacctgccttgtcctcaagaagttagtcttctgagggaaagaaattagcaaacaaattgtaatctcagttatgtgccatgttccatgctgggcacaggggatacagtagtttaaaaaaaacacaagatctataaggtgtttcttcttgtggaccttacagtctagggtgcttggaaacatggggcgttggcagacaagtaaatacacattttgtggtaaaggctcaggtagaagaagtacaggatagaatagagcacaccatggggaattaatctagacttcagagaggctcacacatacataatttatgtgtgactatttcaatgcatttgaggtttcttggaaatagaggttaggttttattttaaggaagttaccattttttttttcagtgtgatgtggttgaaccaaagaatgccatgcccagtgatggtaataggataatctttttaaaaattaagagccacctaataaatcaatagtttcattcagcgggagctcctgcagagttcaaaaagaagagaatctggcacagcgtttcctttaaagttcattttcctagagtgtgaatggaagcaagagattataacattttgaggtcaaaaaaattctgaaatgcctataaaaattattttctccaaattatcatcatttgtgcttttaatgacctgattgcaaagatgaacattttgaattcttaaattgcttattaggattggttaatgaatcaattatctattactgtatgttttgctattggaaaaaatagcaacttaagtgttttgcagacctttacttaggtatatgttgcttttatgaaaaaaaagatgtaaatattaagtaaaagggatttaaagcaaggcttttgaggtagagtcttattaattccttggtaaaccttgagccaattgttgtctatgttctctgcctctgtcttgctccttccttctgggattcactgtgggaatgcgggattgttaatctggggatgctgtccaatcctgcctctctcaagctttgctattgatctccctcccagtgataataaagcttgaagaaaatgaaagtagcgttagtattggtcctcaaactcaagaacaggatgaaacttaaatcttgagtcatacaattgtgtctacatactgctccccaaaaagagaagtaaagaagatgctaactttcccttttaatttgcagtacttagcaatttgttttcttgagggttaagtaataacagtggaagaaaaaagggttaaaatgccaccaagaacccaattccatgtttagtttgaaagtgggaaatcagctgccactgggaagtctgaatccaatgccatgatgttctttgaatccttctgagaaataatcatgtgtagccataacatacctgtataacagagcagagaacataaacaaatgaaggtgaagggaagattaagacagaagagaaaaattccagaatcgactgatcatttttatctgtttagatgatttcaggcagaatcctagagaccaactttatcacaactgaattttaaaaatcaccagctttgtcattgtgatgcagcatcagtttcagtattatccttggagtattaattcttaatcatcttcatcttagaacatttttgaggtcacttctagtctctatttcaccagtgaagaaacaaaaatccccaaactatatcaggtggaattacacagtattttttttttaattttggggaaagtcgattcaaggcagtaacttgcaagctagtgttagaaaggatttaataaatagtggtttttctgtacacatagtgagaggtcattacatcatttggttgttgaaagtcataaggatgtctagcatgcgctttgcctgtagtggttcatgccaggcagattcctgactcctataacccagagcttatcagagcatttatgtccccaaagagaaatgtcacctccatctttcaataaacactttagcaaagaaaaatcaagtactttaattccaaatcttgagttaattccagaataacaatgatggctcggaaaaatatgggtatttctgtcaaaggacagagaaacctagtagagagtatttactttgggtcctagtgatggtatctgaacaagctaggtgaacaaagagcctcaataagggattttgaggtctagaaaaagagaggaaataccaaataaatggaataattataaaataaataccagcaaagttaaatcaatatatcatgtgggagatatccttatatcactcatgtgatttctattttgttcctatattaggccaaggagaggtggaacttgttttcctttttccctctcagctacgaatggacatacttaaaactgtttctctgcttctgttctctaaaatgtgattgtctaacagtaaccgtgatgacgttttgacagttgcacaagtttctttctttaagctttaaaaatgccagccagtaacccagtggcatttctactataaaatcttaaggccaatccatttccccttttccttattttcttggtttcaaatatatttttattgccaatggaaataaaaatcctaaattagagagcaatggcatcccttgtcttgtgaataaagagctcctaaatgtgaacttatacaggatgcagcaatttatagggtagttaatcattcttctttctagccagttgttccagctacagttttgtggctcttgttagtggcttcattcccagatagaataaaaatcaaaccaaaatcctggaaaggcactctgaggatgcttctctaaagtagatgggcatcaactataaatcacaatgctttgtttcctctgttatgtttcaagatgggtgggattttttttgtagcattacttattattgcctctcaagtgcttgagtctttgaaatccaagtcatgtgagtgaattagatacagctgttagaagtggcctttcaatgccaatggtacacattccttggtttctttacgatactattgctcttacaacttttatctgaagtcataaattcatagttgtcccagaagttaagttccttgcttctagaggacagaaaacaaacaatttacacaactcatggtgcatgtcaccagtccttagatctcatgaaatatgcatgaaatcttaaatcacttgctgtagccacccagccattgacatatttgaaagactttagtgtatcaaagtcactataatgaaaattttgatttcaccagttctaggagtgaaaaatcaaatgtttagtaaaactttctaaaattaacactgacagttgatttctgtatactgttgttcttaataatagctttattgagatataattcatattcaaaacaacttacccatttaaagcatacaatccaatgattttttagtatcttcaaagagttgcctatcaccataaccaattttagaacactttcatcactgtaaaaagaaactccattcctattagcagtcattccttattccaaatccccctgctcgccctagacaactacaaatgtactttccatctctatagatttgcctgttctggaaattttatgtaaatagaacaaagtgttcttttgtgactggcttatttcacttagcattttttttcaaagattcatccctgttgtagcgtgtatcagtgcatcattcttttttatttttttagagacagggccttgctctgttgcccaggttggaatgtgcagtggcatgatcatgggtcactatagctttgaagtcataggcgaaagcggtcctcccacctcagtctcccgagtagctgagactacaggcttgcaccacatgactgtctaatttataattttctttagagacagggtcttgttatgttgtctaggctgctctcaaactccagggctcaagtggtcctcctcccacagcatcctaaagtgctgggattataggtgtgagccacagcacctggcttgcatcattctttttattgttgaataatatcccacttgtaagaatatgtattttatttatcctttccccagttaatagatatttcgattgttcctaattcttgtctattataaataatggtgctatgaacatttgtgtacaagtttttgtgcagacatccattttcctttcttttgggcatatacctacgagtgtaatggatgggccatatagtaactttatgtttaatattttgaggatttttcaaactgttttccaaagtggctgcatcattttaaattccttccaccattgtgtgagtgtttcaatttctccacatatttgcaacacttactattatctactcttaaaaattacagccatcctactgggcatgaagtggtatttcattgtgagttttttttttctttttctttttttctttttttgctaatgtttgtggattttcttttcattttcttgatggtgtcctttgaagcacaaaagtatttaattttgataatttccaatttattttttgttattgctgtttgtgcttctggtgttgtatctaagtgtatgctactttaaaaaattagttgtaatatggcaaattggatacatgtgtaggctttggtgtcacaatcctaattttaaaattctgactctgcccttgacaaattaactaattaagcttccttagcctcagtttctcaactgtaagttggagatattaccaagacctacctcttgaattgttgtggggatcagatgaaataatgtatgtgaaatatttagaattatgcaagtctgtggtaatgaatactaatgttagctatcattattgttataatcccaataataaattctggtgctttgaaaattaaaccaaagccaagcagttgatatgaagaagcatgtaataatgtacagacataatgctttatagacaacattgaatttggctctcatgaacatcaggaatagtggtcatggtagttattatctccagcaggaactgtagctgagagatcttcagagctttttccaaggcgatatcactgggaaataatagagacaaggttacaagctagggctgtgttttcttcttaaaatctttagttcagtttttttcaataacagatttgtagtaggcatcaggtgactggggattcgtattcttcaagttgaaatattaccttgttgagaaagaaaccatgtgtgagacaaccatgttgagaaagaaaaagtgattttatagaaaattaatattgatagtgagcattatatgaaaatcatgaagttagaacatatttggccagaaaatttacattaatagttacccatagcaattaatgcattataattacacataccttttctttaatgaaaaagaattctttccttccaaagttatgcatgctattgttaaacattagagaatatagagaagcaaaaaagaaaatatcttttttgatattttcttaacatacgtctgttcctaataatgtttatagtttagaagcattgcatgaaatgggtagatcaattttctatttaatgtttggattcattaggtacgaagttagcaaattaatttccattagggtgcctgtatggttgtaaatcctggacctgcagaagatttttcagtattggtttgtagtcttttgtttagcagcaaataattagttctccagagcttctgaaattaattgaccactttaatggtgtttacctacctagagaaagaaaaagaacttctccaagtcccttggtaaaattaagcctcatgaacaattaactcaaatatacacaaggcttgtctttagcgagcatatactccctaaagttgattaagctgaccaagtgattactgcttataaattcaccattttatggagaagaagcaaacactgctaaataccttgtggaatcagaggaggggaaattagtaacttgaccccaatactgcgattttaaattgaattcttgaagcctacaagttttacacaggactttagagagctggatagtatcactttgtcaagtcctacttttactatgattctttgagaaaaatacatctgactaaataactctgaatctaaattggataaaataaatgtgacattcaaaatgttatttatgattttagaaaaatatccttatagacactagatgagttttagtctcaaatcaatcctccctatcatagtcacttatcaaaataactaaagcaaagtggtagagctgtgctctagaagtttgggatttatgatcacaatcttttccaatgagtcccctctttcctctgcctgtcttcaacatttgtttttttttttttttggttaggactatccagattgtgtggcctatttcaaactcatggcaaatacattggatgatcagaaattttctaatgtatttgaatttgtctacacaaactagagtaattgctattaattcctcaagtgttaattatttcatgcaaaaaggaaaaaggctattagtctttaagtgtattagtatgtcaatatttgggagaagtgtcatgcaattagtggtttgaatttcctattttattttattgcattttattttatttgcctagtcaaataaaaagtaatgttaaatacatggaagcatgattgttttctacactaaaaatcattttgacttgaaaagatctgatatccatgaccttcatctgaagttttggcagatgaaaatgtcagatgcgtcttttggattaataaaaggcaaaagtcagatcgaaaaatgagtataagctttaattatatgactttaggaggatatgttatgaaaatcaaagctttaatagtgattataattggcaagttctttttttataaggaattacaagtcactctatacaaaaattggaatttttgtcctaagaaatgaaatttactatagtttcatctgtgtgtgtgtgtgtgtgtgtgtgtgtgtgtgtttaaaaaatcaagtgatagggcttttcctcaataaaatctgaaatctcttatagttaagtgaacagaacagtgtatctaggatgctagacttttttttcaaagttagtttaaaacttatacatagtaaaatctgtatgccttagggatctctgtttgctatcccatagtgaatgattaattagtttctgttagaaatagtcagaactaggctgggtgtggtggtggctcatgcctgtaattccaggactttgggaggccaaggcaggaggatctcttaagcccaggaatttgcaaccagcttgggcaggctggtgagatcctatctctacaaaaacaaacaaacaaacaaaggacaataagaaagaaagaaatagccagagctttgaacaaaatttctaagtagaccaatgtaaaagtctgtcgtcaatatgtagtggctatgaatggaggttatgaatgaaagagaaggataagatgaactagaggtgagaggggaagacagcaggcccaagtgaaaggcagagccgagtttattgctttttggttattccaggtgtgtctgctttgtctcatgaaacacctggatgatcactgatttctagtggaagaaatgctgaaaagtccttactgtgcatttaaacattctaggtttaatatactcagggtttttcaaaagaaagggtggctggagttttgcactaactaatatttcataaagtgtctaagtatagatgtctggtttttttttgtatttctaagactggcttgaggtaggcatggagaattctttgatgggacataattttcttcctttctttttttttttttttttttttttttgagacggagttttgctcttgttgcccaggctggagtgcaatggcacaatctcggctcactgcaacctccgcctcccaggttcaagcaattctcccacctcagcctcccgcgtagctgggattacaggcatgtgcccccatgcctggctaattttttttgtatttttagtagagatggggtttctccatgttggtcaggctggtctcgaactccttacctcaggtgatccacccacctcggcctcccaaagtgctgggattacaggcgtgagccaccgcgcctggcctgatgggacatatttttcattcaattttattgatttaacctcacaaaataaaatatttccttaagatgactctgtggtcattgttgggcagcataagcttaatggattttagttatcataatttaccttaaacccaatttgtatttcaggatataaatagaggtttattgtagtgaatcttccaggaaatactaagtgatactaataattatagatggtgaacttaagtctttatattactgaatttgtttggtttgatgatgctaggctatggcattcttgctaatcaaaacgatgtgtcatggtgtaacataacttattaaaatgggcacagataacacaggaagctttttataaaagcagctcacaaattgtgttactttgaactgaactggccatttatgggaaaggtcactgggttgtaaataaggaccaaaagagttacgtttatattttttaaaagagattgaggagatttatttttacatttcttgaaaatgccttattttggtatggtattgacagatagtgaaattctgctcatttgtaaatatagtgtcatattttaataatttcaaacatattgaaaatgcagaatttattaatagtgggagcacattttcctttttactaaatgttctacaggttcttttctttccatccacacacagtgccattaccctcattctaagcctttcaaacatctggcagtaagtgatctgctgcacttagctctttccagctgagctgatttttaaattttcagaaaatttgtgagctaattgttaaacatggccattattaaaaattaaattatttcaacttataattaaataaattatattaaaacaaaagtattaaaaactcaaaagttggctgggcgcactggctcacgtctgtaatcccagcactttgggagaccgaggcaggtggattgcctgaagtcaggggttcgagaccaacctgaccaacatggagaaaccctgtctctactaaaaatataaaaaaatagccgggcatggtggtgcatgcctgtaatcccagctactcaggaggctgaggcaggagaattgcttgaacccaggaggtggaggttgtggtgagctgagattgcgccattgcgctccagcctgggcaacaagagtgaaactctgtctcaaaaaaaaaaaaaaaaaaaaaaaagaaacaaaaaaaaaaaaaaaacaaaaagcaaacaaacaaaaaaacaaaaattatcacttcctaattattttgcattttactattatctatgctattaacgttatttgccttcattgtatttgaaaggtggactatattctattgcactttcattgtactatattctaatatgcaactgtgtatcccttcccaactctgtgttcaatgactttatatttggttgctttaaaatgatgacgatgagagtatttatatcatagaaattggcaaatgccgtaagtcagtttttgtttttgtttttgttttccggagaggggattgttaaatatttgcctgcatgcaacaccactacatgcagtctgctatcttttgttcttcctgctttcaggctcctctcccagctgtctgtctagcacaacccagcataccaaattttcttaaatagggaaagttgaacatggtaaaagaatgaatgaagtcaaaagaatgtggaaagacctaggctttgccatttagtaaagtttagcatctctaagcctccatctctttatcaataaaattgagcaatgatcccttttagttctacccatttaagaagattttcaaatgaaaaccacaacctgctcatgtttatgaaggcactttggaaagcgctaaatacacgggtttttattagtagtaaacacttacttcacctttttcacttcttgactttagtttacaagggctcataatctaaattatatcataaattgctgtcccagatttttttacagcctaattgccacctgtatgttcgactttccttctgttctttatgttagatactgggatagtatgcaccaggtgggtgtgccatcactttctcagatgatgtccactgaagaccttgcatgatcatggcattcattttcctgctgtattcagactggcctcaactattttctttattgctctccaggaaaaattacaaatgaatcagactgggcaatgaagggtaaacctaattatcgctctttgttaaagacagctcttgttaaaatgcggatattgcaaattaatggaaaaaatatgacatagtaaaccatactcacttattaatatcttagtaaggaataattgatgaagttacttaaccttagagccctaattcagttaagttttaatgaaggacaagttgtagagatatcgagaacccagggcaggtgcctactgaagaagttccagaccaaggaagtataaagaaggacctgggtgggagcagtgagattggatatgagggccactggcaaagttttgccccagaacagtgtcaaaatgtttgcatttggcatagccctttctctttttgttctgaatggctttgctagaatatcttttctataatgaatttatcctgcttctcagatattgctaaagcactcccttttgaattttggtgctttaacatgcattttgatacattaccaaataaggtctgaatgacacaaattttagaactctccagagaaaagaaagatgctgagggaaaaagcataggtttgggactcactaaatcccagttcaattcctttctttaataaatatattcaattttacctgagaaagctctcgtgctctcgaattttatttagaaatttctctttgtacatgattgatttcacaatccttcttctgcctcctcttctactttcttctttctagattttcctatctttatgaagattattctgccttatcctcaacagttagaaacaatatttttgaaaatcactacggtatcctgcatagtgatttcccatgccaactttactaatttccattataaattattatttattgatgcctagagggcagatgagtgtagctgctatggagtgaggagacaaaacataagaaagttatgatcctaccctcaggtaatgattcagacatgataattaagtcaacaaattgatagaaactaatcactaactctctggctatagtcattctttcaatgaatagctcattactgagtatgcatgctacagtaacaaaattatataaggctgttgattaaatgttgattaagtgcatgtcttattcagagtttttttatatttgaaatggaagaggctggacttcagtaatttgctataaactgctagtatatgattatttgggggcagttattttttaaagaataatttaaatatggaatgtttagcagtttgttttttccctgggaaaaaccatactattattccctcccaatccctttgacaaagtgacagtcacattagttcagagatattgatgttttatacaggtgtagcctgtaagagatgaagcctggtatttatagaaattgacttattttattctcatatttacatgtgcataattttccatatgccagaaaagttgaatagtatcagattccaaatctgtatggagaccaaatcaagtgaatatctgttcctcctctctttattttagctggaccagaccaattttgaggaaaggatacagacagcgcctggaattgtcagacatataccaaatcccttctgttgattctgctgacaatctatctgaaaaattggaaaggtatgttcatgtacattgtttagttgaagagagaaattcatattattaattatttagagaagagaaagcaaacatattataagtttaattcttatatttaaaaataggagccaagtatggtggctaatgcctgtaatcccaactatttgggaggccaagatgagaggattgcttgagaccaggagtttgataccagcctgggcaacatagcaagatgttatctctacacaaaataaaaaagttagctgggaatggtagtgcatgcttgtattcccagctactcaggaggctgaagcaggagggttacttgagcccaggagtttgaggttgcagtgagctatgattgtgccactgcactccagcttgggtgacacagcaaaaccctctctctctaaaaaaaaaaaaaaaaaggaacatctcattttcacactgaaatgttgactgaaatcattaaacaataaaatcataaaagaaaaataatcagtttcctaagaaatgattttttttcctgaaaaatacacatttggtttcagagaatttgtcttattagagaccatgagatggattttgtgaaaactaaagtaacaccattatgaagtaaatcgtgtatatttgctttcaaaacctttatatttgaatacaaatgtactccctgggaagtcttaaggtaatggctactggttatcaaacaaatgtaaaaattgtatatttttgagtacctgttacatgccaggtagaatatctcctctcagccactctgagtggaaagcatcattatctctattttacagaaaagcaaactgaggctcagagagataatatactttgccagttaatgaatgatggagccatgattccagctgaggtctgtattgccttgctctctaggaatggtagtcccccccataaagaatctctcagtttcctttccaatcaaaaggttaggatccttttgattgccagtgacagaaacccaatttactagcttaagtaaataaaaggaacgaatttattggctcatgaagcctgaactatgtgaagacctaggtggagaactggccttaggaactcaatgggaccaaggactcaaatgccacctggtggcatttgccttatgctggttttattttctcagaccggaccagctttctacataaagtgggtccctggttagaactctttgctcctatctttaaggaccacgaaagaaggagccctttgtccttggctaaatgtgaaaaatcccagagactcttgagtcatagtgcttaccccttgggccactcatagtctagaatgaactaggctgagtctcgtgccaacagcacaggcctgatgccagataaaagggtgagtgaagggggataaaaaataagacatagctactaaattattgcaccaaagtaaaaacattgagttgacttgcaatttgtttcttttaattaaattcatttcctttttttggcattttgaaggcaaagtaagatattaaactttatttttattgattttattcaaagaattaagctagtgggagtagcagattcacacttctaagatcaagggccagcttctattattgaacacttggtgtgtgcaaatgccatgaggtagggatactttgttttgttttttattttttattgggttcgatctcttttgtttatgatgtatccccaagtgcctagaatagggcctggcatatggtatatactcaataaatatttgttgaatgaatccatgatggaatgtgaaatggctagcattacatagaaacctgtagcattgctggagagataaaatatataaacataatccattgcaggtatattgacaagttcaaaataatataatgggtattgaatatctaaatgtttgttgttgttgttgctgttgtttttgagacagagtcttgctctgttgcccaggctggagtgtaatggtgcaattttggctcactgcaaacttcgtctcctgggttcaagtgattctcctgcctcagcctctcgagtagctgggtttacaggcactcgccacaatgcctggctaatttttgtattttagtagatgtggagtttcgccatgttggccaggctggtcttgaactcctgacctcaagtgatctgcccaccttggcctcccaaaatgctgggattataggtgtgagccactatgcccagctttgaatatctaagttttaattggatgctgagggaatgattaatcagagtagggctgggttaattgaaaaatgtgatacatttgtatttatggccagatagagaacatgaatctgaatttgcagaattatctggcttaacatttttttctttccagttttcactgtatcccccatgttgattcaatttaaaaaatatacctattttacttcaattcaacaatgctatgccagtacaaacccatacgttctattatttttgttttgttttgtttttgtatctccaccctgttacttcttttcttataaaattggtatttgaaatttattgaaatattttggaagagtgacataccatttttggtactttgtacctctgcacccttgggaagtgaccctggcttcacatttcataactgccttgtgaccatggccctcaagtggttgccagatggttgaagaacattaacctatctggctcaattttgtgaccatggattgaatcctctacataactgcagtgtgcaaaccacacatccgttccaagattgtagtcaggatatgaactttttaagaataaaacttcttcccttctgatctgggcctggtatgtggtcctactagaaccacatcacctactcttggtgctaacaatttgtggcaccaagttgttcaagtttcacccattaaagaaattccccgaccttgccttctcctcaggtaactaccccattctattttttctttcatagctaacattctctgctctcctggtctctctacttcactttcatttacatctcagctcctgaagtatggtttccaccatgttcctaaaactacattgcccagggtcactagagacctcttatgaaatataacaacacctttctacattacttccgtgtggaccactttttcacattgaacccattttgttggtttatgtacacaccccttccttggctttcccatctgatccatttctcctttgatggagaaggtgagtctgctccatatttagcttcttactctgagtaaccaaatgttatggatgggaggttagctctgtgtgtgagagaaaggtggagaagcatgtggggagggaaatagatgggaaaaggtaattaggctttatagaagggctctcattagcaagcttctaggggatgccaagatccatgcttagagattgccaggcttgtcttcaaatctcagctgtgtattactcctttatgttttttgtttgtttgtgttgtttgtttttgagacagagtctcgctgtgtcacccaggctggagtgtagtggtgtgatctcagctcactgcaaactctgcctcctgggttcaagcgaatctcagtctcctgagtagctgggactacaggcatgcaccaccaggcctggctaatttttgtagagacggggttttgctatgctggccaggctggtcttgaactcctgacctcaagtgatctgcccgccttggcctcccaaagtgttgggattagtggcgtgagccactgccccggcctattactcctttagagtgatttagagccatgtttacttatggtaacttgacagtaatgggaataaccactgatgaaacgtaaagcctttgtctaattgtttacctagttcttccttgtggttcatgaaatttttcatctctgtacagtttgaaaattaagatgataatatttagagatattttattcctttgtgaagagaaaaaaggctttcattaacagaaatcagtggcaataacttaataaatacaatcagctggtgttcctatagtatttaaaagaaaacagaaagtttactagatttcagccagttttcagactatttaatgtctattcttactataatagaaaatatataatttgatcttgttctcatttttcaaagacctttaatacatgattttagtagttgaaaatgaagtttaatgatagtttatgcctctacttttaaaaacaaagtctaacagatttttctcatgttaaatcacagaaaaagccacctgacattttaacttgtttttgatttgacagtgaaatcttataaatctgccacagttctaaaccaataaagatcaaggtataagggaaaaatgtagaatgtttgtgtgtttattttttccaccttgttctaagcacagcaatgagcattcgtaaaagccttactttatttgtccacccttttcattgttttttagaagcccaacacttttctttaacacatacaatgtggccttttcatgaaatcaattccctgcacagtgatatatggcagagcattgaattctgccaaatatctggctgagtgtttggtgttgtatggtctccatgagattttgtctctataatacttgggttaatctccttggatatacttgtgtgaatcaaactatgttaagggaaataggacaactaaaatatttgcacatgcaacttattggtcccactttttattcttttgcagagaatgggatagagagctggcttcaaagaaaaatcctaaactcattaatgcccttcggcgatgttttttctggagatttatgttctatggaatctttttatatttaggggtaaggatctcatttgtacattcattatgtatcacataactatattcatttttgtgattatgaaaagactacgaaatctggtgaataggtgtaaaaatataaaggatgaatccaactccaaacactaagaaaccacctaaaactctagtaaggataagtaaaaatcctttggaactaaaatgtcctggaacacgggtggcaatttacaatctcaatgggctcagcaaaataaattgcttgcttaaaaaattattttctgttatgattccaaatcacattatcttactagtacatgagattactggtgcctttattttgctgtattcaacaggagagtgtcaggagacaatgtcagcagaattaggtcaaatgcagctaattacatatatgaatgtttgtaatattttgaaatcatatctgcatggtgaattgtttcaaagaaaaacactaaaaatttaaagtatagcagctttaaatactaaataaataatactaaaaatttaaagttctcttgcaatatattttcttaatatcttacatctcatcagtgtgaaaagttgcacacctgaaaatccaggctttgtggtgtttaagtgccttgtatgttccccagttgctgtccaatgtgactctgatttattattttctacatcatgaaagcattatttgaatccttggttgtaacctataaaaggagacagattcaagacttgtttaatcttcttgttaaagctgtgcacaatatttgctttggggcgtttacttatcatatggattgacttgtgtttatattggtctttatgcctcagggagttaaacagtgtctcccagagaaatgccatttgtgttacattgcttgaaaaatttcagttcatacacccccatgaaaaatacatttaaaacttatcttaacaaagatgagtacacttaggcccagaatgttctctaatgctcttgataatttcctagaagaaatttttctgacttttgaaataatagatccataatatatattcttatggaaatctgaaaccatttgggcatttgggggtaaaaagtattttattagtaaatttaaatgaggtagctggataattaaattacttttaagttacctttgagatgatttttctcaatcagagcaccacccagagctttgagaaacaattttattcacagcttctgattctatttgatgtaatttttagaaaataagttttgctggttgctttgaatcagggtatggagtacagttcactctgatcctatcatataaatcatgtaagtatataacattttcaataagtgattgttggattgaagtgaatgatatttcaagtaattgttatgtcatggccaagatttcagtgaaactcaaaatttctcctggttgtgttctccattgcatgctgcttctattgattaacctaagcactactgagtagaagctggaagaggggtctaattagaaggcccctttctatgctctgcttggcttgtaaaataatttatttctctagatcccaccaacatagtagtttcatgtatgcaaaaacacccacctaaatgtcaaagtttgtatgatacatggacatatctatagaattttttttggtctggtgcatgccaaaaaataaacatgatatagaagaatttaatatttattgagtacctaatctgttccagttcaatatgaaggtctttatgcagattattttacttaattttcctagtaactccatggagcaaaaattatctctaatttatataacaggaagttgagcgtgaggcaaattaagtaactttcccaaagttacacatatggtaagtttgagagatatcccagtctctttagctccaaagcctttgaccctttcaccataccagattatgattgctattaatatataattataattataatgattgtatttaggtactcaacagaatggtgactctagtaaccagccttggttctgctgagcttctctgcgtcttctcaggagacacaggctacagagcttgaaggctgaggattcttccagggtcacttcaggggcaaatctgaaactttcttcaggacaggaatcaacgagatcttctcacttacttatacctgggggaggaactgtatgaaatccacccaagaaccagtcatgctaagggccaaacctatagacaaaaaaagggataggagaatggagtatgtatggagaaagactaaattgttcttaaacttctcaagcttaaaaatatcccagcaaaagagatcgtaaaagcccttcatggcgtattaattatccatgcatgggggtgagtggaaaggtactcctgagcccgaggctacagctttggaactagcagcacctttgaaggggaaagcgtgtttccatcatctcaactcctactgataaccaatggaatattggtgagtaaaggatcctgggggaagaagcagctgaaatgtgtaggtgagaaggcagagagaagaatatttatattgggaatggcacaagtgtgatgaggctgcaggtttttcacccttgtcatagagaaaaaaccacgctgacaccatgcagttttaaatagtgagaaatttgcaaattgttagatcttaaataatttagataaacatagtggccatttagattattgcagttttttcaggatatctgatctcttgatttcattctttttgtctcttataagaataaaagggggggagaaaatttagccattatagtatttctctacattttctctgtccttttacataacttacaccagtgccttcctatttatggtattatttatgggtatttcttcttttctttcactgagcaaggataaatgagccagggattcttgaaactactgtaacacttctcttagaaatagatggtcatactttcagaatctctacacattcttagtccctctaaacaatgatagttgtggcataaaaatatttgcttggtttcaggactgatagagaaaagtactataaaatttgctgttaactgtgaaaggttaaaaaaaaggaggtgccatcatgaaggagctaatctttctgaagtactgctgtagttttaaatattattagctatgacttctcaccattaactatgcacttgctttttcttcatctgactcagcagccagatagatgcaacattgtctttaacatttaagactcctagcaagtccgggcacgggggctcacacctgtaatcccagcactttgggaggccgaggtgggcaaatcacaaggtcaggagtttgagaccagcctggccaatatggtgaaaccctgtctctactaaaagtacaaaaatcagccaggtgtggtggcgtggtggcgggcacctgtggtcccagctacttgggaggctgaggcaggagaatagcttgaacctgggaggcagaggttgcagtgagctgagatcgcaccactgcactccagcctgggtgacagagcgagactccatctcaaaaaaaaaaaaaaaaaaaaagactcctagcatggaagagaaactggctgttgaaaacctgaatgtgagagtcagtcaaggatagtttgagggaagccaagtagaggaagctctcacaagcagattggtgagagaatatgattatacaatgcatttattatgataagaaattcacaagcattcattcaaaatactcttgattcctaggcagctctgggcatatttccaccaacaaattgaggcatatgtcagtgcagcctaggtcagactaccttttttcattaaacctcacaaaattaaaggacatacaggagaagtcctggtactcatgttgcagactacagtctatatggcaaaggaggatctctgtcccttatgtttggatgaaaacattgggtaggcatttgaatacaagcctactgctaatatggggctaaggtctttggccccctaaaggtttgctgaaatattactgacaggaggcagattgataagaggaaaagcacataaatgtatttgacatgtatacatgggagccttcaggatgaagacctaccctctcagtgcagtatggaagcttgtataccatcttgaggttacagaaagaatgggggtttggatctttgtaaaacaggtttcagtggcaagacaggttatgagaaggagaaaggaagagacttgggtagcaaagggggtcttgttttgtaggtaaatcgttggcagcccacagagaaaatagatggagaatgtttcttttcagaccttggcaggtgtcagattctcagttaatctctcctagatttgaaaaaaaaaaaaaaggtctagaaagggagagcctggctgcactaacacattttctacagatgcaaatttctcccacaaaatacagctttgcaggtccacttctatctgctgggcctgtggcaaccatttcaaaatatgtgaatgaaatatatgtgggggtaaactatttttatttacttccctaaagaagggatggtgttctctcgggaattctgtgcatagagagcctgtggcttaggcactttgatttatgtatatctcttcctgtgattggctatctagggactgctatctccagcaaatcttctaaatgtctgccatgtagaattcctttctcatctttctgtctcacccccttatctagctgcttctctaaccctagagtgacactgcactccccacaatctcctatgtcctgaatattttaccccatcctaaactccatctctaacacagatgcactttcttgtgctgcctactgcattgtacatcttccccttagttcccatgatgcaactctgccctaccccagaaaatgtaatttaattggtctgggataaaacctgggacactatcattcttgaaatattccccaagcgattctaattatatagccaaagttgagaactatttgtagacaggcatcagcatgatcacttaatgatttgacttttgctagatctaaggtgaggaaattggagagtggtatccataggaagaactgtttagtttaattttttttttattttttcttctaaaaaaaaatccaacaacgagatacatgtgcggaacatgcaggtttgttacataggtataatgtgccatggtagtttgttgcacctattgacccatcctctaagttccctcccctactccttacttcccaacaggccctggtgtatgttgttcccctctctgggtccacctgttctcaatgttcaactcccttttacgagtgagaacacatggtgtttgattttctgttcctgtgttaatttgctgaggatgatagtttccagcttcatccacgtccctgcaaaggacatgatctcattcctttttatggctgcatagtattccatgatgtatatgtaccacattttctttatccagtctgtcattgatgggcatttgggttggttccatgtctttgctattgtaaatagttctgcagtaaacatatatgtccatgtgtctttatagtagaatgatttatattactttgggtatatacccagtaatgagattgctgggtcaaatggcatttctggttctagatacttgaggaatcgccacactgtcttccacaatggttgaactaatttacactcccactaacagtgtaaaagcgttcctatttctccacagcctcaccagcatctattgtttcctaacattttaataactgctattctgactggcatgagatggtatctcattgtggttttgatttgcatttatctgatgatcagtgatgctgagatttttaaaatatgtttgttggccatgtaaatgtcttttgtgaagtgtctgttcatatcctttgcccaccttaatagggttttttttttcttgtgaatttgtttaagtgccttgtaaattctggaaattagatctttgtcagatggatagattgcaaaaattttctcccattttgtaggttgcctgttcactctgatgataggttcttttgctgtgcagaagctctttagtttaattagatccaatttgtcaattttggctttttttgcaattgcttttggcattttcctcgtgaagtctttgcccgtgcctatgtcctgaatggtattgcgtaggttttcttctagggtttttatagttttgggttttacatttaagtctttaatacatcttgagttaatttttgtataaggtataaggaaggggtccagtttcagttttatgcataatggctaggcagttttcccaccaccatttactgaataggagatcttttcctcattgcttgtttttgtcagatttgtcgaagatcagatggttgtagatgtgtggtgttatttctgaggtctctgttctgcaccattggtctatatgtctgttatcgtaccagtcccatgctgttttggttaccgtagccttgtagtatattttgaagtctggtagcgtgatgcctccagctttgttctttttgcttaggattgtcttggctatatggagtcttctttgattccatatgaaatttaaaataatttttttttattctgtgaagaatgtcaatggtagtttgatgggaatagcattgaaattataaattactttgggcagtatagccatgttcacaatattgattctttctatccgtaaggacgacactttttccatttgtttgtgttctctcttatttccttgagcagtggtttgtagttctccttaaagaggtctttcacatcctttgttagctgtgttcctaggtattttgttctctttgtagtgattgtgaatgggaattcattcttgatttgcctctctgctgcctgttgttggtgtaaacaaaattcatttcttgttcttatttgtgaaattttggaaccaaatctattttcaaattagaaattgcttgtgataatggttttgcaacttagactggatatgagacgatgagatattagttctttcattcctttgtaggaatatggtgcatcttgcattattttagctaactagtgtcctttaatgactaatgaatatgacatggtgaaacaaagtaaaatatatatgatgcactaagtatgcattgtttccaaaggttcagcattttttttttgttaactctgctgggatctgctttatgcactgataacataacttattttatgatcttaagcaaataaaaacacttatctggacctcagtttccttaactgtacaactgagggaaactgtatagtatagctatagtacagtataccatctttaccgtcacttccatcttttaaattatgtgtatataagatagggcctagataaatggtatttatcttaaattacagtgatactagcttataacttaatttgctaggtcatgttgaactgataacaatgtgtgaactgatgagcaactgagaagtaaccaggttgtgttataacagtttgtttttgatttagggttatcagtgagggtggcggtggggaggggactttggagtctaactgtctagttcaaatattagtttttgtttatttttatttttaatttttgtgggtacatagtagatgtatatatttatggggtacatgtgatgttttcatataggcatgcaatgtgaaataagcacatcatagagaatggggtatccatcccctcaaacacttatcttttgagttaccaacaatccaatgacactctttaagttatcaaatcacagttttgccagctactagccatgtgattttgggtaggttacttaaattctcttcatctcaatttcattattgtaaagtggagataatgatagcacattttttctttttcttttttcttttattttttattattatactttaagttgtgtgatacatgtgcagaatgtgcaggtttgttacataggtatcaacaactctataaaacatgttctatccaggaaaagaaactatcatcagagtgaacaggcaacttacggaatgggagaaaatgtttgcaatctagatggcgattgcaatggcggttcgctgcatccatcagcccatcatctacattaggtatttctcctaatgctatccctccccttgctccccaccccctcacaggcccctgtgtgtgatgttcccctccctgtgtccatgtgttctcattgttcaactcccacttatgagtgagaacatgtggtgtttggttttctgttcttgtgttagtttgctgagaatgatggtttccagcttcatccatgttcctgcaaggacatgaactcatccttttttatggctgtatagtattccatggtatatatgtgccacattttctttatccagtctatcattggtggacatttgggttggttccaagtctttgctattgtgaacgctgcagcaatgaacatacataagcatatgtctttctagtcaaataagttataatcctttgggtatgtacccagtaatgggattgctgggtcaaatggtatttctggttctagattcttgaggaatcgccacactgtcttccacaatggttgaattaatttacactcccaccaacagtgtagaagcattcctatttctccacatccgctccagcatctgttgtttcctgactttttaatgatcaccattctaactggtgtgagatggtatctcattgtggttttgatttgcatttctctaatgactagtgatgatgagcttcttttcatgtttgttggctgcataaatgtcttcttttgagaagtgtctgttcatatcctttccccactttttgatggggttgtttttttcctgtaaatttgtttaagttccttgtagattttggatattagccctttgtcaggtggatagattgcaaacattttctcccattctgtaagttgcctgttcactctgatgatagtttcttttgctggatagaacatgttttatagagttgttgtgagaattaaatgcattaagcacatagaatagattctggtacatagcaagtgctctctctatatatggaactctatatgtagttggtgcaaaagtaattgtggttttcaccattgaaagtaatggcaaagaccatcattaccttttcaccaatttaaatatatggaaggaatatatatataaaacctatatatatatgtcacatatatgtctctaacccattattataatatataatacaatatatattataattataattgtatataacatatgttatataataatatagtaatatttattctaaataaatatataatactataaataatataataatttatatatatgattataatatataataggctatattatatattattaacatatacatatgtgtatatatatgtctttcatagacttaaatatatagagcaataataggttagaaaatagcaaacatgtatatataaacatatatacatatagaaaacatatataaaaacatatatatatatatatatatgtgtgttttctgcctttcatttttagagacagggtctcatcatgttgcccaggctggtctcaaactcctgggctcaagtgatcctactgctttggactcccgaagtgctgggatttcagacatgagacactgcacccagtccagtccctgtctttttaaatagactctctacctaagtgcacaaatactcattatttacatttagttatttctgtatatatgctataagcaaatcttgtagcaccagtttgatttttataaggcacaagaatatattttactaatgctttaaaatggcagctagattctagtattactttagaaattaaaattaatattttaacacatctttcattattgtgttatctgaaccaaacctattattgctgctatttcagcaaatccaggggctttttcttataaaatatgaagaatatagcttagatttctagtgaagatgttaccagtaataattaataaaatcagtaagcactaaaaggaaaataccaaaactaaagcattttgaattagtcattgaatctaaaagaaaggtagatttttttctgagattctgttctaggtgtggtatatgtgtatttttgcaaaaactataaacaattgtggcaaaatgaaggaaatatttaaaaacaaacctcttaattcttcagtggattaagcgtgaatatgtttttattttctatgatgaatatggaaaaattcatttccttagcaatttgtatgagcccaaaaactattgtcagactctgctgtatcaaaatagacaaaaaattgacactcacttttaccctgccaaaagcaaaatcttaaacttttgctttagtatataagccagcattcattgtatcctatgatgggttctgagtgtaggtgtatttgctttcttccattttttgtatgcatgttttctttttatttattattgtaagttgtatgaaatttttatccaaatttttattttcttctgattaataatcagaataatcagataattactggtaaatttgatgttaatccttccagctttttcccatgggaatttatacttaataaaggggagaagtcatcattacataatgtgcatattaatctgcttctccctttaatgtgttgtgaatgcctttccatgtcattagatgtttttctacctagttactttcatgaatcatatggctgtaccatgatttatttaatcagttcctcatcattgagtatgtaaattgcctccatttttttattactataaaaggtccttcagtacacacccctttaaaagctgactcttagaaggtgttcttgactctctacctaagtgtaaaaatacaaataaattgctttccagaaaaggtgcactactattttactttcctgatactaaactatgaaaattcagtcctaacaatagatatttaaataaagttttaaaaatgccaagtgaaaaagagcatattattattttcatttgcattacttttggttcctggtgagtttaatctgtttttgtatattaattatgcatttatatttctttttgtgtgtgtgaattgcctttcatgttctttgtgtgtttttattttgttgtatttgtctctttcttgatatatgagagaatattttccctagcctgtcaattgccttgtaattttgtttctagtgagtttttttttttttttttacaattaaaagctttaatttttgaaaattttgctggcaaatctatatatctttttctttgttttctgctttgacattattcttttataaaggcccatgccacccaaatattatgtaagcatgcatctatgtttttattacttcatcttttacatttaaatatctactctatttagaattcattgtgatgcatgtatgaggtagaaatctaatttcaaaaagatgagtatccagtttgtccatcatttattgcatgatctctttctccactgaattaaaatgccgtattttataatatattaaagtattacatgtgcttggacatgttcctggacttttgagataaatcagtctatttctttgtcatgtcacatattattatggctttatgatttaatatccagtaatgtaaaccctctgacacattattcttattcctcaaatgtttttgatgagttttcttccaaatgaaatttataatcattttattcattgattcaacaaatatttgttgaatggatattctgtgcttggtattgtgcatggtattaggattgttgcaaaaattgagactgacagtccctactcttacggtgctaaaaattcacttccaaaaaaatctttaaatgttgatgaagattgcactaatcttataaaataacttggaggggaatgtaatctttgcaacattaagttcttcattttagaaagttttaagactctccatttatttgagacttttaaaatatgtcccaataatgttttgtgagatgtatattttaagatatatatcttattgctattacattgtatcttttgttatattgttactatgaatgggatactcatttaattagatgtcatttttggtatatagaaatctattttcttagcatagtcattttttaaacctcgatctattaaattcttgattcatttacatttgttacacaatcatattctatgctgataatacttcttgcttctttccaatatttgtacctcgatcatttttcttgttgagttgtattagctagaagttctagaaaaatgttaaatggtagtaatagctagtattctgttttttcctgactctaaatgtaatgcatctagacttttataattatggcattgattgtaacattttgaggaagaaatcctttttcaggttaataatgtatctttatattcaagtttattaagaacatttattggaaacatattgaaattttatcagattccttttcagttgttactgagataatcataggttcttctgtattcttttaattaatttctcaaaattaaactgtcctattattcttggaataacgacatataaagtactgtatatttaaaagaagttaaaatgataatggtgattttattaagtgacctcacacaatagaaaacagtgtagccttagaagttttccaagtgaccattctacttagaaacaaccctgctttgggatcagaactgtaatttttaaagtaaagttttctgggtttaattcatttagtgtaattacaagcatgagttcaggtttctatttttttcacctgaactttccttcatggtttgaatatctagaaaaagcagactttcctatctctagactaaacatttgatcctatcttaggtatgcattacaattttttaaccataaatggttaaagaatttagactcatctacaataactttgaagctctggtcttgaagaacatgtgagaaatgagatataactcctagaagatataggagacatttttagtcttccaaattttccctgggaggctgatctaaattgagtcacaaaattgttcccaccaggaatgcaatcacttgagctgttttctaatctgagcccctctacccagatgatcttctgaactcatactgttcagactttcatccttctgagtagaaaacagccatagtcatggcaggatgagggctaggacaattacccaaggaattcttggcctctgccatgggactctgcagactcagatcatataatcagagatgttagcactggaggggacatcacaattagctttctccacctcttagtttatcagtgaggaaaactgtccagagcgcggaagagactaaaataacacagccaatgtaggtaatgtgctggataagaatttggaattcacgattttgaattcagtgtttatttcaccatcacgctggcttacacgttggtatcaggcttcttctattattgaagtgagccattaagtgaattccatcttgatttgtgtctgatacagagtaataaactattttattaaatatccaaataattatacattcctccttcttacatgcaagcctaagtttgcttgtactatttcatgtggtagcaaatcaggacgcttcttgtgtctctgaaaatactctgagtaatggagtacagtcagctttcttgtaccaagaatatagggactatgtttctcccagtcattctggggataatttttgtgaaggattgcacttcataggttaagctaggtatcagttaccagtgttttttccaaataaaaaaaaaatcaggtgatatctgtaaatggttccattgtaaatattaaagaacatgatgcttaaaacagattagggaaaactatagaaggggtggggtttcggagtgctaattttgtccttgaatggtaacagctccatgtggtggtgaggtttatgttggtttgctgtttgcagatgatcttattattagaatttttcataccgaaaataaactgcattttagtttgtaaacatgcccttccagagtaatgctaccagttctttgtgaaatagctactgttgttcaaaggatgactatgtcctcttcggttgaggaaagatgacaacaaactcagtaatgacatgtaaaataggtattacaaaccaggtatggtggcatgagcctgtaatcccagctacttgagaggctaaagcaggaggatctgttgatctatggatttgaggctgtagtgtgttgtgatggcacctatgaatagcccttgcactccagcccaagcaacaaagcaagactgtctctgaatttttgttttgttttgttttttgtttttttttttttgagacagagtcttgctctgtcacccaggctgaagtgcagtggcgcgatctccactcactgcaagctccgcctcctgggttcacgccattctcctgcctcagcctcccgagtagctaggactacaggcgcccgcctccacgcccagctaaattttttgtatttttagtagagacgaggtttcactgtgttagccaggacggtcttgatctcctgaccttgtgatcctcctgcctcggcctcccaaagtgctgggattacaggcgtgagccaccgcgcccggcccctgtctctgaattttttaaaaaggcattccactcaaattaatacacattttaattgtgttttgttgtaaattacaactgaataaaaattcagcaaataagtctgttgtggtagggaaaagtctattgtgatctggaaaatataatggagaaatccagtggaagagattttatttcacattactcaaaataaaaaaatcttatacaagtctttacacttgtaacttgaaaaattctgtgctaaaatttagcttggttgctaaaatatttctctttttttctcagaagcttctttttagcatcctatagacacaagttactttttaaaatatttgcatacttgctttgcaatgtattgtttatcagtagttctatattctttgagatagtctatccagtctttctgtatttatcgtatgtctgtatagatatatattagcagataaatgagttctgaaaggggagaaatgtgattatgctaatcatgatataaagaattgactttataagcagtgttcacaggtcatacctttcccgttactgtcttacagtgaacaagaaatgatgctttgtctggtatgcatggtaaataatgccccttgctctctgcttcatgatcacatgtgatacttctaacatagatagcacatgtaaatccagtggccttgactgcaactcaagagagcattttggccaagtacaaacccactagtcatgaaaaaaaaaaaaaaaccaaatcaaagtaaattgatggtattgacatttgtctatgaaaaacaacataatatagaacaattctggggtaaaatattgatctaaaataattttaaggattaaatattgccattgtaagcatactatgagcaattatgtttgtaatgcagatatatttataattttaaatccaagatttaccttaattgtacattttcctaatttaaaaaagttattttgaaaaaaaaatcctcgaatctagagaaaggttggcaaatacatatggaactttgtaaaaaacatccagggcagcactttcactgattgcagtagcttaggagtgaaaaacaacacaactgctccaatgtatggcaatgggcaaatatcccgatttattcacagggtggcatgttaggcagtgcttagaataaatgagttggttatacaagtatcaatagggataaatgtgaaaaacacagtgttaagtttttaaaaagttgtaaaaagcacagtaggatgttatttatataaaatttaaaaacctcaaaaaccattcttctttgatatatattctaaagatgaacatatatgtaatagaagtacaaaacatacataaaataatatacactatgcagtcatttgtgtacttacttttcaaaaatatttcagtagatatagcaaacagttaacatgtaatatttggataggaggttggcaattttctttttagcacctgcctgtctgctatcattcaaactcacatttaaaatgtggctatgtgagatgagagaactataatattccaggtttgtgattagtttggaaactttttaaaagtttgaatgtggtctgagagatagtttgttataatttctgttcttttacatttgctgaggagagctttacttccaactatgtggtcaattttggaataggtgtggtgtggtgctgaaaaaaatgtatattctgttgatttggggtggagagttctgtagatgtctattaggtctgcttggtgcagagctgagttcaattcctgggtatccttgttgactttctgtctcgttgatctgtctaatgttgacagtggggtgttaaagtctcccattattaatgtgtgggagtctaagtctctttgtaggtcactcaggacttgctttatgaatctgggtgctcctgtattgggtgcataaatatttaggatagttagctcctcttgttgaattgatccctttaccattatgtaatggccttctttgtctcttttgatctttgttggtttaaagtctgttttatcagagactaggattgcaacccctgcctttttttgttttccattggcttggtagatcttcctccatccttttattttgagcctatgtgtgtctctgcacgtgagatgggtttcctgaatacagcacactgatgggtcttgactctttatccaatttgccagtctgtgtcttttaattggagcatttagtccatttatatttaaagttaatattgttatgtgtgaatttgatcctgtcattatgatgttagctggtgattttgctcattagttgatgcagtttcttcctagtctcgatggtctttacattttggcatgattttgcagtggctggtactggttgttcctttccaggtttagcgcttccttcaggagctcttttagggcaggcctggtggtgacaaaatctctcagcatttgcttgtctataaagtattttatttctccttcacttatgaagcttagtttggctggatatctctcagaccacagtgcaatcaaactagaactcaggattaagaatctcactcaaagccgctcaactacatggaaactgaacaacctgctcctgaatgactactgggtacataacgaaatgaagacagaaataaagatgttctttgaaaccaacgagaacaaagacaccacataccagaatctctgggatgcattcaaagcagtgtgtagagggaaatttatagcactaaatgcctacaagagaaagcaggaaagatccaaaattgacaccctaacatcacaattaaaagaactagaaaagcaagagcaaacacattcaaaagctagcagaaggcaagaaataactaaaatcagagcagaactgaaggaaatagagacacaaaaaacccttcaaaaaatcaatgaatccaggagctggttttttgaaaggatcaacaaaattgatagaccgctagcaagactaataaagaaaaaaagagagaagaatcaaatagacacaataaaaaatgataaaggggatatcaccaccaatcccacagaaatacaaactaccatcagagaatactacaaacacctctacgcaaataaactagaaaatctagaagaaatggatacattcctcgacacatacactctcccaagactaaaccaggaagaagttgaatctctgaatagaccaataacaggctctgaaattgtggcaataatcaatagtttaccaaccaaaaagagtccaggaccagatggattcacagccgaattctaccagaggtacaaggaggaactggtaccattccttctgaaactattccaatcaatagaaaaagagggaatcctccctaactcattttatgaggccagcatcattctgataccaaagccgggcagagacacaaccaaaaaagagaattttagaccaatatccttgatgaacattgatgcaaaaatcctcaataaaatactggcaaaccgaatccagcagcacatcaaaaagcttatccaccatgatcaagtgggcttcatccctgggatgcaaggctggttcaatatacgcaaatcaataaatgtaatccagcatataaacagagccaaagacaaaaaccacatgattatctcaatagatgcagaaaaagcctttgacaaaattcaacaacccttcatgctaaaaactctcaataaattaggtattgatgggacgtatttcaaaataataagagctatctatgacaaacccacagccaatatcatactgaatgggcaaaaactggaagcattccctttgaaaactggcacaagacagggatgccctctctcaccgctcctattcaacatagtgttggaagttctggccagggcaatcaggcaggagaaggaaataaagggtattcaattaggaaaagaggaagtcaaattgtccctgtttgcagacgacatgattgtttatctagaaaaccccatcgtctcagcccaaaatctccttaagctgataagcaacttcagcaaagtctcaggatacaaaatcaatgtacaaaaatcacaagcattcttatacaccaacaacagacaaacagagagccaaatcatgagtgaactcccattcacaattgcttcaaagagaataaaatacctaggaatccaacttacaagggatgtgaaggacctcttcaaggagaactacaaaccactgctcaaggaaataaaagaggacacaaacaaatggaagaacattccatgctcatgggtaggaagaatcaatatcgtgaaaatggccatactgcccaaggtaatttacagattcaatgccatccccatcaagctaccaatgactttcttcatagaattggaaaaaactactttaaagttcatatggaaccaaaaaagagcccgcatcgccaagtcaatcgtaagccaaaagaacaaagctggaggcatcacgctacctgacttcaaactatactacaaggctacagtaaccaaaacagcatggtactggtaccaaaacagagatatagatcaatggaacagaacagagccctcagaaataacgccgcatatctacaactatctgatctttgacaaacctgagaaaaacaagcaatggggaaaggattccctatttaataaatggtgctgggaaaactggctagccatatgtagaaagctgaaactggatcccttccttacaccttatacaaaaatcaattcaagatggattaaagatttaaacgttagacctaaaaccataaaaaccctagaagaaaacctaggtattaccattcaggacataggcgtgggcaaggacttcatgtccaaaacaccaaaagcaatggcaacaaaagccaaaattgacaaatgggatctaattaaactaaagagcttctgcaaagcaaaagaaactaccatcagagtgaacaggcaacctacaacatgggagaaaattttcgcaacctactcatctgacaaagggctaatatccagaatctacaatgaactcaaacaaatttacaagaaaaaaacaaacaaccccatcaaaaagtgggcgaaggacatgaacagacactactcaaaagaagacatttatgcagccaaaaaacacatgaagaaatgctcatcatcactggccatcagagaaatgcaaatcaaaaccactatgagatatcatctcacaccagttagaatggcaatcattaaaaagtcaggaaacaacaggtgctggagaggatgtggagaaataggaacacttttacactgttggtgggactgtaaactagttcaaccattgtggaagtcagtgtggcgattcctcagggatctagaactagaaataccatttgacccagccatcccattactgggtatatacccaaaggactataaatcatgctgctataaagacacatgcacacgtatgtttattgcggcactattcacaataggaaagacttggaaccaacccaaatgtccaacaatgatagactggattaagaaaatgtggcacatatacaccatggaatactatacagccataaaaaatgatgagttcatgtcctttgtagagacatggatgaaattggaaaccatcattctcagtaaactatcgcaagaacaaaaaaccaaacaccgcatattctcactcataggtgggaattgaacaatgagatcacatggacacaggaaggggaatatcacactctggggactgtggtggggtcgggggaggggggagggatagcattgggagatatacctaatgctagatgacacgttagtgggtgcagcgcaccagcatggcacatgtatacatatgtaactaacctgcacaatgtgcacatgtaccctaaaacttagagtataaaaaaaaaaaaaaaaaaagtttgaatgttttcttgcattcagagccttggttgacatagttaattaaaaataaaacattgtatataaagcacagaatgagcagctacacaaagctgctcaatcaatgacagctctatatgggttagggtttcttgtggggatgacattgatgtagaaagcatggtcatctattgagaatgatggggctggaggtattggatacttgaggtttagaaaatacattgtagaaaatggacaaaaacccctcaaattaagggatgaggcagaataatgcttggcaataccaggggtaggctgcagtctttcttggaaatatatattttaaatggaaccaattatcatagcatcatttcctctcagggttaccctctgatccctattttactaaatcgttataaaacaaaatgaggaattatgtgtccttcccttttgaagccaatgtaacaagatgggtaagaattagacctcctgagttcaaaatccctggattcagatctattcctgtatattcaggagaagtggtaataaattcgatggacaatttggtttagtagtcgattgaggaccctgatgaggtatatttgggaaaacataacttccgctctctctcattgactcacgggcctttgaggagtccaggagtcattggaatctggcctgaggttgaggctgctggcaaaactccttccccaaagtccattcctattgctgactgagaagggactagcattggaagtggctgattttaaataccgctagtgctggtgtgctcctccctcccattcccagctctgctttgtgtagttgccttgagaagctaagttcattctgaaaataatgccattgcacaaaacacttttgaaagttctagtttgaaattacatcaggtcacttggtctgtgtggcctcagtttcttcatctgccatgtgaaaataataatgcctactctgtagcaaagaaagtctctatagtaaacaaaaaaaaagcctactctgatactgaaagttgttatgaaaaataaaaaagggaaatgctttagaaactgttaagtgctatgtagatgttactaattaacaaaccatttcagaaactatactttttattttatggccactattcactgtttaacttaaaatacctcatatgtaaacttgtctcccactgttgctataacaaatcccaagtcttatttcaaagtaccaagatattgaaaatagtgctaagagtttcacatatggtatgaccctctatataaactcattttaagtctcctctaaagatgaaaagtcttgtgttgaaattctcagggtattttatgagaaataaatgaaatttaatttctctgtttttccccttttgtaggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaaggtaatacttccttgcacaggccccatggcacatatattctgtatcgtacatgttttaatgtcataaattaggtagtgagctggtacaagtaagggataaatgctgaaattaatttaatatgcctattaaataaatggcaggaataattaatgctcttaattatccttgataatttaattgacttaaactgataattattgagtatcttctgtaaactgcctctgttgtagttttttttttctcctaatcatgttatcatttttttggaatccatggtttcctgttaagatgactcacacagcctacataaaagtaattgacaaaatatcatcttatagtaaaatgccacatatctttatgttcagcaagaagagtataatatatgattgttaatgataacccaaacaacaaaagatttcaccttaactggttgtcataagtagtagtatccaccgccttattttgagttggatttttatcatcctatgagccctacaaatttaaagtttttggaacagcacgtgcattgaacccataagaacctactctgcttttctgcatgtattgtccagacaagagaccaaattgccgaggcatcatttaggtgaattctaattaacatttagctaccttacaaccacaattcaaggttgtttcaaaggcatgtgcttgcatcatcctgattcactaccatgtgttactaacttggatctgcaaagtcattataaaaagctgttttgatggacttatttggatattgctttacccttcttctctcttttcttttatcaatgtaaaaacattatatgttaaatacttggcttttaagagcatagatctgaaatctgcctctagcaaataacccataacacttctaagatatacctgcaaggtcaattgtgttgtaaaaccttgataaccatactttattgttcaaaaaagccttttatgaaggcagaagttaaaaaaaaaaaacaaaaaaaacagagtccacagttatcacctcagctacaatctcatcagttcacaagtaccagcaaaacatgtgataagtcaacaaatgttttatttcaatctgaacattttacgtaagtgaagactttgttagatatcatttggaatgtggaatctacacagttggcatatcagagaaggttgaattcagtttaataaatgtttatagaaagtgcttgttatcataatgataatagctcaggatgtgcatgacaagcttttaagcgattgggtacactatctcatttgatcttctgcacaactattaatggtaggtactattatccctatcttatggataagtaaactaagatttaaaaagtacagaacatggtgtgaacactgcttcaaaatttctaaaataggtaaatcacgatctctaaactggagggttgtccaaccactagggacaatagagtactgatatttagtggtcagactgtaatgcgggaagagacaggcatgggctaaacgggtgtagagatcaaataaggggcaggttagtttgtaaacatgtccatatgtaacatttagcacaaatacaggatataggtgctttcagacccagctgcattgataaaaagttaggtggtattgtatctgtcttcctttctcaatgttgcatatctgtgttcttgcccagtttgcttcatctctctagccacacttattggcctacaatggcatcatcaccaaagaaggcaatcccatctccgtgtggctttggtttgctccctaaagtaaaccttgtgtttacttttcccaggtctcatgctttcccatatctgacctgttttgtcctcatggccaggatatgtgggacctttcctacaatgttccaaagtttgtaatagagctcttctctgctttgttccaaattctgcaacattttactttaaataatgaatttaaatacaaacaaacttgagctttgcctatacttttcaagaatgcagagataactaaattaataaaaatattcattgagtccttactgtgcacacagctctatgttaagccttgtgcagaactcaaagtcactcgagattaagcctgttactaagttatgtgcaatttagctcagtggatttcccccacttcatattgctctgataatgttttggaattaactgccttgattccttcttttctctgcttgtctatacactatttattattctacaccatctcaaattctaactcctcaagaaaatccttccagatgatttttctaaccaggagttttaacttccttttaactaccctattactttctacttccttaactcatctatcatattatatttagttatttatatactaggtcgccttgaagaagggattgtgttttcataaatcttaataatccctgaggcatcaagtacagtgatttgcatttactaaatgctcaacaaatatgtgagggattcacttgaaactaatattagataattcccagtcaaagtgatctaatagcaaatcaattcttcagttttataggcaaagtatgactctggttttccataatcataattaatttgtcaactttataattttaattaagtaaatttaattggtagataaataagtagataaaaaataatttacctgcttaactacgtttcatatagcattgcatttttctttgtaaaatttaagaattttgtattaataaacttttttacaaaagtattaattattcagttattcatcatatacttttattgacttaaaagtaattttattcaaaagagttagtataggactacatgaaaaattcaaggccaaggcttaatttcaaatttcactgcctttggctctatcttttaaaacaaaacaaaaaactcccgcacaatatcaatgggtatttaagtataatatcattctcattgtgaggagaaaaaataattatttctgcctagatgctgggaaataaaacaactagaagcatgccagtataatattgactgttgaaagaaacatttatgaacctgagaagatagtaagctagatgaatagaatataattttcattacctttacttaataatgaatgcataataactgaattagtcatattataattttacttataatatatttgtattttgtttgttgaaattatctaactttccatttttcttttagactttaaagctgtcaagccgtgttctagataaaataagtattggacaacttgttagtctcctttccaacaacctgaacaaatttgatgaagtatgtacctattgatttaatcttttaggcactattgttataaattatacaactggaaaggcggagttttcctgggtcagataatagtaattagtggttaagtcttgctcagctctagcttccctattctggaaactaagaaaggtcaattgtatagcagagcaccattctggggtctggtagaaccacccaactcaaaggcaccttagcctgttgttaataagatttttcaaaacttaattcttatcagaccttgcttctttttaaaactttaaatctgttatgtactttggccagatatgatacctgagcaattcttgttctgggttgtcttatgtgaaaaataaattcaaggtccttgggacagataatgtgttttatttatctttgcatatccattacttaaaacagcattggacccacagctggtacaaaattaattactgttgaattgagcaaatatttattctaaatgtctctgtcaaatgacagagtgtggttgtgtggattaagtccctggagagagttctttgttctctcatgttctatgctgtggttcttgctttatgcaaaaagaagtaagttacttaaaacctggacatgatacttaagatgtccaatcttgattccactgaataaaaatatgcttaaaaatgcactgacttgaaatttgttttttgggaaaaccgattctatgtgtagaatgtttaagcacattgctatgtgctccatgtaatgattacctagattttagtgtgctcagaaccacgaagtgtttgatcatataagctccttttacttgctttctttcatatatgattgttagtttctaggggtggaagatacaatgacacctgtttttgctgtgcttttattttccagggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacaggtagcaacctattttcataacttgaaagttttaaaaattatgttttcaaaaagcccactttagtaaaaccaggactgctctatgcatagaacagtgatcttcagtgtcattaaattttttttttttttttttttttgagacagagtctagatctgtcacccaggctggagtgcagtggcacgatcttggctcactgcactgcaacttctgcctcccaggctcaagcaattctcctgcctcagcctccggagtagctgggattagaggcgcatgccaccacacccagctaatttttgtattttagtagagacagggtttcaccaggttgcccaggctggtctcgaatgcctgacctcaggtgatccgcccacctcggcctcccaaagtactgatattacaggcatgagctaccgcgcccggcctaaaaaatactttttaagatggtgtaaatattactttctgtatcaatggtacattttttacttgtcagtctctagaatttctttataaatatgttgattcagttcatttttgtagattataaaacaggtaaaaaaggataaaacatttatgtgaattaaagggaatacctaatttttgtgtagagtttattagcttttactactctggtttatggatcatcacaccagagccttagttactttgtgttacagaataactaatatgagtgaatgaatgacttacacaagtcactgcttaggataaagggcttgagtttgtcagctagagtatgacagaaagtatctaagttttggagtcaaatagcactttgtttgaatcccagattgcatgcttactagttatgtgaccttagtcaagccacttcacctcactgagtctttgcttttttcatctctaaaatagagatacccaccgctcataggctgtcataagggatagagatagcatatggaatgagtctgtacagcgtctggcacataggaggcatttaccaaacagtagttattatttttgttaccatctatttgataataaaataatgcccatctgttgaataaaagaaatatgacttaaaaccttgagcagttcttaatagataatttgacttgtttttactattagattgattgattgattgattgattgatttacagagatcagagagctgggaagatcagtgaaagacttgtgattacctcagaaatgattgaaaatatccaatctgttaaggcatactgctgggaagaagcaatggaaaaaatgattgaaaacttaagacagtaagttgttccaataatttcaatattgttagtaattctgtccttaattttttaaaaatatgtttatcatggtagacttccacctcatatttgatgtttgtgacaatcaaatgattgcatttaagttctgtcaatattcatgcattagttgcacaaattcactttcatgggctgtagttttatgtagttggtccagggtgttattttatgctgcaagtatattatactgatacgttattaaagaatttcctacatatgttcactgctgctcaatacatttatttcgttaaaacaattatcaagatactgaaggctgattggtaactcacatggaactgggagagtatacaattctgaaccaaatagatgattctctattattatatcttaatttatgtgttatggtatattaaacatgaaaaaaattgtatttggttagaatatgtttgctcttccttaactcgggaatgacatagggtaatattcacagattgggttcctataaatcctccacttgaagtgaagtcagttcaagtaatgaaagctacctcctgagatagaatcagtacttggcacctatctctagtgttctttcacctcatataacctttcactgattagtaaagattatatccaacaaagaaagtacagcacagactgagatatgattactgagataaatttgggcaaaatataaactacagcatttctgtagcaatgagaccatttttcttcagttgagctccatgttctacaaacttcaatcaaaaaaggttctaggagactcagtgaaagttgatacactgttcaaggaacaaataatttcagcacatgggaatttcacagggaaaaatatactaaaaagagaggtaccattttggatggtgtcaatatgggttatgaggaattcaggctgctgagtccagtgtacaatggaaactgagctgcaggtgtgtgattgtaacaacaaaagaaatgctgaaatattaagtcctttgccatgtaaatagaaaaagagtatttatttcccaaacattattgctcacctgtttttgttatgcctttcaagataaatccaggaaaggaattgcattttctttccagaaaacaagttcttgggggaattgttcaattggtagatgttgtttttctcattaacaagtgagtgctccatcacacttgctgagtgctccatcacacttgctctctgcattactcctctgcctgcaaacacatatatagcaagggtgatgacaaggatatcagagggtctggttttctcaaactcatgataaactcatggctgggtcattcttggtgctgattttactttgttttttgttgttattgttccctcttcctcaaaagatgaaatctatccctcttacttggaatttctctttgatatatagcgaatgtttggttgtaacctgtataatctggcatgaaattgtcactcgaaaaggctagaagtgttgacataaatatgggacagcaagagttgctcctactcaagagagcaaatataatgttctggaagagattggcagaattcacatcaaaggagtgattacttcagcctgggccactgttgtactggtcaaaaggctgtgcaaagctctctgaaaatccactcttttattgctctttagtaataaagtcactttcaattttaaaaataacaaactgatatatttttatgactcataaaatgttagcaattatattatggagaatctactttctgggtgattcttacaaatgttcttggatctatttttttttcttatagtacctattcttcccatttttctcagctctagttaatatatttcaacaacagttcaacaaatttaacatttttataaaaagtgtttcctatcattttataaataccagcctagtccatgttattccttttcttgttgaggagaaaggacacacattgtaaattcaaatatagacctctactgtgctatttaatcttggtaacaactccacaaaggagatgacatgttttccttctatagaggtagattctgtaaagttagagggaagagtgacttgcttaagatggcataagctgtaactggcagaaccaggattcaaagccaggtgggatgccaaaatcataatctgtcttcagtgtcaagttactgaaattggtaaacattagacctaaatagacggaattgcaatccgggttgggcacattaaactccattttcttcatcaatgtgctcagattacattttacttttcaggctaaaaatggaaaaaaagagtccctcttagttctgcacttgagaatgagaatagcttttctgaattatacaaggaagaagaactaatgcccaaatgccaggtacccacatgcactatgccatggcacagctgttgccccctttcaccagagccctctctctgtatcctggttgacctttccttgggcaagagctgggtggggaggatcacaagtgactccaatttggatggcttcgggaagactgggaccgagctgaaggcagtgttgtcctctgcactccctgttttctgtctgctggagcactgaagcctcacatatgtattaaaaaaataatttccatttgcatttcagactagaagattgaacgtatagtgtaatgtgattgcaaataattatattgaaatgagacagagaggatgtagtatctactgtcataatttttcaaaacccacctgcaacttgaattaaaagaaccacttgggtttttttttttgtttcaaacgcaaatcctggaaacctactgagactcattcagtcagtatctctaagaggcaagcttgagactgtatatttaaaaagcatctcaggtgatttttacacatgctaaggcttaagaaccacttctctgtagcttatatgttattttcaatgttcctcaaagccaagttagaatttccaaagtgttaagaatccattagacaatcacagaattgtctttttcctttataaatcttgcaatgttgttctcatttccatacttaattacttaaaacaccaaccaaccaacaagcaaaaaatgattagtctaactaatattacaagttaataatgaagtaaaggtttaaaaataatgtcataataatgttaataacaaattattaattataatttaaaaataatatttataatttaaaaataatatttacaagtactacaagcaaaacactggtactttcattgttatcttttcatataaggtaactgaggcccagagagattaaataacatgcccaaggtcacacaggtcatatgatgtggagccaggttaaaaatataggcagaaagactctagagaccatgctcagatcttccattccaagatccctgatatttgaaaaataaaataacatcctgaattttattgttattgttttttatagaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggtaatgtaccataatgctgcattatatactatgatttaaataatcagtcaatagatcagttctaatgaactttgcaaaaatgtgcgaaaagatagaaaaagaaatttccttcactaggaagttataaaagttgccagctaatactaggaatgttcaccttaaacttttcctagcatttctctggacagtatgatggatgagagtggcattttatgccaaattaccttaaaatcccaataatactgatgtagctagcagctttgagaaattctaaagttttcaagtgataagactcaatttatacaaagctaattggataaacttgtatatgattaagaagcaaataaatacttattatgcttttttgctgtttatttaaatatttaacccagaaaataagtcactgtgacagaaataaaaatgagagagaagggtgagccactcttaggtagttctggcattatttaatctaggccagaggttgcaaatggtgtcccatagaactaattttggctcctagacctgtcttatttaacctttcatttaaaaaatttgtattggttgccagcaattaaaaattgggagatgtctcacacacacacacacataaacacacacactcatgtgtgcagcctcttttgaagaattggaataactagtcaactgcgtcctccttttccacaagctgtgacagctccctgctcacagagcacctgccctctcctgttcatcatgctctcttctcagtcccattccttcattatatcacctatttggtcctgagactaagtgagtttgagatctgtgatttagacaaagtggtgaatctagctctgaatcatagtaagtagctctgggaatcatcttgtcttctgttagcccattgagagagaaatagagagagagagagagagaaagaaagaagaagaaacagatctggggagagtcactgaatgggagcatagagacagagaaacagatctagaaaaccaaactgggagaaaatgagagaaaccaaaagagaggtagagaggagcagagaagaaaatgaagaagcaaggcaaggaccaggctttttcattatttcttatggccaagacttcagtatgcgtggacttaattcttccttatgctcctaccttccctagggaaactgatttggagtctctaatagagcccttcttttagaatcacagtttgatgccttaaaactagttatataccttcacatgcttccttaacccacagaagtgatgctaatgaggcccttaataaggagcgtgctattaagatgaagacattcattttttttctccgtccaatgttggattaaggcacattagtgggtaattcagggttgctttgtaaattcatcactaaggttagcatgtaatagtacaaggaagaatcagttgtatgttaaatctaatgtataaaaagttttataaaatatcatatgtttagagagtatatttcaaatatgatgaatcctagtgcttggcaaattaactttagaacactaataaaattattttattaagaaataattactatttcattattaaaattcatatataagatgtagcacaatgagagtataaagtagatgtaataatgcattaatgctattctgattctataatatgtttttgctctcttttataaataggatttcttacaaaagcaagaatataagacattggaatataacttaacgactacagaagtagtgatggagaatgtaacagccttctgggaggaggtcagaatttttaaaaaattgtttgctctaaacacctaactgttttcttctttgtgaatatggatttcatcctaatggcgaataaaattagaatgatgatataactggtagaactggaaggaggatcactcacttattttctagattaagaagtagaggaatggccaggtgctcatggttgtaatcccagcactttgggagaccaaggcgggtggatcacctgaggtcaggagttcaagaccagcctggccaacatggtaaaacccggtctctactaaaaatacaaaaaattaactgggcatggtggcagatgctgtagtcccagctgctcgggaggctgaggcaggagaatcacttgaacctgggaggcggaggttgcagtgagctaagatcacgccactgcactccagcctgggcaacaaggcgagactctgtctgaaaaagaaaaaaaaataaaaataaaaataaaaagaagtggaggaatattaaatgcaatataaaagctttttttatttttaagtcatacaatttgtttcacataacagatcaggaaataatacagagatcataagttttggagctgggtttgaatcctggctctgccatttactttctgtgtaatctaagtcaagttactgaactttgtgggccctctggctctccatgtgtaaaatggagaatattaatatttaccttgcaagtttgttgtgaagactgaaggagagaatttaggtaaaacattcatcagagtaccatgcacacagttgttcctcaataaacattagcttctctgattgcaagttccagtctaaagtgctttatatataccagccaataaaaggatgcgagagagatataccagtgtattgttttctaccattttaaacctattttcatccactgttacaaattctatcatactgctccacataaaaaatattatcaatgatttttagtctctgaagtgcaatatttgattattgagcacacctgttgaagttttagtttcttctcacttacatgggttgtgtaaaggtaggaggtataaaaccagtgtcctaggtctaaatctttcttaatgtcatactttggattcattgatataagtaacttgagcaccagcgcttcattttacttcattttttaaagatatagtaagagtaattcccatctgcctagcaaaattgttttgtagaaaagtttgtggatcagatttattttactttgattttaggaatttcaagtgtcttcgtcggcatgaaggaaaaatatgcagtttgacattttctactactttcaggtcattattttcctactctggtgcaaaaaccctcaattcctgtctcactccatctaatcaaataggtagcatgcttgagcccttactatgtgccaggcactaggataagcactttatatgttttgtcccaattaattctcacagcatttctatgacctaaataaaattaatattttcatttcaccaataataaaatggaggcttcaaaaagtttagggacttggctcagctcacacaactggcaaggactgaaaatggattttagtcccaaatgtcataggctagagccctttcactaaactgttgtcttccatctggtggcatcctcttcctccagtctttgtcacctaaactctgggcaccccttgatggcatttacttatgatggtgatgcttgttaaacttcctgtttgcgacttcaacgtccatataaatgagtcttccaatactgtacttagaacttatattttgtagtgacttctttaaaagctttctctcttagtcatatcctgagttttgttagcacctggacttaccttactttggaaatgttgcactctgaaatctctttctcagcttggaatttcctaatcttccaactgtttgagtcttttaattctacatttactgcctttccatttcatcaggatttctagtctctttaattcttccttttgaactcctcctgatttaacctctgcttattcgaagaacaataattttattctctcagctgcactctcaattcccttttccttttggtgatttttctttttcctacagaacacttactttatcagttttggagaaggaagtgctatctgggtaacagtagtgctatctgttgactctagtcaactgtaagttttatacatttattgtttaaaccttatatgggtctataatccttcttgggaaatcctttcatttgtctttaatttcctttaccatttccctaaaggctattccagatttttatcacattcacaaaattcccgtcttttctcaggatctgttcacccccagtagatagccttgtctcccacaatacatggagaaaatagaggccaccgtcatatttgaatgtttccaacttctctcttcacctttggaattatctttttcttcttttgtgtctaagagaaagatgtatacttcttcttacccttgtctgaactactctattttgcttcatcttctcagaacaggggaccagcaattattcttcctccagaagcttcaacatcttttgtcaactgactccttctcatgtttaaatattttcaagttaaacaatttctttcctgactttcgctcacgcaacctcatgcccaaaaccttatcactcttcttccctttgctgtcaaggctgttctcacttcttcactttttgtggacttctccccactacaacatagattctgctatcaccaatctattaaaactgttatactcttgtggaatttatcatttaatttagcttcagtgaaccgttctttccagattattttggcctcagaccatgacttctaagtctgccgtgcttgccacttaagtgatgatgggccagtgggtccccacctaggcctctgtgttagtctgttttcatgttgctgataaagacatacccaagaatgggcaatttacagaagaaaggggtttgagggactcacagttccatgtgactggggaggcctcacaatcatggtggatgatgaaaggcatgtctcacatggaggcagataagagcatagaacttgtgcagggaaacttccctttattaaaccaccaggtcttgtgagacttcttcactatcacgagaataggatgggcaagaccctcccccatgattcaattatctcccactgggtccctcccacaacacatgggaattatgggagctataattcaagatgagatttgggtgaggacatagccaaaccatatcagcctccttctggctttttatgttctccgtgggtgacctctctcaggctcaagtgataaccaatgtgctgatgactctcaaatgcgcatctctggcttcagtttcttccttgaacttcatacatatgtttccaaatttcctgcgtgtacctcaaggttcttgttcatcacttcccaagcttcataaacgcactcattttagtgtattctctgtctcctttgatagcatccctgagaggcaagtccctggtgagttatatacaactcctcccttgctccaaacctgagagtaagtaacattcctattaacatattaggaagctgaggcttagacagtttaagtaactcaagcatggttacacaactagctagggcagagctaaaatgtcaggctaggcttctgtgactccaaagccctttctcacttagcatatcatcacttatttttttttttaatcacatatatgatttttttttctttaagagatagaatcttgctctatcacgtgggctggagtgcagtggcacaatcatagctcactgtaaccttgaacttgggctcaagtgatcctcctgccttagcctactgagtagctagggctacagacacacaccaccatgcctagctaattttattttattttattttattttttgagacagagtctcactctgtcacccaggctggagtgcagtggtgcgatcttggctcactggaacctctgctgcccgggttcaagcgattctcctgcctcagcctcctgagtagctgggattacaggtgcctgccactgtgcccagctaatttttgtatttttagtagagacggggtttcaccatcttggccaggcttgtcttgaactcctgacctcgtgatccactcgcctcggcctcccaaagtgctgggattacaggtgtgagccaccacgcctggccacctacctaatttttaatttttttgtagagacagggtctcactacgttgcccaggctggtcttgaactcctgttctcaaacaatcctcctgcctcggacaccccaagtgcagggattacaggcatgagtcattgcagctgacctgtatatatgatttttagtatatgtaaatatacatatttattaaatgtaaatataaatataaatgtgtggagtgatatccattgaaatgttaaacatagttctcagtggtacaactacaggtgatttctcttttcttatttctggttttctgtgttttccaaatttcttgaaatgtgtcttctgtaatcagaaataaaagttattagtaacaacagtcttccactggtacaagtgcttattggataaaagtcccacttctaagcatgatactcacaacttttaggttaatagcctttgtcaccttgccatatacatctgatccagccactcacaccattcctgagatatattttgttcctttgtgcctaaatcattgtgcatgcagatccatcttcctggaacacctataaccatttcttagtcctgtgaaatcctacttacatccttcatagcctagcatgtatgtcatttatttggtcaagggtgagttggttgttctcttgaatgtactgccatatgacgtggtgtgatttcaattgtagcaccaagctcattgcaatattaattcgtttgtcattctcccatgtaggatgtttgaagtagtttctaacacagagattatactcaataaatatttattagataaataaatgaataagggaataacaaatgcctttgtctcattttaaaatactttcattgttagctacccatataataaaaaactaaaagcagtagttttcaagcatgattgtttatgtatgccttaaaagaattttgaaaacctatgtacccctgacacacttttaagttaacttataaatttttcaacatagttttaagtggtggcaaatgatgtagtttcttgtgtattttaaactgcttaagtatgctatacatggatttcttcaaaaccctgaagctgcagtttcagtgcattcaatttatggaaaagaaattaatttataaaattggttcttattgtcaagtcaatcagctaaatataacttgctttctgtcaggaaaagtctgactttaaaatacagataagtaataactattattaattaattaaattattaaaattaaaataattaaataatttgttaattaaaatgccttattcccctacttatttctgcaatttgactctaagaatagataggacatgtagattgccttaggtttgaaatctgggtgaaataagatactgcctccttcagtatttctgcctttgcttttatgggagcctctttcaagaaaaagtcattctctcatggtccctttgtttgagtcccagaggttttcctactccagaaagtgcaacgtagtgagactagtactatactcccttgcatggtaagtgagaaggctgtctgtataaaatgagggaaggactcatgagagggaagtaggtcaggagaaatgataggttctcaggcaggttaattttaggaaagagtgaatagagtcccttaaaacaaggtgcatctgcttcctcctgatcaatctttaggactgtttactttgatttgaagaccactatgctaaagcttcccacgggggcaatagtgaggcaaggaatttttaaaagggaattacttcttcgtagctacttttgtgaaatgaattcatttgaattatctggcaatctcttcatatttatattcaacaataattacttaaagaaatgctttgagcttctcagaggagggtgctaccagtgtgatggagtagaattcagatttgggtagtgactttaaagctgtgtgactttagtcatttaactgctgagtcacagtctacagctttgaaagaggaggattataaaatctatctcatgttaatgctgaagattaaataatagtgtttatgtaccccgcttataggagaagagggtgtgtgtgtgtgtgtgtgtgtgtgtgtgtgtatgtgtatgtatacatgtatgtattcagtctttactgaaattaaaaaatctttaacttgataatgggcaaatatcttagttttagatcatgtcctctagaaaccgtatgctatataattatgtactataaagtaataatgtatacagtgtaatggatcatgggccatgtgcttttcaaactaattgtacataaaacaagcatctattgaaaatatctgacaaactcatcttttatttttgatgtgtgtgtgtgtgtgtgtgtgtttttttaacagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaaggtagttcttttgttcttcactattaagaacttaatttggtgtccatgtctctttttttttctagtttgtagtgctggaaggtatttttggagaaattcttacatgagcattaggagaatgtatgggtgtagtgtcttgtataatagaaattgttccactgataatttactctagttttttatttcctcatattattttcagtggctttttcttccacatctttatattttgcaccacattcaacactgtatcttgcacatggcgagcattcaataactttattgaataaacaaatcatccattttatccattcttaaccagaacagacattttttcagagctggtccaggaaaatcatgacttacattttgccttagtaaccacataaacaaaaggtctccatttttgttaacattacaattttcagaatagatttagatttgcttatgatatattataaggaaaaattatttagtgggatagttttttgaggaaatacataggaatgttaatttattcagtggtcatcctcttctccatatcccaccctaagaacaacttaacctggcatatttggagatacatctgaaaaaatagtagattagaaagaaaaaacagcaaaaggaccaaaactttattgtcaggagaagactttgtagtgatcttcaagaatataacccattgtgtagataatggtaaaaacttgctctcttttaactattgaggaaataaatttaaagacatgaaagaatcaaattagagatgagaaagagctttctagtattagaatgggctaaagggcaataggtatttgcttcagaagtctataaaatggttccttgttcccatttgattgtcattttagctgtggtactttgtagaaatgtgagaaaaagtttagtggtctcttgaagcttttcaaaatactttctagaattataccgaataatctaagacaaacagaaaaagaaagagaggaaggaagaaagaaggaaatgaggaagaaaggaagtaggaggaaggaaggaaggaaagaaggaaggaagtaagagggaagcagtgctgctgctgtaggtaaaaatgttaatgaaaatagaaattaagaaagactcctgaaaggcaattatttatcaatatctaagatgaggagaaccatattttgaagaattgaatatgagacttgggaaacaaaatgccacaaaaaatttccactcaataaatttggtgtcaggctgggtgcagtggctcacacttgtaatcctagcacttttggaggcagaggcaggtgaattgcttgagtccaggagtttgagaccagcgtgggcaacatggcaaaccccacctctacaaaaaacacaaacaaaagaaaatagctgggtgtggtggtgtgtgcctgtagtcccagctacttgggaggctgaggtgggaggatcacctgagcctgagaagtggaggctgcagtgagccatgattgcaccactgtaccctagcctaggtgataggctcaaaaaaaaaaaaaattggtgtttgcaatgctaataatacaatttggttgtttctctctccagttgttttcctacatacgaaacagcttttaaaacaaaatagctggaattgtgcattttttcttacaaaaacattttctttcttaaaatgttattatttttcttttatatcttgtatattattactagcagtgttcactattaaaaaattatactataggaggggctgatactaaataagttagcaatggtctaaacaaggatgtttatttatgaaaaggtagtaattgtgtttcatagaatttttaaaattaattctgcgtatgtcttcaagatcaattctatgatagatgtgcaaaaatagctttggaattacaaattccaagacttactggcaattaaatttcaggcagttttattaaaattgatgagcagataattactggctgacagtgcagttatagcttatgaaaagcagctatgaaggcagagttagaggaaggcagtggtcccttgggaatatttaaacacttctgagaaacggagtttactaactcaatctaggaggctgccttttagtagtattaggaatggaacactttatagttttttttggacaaaagatctagctaaaatataagattgaataattgaaaatattaacattttaagttaaatcttacccactcaatacaatttggtaatttgtatcagaagcttaaaagataacctaatagttcttctacttctataacttacccaaatatgtttgcagagatcttatgtaaagctcttcattataacactgctttcaggagccaaaaattgggtgggggagccccataaatgttgaataataggggtttgattagataaattttggtgtagttctataatggcgtgttattcagccaataaaaggtttgttaaagaatgactgtgacggatgtatatgatatactcttaagtgaataaagagttacaaaatgttatgtacaagttacaaaatgtatgtacattatgatccatttttcataaaatcatatgtatgtatatatgtgtgtctggaaggataaatttatcaagttgttatctctgaaattttgggtatattttatatttctagattttctgttactttgttactttactgataaagtaataacgttgttgacttttgtcactctcccctattaataatcatctaggctgcaaaaggatcatgtcttctttatttttatattccaaggactgtcaacaagtgcctagcacttgacaggtatattatagaaatttaactgaatatctttaggaaatagatttttgtttgtagttgttctagtctacattaaatgtcttgcgcttatgaaacttccttgaattattttagtgaagcaatattagtatagaattttgcatcactggatgcccttgactgaaagctggcttatggcatctcaccagtgtgtggggagtttcagtccttctgttgtctgcatcacagctgaagcagtgctgttgctgacaattcctgacaccaccttgtctctattattgatcattgcctcactatggtactgagttttagcttattcttgtaataactgggactcatatgtatagaataagctattagctcacgtttttgcttgctttttatacagaatacatgtctgcaaatagttttatcaatattttggaattttgggagatatgaagttaaaaacatcattgaatatatatatatacacacacacatatatatatgacactatacatgatttattttatttaatttttaaaattttattctttttagagattaggtcttactctgtcacccaggctgaacttcagtggtgtgatcatagctcactgtaaccttgaactcctgggctcaattgacctttccgcttcagcctcccaaagtgctgggtttataggcatgagccactgtgtctggtccaatatgcatatatatatttttaacctggattatcagagctatattgtgtttaggtttataaagctgtactatgtgaaaatatcacttctaggtttaattttgtacaaaggaattttatatagaaatgaggtaattcagattttttcccatgtaataagaattgtaaaatttactgaaacaaacatcaaaaagatatctgttacatgaccttcctttcttttgaatatatttcaggtgatattatttattaaaatttaaaaatgaaaattaaaatatataaaaagttgaaaattattcctttctttactgtctctcatctgtccattttccattctcctgcattccctcatccaaccaaggtagccaatccaggtaactttttttagtatcttcccagagatgtttctctctatatatataatcaatatacattttttattattccccacctctctttttatgtaacaatatgcagagttttgcttcttgcttttcccactatcttggacaactttccatattcaaagcacagaggacttgcacatatgttcagactgctgaatatttctgtctctcccctgccattcatatgttgaaatcctaattcccaaggtgatggtattgcagggtggggcctttgggaggtgattagtccatgagggtgaagtctttagtaaatgagattagtgtctttataaaagaaaccttagagagaccctcacaccttagagagaccctcacccctttctgccatgtgagaacacagcaggaagacagctggctatccaggattcaggagtctcttagcagacccaaatctgctggcaccttgatcttggacttcccagcctccagaactgtgagaaataaattcctgttgtttataagccacacagttcatggtattttgttatagcagcctgaacaaggacacacacacacacacacacacatgcacacacatttaaatagatgcatagtattctatcatatggatggatattctatgatataatgaatcactattgattgacatttgggttgtttccaatattttgttaacacaaagaacaacactacaaataactttatatacatatcatttagcacatctgcaattgtatcagtaggcttcctataagtggtcaagcatttgtgtacttgtgattttggtagatgttgtcaaatgtccttccctgaaatttgtaccaattcgtactcatgccatacactctaaatagagtgctgatttccccacagcattactaacagatgatattatctaatttaaaaagtttctcatcttatagggaaaatagtatgtcaatgtattcttaacttgcatttcttttattataagtagtgtaaaatatcatttcaacttatacacaggaggaatttctctctatataaagtgatcctagaatcataatgaaaaatatcaccaactcattaggaaaatgtacaaaggattgaatagatatctcatcaaaaataaaaatataagtggcctttaaacattgaaaggtaacatttgaacaaagacttgcaggaggtgagggattagggaatgcagactctgggaagagtcttccaagtagcaggtgaagcaagtgcaaagctttcagatgggactgactatacctgtctggtttgaagaacagtaaggaggtcactgaggctggcatagagtaagacagggagggtagaatactgtcagagaagtaatcggcggtggaggtagggggtaaaccataaagtgctcgtaaagactaaggcttatttctctgggtgagattagaggccactggagagttttaaacagaagtaacagggccactttggctaatgtttttaggctattctgtagggagacaagggaggaagcaaggagatgagttaggagtctattgtgccagttcaggcaagtgatgatggtggcttgatccaggtagtagtggaagtagtatagtaggaagtgatcagattcaggacatgctttgaaggaagatccaataggattaatggataagttgaacaatggcatatgagaaaagtcacagaggagtcaaagatgattccaagctttctggactgagtaactggaaggataaatgtgccgtttactagaaagataatgggagaaacaggttttggatggagcttggtttgggaatattaagtttgaaatgcctatttgacatccaaatagagatgttagttggatgtacaagtctagtttcaaggaagagggggctggtagtgtgaagatggggctggataagattctaaaggaaagagggttgataagaagagaaaggggtgtaggggttagcctaagggcattctaagtattagaggttaaggaggtgggtgaagaaaacccaataaaataaaagtctgagaagacaaagctagtgaatgaatgtggtatcccggaacccaactgatgtcaagcagaagggtgttatcaactaggtcaaatgctcattcatcaagtaagatgaaactgttataattaaccggtgtcttctgaaatacggagataactcgtgacttaatgaaagcaatagtagagaaggtcaaacttgaccagaatgaaattagaaagaataagaggaaagaaaagaccaaatacagacaaccattgatgccttattcttttgatatactcctggagtccacttgctaatacaattgacccttaaacaatacaggcttgaactgcatgggtccacttatttgtgaattttttttcagttaatacattggaaaatttttggggttttttgacaatttgaaaaaactcacaaactgtctagcctagaaataccgagaaaattaagaaaaagtaagatatgccatgaatgcataaaatatatgtagacactagcctattttatcatttgctactataaaatatacacaatctattataaaaagttaaaatttatcaaaacttaacacacactaacacctaccctacctggcaccattcacagtaaagagaaatgtaaataaacataaaaatgtagtattaaaccataatggcataaaactaattgtagtacatatggtactactgtaataatttggaagccacttcctgttgctattacggtaagctcaagcattgtggatagccatttaaaacaccacgtgatgctaatcatctccgtgtgagcagttctctctccagtaaattgcatattgcagtaaaaagtgatctctagtggttctcgcatatttttcatcatgtttagtgcaatgccataaaccttgaataacatcaagcaatccatacaaagtgccactagtgatgcacggaaaagttgtaacagtacaagaaaaaagttgagttgcttggtatttaccatatattgaggtctgcagctacagttgcctgcaatttcgagataaatgaacccagtataaagactgttgtaacaaaagaaaagaaaatgtgaaaccatcagtgcagctatgccagcaggtgtgaagtcttgcactttttgcaaaatacaaaatatgaaatatgtgttaattgactgtttatgttatctgtaaggtttccactcaacaataggctattagtagttaagtttttgtggagtcaaaaattatacgtggatttttgactatacagtgggttggcacccctaaccttcatgttgataaagggtcaatggtatattatttaatttttttgtatttatattcataaataagattaaatctatatttccaagtaatctctataagattttgttattaatattactattatttttgagacagagtcttactgtcaccaggctggagcacagtggtgcgatctcggctcactgcaacctctgcctcccgggctcaagcaattctcctgcctcaccctcccaagtagctgggactacaggcacgcacaaccacactcagctaatttttgtatttttagtagagacggggtttcaccatgttggccaggatggtattgatctcttgacctcatgatctgcctgcctcggcctcccaaagtgttgggattacaggcatgagccactgtgcacagccattaatattattgttacccaataaaaaaaatttggaaacttgtcttcttttcccctgattctgtttaaatagcactggagttacctgttttgaattttttttccaagcggtcccttatgagttttctctatgttttatttgtttcatttctttttttttttttttttttttttttgagacggagtctcgctctgtcgcccaggctggagtgcagtggcgggatctcggctcactgcaagctccgcctcccgggttcacgccattctcctgcctcagcctcccaagtagctgggactacaggcgcccgccactacgcccggctaattttttgtatttttagtagagacggggtttcaccgttttagccgggatggtctcgatctcctgacctcgtgatccgcccgcctcggcctcccaaagtgctgggattacaggcgtgagccaccgcgcccggcctgtttcatttcttatatcgtatttttgcaactcctttattgatacttttcttcctgattaggtttctactaaaaccaaacaagctttccatgaattagcttttagatttacttattagtttaactgttctgttgtattgtaactcattaatttataattttatctttattaattattctatttttcttcgcttttttgttgtttttctagtttttgagttagatgtttgacgcttttttaaaaagctgtgcattttcctctgggtaatactttagctgtatattatgtattctgatatatagtgtttccattacattgttttctagaaaatctgtagctttgatttatatttgtttcctctttgacctaagatatcctaagggaaaatttaacattttccagaaagaaaacaaattttctttgttttccaagaatgttgttcaaattatttctactgcttggaatttttatcatttttgtgtatccagtaaatagtcaatatttgtacttgctctctgaccacataaaagaatatattcgtgtagtttctattaatagattagagttcaattcagatattaaatgtacatcattattcatgatatttaggtcttctacatcttcacttatcttttttctacttgctttgccattaacagataaagttgaattaaaggcttctactacatacatttctccctgttattccttataggttctgtaatttttgcttcaagaatattgctttttaaatttaatatatagatacttataattacactctagcattataaagagccttttctttttcattgaatgtatttgggcctgcatatgtctaacatgaaaattatagtcctttttttgtttctttgtttgtatttacagttttaagttccattttcaacctttatgcactctttgctttaggtgtgtctcttttagttagcataaagttaggtttgtctttaatttcacctgaagtcttttcctcttaatagatgggttaagccaactgaaaaataaaactgacttatatacttttatttcaagtatgtcctccacaaatattttttgaatagattagcttatatactttggaatttgttaaaaaaagatttttataaaaaataattgtggtgaaatgtacataacataaaatttatcattttgaccatttttaagggcatagctctgtggcataaagtatactcacatagttgtgcaactatcacctccttttgatttttttttactaattttgtaaatttgtttcatctgagctgtcttattatgttttgttttatgtttttctttcctttattatgaagtcactgtattgtctgtaggctatatgtatctgtgagtgtgtgtgtatatgtgtgtattatggtttttaaaaaagtctatatttgttttccagtggctatacttaatactaataactttatgttaaatttttcattctatgtgactctagttcactaatatgagctctgataaaatcagtgctttttcgaggttaggagatcaagaccatcctggctaacacagtgaaactccgtctctactaaaaatacaaaaaattagccagacgtgatggcgggtgcccgtagtcccagctactcgggaggctgaggcaggagaatggcgtgaacccaggaggcagaacttgcagtgagccgagatcgcgccactgcactctagcctgggtgacagagtgagactctgtctctaaataaataaataaataaataaataaataaataaaatcagtgctttttcttcctctgctacctcctttccttctactcagttttagtcagtagtattatcttttttcagatttatctttgtattgttaaatctgcttatgcttctattactttatttattagctttaaatgataccttttgactttcagcttttcttaataaagcaatcagcaaatttcctttacactccacacttataccccatttcctttgtttgtttatttggtttttacttctaacttttcttattgtcaggacatataacatatttaaactttgtttttcaactcgaattctgccattagttttaatttttgttcacagttatataaatctttgttcactgatagtccttttgtactatcatctcttaaatgactttatactccaagaaaggctcatgggaacaatattacctgaatatgtctctattacttaatctgtacctaataatatgaaggtaatctactttgtaggatttctgtgaagattaaataaattaatatagttaaagcacatagaacagcactcgacacagagtgagcacttggcaactgttagctgttactaacctttcccattcttcctccaaacctattccaactatctgaatcatgtgccccttctctgtgaacctctatcataatacttgtcacactgtattgtaattgtctcttttactttcccttgtatcttttgtgcatagcagagtacctgaaacaggaagtattttaaatattttgaatcaaatgagttaatagaatctttacaaataagaatatacacttctgcttaggatgataattggaggcaagtgaatcctgagcgtgatttgataatgacctaataatgatgggttttatttccagacttcacttctaatggtgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggtaagaaactatgtgaaaactttttgattatgcatatgaacccttcacactacccaaattatatatttggctccatattcaatcggttagtctacatatatttatgtttcctctatgggtaagctactgtgaatggatcaattaataaaacacatgacctatgctttaagaagcttgcaaacacatgaaataaatgcaatttattttttaaataatgggttcatttgatcacaataaatgcattttatgaaatggtgagaattttgttcactcattagtgagacaaacgtcctcaatggttatttatatggcatgcatataagtgatatgtggtatctttttaaaagataccacaaaatatgcatctttaaaaatatactccaaaaattattaagattattttaataattttaataatactatagcctaatggaatgagcattgatctgccagcagagaattagaggggtaaaattgtgaagatattgtatccctggctttgaacaaataccatataacttctagtgactgcaattctttgatgcagaggcaaaatgaagatgatgtcattactcatttcacaacaatattggagaatgagctaattatctgaaaattacatgaagtattccaagagaaaccagtatatggatcttgtgctgttcactatgtaaattgtgtgatggtgggttcagtagttattgctgtaaatgttagggcagggaatatgttactatgaagtttattgacagtatactccaaatagtgtttgtgattcaaaagcaatatctttgatagttggcatttgcaattcctttatataatcttttatgaaaaaaattgcagagaaagtaaaatgtagcttaaaatacagtatccaaaaaaatggaaaagggcaaaccgtggattagatagaaatggcaattcttataaaaagggttgcatgcttacatgaatggctttccatgtatatactcagtcattcaacagttttttttttagagccccattcttattttttatacactttgagagcataatgaaaagaaaagctacctgcaaaagttttggacttacctcaaagaggatatacttcattcctcaaaaggccttcttccaggaatagtatttcataacctggaggttggaaaaatctggatttgttacaaaaaaatctgagtgtttctagcggacacagatatttgtctaggaggggactaggttgtagcagtggtagtgccttacaagataaatcatgggctttatttacttacgagtggaaaagttgcggaaggtgccttacagactttttttttgcgttaagtatgtgttttcccataggaattaatttataaatggtggtttgatttcctcaagtcaacctttaaaagtatatttagccaaaatatagcttaaatatattactagtaataaatttagtactgtgggtctctcattctcaaaatgagcatttactaatttctgaacactgtgctaggtcctgggaataccaaattgaataagacatagtctatttttctgaagggtttatagcagagtcccctgtgttaataatgaaggagtgtgtggtatgtgaatcatatatcaatagggttgttaaaaataatgaaaaaaggagaagaggaagaacatcttttttttttctgattgcacgggcagccttaaaattatttttgaagtgtacaattcagtgtttttttagcatattcacagggttgtattatcatcaccatatttttggcctcttgaaaagaaatcctgtgcctattagcatccaattaccgttcctttgtagctaagtctcccccattccagctttaaacaatcacccatctactttctgtctctataaatttgtctcttttggacatttcacataaatgaaataatataatagggttttttgtgcctaaataagcttctaaagaagaataaggtaaggaatcatcattcagcaaatatttattaagacttgctttattttatacagtgtactaggagctggagatgaaaatatgtgtagaacatgaatcatatacttcgggaatttgtggactagtgggaaagattgacatatcaataacaaatcgaattagtgatgtaatagaggcatttttacaggagtaaaatgaggtagcatggactctatctgggtctgaataatgtgaggagtaacctccttacacaaagaggcacaaggctaatgtcctctgatggaatgattcaccatgcaattctaagggtgacaagaatgaaagttagggccttgaagaaatattttgattaagagctgccaataaagtagagtaaagattagattgatgtgaagaagtgggagattaatgagtaaatggtcactggcttgttgagaagattaaatgagatgtacatgtaatgtacctaacacaacgtcttgtacaaagtagccattcagtagagactagcttgtattatctccctttgaggtaaagaaaactgttagaaatagtatttctactactgatagtatttcttctacttatgcctccctttgaggtgaagaatactgttagaaaacatgacataggagaaatacccctgagagacagttcttattagtgactactgtgcagaaaagatggaggttggtgtaattaaggagaaggaaagccatgaagccaaagtattatgaaaaagcatcaatatgaattttcatgttgacaaagtggtataaaagataattataaagatggtcacttataaatacggtagttctgtgtgacacaatttacagaagttggtatatcgtgtggaagaaaacagcataagatcctgaaggtttgaactgtgggcacattggctccatgctcaggaaatggcaatggggttgggaagtgattccactttatgtccctttcagacacataaaaattacttgtgtgagtatcttatgccagacactattcactgtgtagtgagcatggtgggtatgaaatgacaactttattgtctttcctgtcaaagaacttgtaggctggttgggggaaagagaccatttcaatatgaagtgctgagctagaggtacccttagggcactacagaagcctagctgatggcttttagcctggctagacagttcaggatctctaaaagcaggtgccttgaaggctgagtcaaatacaaaaatgtattttggacagaggaaattgtatgaacagaaacacagaacatgaaactacttggttggtgcagggtatcatcagcatagaaccagacagaaccagagtgtaaataagccagaaggccatgtcatggaggccttgtataccagtctcaggaatttggttgtggagagctttcatcaggggaatgatgtaatcagcttggaaatgtagatatatcactgactgtgatagtgaggagcagaattaaggtggacgtgattagaagctttgtgaatagcagaaagaacatagattttgaaagctggcagacgtaggttactgaagaaagttacttaaccttgctatgtctttagttttatcctctgcaatatggggataatactgcctattttgtagagtcttgtggattcttctggcatatataatagaaaataaaacagctattattattattgttgatggtactatttgctatatctgactacaaggagaaagactaataggaaaccatttcaggaatccagatatggtcatgatggacaggaagagacaagagttacatagaggaattctgggaagataagaaatgtcatttttatgtactgtttgcatccatcagacaaggcatcaggaaaaatgatccttcaggaaagagtgattttttttcttcaagaaattagaagaggggagaaattggtttaagattaaggactccatgcataagagaaactgggagggaagacaggtagaaatgctatggggttaggaaggaagaatgcagaggtggattacttagaattgagacatctgatcaagacagagggatcacagcttttgctaacaaagtactagtggaggatgccactaggtgaggtttaataaataattgttgacaataagttccatttaaaaaataaacaatttatgcttcttctttgcctaagtgtcaaataaaacattcagatttttatttcaaagtatccctgagtccctgttcccttttttgtcctgctgacttttggaactgatttaggcttccttagtcatctcataatagaaaaaatcagccaggtatttcctacatttcttgtattttaaaaaaatgtaatggatgtaatgaattttaagcaaatgtaatgaatacaataagtaacttagtatatgctgttttcttctctatgctgaatgtttcatacatgttattttctatacaactacatggtcaattccttgaaaatatcaactccaaaatctttattttggtatactccacgtagcacattgagagagttttaaactcttgttggatgactgtttcaaaagtgttttgaagtaggcatgtcagttgcaaaaagtttgctcagcaaatgttgttctgtctcacagtctcagacattgagcagatgattacatgacagcacgtgattgctgggagtaacagacaaaagtaactgaaagtgctcggttatcttgacagtcaaaatcaaaagtgtcccctattttcagtgacctaagagtttctttttgtgtttttggtattgttgttaaataagtgttctcacctttgaaaaggtcaataagaattcaatacagtataatgtctgtgtgccaaatgaaggtgccccttatttttaagtgtggaggagttttgatcataagaacttgaaatacctacagaatccttgatggttaagcagctggtgccagcacaagaatccctcaatatgttctctatgaagccccgatcaccaaatgcaaacattcatgattcagtatattttcatcttgactgccaaagttgatctgtttcttaatatattacatctagacttggaactggagatgagaacagaatattatcttcctcatttttgtgtttttgttcaactctaatgtctgcaaagcacttgcgtatgtaatgatgctcagtgtcataggagcaggcaggtaagtgtaaatttgtctggataggagaaagcatgcacaacatatttcacatagttttctgatttcagtttgtttttgcaaattattcactcagtgagatagcttaaagacgttatcacagggaaaggcatggagatagttctgtgttgatagaaaacttgtaatgtacagccatgagtgagaagtcaggttcagattcttcaccttcagtcctcctctttcataaacagctccatgtcctattttacatatcctactttaaaacgagattatagaagaatgaatttctaggcaaagtgacacttattttaaaatactattacgtatccctgtgcccattaacttatcctaccatttttcttcccctgtgtccaaaccacctttagaatctcctaaatatttgtagctattgtaaacagcactggagactttgctagtttaaaaggagaaatcaacgcaattaagccctagttaatttacttatcccttatgagattataattgtattttgttattaaaagggggacagagtacactgttctcttgcctttttaatttccagactaccacttctcctgcacttgacaataccgcagtctaccacgtagtcccatggctgacaggaggagaattctaggcaggccagtgtttgagtagtgagtaattggactgtctttacccagcaactcactgttttgtaaatgtacctgagtttggagaagtaattggcttttataaggggtgcggggtggagggttggggtggggagagtgagaaggaggtcagagctttaggatatataattggtctccacaaagttgttgtgatacttttggaaccacgtaatggtcttcattaactaagtgtctgtcatgacagccattacatatgcattataataaaaatttatttacagtgtaagttgaagaaggtaaaatctggatgtagtttctaaactctgcttggcagttttcatatttaagccactagaagaaaaaaattgggagggaagctgagaagaatttactgaaagaaaaaaatacttgggagggaaattggcaagaagtatgaaaaagcttgggagggaagtaagcaaataaatgagttaatgactgttctggaaaataaactctatcatgcagatatcacatgactgattaaatttgaatttgacctcctgctttccaggtctggtaaaaactaacctgtaagaacttgaaacttagcctttgaatggtcaatccaccactgtaggagaatttatgaatgttcagttgagagaactgaaaataaagaagtaccataggaattaacatttgcattcagtagccaagatataatggacatctgaaacaggtatttgaggccaggcgtggtgtctcatgcctgtaataatagcactttgggaggccgaggtgggtggatcacaggaggccaggagttcaagaccagcctactaaaacacacacacacacacacacacacacacacacacacactagccaggcgtggtggtgcacgtttgtagtccaagctacttgggaggctgaggcatgagaatagcttgaacccagaaggcggaggttgctgtgagctgagattgcgccactgcactctagcctgggtgacagagtgagactctgtctcaaaaataaaataaaacatatatttgaaacacattgaattatgtcccttaaacaagaataaacatcactaaatgactgtaccttgaactacctgtaattttctcctgataggtaattaagcttcaaagtactgacacttatttactgtaatatgaagcaataacttaaaaaaaaaaaaaaactattgaaccagaaccaaacaggaatgccatagcattttgtaaactaaactgctatttcatttcatttgagccctggaacttgaaaataaatgctagctaacatctgtgaacagaacatacccatcagtactgtgctaagcacctttcatgaactggtcattaaatcctcactttccatttatttagtgacaacttcacccagagtttgcagtcaaagtgaaaatgtgctgaattccaaaagtgtgagctaggttttagaagttaatcacaattctggaacaaattactagcttaacaaatgagagttcttatgtctctaaaaccaaaatagccctaagtctgtccctcccagtaagatttgggccagtcaatggaacagtaatatacaaatataattacagctgtctaggagcaaactatcctatgaatagataataaaattaagacacttaagccatgttttcatattaaaacacaaagtaaaaaatcattgttttccaaagataaaagccatactgtatcatgacatatatatgcccgatgtttcgaccctcttgaagaattgagattctcgactctacactcttagcgttttctatattgaacagatgtttaatttaaggaggtcaagagaaatcttacacttattttttaatggtaccttagacatagaaggaacctcagaaatctctggctgaatatttccatctgcagatgatcatgtcattaggcttctgactctatagccatagaaaaatattcatgaagacctttcaggaagggaatgttggtatttctaaaaattgagtacaagtattctctagacaaaacagctcttgaaatggcagattgtattcccattattatatttcagaatcaagacattaatacctactttttatttaccaggtttagttatccttgaattagattttataaattaaagaaatagatttcaataaatatttgttgagttcctagtatggaaacatcgtgtttggcaccagggatgttgcctgcaagtataacaggagttcgtatttgtaatgagtttatgatttacagatatttggggggcaaagatatcattcggtaaatacttatgagtgcaaactttgaactagggactgggccaaactctaggaacatatttgatgacagagacacaatccctgtcctcaaggagctttcattctagtagagaagatgaaaaccagtacagtttggtaagttagatgatattggttaatgtagggttcttatgtaagtctagagaagtagcatttaatctgttcttagaaggtcaggaaagatttccctggaggaagtgacatttaagctgagagaggatggataaacaggagtcatctgagtgaacaacagggagaacattccagaaagagaacaaaatgtacgaggcctgatgccaagagagaacattcattgcattggggaactatagtcacttctgtgtggctgggatgtagaatgaaatgagcctggacccaagagagcactttgccctttggggaagctgtaggtattacagtaaggttggagtctggaaagaaaggggtatattgtgagatctgaattgggagaggacagttatatccagacctttatatgctccagtaagaagactgaactttacactgggggccatgggactcactgaatggcattaaatttgagagtggtcatatgaccagatttgcattttacaaagattgtcattgactgcaacatgaagtatggagtattggaggagcggtaaggctggtggcagggagataatttaggaggctttaggtgagggatgataatgacttgccaggtaggaaggagtaaatttcttctcagtggataattagaagattgaatggatggacttggtcactatttggtatagaaggggaaaaaagatgtcaaagatgatgccaatttttaaaaataatttaacatttatttttaaatattttttcagccttattaaggtataatggacaacaattgtaggtatatgtcatttacaacatgatgttttgatttatgtatacattgtgaaatgactgccatagtcaagctcattaacatatccatcactcacataattaacattttgtgtgtatgcagtgagaacatcaggctctactctcttagcaattttcaagtatagattacatttgttaccaactatagtggccacactatacaatagagctccaggacttattcatcctgcctaactaaaactttgtactctttgaccaacatcttcccattcgtctctcctccccatgccaagtttccatcttggtcagttgggtggatagtagtactatctgccgaggcaggttggtagggtgaaaacaatgtgttcccttttggaaatgctgaggtgaccagggaacttccaagggaatctgtctggatctagagcttagaagagatgtttgggctggaaacagacatcaggtattcttcagtatatgggttgtaaatgaagtcacaggagtgggtgatatcaccaatggtgagtgtagtataagaagactggactgaggacagatttccaaggaatttcaatacttaagaggtacgcagagaaaagaggggctgtgaaggacaccaaggaggagactaagagccaggagggaaaactttcaagagagtattgcattatggaagggaagaagagagaacattttaaatgatacgcaatgctcaataatggtatccgctttggagaggccaagtaagattcctaagtacccattggatcaaggtccttaatcttacaaaaacttatgcaaatcaataataaagagatgataacccgataatcaaaaatagacaaggcatataagaagaaaatgaattaaaaatattcaaagcattcaacatatacaaatgcgctcaatctgatatataatgaaagaaaagtaaattaaaacaacaatgggcatgactaaataacagtatgagggagcctgaggagaaggagcatttgaaatttcagtacagaagagaaaaggggtgacttatagaaaaaggagacagaaaccatagaacatgtttggaggataagactcaaacaggtagtggggacccttttctagagtaggatgaaaacaggtaatgtgtgtggatgcaaatatgaggtaggatgtaatgggaagttgagcgaattcatatttagtcattcattcaaaaatacttaattgagttactgctgtgtggcaagcatcattctacaaacagagggcacagtgataagcaagccagtttgtactctcgtgtaacttacattctactttgagaagacagattataaataggttaaaaagtcaataatatgatgtttcagcatcaacaataaaaaattagggtgatatatagagtgccagggaaagtgctttcatggacctcttcattctctcctctcctggtgtcataagctactccttcatccatgctgccatttctcttggtttacggttccagtatagtactcatcacattattactatagagccatccaccttatgaaggtgaaggtgtccatctccttacttaaaaaaaaaaaaaacaaacaaaaaaacaaaaaacccgaaaaacaaaaaaagaggcagaaagacagaaggtcctccactaactttcacgtgccatgtaaccagcgaaatccaattattttacagcattctagctatagaagagtttgggaagcgtagtgcttagtgttctagcctttgtagcacaggaaagggcctggaaggaaaggaattgtgtcttccgcagttgcttttctttatggggaagtgctatagcccaaacaatattttaggaattttcatctattgtcaatatgcaaactggaaggggataatgaaaatgttgtggttagaagtttatgaaatattgttattcacattttaaagtaaaaagagggaatgtttaagagacttgtttaagatcacatgtctcataattggtgggaccagcaatacaatccaaatctaactacttatctttttgctatgccctattagtgttcatattagaaaagaaattctatctcagacactaatgatttgttctttggacaccaatgactttaagttaaaacttcatactagttaatttaattatggtgtagcagtattattaaactatcaagactataaattttctatttgtaaaggagattatgataccaaagattagtgaactaatgatattgagaattctatgacataattttgaaaaatatttgcaggatatttatttttgtgtaaatgatgctttcaagctaccataatcctaagtaagtgtatatttgggaaaaccacctattctaacacacttgaaatttaaataagtcaggaaatttttttccagatcttctcccaaattatcttcatctttttcctctccccttgggaaagaatctcttcatgcctcataatatcaaatttaaactatggaagtccaggtggtggacagtcagcaaaggggaagatgagaagcttgtgttataaagccagctcttgtcagaataaggatctggtaggaacttcagaagtgatgggtaggtaagtatgaaggccaggtcctaagatctaaattacaaagcagaagacttacttaccagggagctggaaaacatgttaggaaatccagagcaggaacagatttcaagatagcacaataatatagcagtgaagtactgagaaaagagtttttttcacgggttggatttattctagcattttaggcagcatttgggcatttctaagtggtcagacttagaggagatagttaaggaattagcagctgctaaatgccaattcttagaccagttgaatcaaaatcatctaaaaagctttcagaaaccagactttttaagggccatttgagagactctcaaatctggaatccagaaatctatagctagatgagtttaaggtagagccagaataagaaaaataaaatagtttgtttgtttcaggtatcttttccaatattatttccgaacctaccccaaacaccttaaatcactgcattctatagccattcttttaaaaatgcttgagttattagttttcaaaaacaaatacaaatctgcacacatacagaaataaacattaaagagacataaagatattaaacagagttacatatacttacaacttcatacatatatattatatataaaactgaatattaagtgtttgatattagtgacaaaatctgtaacatccattatattagtgctttttgtactttttgttgggtgtagtaaaaattgcattcgaatttgagttttctgctatatatttggtcagttcctatcagtgaaggaaaaacctttttttattattttattgtttttttattttttgagacggagtcctgctctgttgtccaggctggagtgcagtggcatgatcttggctcactccaacctctgcctcccgggttcaagcgattctcctgcctcagcctcctgagtagctgggactacaggcacctgccaccaggtccagctaatttttgtatttttagtagaaatggggttttgccatgttggccaagttggtctggaactcctgacctcaggtgatctgcctggcttggcctcccaaagtgctggaattacaggtgtaagtcaccacgcctggcccctttttattttttaagctgattgaagattcttagttctcatgctttctagtggtgattaatctttagccaatatttctatatacagttattagtaatcatgtttgacttaggtcaacaaacaatctttcctaaaaaaacagaaccccaattttaatttctgaattatttagtatctattttctgctgtggaagttgaattatgttgatagatatcatacagggccatgtaacactctcagatacacgttcacatgtatagtagctgtatacaaaaatgttacttcattctctctctctttataatactcttggctctcttacgttctctcacacactctactcttcccttcctctgttctttctacttgttccctctgctcctaccacacttattccccccttgtccattttccttgtgcataaagcacaagtgcttagtaattatcaaatattaataacaatgacactaaccacccaatgatttagtgttaatgacatgctttattgaatggcattacctctaaagttcatgtttcctttacccaaccaagcttcttaccctcctcccttaccacaagcatctatattgtcaaggttgttataaagagtaataagccagccattaaaaaagggtttatggtattttcctatctacaaagtcacaggaagctcaaatgtactcagtaaatattgcaaaattacacaggaccattaaatgtaacactccaccctttctctctctctctctctctcttgctctctctctctctttctgtcaatatagcaacaccctatatcattgccctttgtatgtgcaaatcagagttaataagctttatattagcaattactccttaacaacttctggtttgtttggtccagttgaataatgtaagcacttaaaaaaatgaaattataaacatttatgtgaaaagtgcatatatcacattggatatgttgttatgcactccttaataataaagtaagttaatctttattgcacacttattataatattactttgaccctctctagtactctttatctaagtattctcaagtgctttacaatctcaaacagacccaatgtgttgtatacacagaatcctttgaagctgacatttgcctttctgaccagcttgttgtaaaggaaatcagccaaaaaacaagtatctagatgagtagctcaaacattagtacacatagtaatcacaggtcaaaatgcagatagattaccctgtccaaattctcctgagtaagagtaggtgaaacatttttaaataagctccccaggtgattctgaaattggtccaaggaccacatattaagaactaatgatccaaacaatttgactttttattgtagattaaaccatgctgagaaaattattaaaaattgaaatggcagtggaggatggtttgaaagaaaggtttttcagggccctttcaacaataaaattaattgaacacaatattaaaactctatatttgatttaagactaaggttttcattgtttttaaatctcagtaatttttatgtaacaggtcaattcatacccagcatcttaattccaatgaatgatttcccacaacaatttttgtggataactccaagggaactcgaaggaagttgtagtatgaacaaagagaagtagaatttgtccctgtgtgtaaggcttctctgataagcagcacaggctctcatactgctttttaaaaaaattatgatagcatcaagtggaattaattttttttagattatactttcatggaagggaagatctactgtgaaggctggaaaaccaacacccttaagataaatatattaccagatttgagcgctcttagtaatcagcaaagataaatgtttaacagtgcatacaaaatgaagtgttttatgttaaatcaaatagagaaagccaaacactaataatgtggttacaaatgaacaataaattaggtaatcagaacaggtacagacattaatagcaggatattggtattattaatgtattttgttttaaaataatgaacttaattacaattctcctcatcctaccccactattttattttattccagattcagcagcttcatattatgtctctgaaacacttattattaaagttatccaaatgtacacatttctctttatataaatgtttcagtccagaaaaggaggccaaatacattagctcagaacatcaaatcttctcagatgtgggaatcttttattttcacacttttaaaggtaatctgtatttctagcgtctattatagacagaaaactttcatatgacaacattcctattttcttaactgccttgataggggcgaagacaaattctaagtaggactttttaccccattcttcttaccatcattctttcacaaaacccccagctttagacaatcgctattatgaatttgacatgtactattccaatccattcccataaatttacacccatatatacatatagttatctatgaacaatatttagtagcttttttgtgtgtggctttaaaatttacataaattgtataatttgtgcacattcttctttaatttgccttcttggctacggttatctttttgagatctagctatgctgctggtatgtagaattctatttcattcttttttcattgttgttttgtacccataacgtgtcacattttatttataccttctgttcctgatggacatttagattcttccaggattttactcaatactgcaatgaaaatctttgaatttttctcttttgcacatattcaagagacttttctgacatatatatctataggtgaattgtgtagtcatatgatacatacacacattttaaatttcactagatactgccaatttgccctttgaaatagccatacaatttatagtaccaccagccacttatgaaagttcccatttcctcaaatctttgaaagttcttattataaacagacatattaattcttgccattctgatttgtaaatcagaatctctattgttctacctctagttctaatttggaattccccaattacttgtaagatgctatatattttcatgtttgttagtcattctgatttcatatcctttaccaattatctttttggtaagttattgtggtggccatgagatgtgccttacagaggccttgctagagggaatgtgattgaatgagagccccagatgctgtgtattaaaatcctgcactgagtttgtctcaagatttcttgcacgtgaatgaatgagtacagctgggatactaaagcagatgtgtatttgggagatatgagacttctttagtggctgatttttggctcataaatgactttgccaaaccttccttagactgctcagtgttctaacatcttccatccagccttctacccttctttcctttactaggggattgaatttacattgaggtctcatagccttctctgcctctctccttatttccttttatacaaatatttcccctaataaatccatgcacatttaataccattttgctatttgcaacctgcaggtcctggactaacacagttctatacattgcattaccattctctagagtgggatcttttgttgtagagagttttaaaatttttatgtagtcacttttatccatatttttctttatggtttatatttttgtgtcttctctttaacacatcttttctagcagaattcataaatatattattctatattgccaaaagtttgaaagttgcaatcattagaattaatttttgtatattgtgtaagttaagaatctaattttattgtttttcattggaaagccatttgtcccaagataattttttagtagtccctccttcccctattgtcattctgacatattttttctaggttccgatctatgcatgtgtttctttatggaagagttggccctttgtatctttgagtttcaaatccatggattcaatcaaccacagatagaaaatatttagaaaagcgtcagaattgaacatgtacatacattttgcttgtcattattccctaaacaatatagtataacaactatttatgtaggatttacattgtattaggtattgtaagtaatctagagatgatttaaagtatacaggaagatgtgcatatgttacatgcaaatactaccccatttatataagggtcttgagcattcatggattttggtatccacagagagtcctggaaccaattccccacagatgccaaggcacaactgtatttattctatcatctacttgtttaatctcacatcagtatctacttttgaaataacaataactttattatttaactttttttattacttaggattagagaatttcctctggtgaggcatcatagtgtctcaagctggccataaagacaagtgagggctaggatcggtaagactgggcagaggaagatacaacagatctcctatgcatgaagcaaaagtgcagctcagaagccagctctttcattaagttgtcctctataccctcactagattgtaagctcttgaaatgagaggctataccttaattgtctctgttatctaaaatacttccactcactgcttggaacatattgcctgcaataattaagcttgccctggctcccaaagcatagagcaaatcacactcctccccttgcctttgagaagctcacagtcttcgaaggtagagatatgtgaacagataagaaaatggatgacaggagaacagaaacgcatgactgtcagagaagtcattggagactttacagaggaaattaaatttttattgatcttgaaagagtttgccagatgaagtagaggacaggcattttagacaaagggaacaggaaatgtgaaaacacaaagtgatggaagtcatggtgagtttggagaactataaaacttcaatgtggctgaagggtaaggtggatatagaggagtgctgggaggtgaggctgaagaaataagctaggaaatgtctttttatgccattttttaaagtttggactttattctgaagttcacatggatccaatattttttgttttgtgttgttttaagcagaagcgtgacatgatcagcttgaatgatgaacaacttgaattgtttaaagtggatcacacagtctactgttttacagttattctttgaccaagatattctttattaactgaggaaaaaaagggctttcctgaattttgcagtcatgggatatatgataagcattcttgatttatcatcttcaatcctgttacataacataataaccattgttattacctttagcaatgctttcctcagtattatctaatggcctataaaatgtgactttcatttgcaaatacagtacatctaacaagaacttaccacagctgctatgcaaaataccaatacaattgacccttggacaatgtgggggttaggggtgctgattccccatgcagttgaacatgttacataacataatacataaccattgttattatgtaacaggattgaaaatgataaatctttggaaagtggggcaaatgaattcttatgaattccatatcttccacatgtgttttacttttttgataagaagtagtaacctagttcagaaagaaaataatcatccccttttacttatgcaggataccaagtctatcttagcaccataatagtgaatgataggaatcaagctctatgaatacattcacatgtacatatatatggctatataggacacatgcatgcacatatacatatatacacttgcatatatgtgtatatacatgtacatatatgcatgtatattcaattgtatatgtgtatatagccaagttattgtacagttgacctttgaacaacacgggtttgaactatgcaggtccacttacacgtattttttttttccgtttctgacacccctaaggcaacaaggccaactcctccccttgctcttcctcctcagctgactcaacatgaaaactatgaggacgaagacctttatgaagattcacctccacttaatgaatagtacatacatttctttttccccatggttttcttaataacattttcttttctctagcttgctttattgtaataatatagtatataatacatataacataccaagtatgtgttaattgactgcttatgttatcagtaaggcttctggtcaacagtagactattgctagttaagtttctggtagttacaagttatatgtgggtgttcgactgcatggggagtcagcaccccaaccctcatgttgtccaagggcgttgtccaagggtcagttgtaattggtattttggatagcagctgtggtaaattctggttagatgtactatatttataaatgaaactcacattttataggccattaaatattattgaggagagcatttctaagggtaaaatcttgtctaatgcttgaaacatcttcattttcctgtcagtttagatctttttgaagtaattctgaaaatctctcttttaagctaaatttaacacaaccaaatagccaaatatttaagttccactaatgaagatatctaaatttctgttaaaaatttaagatatatgttaaacccttctaatataactcttctctcagtcaaacttttttttttaacagttgctttgcttcttctttcaaagtcatacttcaacaaagttgctattgaatatgtctgactaaacatgttagctatatgataagatggctggataagagataaatatagaaaatgtagctttttttctacttgcaataaccctttaggaattaaaatggaaaactaataactatttgattcataatagtagcaaaccgtaaaatatttagacataaatctactaagaaatttataagacatatatggagaaaattcaattgaataaaccgttattgaagtatataaaataagatctggatgaatagaaagatcataatttttaataaaattttgcatcttaaaaagtgaaccctctccaaatatatgcacatttaataaaattataaatacatcccaatgaggttggttttgaaattttgttaattggaacttaaatttcacctaagaagaaaaaataaagaatagttaagagtgcatgctttgtagacaaattgccttagttagaatcctggctctatcatctattagctatgttatctttgggataacattcatcttttcttatagatatgcttaaaacagtgcctgacatatagtaagcacaaatatccattagctattcttcttattatttatgttattagtattgttaatatttgttattatatggaagactaaatgaccaaagagagtcaagaaatttatgaataagatttatgcgttgttagatattagagccattaaaaaaaaaaaaaccaaagtgccaaaaaacctagcacagtgttaatacaggaataaaaaaatggatcagaggaaccaaacagaaaagccagaaatggatcttaggaaacatgagaatatgatatatgatagatgctaaatgaattcagtataaaaatattaatgtaataaatcatgcttgctattcaagtaaaagaaaatgaggttagattcatgtctcataccaaatataaccataaattataccttgattaaattttttaattaaaaagcaataatatttgaaaagaaatataggatactcaatgtataacctgaaggttgggtagtacttttcaacaaatataggaatttttcacttgaaatactagaagaaaaaaagatagcaaacaaatacaggaattccaatttcaagcagatataatgatttcatgaaatgttaactgtgcacatgatagatggtctatggatagtgcaaaagaaaaagagaaaagaaaaaatgttttttaacatatgcagcaaaaaaggtttttaacatctattacatacaaataaaaatgaatgtataacacagacttcaataaaaataggcatttcacaggagaacaattcagatggccagtatttacaatttcataggtattaaggaaaatacaaattaaaatggcaaattagcaaaaattgaggtgtgattatattaatatctgttggtggtggtgattatggggaaaagggtactttcaaaacttgctaatataaatataattcttttggttgttttgtaaaggaacctgacaatatcttttaaaaataaagaaaacgcatacttttgacctagccatcccattcatgagggtatgtcttagaaaaataagatcacaaaatcatagagatttatgtgcaatgatattattggtaggtcatttttatgaggaggggtgtggatagtaaatgccagggtaaatcacatagcatctaataaacgtatttatgaactacaaaagcttacactttcagtctagtctagtccagactgcaaataaatgtgagcaagtgaattcaagcacagaagtgcttgaaggcaggtttcataaatctactttcttacagtatcctgatattgacttatcgagacagttactgtggggttgattattaaaatatttatgtatctaggtatttttcattcagtagtatgttattcaattagcaacaagtgtggggatttaaagatattcttgtttgtttttactgctgaaacatattctagtggaaatttcgaataaacgattagtcatcctaaaagcaagatacattttctcagaaaagacaaggtaaagaacttgtatatcctccctcaattcgtttataaggtaataagatgaataaaaatatcatagtacaatttagcattgtaaaataaaattaattggtcatctctagtgtggtcgtgcttggaaggtgaaagaagccaagatcttgtctgggaatatcatgtctaccttgacctcacccttaagaatcctagcctttagtttaaaatcacatggctacatacataccaacttcaacaatagtacatctggcaaggtcatgcaaacctgggacttgagcttctgattctaagtccagtgctttttgtgtacatcatctcttgtacataccttatgatgatatgctaataaaagctacgtgatcaggccttaaaaatctgctttttttttgtaatggtagaatggggcatattatcacatcaggtaaacactctattcaaggataaatggaaatgaatgtcatatatagatcattgataaatatctcattacaaaattatgagagttaccaatgtttgagtgtatattatgggccagccctttatattaaattacttcaaatttttacaactgttaaaggaagatattattatacccattttatagatggacaagttagggccagaaaagacttcctcaaagctgttagtccagtaatggagacagggctagaaaacaggtcattttgctctttgactaatgttactactcatgttttgtattttgtttaaagttttattttattttgctttatttattttttgagacaagatcttactctgtcacccaggctggagtgcaatggagtgatcacggttcattgcagccttgacctcctgggctcaagcgatcctcccacctctcaatctccagagtagctaggactactacaggtgtgtgccaccatacctggctaaattttgcattttttgtggggacagggtttcactatgttgcccaggctggtcttgaactcctgggctccagcgattcacctgccttgacctcccaaagtgccagtatcacaggcttgagccaccatgtccagccaagttttattttagaattaaaaaaaattccacttggattgttacattttatctcattgctttatatttatagaattactttataaatgccactttcttaattttcatagttagcactctttatgaaacataaactattatttgacccaggtttttgttagaggaattgagtcagagagctgttaagtaactgagatttcacaataagccagacagaccagggttcaaattctgggtctcacattatccaattcaatattccagctttgttacttattgagcaaccactacaagcacagtttacatgacatctgatagctctcaaaatgaattttacaaacataattcagatttcaactcagcagtgactcaggagaaaggacacttggatgcatttctttatggcatttttcccagggtacacgcaacctggaagatctcccaagtatgggggaaggtttcaccctgaggaatcccattccctctaatctgggacaagggggaggagagtactgtctcttatcagccatctccccagggaggcctgggccctcctggaatgcataccatggcttactgactcaaagtgttgaaaagaccaggcattgggacacacaacactactcttaaaataaaaaaagaatcagagtagcttgtggttataattgaaatggacagagtaacatggtaccaagaaactattagcaattccttccctaaatccctcattttcttaaagcattttctccttttcctcaacaagctttaagttggatttgaagaatgataagactaaaaggagggctgtttctggtctttggaggaatttgatattccattcgatctgagtgtgcaaagcctgagttcacatgaactcttctgatctctttctctaatattttttcaccttattcatatgggaaagaaggaggggaatactttagttccattctccctcctcctatttccttgacttgtttaaaatataaatgttatagacacctaagatagaaatttgactgaaacagcctcttaattattgtcttaaaaaattggtataatgaaattgcatttgtagtctttggacatttaaatccagaagggatattttctttttcttttttaaaaatttaattcaatagtttttgggctacaggtggtttttggttacatggataagtgctttagtggtgatttctgagattttgatatacccatcacctgagcagtgtgcactgtacccaatatgtagtcttttatcccccccccgctccacccttcctttatcgtccccaaagcacattatataattattatgcctttgcagcctcattggttagctcccacttgtaagtgagaacatgcgatatttggttttccattcctgagttacttcatttagaataaattgtctctagctccattcaagttgctgcaaaggccattatttcattccgttttttggctgaatagtattccatagtgtatatatgccacattttctttatccacttgttgattgataggcatttaggttggacccatattttcgcaattatgaattgtactgctgtaaacatgagtgtgctttttttttttccatataatgacttcttttcctttgggtagatacccagcagtgggactgctggatcgaatggtagttctccttttagttctttaaggaatctccatactgttttccacagtggttgtactagtttacaaccccaccagcagtgtaaaactgttccattttcagcacatccatgccaacatctattattttttgactttttaattgtggctattcttgcaggagtaagatggtatctcattgtggttttaatttgcatttccctgataatcagtgatgttgagcattttttcctgtgtttgttatttgtttgtatatcttgagaattatctattctgtcctttgcccactttttgatggaattatttgtttttttttcttgctgatttgtttgagttccttgtagatcctggatactagtcctttatcggatgcatagtttatgaatattctttcccactctgtaggttgtctgtttaccatgctaattatttattttgctgtgcaaaagcttttcagtttaattatttcccatctatttatttttgtttctgttttatttgcttttgggatcttagtcatgaactttttacctaaaccaatgactataagagtttttccaatgttatcttctagaatgcttatgttttctggtcttagatttaagtctttgattcatcttgagttaatttttgtataaggtgagcattgaggatccagtttcattcttctacgtgtggcttgccagttttcccagcaccatttattagatagggtatcctgtccccactttatgtttttgtatgctttgtcaaagatcagttgactttaagtatttggctttatttctgggttctctattctgttccattgtctacttgcctatttgtgtaccagtaccaggctgttttagtaactatagccttgtagtataatttgaagtcgggtaatatgatgcctccagatttgttctttttgcttagtattcctttagctatgtgggctcttttttagttccctatgaattttaggatttttttctagttctgtgaagaattatgatgatattttgatgggaattgtattgaatttgtagattgcttttggcagtatggtcattttcatagtattgattctacccatccatgagcatgggatgtgtttccatttgtttgtgtcacctgtgatttctttgagcagcattttgtagttttccttgtagagatctttaacctccttggttaagtatattttcatgtattttagttttttttttttgtttgttttgttttgttttgttttgtttttgcagctgttgtaaaagggattgagttcttgatttgattctcagcttggttgttgtcagcagggacattttctaaagtatagactgtagttccttatcttctatctgtttcttactgtccccttcagtattcttgtccttttttcccgctattatctttttgaccttttaatatatagatatctacttctacttctgacaatttttgcttctccaattttctttctttttctcctctgcacacatttatttattttcttctatgtacttctttatttttaacttaatatttgattaacttcccttccctgtctcttttccttctttccataaatcttcattaattgcctgcactgagctaggattctatactctctaaatcaataatctattttctatagtcaactgtgttataatcgtactgtcaagataactacttatttttaatacttaaaaatattttgaaattttaaccaatttaattaatacaatgttgagttcaaatttgaaaaaaacaatggaaaactgtaataattctagcaacctcctgctttttaataatgtattagaaaatttgcctctttttcaaaagcctacagtgaatctattcatacaaggcaaaagcaaaccattctcttcattctctttttttctccaaaagatttaagtgttttttgtttgtttgttttgttttgttttttagatattgagtcttgctctgtcatccaggctgcagtgcagtggtgtgatcatagctcgctatagcctcgaattcctgggttcaagcaatcctcctccctcaccctcctgagtagctggggctacaggtgcatgctaccatgcccagctaatttaaaaggaaaaaaattgtgtagagatgggtcttgctatgttgcccaggctggtctcaaacttccaatctcaagcatttctcccacccagcatcctgaagtgctgagattataagtgagccactatgcccaaccagatttagtttttaaaaagagaatacgatttgaaaaaggaaaaatgtgaggcaggagagaagaaatacacacacgagctgttttgtaattgctgtaaaactgaaatcttcagcctcactaaaggagcacttgcatgaacacctctaaattaccttattaccttctaaattaggtgtgaagtctaacttctaaattatgagtgaaatccactgcaattcttgttatttggatggaatcctaggtatgtggtccagttcatgagttgaacaaaagcatgctcatttaggccaggtagaaagaaataaagacctatgttttacatgtctcataaccactgaaggtccttctcataagcagtgcttatgggtattaacgacctctctatattttacttctccagtgcctaagtagccgagtccactgagtcctgctacatctcctccaacatgtcagcatttttttcacaggccttttgttactctagatcagaaatgttgatagcaacagttccttgagggcagcagctagcatgatgccagccaacaggaaccaccaaatggttcttaatataaattactacttattaatctatttactttgtgcatttggagttttgcatgtaaagtcctatttatgtccatatggtagataaatggaacaaatgaataacagaagtaaccattttgatactttagatatagataatattggattatttctggattgtgaaagaagaaggaagaagcatatggaagagaagttttagtagaggggaggaaggaggaggtggaaacgaatgtacaaggatgggaggagaaaagggagagagacttttttttttttaaggcgagagtttactacctatctaactcttcgcattcttgaagtctcagaccaaatcccatcggtttgaaagcctctagggtattctatctattgtatacttctgttatgtacaaaattaatttgccaattaattgtgaactgttttataaactatcttaaaatggttagttaaatctttgggatagtatttagctttctccaggattatgacttaccttctaaattagacatacaatgcctaggagtcaaggactattttgcataaattccagtcttcttttacaatgcctagaatgattgttaccacagaaatattcattacctgggagaaaggatgacaggaggggcagaatgaatggagagaggtcgtgagaatgaggtgctgaggatggacgaggaagaaagctgttttagttgggaggataggtgacagaagcatggaaaggaattgccttggacccatggaagcccagtgaagatacttagatcctgcaggggtgtgaataatgttcttttagtttctcttcttaggaggtttgttcattttgggagatttcttttgaaaagagtgaacttaaattggagaaaagtacattttagtatgttgataacatttgaatttgtaaaatggacctatggatgatctacacatatttatatacccataaatatacacatattttaatttttggtattttataattattatttaatgatcattcatgacattttaaaaattacagaaaaatttacatctaaaatttcagcaatgttgtttttgaccaactaaataaattgcatttgaaataatggagatgcaatgttcaaaatttcaactgtggttaaagcaatagtgtgatatatgattacattagaaggaagatgtgcctttcaaattcagattgagcatactaaaagtgactctctaattttctatttttggtaataggacatctccaagtttgcagagaaagacaatatagttcttggagaaggtggaatcacactgagtggaggtcaacgagcaagaatttctttagcaaggtgaataactaattattggtctagcaagcatttgctgtaaatgtcattcatgtaaaaaaattacagacatttctctattgctttatattctgtttctggaattgaaaaaatcctggggttttatggctagtgggttaagaatcacatttaagaactataaataatggtatagtatccagatttggtagagattatggttactcagaatctgtgcccgtatcttggtgtcagtgtatttgtttgcctcatagtatagtttactacaaatggaaaactctaggattctgcataatactggacagagaagatgtaaatatctgttagttccatcatagaccctgccactccaatgtacacaccagctttaggcttcttggtatagataaacatacattttcaaaatttttcatcataattttcataacaaaataggaaggcaaatgatgtcacttggcttaaaatctataatatttaaaataaacaggacaaatgcattaacattgttgggggaggaggtcccttagtagaaacactcttggtccaagcattttaaagctgtcaaagagatgtaaatatagataatgtatgtcaaggagagagctttgtggttaaactgtaactttcagtttaaacaattattggtgactctgatgtcaaatgtttctcaagctttatctgaacaaaattcttctcactttgttgccaaagtcgttaacaagaaatcacattgactcattgatgttttggctcctttcccttactttctgttgctttccaaaagctgagacaggaaactaaccctaactgagcacctgcaattgcctggtagtattctagtcatgtgtgtacttttgtgtgtatgtaatccccttacagctctgcaaagtaagaattgttctccctgctttacagaagagatcataagataattgaggctgttagatgttaacttgccaaaagccatacaggaaaatggtagagtcacagtttgaaccaggtccttttgattctttacattaaaccatgctttgatcttggaaatacactgtaaggcaataaatcaatagatacggataattcacaggcttctaaataaatggaagttgattgtttttatctgtgagccaaagtaagacttattctaagaattccacaaatttagataagatagagtatatggcttctagacatccaacatagaactgagtttgtgttatcagtttaagatttggttttgctgtaaggtgcacacactttgaggaactaaaaataattgtctgttcttattctgatcagaatgtgtaatgtgttgtccagttttggatgatgaatttcttatttctaatctcataagaaacttgtcatagatgtgagggagagaattaagaacagagtgtggggaagaaactgtgtacattttgatgggatccattatgtagctcttgcatactgtcttcaaaaataagttacactataaaggttgttttagacttttaaagttttgccattggtttttaaaaaaatttttaaattggctttaaaaatttcttaattgtgtgctgaatacaattttctttattacagaagtaccaacaattacatgtataaacagagaatcctatgtacttgagatataagtaaggttactatcaatcacacctgaaaaatttaaatgttatgaagaaattatctcatttctattaatatgggaactgtgtcttcatctttattactgttctaaggtcaactcaatgtagattttacttgcttatggtttcatattttagctaaatagtaaaataatatggatatacattttgttgtgacttactcatactttccttatttggaacttttatgaatatgatatagagactgaaactacaaggaacaaaatgcaatatcaattatacagttgtggcagcactgctatcaatttgttgatagtggttaacacttagaaaaacattttaaaaataatttcacataagtaatgtaatttattagctgtctctgacattttacagtttggaatagtttattttctttttggtgtcctcaccaaaacccaacatcttcaagggcaggaactgtataatttttgccattgtattttgagcacatagcatggtacttgcctctaaatagatactattgttaaaatattttttaaggtaatattttaaagtgtatgctatggtacagttcagtttgtgacttttgctagtttatgccacttacagttagcaaaatcacttcagcagttcttggaatgttgtgaaaagtgataaaaatcttctgcaacttattcctttattcctcatttaaaataatctaccatagtaaaaacatgtataaaagtgctacttctgcaccacttttgagaatagtgttatttcagtgaatcgatgtggtgaccatattgtaatgcatgtagtgaactgtttaaggcaaatcatctacactagatgaccaggaaatagagaggaaatgtaatttaatttccattttctttttagagcagtatacaaagatgctgatttgtatttattagactctccttttggatacctagatgttttaacagaaaaagaaatatttgaaaggtatgttctttgaataccttacttataatgctcatgctaaaataaaagaaagacagactgtcccatcatagattgcattttacctcttgagaaatatgttcaccattgttggtatggcagaatgtagcatggtattaactcaaatctgatctgccctactgggccaggattcaagattacttccattaaaaccttttctcaccgcctcatgctaaaccagtttctctcattgctatactgttatagcaattgctatctatgtagtttttgcagtatcattgccttgtgatatatattactttaattattattatacttaacatttttatttactttttgtgttagtattttattctgtcttctccttagatagtaaccttcttaagaaaatatatatgctaagtgttttactggtttaatatgcttagactactcatctacctcaatacttccttggagatctcctcctcagtcacacagagctcaggacttatatttccttggaactcctgttagggtccaatgtacatgaaattccctagacagacagacagtcagttatatggcttgatttcaaagtttcaaaatgatttaatggactatcaagtagtttattaggagaacagttattatactcttctaaaaataaagactttaagcaataaagatgtatatgtatataaaatggctgggttattcctagaagtacctttcttagaatttagttaaatttaatatccaagatactatcttttcaaccctgagattgtgaaaagtaacttctatcaatataaactttactacatttgtattgtgttagtgtgttacagtataatctagaacaatgtgtctttctatatgatatatgacattttaatgcctaaaaaaactgatatgtcttagatgattctagtcaggatttacttctagaatagattaaaattctatttgaggagagtcaaattaattatcgaattctcagttgttattattgctgttttatttttagtgaaacagattagtcttaatgtaaacacttgagaaataaattgatggtcaacctaaaatgtaaaaaagaaattaatagaaaatttaaagagcaacaaagctctgacatttaaaagaaatgaagtacaaatctctagggaccttaaagatcatctaataatttcctcattttctagataaataaactgagagaccccgaggataaatgatttgctcaaagtcaaatatctacttaatataggaaatttaatttcattctcagtctgttaacatgcaacttttcaatatagcatgttatttcatgctatcagaattcacaaggtaccaatttaattactacagagtacttatagaatcatttaaaatataataaaattgtatgatagagattatatgcaataaaacattaacaaaatgctaaaatacgagacatattgcaataaagtatttataaaattgatatttatatgtttttatatcttaaagctgtgtctgtaaactgatggctaacaaaactaggattttggtcacttctaaaatggaacatttaaagaaagctgacaaaatattaattttgcatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctatacgaaaattttccattgtgcaaaagactcccttacaaatgaatggcatcgaagaggattctgatgagcctttagagagaaggctgtccttagtaccagattctgagcagggagaggcgatactgcctcgcatcagcgtgatcagcactggccccacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggtaggtatacatcgcttgggggtatttcaccccacagaatgcaattgagtagaatgcaatatgtagcatgtaacaaaatttactaaaatcataggattaggataaggtgtatcttaaaactcagaaagtatgaagttcattaattatacaagcaacgttaaaatgtaaaataacaaatgatttctttttgcaatggacatatctcttcccataaaatgggaaaggatttagtttttggtcctctactaagccagtgataactgtgactataagttagaaagcatttgctttattaccatcttgaaccctctgtgggaagaggtgcagtataaataactgtataaataaatagtagctttcattatttatagctcgcaaaataatctgtatggaagtagcatatataaggtatataaacatttagcctcttgataggactaactcacattctggtttgtatatcagtcttgcctgaatttagctagtgtgggcttttttttatcttgtgagtttgctttatacattgggtttctgaaaagatttcttttagagaatgtatataagcttaacatgtactagtgccaatcttcagacagaaattttgttctattaggttttaagaataaaagcattttatttttaaaacaggaaataatataaaaaggagagtttttgttgttttagtagaaaacttaatgccttggatgaaatgagccatgggcagggttgtaatgaattgatatgtttaatagtatagatcatttgtgaataatatgacctttgacaagacacaagccattaacatctgtaggcagaagtttccttctttgtaaaatgagggaataaaatagatccctaaagtgtgtaattttagtatttctaaactttatgaaggtttcctaaatgataattcatctatatagtgtttttttgtgtgtttgtttgtttgtttgtttgagatggagtctcgctctgtcacctaggctggagtgcaatggtgcaacctcggctcactgcaacctctgcctcctgggttcaagctaatctcctgcctcagcctcctgagtagctgagattacaggcatgcaccaccatgccgagctaatttttgtatttttagtagagaaggggtttcatcatgttgaccaggctggtcttgaactcctgaccttgtgatccacccacctcagcctcccaaagtgctggtattacaggcgtgtgccaccacgtccagcctgagccactgcgcccagcccatctatatagtttaatatcaatctaaatgaatttctcagtcctgagcctaaaaatttagttgtaaagaatgatatccttgactaataatagtttctattaatggattgcatctagtgctaggtggcatatatttagtccccacaactaccctggaaggtatttaaaatttttcacatttgcagataaggaaactaaagttcagagttcggcaacatgcttgaattcaagcagctcctaggatgttaatggtggaggttgggttcaaatccagatctgtctgactcaaaaaatgcatactcctaaccagtgcactatatcccaattccataggagcccttctttgtgattcatagcactttcccatgagttttgttgattttgtgagaaacaaaactctttttcctttggactgtctggaatctctctttttcaaatttttgaaatgtatttctatgccaaaagacaaagatttctagaggaatatgcctaggatgagaattatgtaatttaaatcacagctggaaagagagaaagtcctaagttactaagaaatgttcaaacacaaatgagctttcagtctattggaagacctttatagctagaagtatactgaactgtacttgtccatggacccctgaagaaacaggttaaatcaaagagagttctgggaaacttcatttagatggtatcattcatttgataaaaggtatgccactgttaagcctttaatggtaaaattgtccaataataatacagttatataatcagtgatacatttttagaattttgaaaaattacgatgtttctcatttttaataaagctgtgttgctccagtagacattattctggctatagaatgacatcatacatggcatttataatgatttatatttgttaaaatacacttagattcaagtaatactattcttttattttcatatattaaaaataaaaccacaatggtggcatgaaactgtactgtcttattgtaatagccataattcttttattcaggagtgcttttttgatgatatggagagcataccagcagtgactacatggaacacataccttcgatatattactgtccacaagagcttaatttttgtgctaatttggtgcttagtaatttttctggcagaggtaagaatgttctattgtaaagtattactggatttaaagttaaattaagatagtttggggatgtatacatatatatgcacacacataaatatgtatatatacacatgtatacatgtataagtatgcatatatacacacatatatcactatatgtatatatgtatatattacatatatttgtgattttacagtatataatggtatagattcatatagttcttagcttctgaaaaatcaacaagtagaaccactactgatattttattatttcatattacatataaaatatatttaaatacaaatataagaagagtttttaatagatttttaataataaaggttaagagattcgaaagctcaaagtagaaggcttttatttggattgaaattaaacaattagaatcactgttgatattttattatttcatattacatataaaatatatttaaatataaagataagagtttttaatagattttataataaatgttaagagattaaaaaactgaaaatagaaggcttttatttggattgaaattaaaggccaggcatggtggttcatgcctgtaatcccagaattttaggagactgagtggggaggattgcttgagcccaggggtcaagaccagcctgggcaacacagtgagacaccgtatctacaaaataattaaaaaattagctgggcatggtggtgtgtgcctgtatgctaccattaactaaggaggctgaggtgggagaatcgcttgagcctgggaggtcaaggctgccctgaactgtgattgtgccattgcattccagcctgggtgccagagagagaccctatctctaaataaataaataagtaaataaataaacagcaacaacaaaaacactcaaagcaaatctgtactaaattttgaattcattctgagaggtgacagcatgctggcagtcctggcagccctcgctcactctcagggcctccttgaccttgacgcccactctggctgtgcgtgaggagcccttcagccctcccctgcactgtgggagcccctttctgggctggccaaggccagagccggctccctcagcttgcggggaggtgtggagggagaggcgctgggggaactggggctgcgggtgccttgtgggccagcgcgagttctgggtgggtgtgggctgggcaggccccgcactcggagcagccggccggccccgcgagccccaggcagtgaggggcttagcacctgggccagcagctgctgtactcgatttctcactgggccttagctgcctccctgcggggcagggctcgggacctgcagcctgccatgcctgagcctccccccaacctgccgctgcagtgggctcctgcgtggcccaagcctcctgacgagcaccgccccctgctccacggcacccagtcccatagaccgcccaagggctgaggagtgtgggtgcagggcgcagggctggcaggcagctccacctgcagccccagtgcgggatccactgggtgaagccagctgggcttctgagtctggtggggacttggaggatctttatgtctagctaagggattgtaaatacaccaatcagcactctgtatctagctcaaggtttgtaaacacaccaatcagcaccctgtgtctagctcagggtttgtgaatgcaccaatcagcactctgtatctagttaatctggtggagacttggagaacctttatgtctagctaagggattgtaaatataccaatgtgcactctgtatctagctcaaggtttgtaaatacaccaatcagcactctctgtctagctcagggtttgtaaatacaccaatggacactttgtatctagctaatctagtgaggaggtggagaacttttgtgtctagctcagggattgtaaacgcaccaatcagcaccctgtcaaaacggaccaatcagctctctgtaaaaccaatctgctgtctgtaaaatggaccaatcagcaggatgtgggtggggccagataagagaataaaagcaggctgcctgagccagaagtggcaacctgctggggtctgtagaagctttgttcttttgttctttgcaataaattttgctactgctcactttttgggtccgcattgcgtttatgagctgtgacactcactgggaaggtctgcagcttcactcctgaagccagcgagatcacgaacccaccagaagaaagaaactcctaacacatccgaacatcagaaggaacaaactcaggacacgcggcctttaagaactataacactcactgcaagggtccttggcttcattctcgaagtcagtgagaccaagaacccaccaattccggacacaatttgactgcagaaaatggatgtccaaccctgtggtttccctgggccacattggaagaagaaaggagttgtcttgggccacacataaaatacacttactatagcagatgagctaaagaaaagaaaaaagtccatgcgtaatctttgtgatatgtgccaccaccaataagcaaaattgttctcttattcaaaaggttggacacagctgctctagatattttattattaaatatgcaggcaattactgtttaaatgaagatttcctcacagaatgagattaaaagtatatattagtggcttagcattcattttagacaaccattttagagattcaaatcacacacttgcttacagaaattttgttgtcttcaatgtccccattgtggtttctttaccaagcctctactgttcttcacatcaccaagttaaaaaaaaaaaaggggcgggggggcagaatgaaaattgcatggtaggccacaagttcagatcctcatcgacacaagaggtgcctgaagcagtggatgaggcttttctatggatcatgagcagccacataaatgcttaaaagggcctggcagggagcatcagtgggtgatgtggctgggaggctgaatggagagcatttgttcttcagttatctatagaaggcagctgtcactcagcaccagctaagggcttcccatgagggaactggggatcaggtttcccagatctttttatgtaacaggataagacagagatccagctttttttgggtaattatttcctattttaaaatacgggtagttgattaaataaaaacaaacgaatgaacaccatatgggcacaacaaaacacatctgtggcttggattcagcttgtgaatgattactgcagatatttattctagaggacacccctgggtatgtcctaatataaaacctaaatctaaactcaagtcccatgctaccttcagagaataaatgacccagaaaaagaaccacctctcctaaggaagtataaatttgtaaataactgagacccaaacttacaactatacatttttcttattgttgggctgttgctaacctcaattaagaaggcttgatgatatttgtaaagtgtcatcactccaccatggtccagtaacatctgatcactccaccatggtccagtaacatctgaatggtcaagaaatatctaaacgtatgtaccaaaaatttgtgtatactactgtaccaataaaccatttgtttccatttgatctctgagtgtggtaatacatgttatttgccctgctgttgtaaataaacaaaccaaatggaggcttgatgcaagatgcagtgtagcatagtgccaactctggactccgactactcagggtgtaaattctaactctgttctattaacaccatgaaactgagcaagttagttaaaactcgctgggcccattttctcatttatacaatggagattttaatagtacagctacataggccattttgtggtttaaaatacatcatgattatgaaacacttaatgtagggcttgctacataatgagcaaggtttgttgctgttatcattaatatccttaattctcattattataaaacttgagatagtatgaggtgaacaagttcataacagcaatataatgaaaattttaataattccttttatactttaacaaaaatacgagattgggtaatttattatttttacatgagtaataaatattgcattaaaatatatttaaaatttaccacattaatgtctgccagtcatgccaaatgaccaacatgaatgtgaataaaactcagtctgtgcccatttaatcttaaccaaccctttataattgttaatgatttgaacctctgccttgaaagatcacattacttgattgtcttcaacttatctgaatgtggtagtgatttctgtaaatttataggacctttgtctcatgcagctccatggagttgaacttatgcacctttaaaatggtatatacttaattaattaagtgttgatctgcttcacatgtgtataatattattagctcactaaaccaagaaaacagtggtcctttagggaaagaaactaaattacaacagagaatataaataccatataaatatctattatttattgaactgtcacaattattgcaaaaaattaccttttagtggacaaaacaattgatattgcccttttctggaaaagaaataatgtaatatatgatgaatagttttggccagtatcctctagaccttgccagttaactggctctcaaaattttgaataataaaaacttggtgatagtagaaaaatagtaattttttaaaagtatgtgcacaattatacaactaaacaattcattcaccagtgttcacaattctattgccttctttgaatcaaaatttacatagtttttcttttagactaagctcctttatgataccagtgtgcccatttctcattaccattgaaatgtctcatgagcatgtcacattctggtacaactgctaatccaggatgacagtttagttcttttaaatccaattgagagccttctactcatgaccagagaacctaaagaaaggttaagatacatttattccttggtgtaagtgatttgtctatttttagttttcctaagggtcatatttcaatttagatttttttttataggttaggtaaaataggcttcccttttgcaatatgaaatatgtagtcttttaaaaaatttcttcaaagctattaaactgaaaaaaaattaatttggtctattcagtttgttagcacttaccattttggaaagagagtgactctacttttgtatttggtaacattttccctactacagggcagtatcttttgtaagttcttagatattagcaccaaataaataggcaaaaaaaatctattatgttaattcttagaacccctgcttggcagtgcatcattgactagatggagaagaaatgaaaataatacattaggaagcagtttcctggttcttttgaaaacaactagagagtcttgttgttgactggaatatctgaagatcctgtttaatgctttcattctatgattgttaagaatatgtcatagaactgctgtatcctgtttctttatgtcttcccttctgtttgttgattagaaatccctgagtggctttacattattagtacagtagatatgtagtatattcccataataccactgctgctattgactaatagtaataattttagggcagctttatgacagttggtttatgttttagggtgtcatttgacttgtgaagcattgaaatctgggtattaagcacactgttttctatgtggtatggaatgattcttaaagccctgagaaaatggaaaataaaaatatttttcctttttaccataatcacctatgactgtcactctatcataaactgcataaactttataacctcaaaacattttggaaatgaaatgacagaacttgcttactcaattgcttctatatacaccaaatatttttttaaagtattatgttaagtccttgaaaatattttgttctactcaatagaagcagtttaggttggtagttctatgtggaaaccgtgaggaaataattttatattatgatgactagaccagtctttgaacatcactttggttattgttccattagtaaatattataattatttctgagatttactcaccttcaaagaatgttggcaatgccagcattattaacactcctctagttagaacaaagaggaaatgtaataacaaaacataataatagccaaataaagagtgacttagaatgtacacccttatctaggatcctgagtaattcgattattcttaggaaatacacttttgtgctagaacaaagacttttgaaatagctaatttctgggtttcttttcattttgaattaacttgaatttcaaggaaacaagggtagtttttacagatacagtgcatagaagctctgtgtacaatgaagaaaagtaggaaagtgagaaaaatgccattagatttttcatcgttatactatctgatatgtgaatttaactaaaacttatatacctcattatagtacttcctaatgtaatttcttaatttaagtgttccccataaggtttttttttatataaacttaagtactgttaaatatttaaggcaaattcaggtataaaataagacttgttgatatcttattccaagcatatttgtttctctcctatttatttttattctgtgttcatttccaaaattgttttactcacaactgtttgttttttctgtttcattctgtggtaaaggtatcatttggctaattgtataatttcagtgtcatttctaatattccaattgtgatagtatcaacacaagattaaatttctctacatggtttatgagaatggaatgccaaattgaaatagaacagagcacagatgatctaaatataaaaagaactacaaaaatcacagttgtttaaaaaggttttttgtttgtttatatatggtgcagaacatttgttccttagccaaatgtttccaccttgagaaagctatagagattctatgtagtcctagtaccaataatatgttttaacctgaatgtaccttatctttattcataaactgtgactttttacactgctgaaacttttttttttaagacaatctcactctgtcgtccagtctggagtgcagcagtggtgtgatcttggctcactgcaacctctaccttctgtgttcaagcaattctggtgcctcggccacctgagtagttgggatcacaggtgtacaccaccaggcctggctaatagtttttgatatttctagtagagatgagttttgccacattggccaggctggcctgaaactcctggcctcaagtgatctgcctgccttggcctcccaaagtgttggtattacaagtgtgagccactgtgcctggcctgaaactcataattcatttccattaatattaatctcaccttttccaataattaattgatttcacaagtattagtcccctataatcattgaatggctaataaaattatttatagcaaacagattaattatctgccagcagtctgagattagtttctttaaaaaatgtttattatttaaaacattcagctgtgatcttggctttcttgtgaggttcaatagtttctattgagtaaaggagagaaatggcagagaatttacttcagtgaaatttgaattccattaacttaatgtggtctcatcacaaataatagtacttagaacacctagtacagctgctggacccaggaacacaaagcaaaggaagatgaaattgtgtgtaccttgatattggtacacacatcaaatggtgtgatgtgaatttagatgtgggcatgggaggaataggtgaagatgttagaaaaaaaatcaactgtgtcttgttccattccaggtggctgcttctttggttgtgctgtggctccttggaaagtgagtattccatgtcctattgtgtagattgtgttttatttctgttgattaaatattgtaatccactatgtttgtatgtattgtaatccactttgtttcatttctcccaagcattatggtagtggaaagataaggttttttgtttaaatgatgaccattagttgggtgaggtgacacattcctgtagtcctagctcctccacaggctgacgcaggaggatcacttgagcccaggagttcagggctgtagtgttgtatcattgtgagtagccaccgcactccagcctggacaatatagtgagatcctatatctaaaataaaataaaataaaatgaataaattgtgagcatgtgcagctcctgcagtttctaaagaatatagttctgttcagtttctgtgaaacacaataaaaatatttgaaataacattacatatttagggttttcttcaaattttttaatttaataaagaacaactcaatctctatcaatagtgagaaaacatatctattttcttgcaataatagtatgattttgaggttaagggtgcatgctcttctaatgcaaaatattgtatttatttagactcaagtttagttccatttacatgtattggaaattcagtaagtaactttggctgccaaataacgatttcctatttgctttacagcactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtactttactaggtctaagaaatgaaactgctgatccaccatcaatagggcctgtggttttgttggttttctaatggcagtgctggcttttgcacagaggcatgtgccctttgttgaacctccatttgactggcatgcacatgtctcagatattataggttatcatatattgttgctcctaatatttctgtgttagataattagagtagcttggtttgtaagaatgtgatgttggtgggactgtagcagaacaagaaggcccttatgggtcagtcatacctctcttttcaaatatttggtctagctctcttctgggcatcttgttgccaatatatagtattgctcaaaagggcaggagatttgaagtgatcaaggaaaatatattttttctattgattaagtcttttgatggggtagaataatctaatttcatgtaactgctcaaagttatatggtagggggatcccaaatgtattttaaaactatttttatatcatcatatttgaagtaatagaaagtcagagtagcagaataaaggtactaaaaattttaaaaactaataaggtactttgaaagaaatcaattatgttgattcctcattaaacaaatttgcacttaaagactgaggttaataaggatttccccaagttttttcatagcaacctgtgagcactttctctgttgaggcatttatggtatgaaaagatgagtaaggcacagttcttgccctggagaaggtcacaggtgagaggaggagttgacacagaaacatttgatataaagcaaggaataaattccaagactaaaattttcagaaatctaaaaaactcaagataagaaaaacccattatattttctgggtaacaaaatttcagtgttattaacatgtaggaagatcttgatatttattctgaagcccatgtgtgttgctgaaatattgccgcatttgcatatactcatcaccatcctctgttttggagctaagaattttagactcaagatgtctaattaagttgatccattgattttattttttatggaaatctgagacccacagaaggcaggggatttgcccacatttctagaagagtcagacatgagcgatgaggcacagtggaaagaacatgagcattgcctgagctctgagttggcgctataagagcagtgatcatgggcaagtgactcttctgagccttggcctcctcacctgttaagtgaagaaaagaatatttcagaagatctttgtgagaatgaaacaaggcaatttacttgcctgctacatagccaatgggaaatcaatataagttccccgtggttcccttctgtggggttttgttcccacagagggtgcactggccattccacttcttcttttccaagctcctcattccctttaacgctgttcatagttggttccaaaccatttgaaatataataagcaccaggatggttttttctttccaccaaagcaaatttcattttctaaacactgtttataaatatcaatggctattttttcaatttttgattatcatgaaaatatacaaatatgtttaattaaatatgctaaagaatgtattaataaatatgtattaaataattcctacatataaggcctttttgcttggggtatgggtgatacaaaataaatgtggcatgaacccactgacctctagcaatttataacctagaaaaagagttatgatatgtttataagttcctgtgatataagacatgcatatagtcattataacagaggtgcaaacaagatgtatcaagtatgtccagaggaggaagagattaatcccagctggaggaaacactgatgctttcttgcagcaggggcatttgagttgagaaagggaggaaacatagattttgacaatgagagctgaggggaaaggggtttcaggtggagggaaccgcatgtggaaagcagggaggtaggaaagtgtagagtgtgtttaaagaatagaccagtttggctgaaacaggatatttgagcagaggaagcttgtactaggtaggtgggttgaggccaaattatgcaaggcattaaatattaaactaggaattttggactttatcctgcagtttatggggggtaaatgataagattcaatatcactttatttgtacagtattatgttacattttatctaattgtttgtttaattcctgtctagacaatgaattcctcaagggcaaggagcatggcttattcacctcagtaatttcagtgcctagcattgtgcctggtacaaagtggacacttgtatataaccttttttaattgaagcaacaagttgtcaaccttacaaatgtgaatccgtgattcagatgacaggttgaaatgtagattgtctgcgaagagggcagaaagagagtatgacaaaggaggacaagacagtggggcaggcagggagagagagcagccagggtttcggtagaggtatgtcaaaaaggtatggaagtcagaggagaaggagacccctatgttatagaatacaaatggaagggaaatgatgacaacagtaagttgtcattaaatgcaaggttgcaaaagtaagattgtaaagcaggatgagtacccacctattcctgacataatttatagtaaaagctatttcagagaaattggtcgttacttgaatcttacaagaatctgaaacttttaaaaaggtttaaaagtaaaagacaataacttgaacacataattatttagaatgtttggaaagaaacaaaaatttctaagtctatctgattctatttgctaattcttatttgggttctgaatgcgtctactgtgatccaaacttagtattgaatatattgatatatctttaaaaaattagtgttttttgaggaatttgtcatcttgtatattataggtgggattcttaatagattctccaaagatatagcaattttggatgaccttctgcctcttaccatatttgacttcatccaggtatgtaaaaataagtaccgttaagtatgtctgtattattaaaaaaacaataacaaaagcaaatgtgattttgttttcattttttatttgattgagggttgaagtcctgtctattgcattaattttgtaattatccaaagccttcaaaatagacataagtttagtaaattcaataataagtcagaactgcttacctggcccaaacctgaggcaatcccacatttagatgtaatagctgtctacttgggagtgatttgagaggcacaaaggaccatctttcccaaaatcactggccacaaagtgtgacattttggcattggcatcactatttgatggaagccaacctccccccaaaaggcctgtattagaatgaagatggattccctgggtgggttacacttgaaactagcctcacccatgaacactttggcacagattagctagcccattcccccacagtaaggaccataaggaagggacagaagcaaagataagttttagaacaaaagagaggggaaagaaaaaatctagggttttatgagggctgtccctgagtgatagatgtgaataggcctccagggcaggctggctcagaggctgactctttgggttggggtgactgattggtggtgaggatggagaagaaaaggggagtggaggaggtgaaagtgaccttgggacattaggtctccataagtgacaggatttaaggagtgttgtaagctgtggttgttggaccaggtttaagcacagcttcctgagcttcctgactggtttaggtcaagctccagagagcaaatgccacagtctcagtgatctccttggagaaacagttggaataggatgttgcccatgttgggatgagtcattgtccgctcttgctctttccctacccctgcaaaataataatactgtatttgattgaacatataaaacaaaagaaggattatcacataagtatgtatatataaccaacattggcaggtgcagaaaaaccagactgtcagtttgcctcatctgaaatgattgacacaaacaaatatatttactgtcccaagtgaactttggcattttggatatccttcagttgttctgtttaaagatataacttagaagcagctgatggaatatttaaatccatgcgttgaattcatgcattcaaagaaacatgtcctgagtcactaaatgctgacatttgtttttcatgttaagagtgtaaataactggtcccaaatataatattattacatcagataaaaactggaatgtgaacctcttaacttgattgtgaaagtatttgccaatggtgcctcttgataattatttgaggctcacttcagaactcctctggaagggttaatttttaaatagtcattttataaattaacatttttgacatatgtgatggctctcaaattttttcttttatgccagtttgaatcatttctgctcaatttttttttttaattgggatggagtctcactctgttgcccaggctggagtgcagtgatgcaatcttggctgactgcaacctccacctcctcggttcaagcgattctctcgcatcagcctccagagtagctgggattacaggcgcgcaccaccatgcctggataatttttgtattattactagagatggggtttcaccacgttggccaggctggtcttgaactcctgaactcctgacctcaagtgatccacctgcctcagcctcttaaagagctggaattataggtgtgagccactgcaccaggccctgttcaacttttaatgctaagattcatttgttgttgtttcacaagtgattaggcagaggtcttttatattaatttacccattttatttgtaagagagtctcatattaaggaagcataatatatgacaatccaaatacagtacaaatttggttaattttgattttgttaaataattaatcacaggggtccttcaaattgtgagctcctctggttatacttatgttttacctctggttatacttaatttcaaacaaatgaaatttcattctattcatgatatttcagaagcagatctgttgcacaaaataaagcatacctataaattttctttttttaaaaaaaagtctctgttcactctattttctattatttttctctttttaaaatttgaattttattgtggcaagtccacttaacatgagatttaccctcttaacagatttttatgtgtaaaatacaatattgttcaccatgggtaaatgttgcacagcagatctctggaacttattcattttgcactactgaaattttatacctgttgattagtatctccccatttccctctctcccctgtcctgttacccatggttctgttctttgcttctttgagtttgagtattttgatacctcatgtaatcttcattctattttctaactttgacaatgttctgacaaatttgctttccggattggagcactgtatagtgaaaattgaaaatcttggttattttctacagattcccactattttaccttgagcagacacttatcttgaagggtctcagatttgtcacttgtagaatggggaatataaacctgataatggtccctttcagttctaaagttatatcagttgaaaatacatgtgtcacttatggtaacgggtagagaactggctcactgaacagcatatggatattataaagtggttttttttaatcctttctgcagacagttactttatactttattcaaatggattattgtgaagtacatgttagcggactttgtaccttttaaaaatgtatgtatttggtgtaatgtagaaatatagaaatttattaagtatgatttatttcaatgttaagcatgagaaaatatgctccgaaaggttagatagcttgcctaaatgacaagcttgtatttcaagcagaactttctgaatcaaaagactccaagacgaatgcccagctttcaaaaactgtctaaccaaaataaatcctaagattcaccttcatactaaaattatttaaaaatagtttattttaaattaatattcacttaaaatgtatttatcatgcaatactttaaagtgtctgggaaatgaaaatatccaaagatcaaagaacaccatgttttcaaacttcaaaaatgttatcagtgacctaaacaatttttaaaattttcatagagcctatgaaaaatgtacttgcaaatggctactttctgactaggaatagaatggggagagtatttagtccaacaatgatagactggattaagaaaatgtggcacatatacaccatggaacactatgcagccataaaaaatgatgagttcatgtcctttgtagggacatggatgaaattggaaaacatcattctcagtaaactatcgcaagaacaaaaaaccaaacaccgcatattctcactcataggtgggaattgaacaatgagatcacatggacacaggaaggggaatatcacactctggggactgttgtggggtggggggaggggggagggatagcactgggagatatacctaatgctagatgacgagttagtgggtgcagtgcaccagcatggcacatgtatacatatgtaactaacctgcacaatgtgcacatgtaccctaaaacttaaagtataataaaaaaaataaaaaaaagtttgaggtgtttaaagtatgcaaaaaaaaaaaaagaaataaatcactgacacactttgtccactttgcaatgtgaaaatgtttactcaccaacatgttttctttgatcttacagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggtatgacagtgaatgtgcgatactcatcttgtaaaaaagctataagagctatttgagattctttattgttaatctacttaaaaaaaattctgcttttaaacttttacatcatataacaataatttttttctacatgcatgtgtatataaaaggaaactatattacaaagtacacatggattttttttcttaattaatgaccatgtgacttcattttggttttaaaataggtatatagaatcttaccacagttggtgtacaggacattcatttataataaacttatatcagtcaaattaaacaaggatagtgctgctattactaaaggtttctctgggttcccaaatgatacttgaccaaatttgtccctttggcttgttgtcttcagacaccctttcttcatgtgttggagctgccatttcgtgtgcccccaaactctacttgagctgttagggaatcacattttgcagtgacagccttagtgtgggtgcattttcaggcaatactttttcagtatatttctgctttgtagattattagctaaatcaagtcacataaacttccttaatttagatacttgaaaaaattgtcttaaaagaaaatttttttagtaagaattaatttagaattagccagaaaactcccagtggtagccaagaaagaggaataaatattggtggtaattttttaagttcccatctctggtagccaagtaaaaaaagagggtaactcattaataaaataacaaatcatatctattcaaagaatggcaccagtgtgaaaaaaagctttttaaccaatgacatttgtgatatgattattctaatttagtctttttcaggtacaagatattatgaaattacattttgtgtttatgttatttgcaatgttttctatggaaatatttcacaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggtactatgaactcattaactttagctaagcatttaagtaaaaaattttcaatgaataaaatgctgcattctataggttatcaatttttgatatctttagagtttagtaattaacaaatttgttggtttattattgaacaagtgatttctttgaatttccattgttttattgttaaacaaataatttccttgaaatcggatatatatatatatatgtatatatatatatatatatatatatatatacatatatatatatagtattatccctgttttcacagttttaaaaaccgatgcacacagattgtcagatagcaattctgtgattgaaggggaaatatgtcacctcttcatactcatattggtgaagggtcctagcttcaaaattaatagattcctaaagaggggaaatgaaacatccgcatttacacacacacacacacacacacacacagagttcctcttgtcggtaagttttgttttttttaaatctctactagataaaatttgttatctaattgtgagttttacacaaagaaaaactgtcacagaaaagaaagacagtgtcacatttttcaaaagaaaaagaagaaaagaaagtgccatgtttttcaaatacaaatgttctggattgattttaggatctttagtgaaaaacaaagtatttcataataagtaaaataaaaatctatgtaggtaaatttgtttctctaatttaagaatttgaatttctgagtatttatgataagtgttgaaataacttcttatatgtgacagtgaatactggcagagcaaatgccaaatcaatgccaaatctgtaggatcatttgattgtaggaacagaattctactcaaaccgaaagcaggcatttgctggagttacagaaaggcctcatggaacaccgagaaggtggtgccattcgactcttaaagaagctgcaacaggcacaagagagtcagctgcagctcttcttcttgagtctatatctgtcctgggtccattcctttttgtggttgcttcattcctttctctctctgaagactggtttttctggtctaccagggctatgccacattgactttatgtagtgtctccattctggcctcctgaatttacaggagagttcctctgtacaaactcaaagtcctggagagaacagaaaacagcttccttttggctcaggggtccaactgcagtctactctgctgctatgaggatagtgggttcaccacctttgttgttctctcagctagggcagtgggaaatgactctatgaaaggaatatacatgggcaggcaaatgtactaatcctcatcagtactgtaattttaagcaactttaaaaaattcttttaagttatttgaaaataagatcaaagaaggctgaattacataaatgaagatttgttaacaattaattcaaaccaatataacacatgctataacatggttgagtgtgattgagtcttgatttattaggggcaataatcaaaacatttaacaatcattatagtacagaacttaccaatcaaatcagatgctcagccggagtggatgttggccacccagctattattatccctggctcaattggtcttcagctgtgttaacttgcaaacattaattaactatctaagcccctcattttcctcaagtgtaaatagacacaataatattacctattccataggtgtggggtgaatagtaaatgtaataatttgtccaaaacacttagtatagtgcctggtccatggtaaatactaaataaatgttatctgacttattattaaaattttatcttctcagcttaaccttcagaacagtaatatattggggtctagataaatcttgcctatatgaaaataatttaatactacatgcagatatatgctgtgtatattatgccttctgttagaggaattgcagaaacaaaaatttcaattaataataagatgaattatttctcccaattgtagaatcttttgacaattttatcatgcattacagatgtaagaactcttgattgggacttgatagtctaactttataataatttaagaacattcctcttagagaatttctatggccataatactgaacacatgaattttaattagctgtcctctttagccctaaaaaaaaaattactgtaatttaacacttaagtgttgttcttcccaggtacagtaatctttttttttttttttttttttttttgcatagagggtaatcttttctctttccaaatggcagaactgttagttttctgactgtccggtgaaattctaagtccacttacttcccaatagcatgcaattagcaaaggtcctccttgcaaaggcacagaacacacctaaacatcttgcagatgctgtttggacactcttcccctgcttttggtctctttgtaaagcagctcatctggatacaggatctcttttccccattgcccattctaatatatgttaccgttattacttatagaataatagtagaagagacaaatatggtacctacccattaccaacaacacctccaataccagtaacattttttaaaaagggcaacactttcctaatattcaatcgctctttgatttaaaatcctggttgaatacttactatatgcagagcattattctattagtagatgctgtgatgaactgagatttaaaaattgttaaaattagcataaaattgaaatgtaaatttaatgtgatatgtgccctaggagaagtgtgaataaagtcgttcacagaagagagaaataacatgaggttcatttacgtcttttgtgcatctataggagaaggagaaggaagagttggtattatcctgactttagccatgaatatcatgagtacattgcagtgggctgtaaactccagcatagatgtggatagcttggtaagtcttatcatctttttaacttttatgaaaaaaattcagacaagtaacaaagtatgagtaatagcatgaggaagaactatataccgtatattgagcttaagaaataaaacattacagataaattgagggtcactgtgtatctgtcattaaatccttatctcttctttccttctcatagatagccactatgaagatctaatactgcagtgagcattctttcacctgtttccttattcaggattttctaggagaaatacctaggggttgtattgctgggtcataggattcacccatgcttaactgagtggtgccaaattgtcctcaagtctgttgtactgatatatatccccatcaagagagtacaagaattctcatagctatgtatcttcaacaacacttggtgtctggtagatgtgaagtgattactaaaaatatagggaagctgcatacataattattggcttttgctgttctcttacattaatttcttattcatgttgattactcatttgtcacctagttttttcttccttaattaaattgtaggaatttatgaattatggattgatcatcagctctatacatttcaaacataatccctcagtcagtggcttggcttatagagtcttttgatgaaaagaagcttttaagtttaataaagttcaatttattgtcttttcctttatgttttgtgcttttggtatcttgattaagaactccttccttatattgggttctcaaatttagcagcataacattttcatactattatttaaatttttttcacattatttagtgatagcacctttcttattcctaaagtgtttatcattgccttctgtctttctgcttgataaatattgccacacatttgtatactttattagtgtgtacaaagaccacattttagttgtgttatttctcttgttttggttttctagaatgcagagccattaatattatagtaatgcttatgtgctaataccatatcaggggcacaaatcccattgcagcgggactgagaaattaaaggaaatgatgcacatttactcatttttgtttaaaaaatcaaatgcatatttttcaatcagactatatggttggtctggatagcttcatcattgaatttttaaagtatttttgtactactgtatttaaaattattcattcaccactgcttttgtagatggtttagaaacccaagttaggaatgactgtgcaacactattattatactctttttaaaattatactttttgcttaagtttctttccttgttctctgagacagtgttcatgttcccaaaccacacacatttattcagctataaaatttgtatgatcaactcctgtcagaacaaacatcattataaaaaatatctccaggaaaaagaaaacccttttaatgctctcttctggttcatgtgtcttcttattttctttaagcattttcataacccattgagctgtaatttaattggaacatgatttatactaaagttggtttctttacctttaacttttttttttagtttgatcagctctctttagcttctgtagttcggtctttaattccattccagtatgcttttggagttgggtctcataaatgtatagaaatgtttctgttgggaaacagcaggagaatattaaataaatattgtgcttacatctatttaattctttgcccaactttctacaactttgactttacatttaagctcctcatgcacttacatgtttctttacctaaaaatatcttttcaccatgggtgtgtacaattcctttgtccttgctgtattaattttcttggtttacatagtagcctctacacattgatgtcaaaacctctgtttggtgcatttctactctgcgtgttcaatctccatgaaagtttctgtaaggtattttcattcctctagtttttcacatgtgcatcctggctttgtgacctgtgctttgatatcgtgcctttcatcttgtggcattgaaggatctttgcaaggacctattgtgttataatacagtctatgaaaaatatcaatatttgcatttgatcacatttaaaaaaatcacattcttttgtttgaatatcaaagctaatatgtgagtgatttccctgccaaatagcacaagtagcctttcctgggtgtttatgggcatttatctggttaatgattcccatcatagtgctgtcacccatgccattgctaaacttatacagtaacttttttgttttcacctcagcatatgttgagagtaggaaatagataggactatgccctcaaattttacgtttatatgatgttaatcctaaaggtccttgtgacttctgaagtaaaaactcagtgttgtcattttacttactgaattgttagctgagtttagagttgagtttacaatggagtaaacaaggtgtttagtttgatgtatgcttttagtctttcagaaaaaaatgtttatacttggaaagaatagtttatttacccatctggcctagtttagacaaaaacacagagtcaaatgtcaacagaattctgaagttataaaaatgacagtgtggctttttttttttttaaccttccacctggtgcttatgcccaagtgcctagctttctttagctctcaactaataaaggtaatgtttagataacatttaacgttaagttgcattgtgtttatgatcacatatctcaaatattggtacacgaaactgtacaacaaccttttttattagattttcctacgaaattccttattatattccctaagatagctttttcccaccttcttcttccttctcccttctcaggtgctccaataattccaacccctgcagccagtgactttattatatctttttttaaaaatctaaaaaaaaaaattgatgcaaccaggaagaattttctcatttctctccaccagttgtaccagcctactgcacctctcctcatgcaccaccttctgcctgtgttcttgctcctatattcaggagcaagtaatatgcaatacctccctctttgtgggatctttctcattagcataaaaatactttcccttgatctccagctactaccccatttctttgacctacatatagcaaaatatttgagaaaggaccactttccatcttttcctcaatctacttccatttttttctcaatccactttcatttcattgttctcctcaacccattctttccacaacctacttcattttatttccatcagccccataactcaggatcaacatcttgccagagccaatttccttgtctcccttaacagctccagcagtatttatgccatggacaaattattcttcttgtgatactttctctcttgcttccatgacactactcccacttcattttctttctacctctctggctcttccttggtcccttttcctggccccttctctctttcagatctctaaacatcagctatatctcagccctgttctactgacactctctagctgttattttctaaacccatgtttcagaaaccatatcttgatgaatcttggaaggccgaggcaggcgaattacttgaggtcgggagtttgagaccagcctggccaacgtggtgaaaccccatctctcctaaaaatacaaaaattacctggccgtggtggcatgcacccagctacttgagaggctgaggcacaagaatcgcttgaacctgggaggtggaggtttcagtgagccgagatcctgccactgcactccagcctgagcaatagaggagactccgtctcacacacacacacacacacacacacacaaagaaaataaaccatctcttgatgaatcataaatttgtgtctctagtttagacctctatcctgctctctaaatgatgtatccaactatcatcttgacaccatcatatgttcataaaacataattatagaatatctttcagtaggcttgacattttaaggcatgagtttccgttcagtatctccttaaaatatacccagggtctcaggagactattcaaacaggacaaagcttctattctacttactaatgtgtctggccctatttggcaggttggataaaaagtcatctgaacattgtcactttatgaataatatagtttaatagtttgtgaatcacccctgcaatttaaaaaatagtaaaattatcagaatctaatttaataattcctattggaacaccccatgttaggggatttccagttatttcaattgatatctcaatgttttaaagattgtttatttctattactaattcactctttattttaacataaattgtggctatctatctctattcatttcaattatatttctcataccattctatagatggggtgaaaagaaaagtgttaattttttaaaactccatacctcaaatactatatgaatttatagttgttattgctaaagcaattatcttacatcttttcctccaaaacaaagttatgtgctggtttattttctttgtactcataagatgccttccatttttagtaacataagtcttgtctttctcctattcttagctacttaagcattatgtagcttaaataagcactaaagattcctatctgtatgaaaaaataaagattaaataaataagatctagaaagggtgacaaggtgatgcttcaaaatgaaccataccaagccatctagcgattgataaattactcacactcataatcacattgttggaaagaagccattgacaattcagtttgtttcacaactgtctatcacatagtgagcacaactaaaagactactttttgtcttttactgcttgttttgttgatcaagtgactgattgtacaatgaccaacaagaagtctgatgtgtagagaaaaggggaacctggcttttctgccttactcctgatgcctaattctgagcatgtgaatattattctgtttctttaattctccaagtgaagcagcagataaaccatccttgtttccattagctgtctaccctgttcaactgtgtgtttctaataacataagaataagaaagccaccagggtgagcagggaaggcaatgagtctgcaaggcttgtggatagatttctgttagtgaggctctagaaagttcttccaagattgatgcaatctgagaagagttttctgtcaatacaaactccctgggtttctcctttgtccttttactgcctgtgtttgttttgggttccagtaaagatcaagtgactgattgtaccatgaccaacaagaagcctgatgtgtggagaaaaggggaacctggcttttctgcattactcctaatgcctaattttcttgtactgaaagtagtttttgctgtaagaatctgaggggaggagtcatttcttcaatttttttttttggtctccttttaatggtttcttgatcatgtctatccttatttttctgttttcacaaatttttgtggtatattttcctctcatgacctctgtctcaagacttctttccatccatctcttctcatttcatcctgtagagtgtctgtggtaagagccctgcattctactctggccttgccatgtgtggccttgggcaagtcctagcctccttgagggtcttatttttctcatttgtaaaatgaaacagtttgatgagaagttttctaaggttccttcaagctttgacaatctctctcttctggatctttttcccatgaaaaatttcaactcttgattagcatgtaggcagggattattccacatccttataggaatcacatttctgctactgtccctgaatgctagagtccattgattaagttattcactgctgcaattgtcagagctgatcaaagaactctgaaccagtgtgttactagaactaacaaagaaaatgccattatgatgttctagagtcttgaattagtagaagaggtttaataagaaccctaagggattgctagaatgttaaaaacaaacaaacaaaaaaaaaggttgaaaagtttagaaaattcactggtctttgtgcccatcattttacttccagggtttagataatctcatttttgcaatgaaggaatggattagatcacaagttctcatcctagtagcacatgcagaatctttataaaaacacagagtagccaggtgcggtggctcatgcctgtaatcccagcactttgagagcctggggcaggtggatcacttgagaataggagttgaagaccaagctggtcaacatggcaaaaccctgtatctactaaaaattcaaaaattagccaggcatgatggcacatgcctcccagctactggggaggctgaggcaggagaatcgattgaacccgggagatggaggttgcagggagctgagatagctccactgcactccagcctggtgacagggtgagactccatcacaaacaaaacaaaacaaaagaaagcaaaaacacagattactcagggtccactaagaccagtgaagtcagttctcttggtagggggcagggtgactgagcatgatgtttgtaattttaaaagtgctccaggtgattctagcgtgtatcaagcaagacttgtgaaccactgaactacatgctaagactcattttagctctgattttctgtgagtcatagcagagggctcagcaaactttttctataaatgctaagatagtaaatattttcagctttgtgggctgtatcgtctttatgacaactcaactcagtctttgtagagaaaagcagctgtacataatatgtaaactaatgggagtagctagatgtgtcctgtgggccatagttttgctgactcctggtctatgtcatagaatttccttttgaattgatggaccaccagcaaatgatttttgtcctgtatcaatcaatgatacatacataaatctctacaagacatgtaaaggatgaggcttaatgacagagtactttggggaagacataatattgcaaaattaagatgcttagagaaaaatcatattaaaatagtgaaaactgtgagaaggtattttgatttgttgttttggattcctctttttgcaaattcttttgaaatattttcagtggaagctacatagatccaattgtattcaccaagctagattgtaattaagctccagagtaagtaatagatttgatgagtgatgtccaaccttttacatggaagagtaagtttgagtcttcctttgcccattgacacacttagtaccatgtttaccaaagttcttagttattgaaatgggcaccagcatattttgaaacgttggtgttaacttgggatatgccttttgtcatgttgcaaatagattttgtttctgttttgtgaagatcaccatctctgtcacttctgatagaaaaagtgacactgacttctcaagtgatttgacacaggttaaaatatgtaaaccatttctgtagagagcaagctgtaataatatactaaagggctaggtttatagtataatataaataactcatttatgctgttaataatttatagcaacatggcatttgactgactttttatgtgctctagtcatgtaagtaatagatgtggaaacatagaccagagtttcaagaacatgttttgggcagagtctgttttcttgctattatctcttaagtttatgttcatggcctaaagattatgctaatggatctgccttggtcttgggtgtcaggtctgtgttagcgagtattgaaaagcatagtttttgcctactgggaaggatttatgatttaaaagccctaaatctccccttttatgtacttcatacttagaaaatttttcctgtaaactgtgtgacttttttacattgtgccagttttctagatgactctcgtcatatttatttcttgcaatccttctataactatcagttatgaagtctctttatagtgttgccagccaggtctcaggtgtgtgaaatgtattttctattatggattttggggtatgatggcacatagtttgggtgttaatgcctaatcttgatgtactggcttctgaacaaccaaaaggatgaaaggaaatagaacaaatatttttgtgagggagaggagtctggcttcttgacttactctagaaaaagcctgtaagcctcctcttccctccttgtcacacaaagtgacaaagaaaatcaagaattgttttcttcttggcttaaatgcatcccttataaagtaaggctgagatcaggctgtgaagctatctttttgtcaagactgtcataattccaaaacactttgttcttctaatgcttaggttagtaactttaaacatttttataaagatagtgaggtccagttttaaggattgaccccttctcaaggggctcagaagaggttttggagaataataaaattaaataatgaaaccaataatttaaaccagatcatgatccttaagaaaaaatcccatcaaatttgggctaaactctaatatacagaggtctgcacaacttatgtcaagtattcttccccacaaatgaagaatggggttcattgtgtcattggttgggtctcattttggcttcatcttctatttctcaaagtctaagaaaagtgctcctacggaagtgggtgttggctatcatgagactttgctgctggcaggccagcttgctgctctagacagagatatccctcgatcctccttggacaactgttttctgtgcacaggaagcagcaggctggggttaaggagtttgccaatccagtcattctgataattgctgaatatgaatttctatccagcacaatctaggtagctacaatggcacagtagtttttatgtatcaggtgaaaatgtttaataggcactctaaatgagagaaaaggttaagtgaggttaaaagctcaatgaaaacaaatagatgagactaaaaatagttcaataggttgtaacttccatctcatccaaacagcaatgaatattttgaggctgaggcgctgaggggtaaaattgcagcctggactacttgctaatgtagacctacagcactgtcattcttactgcacagacactgctttctgcataggaggtagaataatgaattcatttattattaacaaagatttattaagtgactgcatggtgctaaccactagatggggagggatgttttgaactgtccattgtttgactataacaaggaacgctttgaacgaggttactatcataggcagaatttgtttaacatgaagcctatgagacataagccacaggtcctctcacgtgcaggaactcctttgaaggccctatacttaattttatatgcatagtttggatttggattcttttttttttaagagttccccaaattacttaagcttcaggctccacaaaacctggatctacccctggtagcagctatgaatctttgactatgaaattaagtgtacaagaaatatgactttactttttctgtgattgagtttattttctatttgagcacgcattccactgagtgaaagaaataatatcattgaattcagagattttgctgggttctaagtggagtttacagaatgccatgatattaggaattaaggagtgtgttgccctacatcatcttttgtccgtgctcactgtctctgaggcactgatgttcctatgtgacctagaggggcatggtccaggtagatggagtctgtccttgttctcactgtgagctctcgcttgctgacccttcttcagtttcttccatgcccctgaggggtaaaaagattcaaatctgaagctatatcaagccatctgtgcatagacattccaagcaaccatgttcactctactgctcccatgtcatgcaaggcacaggaagcttcactatggcatgagtatttcctgggctttgccttggaattgaggcacgggcctcctttgttctaaaattccccaaatctacttgaggatagaaccaggatttggttgcaaggcagaacttttcttagaggacctggtatctaaaccctcttgttacccccatttatggaccccatttatggggtgaggagagtgactgcttctaatccatcataatttttgtctatggctactgtttttgcatagacactatgttttgagtccttaggctttggcttttggcgcttaatggccaatattcacatggctcaaaattttcaaatgatccatatctgacttgagtttcaaaagtcagtttttgaaacttaaatgatcagaattgatttgttctgctctggttctgatgtggcctctccttccagaggtactggaggtagaatatccaaggtggaaagcccacgactacaaggaattggttagtaattcataatgttagctgtccacatctattcagtaatggcatttcagtggctgcacaactgaccatggtgaaagtgtctgcacaagccactttttcttcctgtcagaaaatgttctcacccactgaattgaatgactgtctgctcatatgctgtgaatgagtgcccagtcttaagattaaatcacacgttcttggctatgcatatttgggcatgctgtggggagttataataggctgtcttagagtcacattaagcagctagacagacaatgagttggaaagttacattttctaaatttgattggtacattccatttgtcacatttgacattagaagttctggattcaccctctatggtgagcttcactaatggagaatgtaatttgcaatgctcaaacacaagtcctaaacagaaaacattgtatgttacattccagtgctaccaaaatagtggttttgaaagtccttattttctaatactactatgtgtaattttgagtcatttagatagcaacagttaaatgttttatagattgtttggaagtattaaaatgtgaaggatttttgttatatagtgtctttcctatcttgcttaataaaatataagtttagaattgtgtatagaattaacatgcaaaaatatcaagtctcaactttatacagttaatctacatttgtgtatacccttcaattatttcaagagagggatactattcttatgcaggataaatacaataagatattttaaatgaattttaactacatctctggcagtttcatctcaatagtagttgtaattttatctcccagaccttattatagactagcagctctctatgaaaattagtgacagtgtgagtgtattttaattcaaagttaatcaagaatgactgagtcaagagttagctacccctgaaagtaactcataattcagaatttaaaatattacatgtggaacaatcatgactatatgccttttactttctctatcattatttaggttgtgggctttgggtccttttcacatccgttaacagtgggcttgacttcaaaggattattttcttgaatcttgaataattgctgaagacaatttgaagatattttcaagatgaaggaaactgaagcacagaatcactagagtgaaaaaagaacttcacaaacagtgcaggcttgatcaatggcatgggaaaacaggcaatacagttagaattgctaagatggaattttaacgttcaattaaggatctatctctaaactcctctgctttatccaccaatcattccatattaaagatgaagaattgttcccatttcaccttttgataaggaaaaatagaaataacagaagcaaatacacttttgcccacatttttttccaaaaagaataatttttgaagtctaaacgtttggtgtaaataagatgatgtgttaatattgtaaaggaaagctagttaagtttttgactgaataaagccagcatcaataattactagtaagactaaaaataagagcagtaaaattgtgtctaatcagctactaatatctgggaaggattgagccacaggatcaaagatggtatcttttaaaaatagaagttgagtgaattcggtcttcaaattctttctttttattcatttatatttatttactcattagtatattcattcctttattcatgtattgttcaaatatatattgggtacttattatatgccaagttgtttttaaaatcacattccaaattcccgtaagtcataattattcagagatgtatgttttttttaaaaaaaattgaacacctttaaaaattatcaagtccttttatttctgtatgcattaaagataaactttactaaatgttacatgaatagatttataaagcagataaatatttaatttcaaatataacccttatatgcaattatattttccttagcactaaaaatgaatatttaagtaatttatattaaaagtgtaattatttaactgcagatgtatgccaatgacttaaattgtttaaagattatagcaaagttgtttaaaattgtctaatcatgaagagttcacttaaccacctggttgacacataaaattatagttagttactaaggtagttcgagagaaagagaagaatcttcagtagtggttttgaggtgtggtacattttattataatataccggttatacagcattgtgcagtgctgctcatagtagaaataaattttctctttgatgtcatctattcccttgtgtggcttacataactgagaattaggtgatcacaaaaataaacaggcctatacagagcccatttatataagtcctggttatttctcttcagttaaacttttaattatatccaattatttcctgttagttcattgaaaagcccgacaaataaccaagtgacaaatagcaagtgttgcattttacaagttattttttaggaagcatcaaactaattgtgaaattgtctgccattcttaaaaacaaaaatgttgttatttttatttcagatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgagatttgaacactgcttgctttgttagactgtgttcagtaagtgaatcccagtagcctgaagcaatgtgttagcagaatctatttgtaacattattattgtacagtagaatcaatattaaacacacatgttttattatatggagtcattatttttaatatgaaatttaatttgcagagtcctgaacctatataatgggtttattttaaatgtgattgtacttgcagaatatctaattaattgctaggttaataactaaagaagccattaaataaatcaaaattgtaacatgttttagatttcccatcttgaaaatgtcttccaaaaatatcttattgctgactccatctattgtcttaaattttatctaagttccattctgccaaacaagtgatactttttttctagcttttttcagtttgtttgttttgtttttctttgaagttttaattcagacatagattattttttcccagttatttactatatttattaagcatgagtaattgacattattttgaaatccttcttatggatcccagcactgggctgaacacatagaaggaacttaatatatactgatttctggaattgattcttggagacagggatggtcattatccatatacttcaggctccataaacatatttcttaattgccttcaaatccctattctggactgctctataaatctagacaagagtattatatattttgattgatattttttagataaaataaaagggagctgaaaactgaattgcaaactgaattttaaaactttatctctctgtggttaattgcaaacacagatacaaaaatatagagagagatacagttagtaaagatgttaggtcaccgttactaacactgacatagaaacagttttgctcatgagtttcagaatatatgagtttgattttgcccatggattttagaatatttgataaacatttaatgcattgtacaaattctgtgaaaacatatatataggatgtgcgaaaagtccctgtgtatcatgtgaaatggcttaaaacagaacaccataggtattcatatcagtgaataccataggtagctgaaagtgttttttcctggggtcgccaagatgaatgccaaaagtgatatcattattataaacaatagccagaataggttggtataaacctggtagaaagccttgataaattgactttctctcctcctgacatcctgccacccctttgctttgctgatgctcatttgtccactaaattaaactcaagcaagccctagtaaagtaatagaatttgtggagtcctcattagtataggaagtttccctgatgtgagattagtaattagagatgtagcaaaatgagaaagaagtaatatgcttagatatttcattttctctgaacctgtatatacaaaataggccatgcgtgttcagtaactattcactgcaaggcactctctaggtactttgggggaattggaaattactcacataaggctatggattgtgccatttgtcaaaagacaaaatgacaacaaatttagtttaaagacctcagtcagctttattttctattctagatttggacagtccttcatttcacaaattggagtaagtgttccaataagttgagcaaaggagcttggctttatagacccaaaaaaagggccaaaggaagcagaaacaaagaacaataagagaattggtcatttcaaagttacttttcttgaaaggtggggacaaggagacagaataatagaaaagtcactgattggttaacattggattaagaattaaaacagaggaaactttaagattgaagtttgaaactgacttgtttgggaaatcaggctgtcttctttcttgatttcttagaaggccggataacaactgagttttgctttggtgaacatgggtgactccatttttacttttagtctggtctgttgaggcctcgtgagagagcttaatctaaaacaatgacttcctataatttttgtttgacacatccaaagagggactctaatatttattgagagcttatcatatcttaagtactgtttaaacacttttatttgctattacatttgatcttattataactctaaaggcagaaatgattgcttttattttccacaatggaggaaactgaggttcaattaagtgagtaaggaagcagggatcttaaacccagataccattgctcctctttaaaggtggaagaacagaaaacatggggcaggggaagagagaaagtttctgtcccaggacatgataatctaaaagggaaaacgtaagatccactgaaacctgaggcagatttattgtggcaataacaaagcttaagtttcacagaccttcatttgcctgagccaactttgaaggccatgtatctaattttgtttttataattctataatctttattcttgaaaagagccctccctccaaatttacaagctttgggcccccaaaatccttgaaatgcccttgaataagagatatccaggtaaatgctatgggaattcagaggaggaagcagttagtatcagttggcggagagttaggctattaagagaaggttttatataggaagtggcatttagaatgaagctttgagaactgagctgtgtatttgaacaagtaaaggtggtgttgcagaattttgctccttagttctattaaaaacccgggttcttgtcacatgatccggaaaatttaggcacacagatacattgaagcatgagtagagcaggattttattgggcaaaaaggaaaaaaagaaaactcagcaaatcgagatggagtcttgctcacagattgaatcccaggccaccacaaaggaactgaagagatcgggcttctcccctgcataaggtgcaaattccccatggctccacccacttccccttagtgtgcatgtggggctccagtccacggtgggcatgcccagacaagccttgggcaggttccctcatctgtgcaaaagcatctgatgtaaacacttgaggggtggttcggagattctctgggacccttttattttcttatctgcctaggcatttggctgtctcagtgggtgggaaagggtgctccaggcaaagggcataacatgaggcaaagggcatgcacagaaaacagtgactggttcagtcaggttgggggatgccaaaggaagtaatgggagacaagattggagcaagatagataagagattgtggattttttttcttttttatctatataaatacagagacagggtctcactatgttgcccaggctggtctcaaactcctggcctcaagtgatcctcccacctcatcctcccaaagtgctaggattacaggcatgaggcactgtgcccaacctccaattttggattttgagagctaaagcaatatagtcgaaaactcagataatccaggtagattttgctattaggtgctatttggttcctggtacagagctaaaacccttggaatttcctaagtgataagagctacaggagcatcttttgttatatgtttccccccctagttcctgaaatagctctagagaaatacaggtgaataacatcctttgttattcatatcaagcccctatcaaccataccccagtttctatttatgaagtggcttttgggaagtccctaaagacaggagtggggaaaggctggttgtcagggggatgggttgaaactttcatcttccccccttgacctccagggagggatgagtggctgaaaattgtgtaaaatcaacaatggccagtgatttaatcaaccatgcctatgtaatgaagccacccgataagccttaactggaactttttggagagcctccaggctggtgaagacattgaggtgctcagaaggtggtattccagagagagcacagaatctctgttccccttcccacattcattttgctatgcatctctcccatctggctgttcttgagaggtatccgtttataataaactggtaacctagtaagtaaactgttaccctgagttctgtgagccattctagcaaattatcaaacctaaagagttcatggatacgtgcaatttacagatgcacagtcagaagcacagatgacaatctgggcttgccattggcatttgaagtgtgttgggaggcagtcttacaggaatgagcccttatcctgtggggtctatgctaataacagacagttgtcagcattgcttggtgtcgaaaacccacattgttggtgtcagaagtattgtcagtaggatagggaaaacagtttgttttctttttttagtggtctttggtcatctttaagagcagggcttctcaaagtgtggtccttgaaccagcatcacctgtaccacgtaagaacttatgagaaatgttcattcttgggccccaacaaagaattaaaaattctgagggtgtgaacggggtctgagtttcagcacaacttcccgaccatgctgatgcattcttgcccaagcatgaaagccctcccttgtttaagaaggccattagggccgggtgtggtggctcatgcttgtaatcgagcactttgagaggacatagtgggaggatcacttgagccctggagttctagacaagcctgggcaacatggcaaaatgctgtctccacaaaaatcacaaaaattaggtgggcgtgtgttgtgtgcctataggcccagctacttaggagactgaggcaggaggatcgcttgagcccaggagattaaggctgcagcgagctgtgatggcaccactacagcctggatgacagagtgagacactgtctcaaaaaaaaaaaagaaaaagaaaaagaaaaaagaaaggaaaatgaaaaagaacgccattaggtataaaggagcaatggtaaaagaccagttgcaaaaggttagggaatgggtggttactgaaataagaagctatgtagaacactagtgttggtggcaggaagtagaaagcaagagcactgctctgtgggggatggtcatagcaaatgcaatatggaggcatttgcctctgcactgaggagaaaactatcttttccaagataggaggaaaggagataagtggaattaaagagaacctttgagcacagagttgggaaactgaaggtatttgtgttgtgctccctcaatcttttaattcaactataagctaaacccatgaaacttgagtagtttcagttatctgacttttttcttctcttttgatacagtgttggctattctgggtcttttgcctctctttatgtacttaagaatcagtttgccaatgtatgcaaaataactggctgggattttgattgtgattggcttgaatctatagatggagttgggaaggactgacatcttgacaatgttgaagcttcctattcatcattatgaaatatttctccatttgtttgattctttgatttcttttatcagaatttagttttcctcatatagtcttttaaaatattttgttatattttgttcaagtattttgtttttgaggaatgccaatgtaaatggtattgtgattttaatttcaaattccaatttttcattgctgttatataggaaaatgattttttttgcatgttagccttatatctttcaactttgctataatcaattattgatagtttcaaggattttttggtcaattattttgaatcttctacatagattatcatcatctgaacttagttttatttcttccttcccaatctgtatacctttatctccttttcttatttcattagctaggacttccagtatgatgttgaaagtagtggtgagaggggatatcttggtcttgttcttgatcttagtgggaaaacttcaagtttcttatcattaagtatgattttagctggagggtttttgtagaagttttttttttttaagttgaagaagtctccttctatttttagtttgctgatttttaaaaagaatcaggaatgggtgttaaattttgtgaaatgcttttctgcaactattgatttgagcactttatttttcttctttggcttgttgatgtgaagtacattaattgatttttgaatgctgaatcaaccttttgtacctgagattaatcccgtttggttgtggtatataattatttgtatacatgttgagttcgatttgctaatactttttgagaatttttgcattggtgttcatgaaaaaatattggtgtgtagttttttgtgacatctttatctgcttatggttttaaggtaatgctggcctcatagcatgagttagggagtatttcctctacttttacatttgagaagagattgcagagaattagtaaaattcctactttaaatattttgtggaattcaccagtgaacccatctggacctggtgctttctgttttggaaggtcattaattattttaaaatagatataggcctattcagattacctattttttctcatgcgagttttagcagattgtctttcaaggaattggtctatttcatttaggttatcaaatatgtcaacgtagagttattcatagtattcttttattatccttttaatgtgcaagggatctgtagtgatgtccccttttttgttttattgatattagcaatttgtgtcacatcttttattttgctttgttagccaggctagagatatctctatttttgatgtttttgatgaaccaactttttgttttattgattttctctgttgatttcgtgatttcaatttcatgatttttaaattatgcttacatttgatttaatttgatcttcttttgctagttatccaaggtggaagcttatattgttaagatccttttgcattcttatgcattcaatgatgtaaatttccctctaagcactgctttttctgcatctcacaaatattcatgagttgtattttcatgttcatttagtttgaaatatttttaaatttctcttgatatttctcttttgacccatgtgttacttagaagtgtgttgtttaatcaccatttttaaaaattttctagctatctttctgttattgatttctagtttaattccattgtggtctgagagcatatattgtataattttaatttttataaaatttgttaaggtgtgatttatggcccagaatgtggtctatcttggtgaatgttccatgtaagctttggaagactgtgtattctgctatatttgaatgaggtagtctatagacatcaattatgtccagttgattgatggtgctgttgaattcaactatgtccttactgattttccacctgctagatctgtccattctttgcagagggacactgaagtctccaactctagtagtgaatattctatttcttgttacagttttatcaacttctgcttcatgtcttttgatgctttgttgctagaaacatacacatgaagaattggtatgtcttttggagcatgacccatttatcctcatataatgcccctcattatttcctcgccctgatgtctgttctctctgaaagaaatatagcctctccaggtctcttttggttggtgttaaaatgacttaactttctttatcccccttacttttagtttatatgtggttttaaatttaaagtgggtttcttgtagacagcaaatagttcagagttgtttttcgatccactttgacaatctttgtcttttaattggtatatttggactattgatattttaagtgattattgatatagttagataaacatctactatatttattactgttttctgtctgttacactacttgttctttgtttatatttttattgtctactctttttctttccattgtggttttaatcgagcattttatatgtttccattttcttttcttagcatagtaattcttctttaaaaaaacattttttagtggttgcccctagagtttgcaatatacatttacaactaatctaagtccattttcaaataatactaaataatttcatgtgtagtgcaagtaccttttaataataaaacactcccagttccaccttccagtctcttgtattatagctataatttagttcacttacatatatgggtatacctaagtatatacattatcatatttatgattgaatatattgatgaaattattttgaaaaaactgttatcgttaaatcaattaagagtaagaaaaatagttctaattttattataaaatgaaataccttcatttattcattctctaatacactttctttctttatgtagatccaagtttctgacctgtataattttccttttctctcttcagcttctttgaacatttcttaccagccagacctactgacaacaattttccccaatttttgtttgtctgatagagactttatttcttcttgacttttgaagaataattccacagggcacagaactctagattggtgatttcttcccctcaaacccttaaatatttcattccactgccttcttgcttgcattgtttctgagaagttagatataattcttatctttgcctttctataggtaagatgttttttcctctggcttctatcaagattttttctttatgaacatgatatgcctttctttttgaacatgatatgcctttctttttgaacatgatatgcctttgtgtcggattttttttggcattattctgcttggttttctctgagtttcttggatatgtggtatggtatctgacactaatttggaaaaattctcagtcattattgcttcaaatatttcttctgttcttttttttcctttattctccttctggtattcccattacatgtatgttacagtttttgtagtcatcccgctgttttggatattctgtttttttcagtttttttttccttcgcatttcagtgttggaagtttctattgacatattctcaacctcagagattctttcttcagctgtgttcagtctaccaatgagtccatcaaaggcattttacatttttattacagaatttttgacctatagaatttcttttgattccatctttgaatctccatttctcttctgcttttcatctgttcttgcatgttgcctactttttccatgaaaacctttagctttttttttttttctttttgaggtggagtctcactgttgcccaggctggagtgcagtggtgtgatcttggctcactgcaacctctgcctcctgggttcaagtgattctcctcctcagcctcccaagtagctgggattacaggtgcctgccaccatgcctgagtaatttttgtatttttagtagagatggggttttatcatgttggccaggcgggtcttgaactcctaacctcaagtgatctgcccaccttagcctcccaaattgctgggattataggtgtgagccaccatgccctgcctttagcatgttaatcatagttgttttaaattcctgatctgttaattccaacatccctgtcatatctgactgtggttctgatgcttgctctgtgttttcaaatggtgtttttttttttttgccttttagtaagccttgtaattttttattgaaaggtggacatgatgtgctgggtaaaaggaactgtagtaaataggcctttagtaatgtactggtaggtgtagcagagggtgagggaagtattctgtagtcctatgattaggttttagtcttttagtgagcctgtgcgcctgcagcttggaagcacttgtgaagtgttttttcaccccttttggtgggacatagtgactagtgtgagcgggagttgagtatttcccttcccctaggtcagttaggctctgaaaaaaccctgataggttaggcatggtaaaatagtctcttttgagggcaggcattgttataagaatagaatgctctggggccaggtgcggtggctcacgcctgtaatccccgcactttgggaggctaaggcaggtggatcacctgaggtcaggagttcgagaccagcctggccaacatggtgaaaccccgtctctactaaaaatacaaaaatcagccaggtgtggtggcacacacctataatcccagctactcaggaggctgaggcaggagaactgcttgaacccagtaagtggaggttacagtgacccaagattgtgccactgcagtctagtctgggtgacagagcaagactccgtctcaaaaaaaaaagaatgctctggcatatttgaaaatggttacttttccctttttttctctgatcttcactgtgagaacctggtaagcatcctataggcaaaattcataaaagtatagaagtcggccagtgacttggacccacttggaattttcttgctctcacatcatgcacactgaatctccagcaatttttcacttacagtttaggttttcctaccctactactggttctctcagaggtttctgcttattggtttctgttttgtaagttgtgattctctgtacctaactgcctgtctcccattttggggggcagtggtttgccctgtgacctcacttctctgacagatctaagaaaagttgtttatttttcagtgtgctctgctttttacttgttacgatgaagccaaccactttcagaatttctacaaaccagatcagaatctggaagtcctgtttttttattttttttatccctttgtttagcatgttacctatcttaacacattttaaataagtgaatgcatagcttatatctacttctaggttatatgcttccttagaataggaattgattcttaaaatgtcgttctgctcacgcctgtaattccagcactttgggaggccaaggcaggcggatcacttggggtcaggagttcaagaccagcctggtcaacatggtaaaaccctgtgcctgcaaaaaatacaaaaattagctgggcatggtggtggccatctgtaatcccagctactagggaagctaaggcatgagaatcacttgaacctgggaggtggaggttgcagtgagctgagatcgcgccactgcactccagcctgggtgacaagagcaaaactccatctcataaataaataaataaataaataaataaataataaaaataaaaaaataaaataaaacaaaaattttattctgagcagtctctgaagaatataaattctactgccttgcctttagaacttataacagcatctcgcaaactatcacaagatgctccaaacatacttcttatgtgctgaattaagaagtcaactcaaatttagtatactagtaatatttttggatatcccaaaacactgccagctcagctttaggctgcccttcttgggggggaaaaaagcagttgaaatttaggacttaagtgggcatctcgtttaatttttaatggatttctatgttgttggttatggtgaagaggtgaaaagaataaatattctgtgcagaaaaattattcagtcttcatgtgaaaacactttgtccatagcaattactttatgaaaaagatgtggtattactttctttgctcttaactgagacctttaatttaaagaacctatactttacaagtttttattttcaatgcatgaaaaatgtagcagctatttcacaacctttacttttaaaatccatttttctttttaatctcaaatagttttttcttaaaaccttttgactttttatctaaattgtaatagccagagcaccttcccacaactagaatatctcatcctttttgtcttttctttttcctctcaaaatgcctactgggaacttaatttggagtcagattcttcatgataaatctggacttaatcaaaattcctcatatggtatattgtatatatcacagtactggatagtcctctgattaaatagatatttgatagtactttaaggtctatacttttggatgaacttaactgctttctccatttgtagtctcttgaaaatacagaaatttcagaaataatttataagaatatcaaggattcaaatcatatcagcacaaacacctaaatacttgtttgctttgttaaacacatatcccattttctatcttgataaacattggtgtaaagtagttgaatcattcagtgggtataagcagcatattctcaatactatgtttcattaataattaatagagatatatgaacacataaaagattcaattataatcaccttgtggatctaaatttcagttgacttgtcatcttgatttctggagaccacaaggtaatgaaaaataattacaagagtcttccatctgttgcagtattaaaatggcgagtaagacaccctgaaaggaaatgttctattcatggtacaatgcaattacagctagcaccaaattcaacactgtttaactttcaacatattattttgatttatcttgatccaacattctcagggaggaggtgcattgaagttattagaaaacactgacttagatttagggtatgtcttaaaagcttatttgcgggaagtactctagccttattcaacagatcactgagaagcctggaaaaacaaatcccggaaactaattattatgtgccagttatataaacaagaagactttgttgggtacaaaccagtgattccttgcctttgaaaaatgtgtcagatatcatgcattaccagcagttcaatgatataaggaaaccagagtaatagctaaaacctttaaagctaaaccaaagatttacaaattgcctcttcatccagtctttcccaacctaaaaactgagttctctaaaaattttagtatttttttctgaagaaaagggaacatggacatttatctaatcctcattagaaatctgactaatgataacaaggatttagacctcaagcacttcttaccaaaattcttgatatgaccttatagcaaattactttcacctgttgaactttcctttcttttattcccctgtacctcacctgcactgggcatattcaagttgcttatacaacactttactattgtgttagaaaaatcatgacacatgatgaatgtgtttgtgcaacatgagctgattcataaatgaaaatgtgcattgaaattccacaatattttaaaattaggagtttatctagcaattgaacaaaattgattaaatccattatttgttagatcagctaaattacataagttcattcatctgctcataaatccatccattcttccatctggctatcccttagtcaattcaaataaatatttatggggcactttgggtaagccaggtgctaagaattcaatgcaaaacaagatagactcccctgtccttgttgaacttatatttttggtacaaacaaaagcaataatcaagaaaaaataaaaaaagtactgattgtgattaataatatgaagaaattcaacagagtattgtacttaacatttgattgatctgattttctcagttgtctgagaacaaacatttgtgaaaatctcattgtagagttcttacgatggatagggggtcaactgtgtcattattgcttatcagcttatcccaaagacctagtttattaccagattgcaaatagtgttcaataaattattcttattaagggttgttatgtactctaaaacatttattgtggtcccttcactggttctggtttacaaacttacttttctatgatgacatagtatagaaattgagagtgaatatttagaagttcatttttattatatatttttgaagtattgatatgtagtgaattagaaatttaaaaagaaaacaaaactgtccttcactacagattgaaaagcattatactaaaagaccatttgctcagttatagtatataaaggccaaatgacttaaaaacaaattatgtaaggagaaggaaacaaccatttattcagtgccactaactgtcagccagttttttcagtggtcagttaatgactgcagtagtgttctaccttgctcaaagcaccctcctcaagttctggcatctaagctgacatcagaacacagagttggggctctctgtgggtcacctctagcacttgatctcctcatgcagtgcatggtgctctcacgtctatgctatgttcttatggtctttaggtaacaagaataattttctttcttttccttactatacattttgctttctgaaattcccttctcgccaatccaggtgaatgtcagaatgtgatttgacaactgtccaaagtactcattcactgaggagtggtaaggccttcgcccaacctgccttctctgggaatatactgctgcctgaacatatcattgtttattgccaggcttgaacttcaccaaattaatttattagggtcaacatctaaatattagaactatttcagattaatttttaagtcgtatccactttgggtactagatcaaattgcaggtctctgcttctggcttgagcctatgtttagagatgatgtgcatgaagacactctttgcttttcctttatgcaaaatgggcattttcaatctttttgtcattagtaaaggtcagtgataaaggaagtctgcatcaggggtccaattccttatggccagtttctctattctgttccaaggttgtttgtctccatatatcaacattggtcaggattgaaagtgtgcaacaaggtttgaatgaataagtgaaaatcttccactggtgacaggataaaatattccaatggtttttattgaagtacaatactgaattatgtttatggcatggtacctatatgtcacagaagtgatcccatcacttttaccttataggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacaggtgagcaaaaggacttagccagaaaaaaggcaactaaattatattttttactgctatttgatacttgtactcaagaaattcatattactctgcaaaatatatttgttatgcattgctgtcttttttctccagtgcagttttctcataggcagaaaagatgtctctaaaagtttggaattctcaaattctggttattgaaatgttcatagctttgatagtgtttttcagaagaccaaatttacagtgggagccttgggcttttgttttttaacagctcttttttgttcctgcttcagtggcctgacctccaagttagcaatcgccaggttgagaaatgctttgcgagacataacagatgctcctgaaataacaaacacttggaatcatgaggtagtggaattgaaaatagaaagtgtagtgattgttttttgttatttggatgggatgaacaatgtcagattagtctgtaactatttttttttaatgtcactctgatttggtcacaaaggatctctagtctcattgccttagtatcattctacgaattagaatgtgttactgtgtaagagcacttcttgtatatgagagaaatagcaacagttccagtttaaagtgatataaatggaaaccaagaaatgtctttactgggaccaaatctggacagcatttactgtatttttgctggtattttctctagtctttccgggtatattcacatttaatgatcacttttctccctttgtgctaatggacactgaatccattccactaccatagttcttgctaatactactctactttttacacaaaattaaaatgccaggagcacctccaggtagactgactataaatctagactgaaaaaaaagcttgtatttcttaacagattaccttgtggaacatttgctcctttcaactaatgaggcactaaatattgtaactgctcaactggtgcttttaatttatttgtctagactttgtcatgttgccagaagctttatcctggttggagttttgaaaacagtattgtttcttcagaaagaaaaaagggattgtcagatgatctaaaaataaagaaacactggaaatacaagtatcccaaggtgatagcattaggcaagataaaaatgttgaaaagcgaaaaagaactggttgatagagaagtgttgttattcagtagaacctaagtcttgtggtcccatttttaatgaaaaatggtgaattttttggtttttattgttcttgttcacacaaatctgcccattagaataagccaagccctaaaaattaatttcagtttcactgggaatcctttagtttatctactatgtagtagagaggttttgttttattgcatgtttgacgtaggaacgtatatatgcaagacatggaggaaaaccaagtgggccagagttttgaaaattctttatcttttctttctgccaaagtgagtctcccaagtttgtcttttttttttcatttccactcttctatggtttctagcattatataaaccaaacaaaaaaaatacgttcagagattccttcagaaatgctggatgatcttgatatcgatgcttttcatatatgtgtttatgatgctggtttctggggctggctctcagtatcacaaagatgtctgtaaacagaatatgctatttcttctttgtgacaaattttgaacattatgtgaatgtccaagaaagagcaaaagagggcaaacttctcatacatttttgatgtcgaaaccaagagacgcttttattttcctaacttttctttgaaagttcaaattaagtaattttatcctgtcctaaagtttaaaaagaaaaaaaaaaggaagaaggaattaaaaatccaaagaaaattatgtttgtttgcttttctgtttttttcttccttccaactccgagactttgcaagggcatagttctgaagatctctgacactgagacattagagatctctgtatcaatggatcatttgttttcagacatatgaaacaggaactttgaacaagaaatttcccctctttttctcatagtgatcctgagacatcagctgtggaatcacaacacgtcattagttttggcaggtccttgcaggtgttttgttttgttttattaatgttcttccctcctgtagctagacagcaatcttggagaatctgccagcttggaagactattgtgtaaatttcaaggtggagcctcctttaatttgttctgtgttacctgtgagctgtgaggtcatgaagaggagacaatgaggctaatcatgagagccccattggtttaggcaattagaacaacaagatctaaaatggtttattagccttgaattgtgttaagcacataattcataaaaaacagaaaaaatatttttaaatgtatgtctaaatcttcagttacaagtttgaaaggtgacaaactattctgaggaaatgattaggcctattcttgcaacgagtctttatgatctgaaaagaatctatgtccacacataactcccacctcaaagatggggcatcttttgctctgggagatatcaaatgcgaccaaaacaagtgtttgtagatttgaatgatgattcagcagtgtagcagttctcactcattttataataattaacaacttaataattaattattaaactcctacatgcttaacattataagtatgataacttctgtggttacataaaagatatacatagcacttgtccttgatctgtcacagtgaggtcccaatccaacctatgagcttcaaatgaaaagttcaaaattacactcattgtcataagtcagagatcaaaggaagaaaggatttaaccaaaatgataaattaaatataggtgattaaatatagtcatggttcaaggcatgggccagttagggagtgtgatgtgggtaattatgaaaggccagctcccaagccctgttgttgctactcccccacatcagtcatccttcctttttttctacttctactgcagtgccttcctcatcttttcccttgcatccctccattatatgagtcatacaaattagacttttcaaagcaacattaacattgtgtgaatttggggtttttgactaatcccaacattccacccccacattccagtcccacatgggatttggagccttgtttataaacctggcacttctaatatatcttatcttagagtaatccttgtatttgtttaatttccacttagcattgtaaatacttgcaggtatcctagttaagaaagcaaggtttaaacacaaaatcatcaccaattaaagcaggctagataaagaatgtaatagaaatgctagataaaacagattttttcttactaagttttctgtcccttatagagtgcataacacaataacttgcttgataagaattcaatgtacattgttttgtgctgaatcactaaatgcttgatttctgtaacaagagattgtggttccatcagtatctggattttagtctgtgtaatcttaggcaagttatttgatttctctgtgcctctgttttcttgtctgtaaaatgagtataatggtagtaactaattcattgtgtttttgtgaggattaaatgagttaataactagtactcctccctggcacatagtaagtacaatatgctgtgctgtggtggttgttattattttttatagttccttgagcaaaagaaataatgtccccatcttagtataatattggaggtatataccatagaagtgaacaaaagaatatagtttcacaaagaaagtgataattaaggcggttcataaagggtcataaagcttgtagattttagaaatgtgggggcatgaggatgtggagagggtattccaggatgccagacagggagattatggatgagtactaagatgagaactagaaaaagctgaggggcaaaaggtcagaggaggccacaagttagggagtattaggaaaaagaagttaatacttgacaagtgccaacatggcttcacgaggaatgggttgggcctttttgagtgaggaagaggctggtgaaagggtggtggaggacactgctgctgctgatggcatggggtgtaggtggcaggagaggcagggacatgagctaggaaactctccagctatgaagtgatgagtctggagtaatataaggacagtaggggtggagtgctgaacttaagggaggagagaaaaataattggtatggaagtaggtacaatgcaattttattatttctgagcctaaaaatgtgaaatttttgattatttggtcagaccagggaagtattttcttttatgctatctctgaaaatgtatacactaaaaagttgtagtataaaaaggttgtaaagcattaagtaattttagaggaaacaataatttggatattttacatgcaatcatttatatgcaaatatatgtaaatattacaaaattattctctatttgttacaaaccttaaatatttttgactgaggaatattttattcatctaattatagctactttgttctaactaatagatattcttgaaaacaaagcaacacttttttggagacagagtcttgcactgtcacctagacttgagtgtgttaccttgaactccagggctccagtgatcctcccacctcagtctcttgggtaggtggattacaggcccacactaccatgcccagctgtattagtccatcctttcattgctataaagaaataccggaaactgggtaatttataaagaaaataaatgtaactggctcacggttcttcaggctgtacgggaagcatagcagcatctgcttctgaggaggcctcaggaagttttcaatcatggtggaaggcaaataagaagcaggcatgttacacgacgaatcaggagcaagacaaagtgagggaggaggtgccacacactttgaaatgagcagatctcatgagaacagcgccaagaggatggtgctataccgttcatgagaaatccacccccatgatccagttacctcccaccaggccccgcctccaacactgggaattacaattcaacatgagatttgggcagagacacagatccaaaccataccaccagctaataccaaaaaaaaaaaaaaattttttttttaagacatggtcttactatgttctacaggctggtcttaaactcctggcctcaagtgatcctcccaccttggcctcccaaagcactgggaattcagacatgagtaacagtgcctggccaatacttatttttaaacattctctaccataaacttaggatcttgatttgttcacattgaacagatttttattatacagattgaatttataagaaaatgttgcagacattgtcaaaaagggacgtccaaaccactgtgatatttataagcatttgggccacattttgatagaactatacacggagtgtgtgtgtgtgtgtgtgtgtatatatatatacacacacacattatttatatatatgtatatatgtatatatatatatgtatttatatatatatgtgtatatgtatgtacacattatttacctacctactgtgtgagtgtgtgcatatatacacgcacacacacacacacaaatatatatatttcccttctgagacaaagccaaacagcactgtatgcttaaagaaaaacagtcacacttcccacttatgtaatttatattacatccagtcaccacaccagccaaactgctttattgttttttgtttgacatccaatgctaaagcataatgcctgttgcagtgaaatatacatgagcaaccctgagaactcaatatagcctcacgtgttgccactgagttgagttgaggagtcaagctgtagcaaaaaggtttgtcaccgggtgagtaatggtgctcttatttttctctgggtctcaagaagtgctctttatgacatatatggcattaaataaatatcagatatttgcacatcctaactttcctattggtgaagtttcttaaaagagagataaagggccattgtgtgattgatagtttcaggtatatttttgctgcacagtcagtccgagtgtaccacgtagggcaaaccacgtaacttctcagggccttgactgtttcatttgtaaaccagagaaaaggacttgggtgacctccaaagacctttcaaatttggagatgagtttgtggaaagttcaaacagtttagaaaacagaactaagacacccactggcacccctggaagcaagagagtgccaggtactatttgtaatacaggaatgaaatacctaattgtatgaaattgaattctaactgaaccagtttgttcagttaaattttttttttcaattagagtgcttacttcagtatctaacactagacagtaaactgtagacaaaagacctacagaatttctgaatggtatcaaattcaccacacttaaaactttgggatgtctaatttcaaccaacagctttctttcttcataatgttgaatatatgtgtatctattttagctaaatttaatatatatcaatatactttgatagatattttatataaactattagactatagtattatgagtaaaagacccaccatttcccaagcaattataaagaacgatcaaaattttaatgggttgttagtattatttctttaaagattgtgatactgataaatatttggccacattttaatagaattatacatgggatgtgtgtgtgtgtgtgtgtgtgtgtatatgtgtgtgtgtatatatatatggcagtagagatatatatatctacacacatctagatatatatatacatgtatatctatatatacacacatatatctgtgtgtatatatacatatgtatatatacctacatacatatgtacatatacatacatgcatatatctgtacatatatatatagtgtgtgtgtgtgtatatatatatatatatatatattttttttttcctgagccaaaacaaaatactaggttgtaatagctgttctttcagaaggaagaaaaacaacatgtgctgaactctgagtttgatgtttttgtattttacttcctattttcatatcagtccatttatttattcaggaagaatttattgagcatatattatgaacacagcttttgctaaggacagggtatgcagcagttatggcctagtaggagatatggatgttaaaaacaaaatgctcacaaatgcacatataatcttaatactcattgtaagctatgaaagcagagtgtgagtattatgagaccatatgttgggagattttatttggtattgaggatcaggaaagatacccctgaggaagtgatatttaatttgaaacctaaagaaagcagttggccatgggaagaaggtagggaatgagattcccaagcaataggaatccaatgtgtgaagaagctgagggagtgaaagaaagctagtgtggtggcaggaagaaagagaagagaatggagaagggcactaaatgagtcagagaagtaggaggggctaaaccatgtagggtcgtgtaggccatcttaaaggcctgagtgtagtggaaaacctttgaaggtttgttaaaaggtcaatgaaatgttctaatttctgttgtagtgaattgctttgattgctgaatgcgaatggatgggtagagatgcaagagtgaaagggaagaaatcaattaggaggctcttgccctgctccagataggactgataattaattttatttgggaagatcagggagaaagataagtcatgaatgactcccaagtttctggattgaagaaatgaaggtaccatacactgagatgggaaagcctaggggtagagtagctttgagaagaaaggtagcatttccccatttcataaaacatggaagaacaaagaggctggattcctgtttgtagacataccttccaggccagaactgcattactacaacatctttgcaagccacattgcctttcataactctgtgtcagtgttgatgccgtaacatctttggccttccccctaccatcctcccgcagtcctccatgataatgccattattccgtttcaaattgtgtgcttccattggatgtgtgagtctccttgaaagttataatgaggctgtagcccatatgaaatgcttcaactcaggtcctgcataggaagaggaagctaatctctccaggaactgagcctgtggctagagggatggataattgtttaaataaagaatatgctgctgagtactgatgggctctttatgtacccatttggctgctgctgcccaacctttaatctttcctgagctttaaataggaaggaaaaaatggtccacaaaggatttgagccattttgctgtggtgatgaggagcacgggtttagagacaaacactcctgtgtttgaattccagctcctactatctcctagctaagtgaccttggacaagtcacttaccttctccaacctgctgtttcttcatgtacgtaataggatttacctcatgaggttgacatgaagattgaaagaggtaacatatagaatgagcctgtcccaggacatggttcatgataagtctgccataaatgggagctatgtgtcccacccttttggaggagataactgttctgtagcaggtaatatattgtttgatacttggttaacccttacaattatcatttcctgttcttctcaataatgctagaaaccttttatttaaagaaccacaatataaaatgaaaaatatataaaaaaagcaaatggaaaaattctattggcaaggctttttaactttatatactaaataaatccaattgcttaaataatgaactgactcaagttctcagcactgcttcttgtttaattctctttagtttttcagaattctccaataatgacctttgtctactctcttcagtttattcagaaattacttttatttacatagaagtttggaagtggatacacaaacatatccctcacatatcttatgatcctatgagtcatatactcatctcttatattccctctgtaaagcaatgtaggtacctttcaggaaggtgatttttatgtaggttgagaaatatcagcatggaggtcctagctgacctctctagagagtttctgagacatttgacaacaactttttctttaagtcatcagttatgccccggggtatgaaatttctaacatgatcctcagtaaacttggctgccttgctgaggatactctccatctgcctgagagacacagacaccattaattgggaattgacttgacttgtgtggttccttgtggaccagatggccactaaatattctcatttcaaggcaattggtaaaaactacacttcaagaaatttcattcttaattccccttagtggatgttattaaccaaaggcaaaagaaaaaaagggtaaaaaaaatattctaaatgttaatatcaaaaatattattttcaattcaccccaggcacagagaactaagtattattattgctattgcaccggcattccccaatgagacagtgattttcttttaagacatttttaaataatataggcagaattaagtagacggtgatctggtaagtagatgtttcagggtaacagctgtgcaatgctccatgcagggaattagattgtcattttattccttaccaggaacatacattcagttaaacaattatttgacttctgctcttccactgatttctaagttgaggctctctcttgtgcctgtctgatcagataagtagagttgtgccttggtttatagatgagataaatgtgtatttgaataagcataagttaaagaaattttaaaatcccttaggaagctaggcttatcagagaaatccaaggaaatacattaacaaactaggaatttgttctaacaggttaattataactcataaacttattgggtttttttaccttttaattttatattacatttgcttataataaggaatattgctaggaataaaattttttaatattctacaattaacaattatctcaatttctttattctaaagacattgggattagaaaaatgttcacaagggactccaaatattgctgtagtatttgtttcttaaaagaatgatacaaagcagacatgataaaatattaaaatttgagagaacttgatggtaagtacatgggtgtttcttattttaaaataatttttctacttgaaatattttacaatacaataagggaaaaataaaaagttatttaagttattcatactttcttcttcttttcttttttgctatagaaagtatttattttttctggaacatttagaaaaaacttggatccctatgaacagtggagtgatcaagaaatatggaaagttgcagatgaggtaaggctgctaactgaaatgattttgaaaggggtaactcataccaacacaaatggctgatatagctgacatcattctacacactttgtgtgcatgtatgtgtgtgcacaactttaaaatggagtaccctaacatacctggagcaacaggtacttttgactggacctacccctaactgaaatgattttgaaagaggtaactcataccaacacaaatggttgatatggctaagatcattctacacactttgtgtgcatgtatttctgtgcacaacttcaaaatggagtaccctaaaatacctggcgcgacaagtacttttgactgagcctacttctctcctcactggtatggctccaaccatcaggccctatcttggtccatttaggctgctaaaataaaataccaaagactgagctgcttataagcaatctttggaggctgagaagtcaaagatcaaggtgccagcaggtttgctgtctcgtgagagcatacttcctggttcattgatggtgctttcttgctgtgtcctcacataatggaaagggcaagacctctctggtgtctcttttacaatggcactaatcccatcatgagggctttgttctcatgacctaatcacctcccacatgtcctacattctaatactatcaccttgggggttaggattttaacatatgaatttgaggaggtggcgggggggacacaaatatttagaccatagcatttcactcctgacctccaaagttcatgtcttcttcacatgcaaaatacattcattccatcccaatagcccccaaagtcttaacttgttccagcatcaacttacaaggctaaagtccaaggtttcatctaaatatcagctaaatcagcacaaacagctaaatcaggtagagtgggacttaaggtgtgattcctctttaggcagattgctctccaactatgaaattgtgaaatcaaacctattatgtactttcaaaataaaatggtgaaacaggcacaggctagacagtcccatttcaaaaaagagaaatagaaaagaaaaaaggagtgacaggtctctataagtctaaaactttaaggcttgagaataatttgctttgctttgcctccaggctcactggggtggtgtcttacctctggacacactggggtggaggctctatcctcatggatttgagtgtctcattctttgtggcaggtctgtgctccaatcccacacctatggctccctgagtgtgcaattgcatgcctggtggttctactggtctgggattgcataggtggcccagccttcatagctccactgggcattgccctaatgtgggctctatgtggtgacctcacccctgggcctctacctgggccctgtgactccctgggttcttgaaatctaggtggaggcagccatccccctacagttgtgctgagtgtagtgcatgagtgctggggtctgctagagctatacctagggtggtggagatgtatggcaatggagtatggggagctgatatggtttgggtgtgtccccacccaaatcttgtcttgaattataatttccataatctccatgtgttgagggagggacctggtgagaggtgactggatcatgggcatggttttcccatgctgttcatgtgatagtgagtgagttctcacgagatccaatggtttcataaggcagttttccctgctcttgcaccctctttcttgcctgtcaccatgtaagacataactctttcccttccgccatgattgtaagtttcctgaggccttcccagccatgtggaactgtgagtcaattaaacctcttttctttataaattacccagtctctttacagcaatgtgaaaatgtgctaatacaggagcaaagactgcagtgtgaggtggcaatgtgaagtctgcaatgtgaggtggcacggggcagttgtagcccctcctttgaaatctttcttccctaccccaggcctctgcactctgaactatgatgggaaaggcagcttggaagatctccaaatggctttggagtcattcttccattgtcttggactataaattctggcttctgtttaggtggctgactaatatccccactgtctgaatgcatagcacctagtttctgttgagatggctagtccatagtaatttacttatcaaatttggccacaccctttgtattctctcctgagcaggctttctcatctttcacaatatggataggctgagaattttccaaattttgaagttctgcttcccttttgatcaataattccattttaaagtcatttctcatcttgaattttactatgagcagtcaagagtaactaagctgctccttcaactttgcttggatatttcctcagtcaaacattcaatttcattgctttcaagttctgccttccacaaaacactaggacacaaacagctcagccaagttctttgacattttataagaaggatagcttttcctccattgtccaataacatgttcctcatttccatctgaaaacccatcagattggcctttaccgtccatatttctgggaacattctgctcatgaccacttaggtattcggtaagaagatagtagctttctctatagctctcctcctctctggagccctcaccagaatggcctttaattgtccattcacagcaatgtaggctttttctagcatgtacctgaaaactcttccagcctctactcattaccttgttccaaagctgcttccacattgagtatttgttacagcagtacccagatcccagtaccaatattctgtcttagtccattggggctactacacgatgtcttataaacaacagtaaaatttatttttcacagttgtggaggctgggaagttcaaaatctggtgccagcagattttgtgtctggtgaaggccttcttcctcacagatggctgtgttctcactgtgttgttacatggcagaagagtgggcaggctagctctctgggatgtcttttataagggcagtaatccaaatcatgggtttagggtagagccctcatgacctaaatcacctcccaaaggccccacctcctaataccagcatctttgaagttaggatttcaacatatgactttggcagggggacagaagctttcagtttatagcaaaccctataggtagcactactttgtcctttcctaatcaatttgcgtcaatgaaacatgaattagaagagacctaggcgactccactatactgggattattcccagtataaattatcatctctccacaccttctcatctactccctatctgagttctgaagctctccactacaagaaggaggctttggtttgacttgatatacttctctgggaaacaggtttagcataaaacagtgatgctcattctagaacacctgcaaatgacaatagttttctttcgaagtcgccaggaatcgtctgcctttgggtatgtggctgtgagcactgccgggcaaaatgccatatgacctagatgaggcatatgccatcctttgaagccattaggacattatataggaaatatattaactaaaatggaataaaattttctaaataacaccttatgtttatccaacaggtggttcattatacttgagagcattatacagaggaatttgatggggaggagagctggagaaattctcgaaattctgggtttctttaacagaatactctagctataaacttataattttaaaaaataagcattatattaaagaaaagggaacataaattattttgttttattaaacttaagtccaaaggtctggattgtggcagaataggatcaggggacctaaaatgttgagcctcaaaggtcttcttagagaacaactgtattccactattagcgcttttggtccttttagcccaatttctgtttatcccaaatgttcttcccttttctgccttccttcacagtggaccctgccaggagctttgaaatgcctgtgagtgttaaacacttacccattgagtgcccaaccttaacatgcccctaataaaatgtacttagattaaccgttttcattatcaaagtttccttattacccaacaaacacaggcgctttaaagaaaacattaactaaattgcaagtgacacattttaagatctttgatatgacttcagagaatgcactataggaacacaatgcaatgggagggaaacttgggagggaagacattagcctttataaaatctgcaagtattgccaaatcaaaataaaatttacaggaaagcaggatcataaatataatctaaaatcttagaacctgtggttatgattttaaatactaatacaatgcaaaatttttacctgtttaggtttttatttcatcagttcatatttaggtatatacttttactgttctccttttttataatttaccattcacaaagatgatgatgttagtctaactttaatgtcatgagtgctttgagtagtagtgctaagtttttgttgagtagtagtgtgcttttttgattagtagtgataggtttttgatgagtaagcctgctagcagcatacaaacaaacaagcaagtatcagcctagagaagcagaaaaggcatttgggtttcaaagtcacaaggcctaggctttagtctaatacagctgataatacaatttgtccaaacaggacatttttgggtgtgtcaaacactaaactggacaggacattatgacaaaagtgcaaagcaggactttccggggcaaaccaggatgtatgtcatctcactgagtcctctctttgtccttgccatgactagtatctctagaggtaaatgaacagagtaatgacaaatagccagacacctgaatcttatcccaacagcacctcctacataattccccattatcccaaatggaaattaaaaatatatacagtgataattccaggccaagaaatgctttatttctagcttggacttggcttccatgtccagtgtagaatcttatccttgctgatctggactgtatctcatgaagccatgacttgtacctagttactagctggaaggcttagaacaaaagctggtccagagagcctcctttttccttatttcctgggtccacacctttaccatggcagtctgcctatcatttgatggaggaatttaaagcaagtccaagggaagggaagagagtttctaaaatctagaacttggatagtttaatttacctatcccaaaacagcttaggcccagacagcttctctccaagattggtgccaaactgaaattaccagctgtgtagaccaaagagaatttcaaaagaaactgaatcccaagagaaaaaaaaaagacttctggcattgtggcccaataaattggtaggattgttgtgacttttcaagtttacatgtaaaatgggcccagcgcagtgcctggcaaatatgggtactaagtaaaagtaactataatcatgtttttttaatctggacttcacttggtcatcctttaaatggtgtctgacagaatcctagttcttgtctcactttacttagtttccctgggaaatttcatgtgtccttttggctttaattaatatctctattttgatgacctccattatctgcctattcccagagctttccacctgatatctcagcacatgaaaagcaccttatgtcaataagtgagttccttccctgccccaccacatacctgtcctgtgttcctaattccactgaatggcatcccatcctccagtttcccaaggccaagacctgggactcatctttcactctcaagttcctccacgggtacccacatgtcacatcctgtcaatgctgtccctggggagtatctgaaatatattcacttttcttcatttccacctgacaccactattaacacttgcacaaatttctgaggttcctggctcatttccctcattgacccccaatagttcattctgctctttgcagctctggtgatctttccaaaccccacatctgatcacttgtttcttcccttcatatggctccttaatgccttctggactaagtccacactgcttaaggtggcttaccaggtccttcatgattttgtctttgtttggctttctacactcactgcccaacttccccttacttcccatgattcagttatactgaatttctttggttctctaaagcacatgtgctttctgttctgcagaggcttttttgttcacttgctattctctacctgggaaactcccccagcccttcactgcctccttctaccatctttcaggcctctccttacacatcacttctttccaaaaatctgccttgacactccaggtctcggtttcctaggtgtaccctataactccacccctttcatagcatttctcactctggctggagatttaccttttaacttgtccatgtcccccactggagtggaagttcctggaggtcagggattatatcctattaattgttgtatttccagtgcctagagtagtcttgcatacatggatggtattcaataaatattggttgaatgaataaggagttctttcatttcatatgtaatagatcatggaaatagccttgtgattgatacacagcaggtattaccatcctcactttagaatgaggactcagagccttgagatgtctgagggccttgactgggacagctggcagatgcaggagcagagctgcatcacccctgtgggctatctcagggttgtctgtaatctaagtacaatgtctgttgattttggactgaaggctttttgggtaattgtttgctttttcaatacttataaaatagtttccatccttactcattgatagtaaggttagttattttagaaaacaagctaaatagcagaaatagtggccttttaagttgaaaatttaccctgaaaaatctacagagtagcaaacagagtatcaaaaggagttgactgtatctatttttataactgccacttatggattattcagtaaaaccacaattcacttttatgattttttttcatgtttctctgtcacaagagcaaactcttgctccataataacattccagaatacagcaatagcaaaagtcaacattttgaatcctttacaaactcttagacatttttttttttttagtttaacatgttacaaaacaaaatttcttcttttttcacagcagtttgggaagtacatactatttattagctcatcagcatgaagctggaaaattctttttcctaaagttctttatatctacaaactgttgatgttttcatttatttatttttaatgctacgttgtaatgaaaatcattggaaaactttagattctagtaattttgaagtcttcttagtttggacaggactgagctaaagtttgtactttttttaatttattgaaaaatggtttctaatgatagtattaacaagattatattgggggcaggacgcagtggctcacacttgtaatcctagcactttgggaggccgaggcggttggatcacctgaggtcaggagttcaagaccagcctggccaacatgtagaaatcccctctccactaaaatacaaaaattagctgggcatggtggcaggcactgtaatcccagctacttgggaggctgaggcaggagaattgtttgaacctgggagtcggaggttgcagtgagcccagatcgcaccactgcactccagcctgggcaatagagcaagattctgtctcaaaaaggaagaaagaaagattatattggggatatatatgtgtgtgtgtgtgtgtgtgtgtatatacacacacatatatatatacatatatacatatatatacatatttaaaggataaaggattctgctgccacagatcactaaatcagatgatctctagcaatttcctgtttgtttgttttttgcccatagtgcttatctctttgaacagtaattttccacttactatttttctccccttttggaccataatttcctttaaggcagagcctcctgttactcatctttgaatctggggtctgtcagagtacctagaatttaataaactctcattaagagccagttgaaagaatatatgactaagcagtcatttacatccaaaagatccgtaggagaattcttatcagcacatgtgattggtaacaataactttgtacttttcaaaaacaattactaatctatcttgctttccattatctcaccaaaacctattagcatgtctggcagaaaatagatacttaataaatttcttaaatgtttactgacttcaattttaagttttattaactatgttgacttttctctaatgaagatgattctaaaaagctttttactatacttcacagtgaataaaacagtgagataggaatattgcaaaatgtcccctgtgttggtcagtcttagtgtcattcattttaaaaattctgttctctaaatattgacagttatatataaatttatgtaattgtttacttctaataaagaatttcatctggggaaaaacatactttgctcagctctttgccacaagtgcaaagtctaagacagtcaaatagctttcctagtacggccttaggaacttagtatatgactggtgtgaatctagagggagcatactgcattctgaccaaaatctccaccctgttactatggccatcactaacttcgcagtattgcagtacttcctgctagcttagttcccaaggcaacttgtgaaggaaaatttttacaaagctgttgtcacacaaaggtagtgtttcagttcctgagcccatgtccttggagttgcccaggctccaataatactaataattactgtacattaggtacttaccatgtgccatattctgtgggagccgctttccacaaattatctctggtaatccttgtaacaaccctttgacatcaatattattattttctccatttttttacatatgagataaatgagacttaaaataatgtgcctgatatcatcagcaaatgagctgaggagggcagattcaaagctgattgtgtttgactctagagctgcagtcttaagccagaccttttcttgctggttaattttactgaaaaaaaaaaaaaaaaaaaaaaaccctcaaatactgctgattgatctaaagtactaacatttctatcagtgttagggaaattttaattttataatttgattttgtgagaaatttatagcatcttgaatactcacatgcaaagtgatatgtcttagataacattttacaatggcagagcttaagccagtgctcagtcattcattcatcctcaagttttgattcatttatcattcatcaaaactctgttttgtttggccacccacattctaggagctcagtacatatttgataaatgaatgaattgttgaggttgacagttacccaggactggcattaggaacacagagctgaagagcacgtttttaccctcaagaagcttacagtctaacgagggaacttgcacaaatactactatcactaggtgcctggttgaatggcttaagagatgatcagggatattcagaaggatatgtcaggctcagcaatggcatcacttgagagcatcaaggtgtttagggaactacaagatgtttggttctgctgggaataagagtgaagggggctccatttggatgcctcatacaccaggtgagagatcttagattttattccaccaggaggagaactaccataggatttaaaacagaaatgatatggtcaaacctacatcttaggaagatccctggggtgtttgtatggtggacttgcaatttgactaattgagatttgtaggatgattcttaagagatgatgatgacccagactgggatcactataatagagttggtaaggaggagaatgatttaaaaagtagttggaagaattctagggatggagataaacatttgaaaattattaacttataggtggtcatcaataccctgaaaatgactgggatctcagaggagagtctggagagttggaaatgacaaagactaatattcaagggggcaggaagagggagagttgttcacacatgacaataggaagaaatggccatagagtgtgtggtttctctcaagccaaggaatagatgttttaagaaaggaaaattcttgtggtgggaagcagtagagatgacagatacacattaatttcttgagatttctagatgactaaatgggcagatgttgaatgatagctaaaggagaacccagaaacaagggagggattttgtttttgttttttaaaaaagatagaccatagcagcttcatagactgaaacaataaaaaagttgaaggcacaaagaaagacacaggtcctctaactccctgcccagtgccctttattcatattctcagcacttgtatttctaagttttatgtttgagtcttcggggatacatcagagtagtcccccttgtctaataaatgtgtttacatttcctgccataccagaaacccttctcaaactttaatgaatttctacaaggtgagattactttaatgagaaaccaaccaaggaaaggagtatcatctgcaatatactttcaaatgttttttgcttgtttgtttcttgtccagctaaaaaaaaaaaaaaaaaacaagccattggtcctaacacaactttcatattctaccccaatatcaaagaggcttaaaatctcctggtcgtgtgatgggcacacagttaattttttgtgaacaaacacagtgttatgggccatttctgaatttatctctgaaatcataagattctttctgagccattatctcattctatattacagtcaggtggagcccatcttacctcctcatactaaattctagacttctcaagggcaggagacaatcatctgtatatctctttggccttcatacactcaggagtacttgccaaaaataaacatttaatgcacatttatttgaataattgataagatccaatacttcaataactttgtcatatttttatagaatgggtttctatatctcatttgcattttcaaactttacttttactgtctagctttaaaaaaaaagcctttgactctaatacagccctcatattctaccccaatatctaagaggctttatatctcctagtgttgtaccactattttaactccagtattttttacttcatagttttacctatttgttacagttagtttttatgaattcaagagatgaatagcaattttccatatgtaatttaaaaaaccccacagttgactattttatgctatcttttgtcctcagtcatgacagagtagaagatgggaggtagcaccaaggatgatgtcatacctccatcctttatgctacattctatcttctgtctacataagatgtcatactagagggcatatctgcaatgtatacatattatcttttccagcatgcattcagttgtgttggaataatttatgtacacctttataaacgctgagcctcacaagagccatgtgccacgtattgttttcttactactttttgggatacctggcacgtaatagacactcattgaaagtttcctaatgaatgaagtacaaagataaaacaagttatagactgattcttttgagctgtcaaggttgtaaatagacttttgctcaatcaattcaaatggtggcaggtagtgggggtagagggattggtatgaaaaacataagctttcagaactcctgtgtttatttttagaatgtcaactgcttgagtgtttttaactctgtggtatctgaactatcttctctaactgcaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtgtgagtttcagatgttctgttacttaatagcacagtgggaacagaatcattatgcctgcttcatggtgacacatatttctattaggctgtcatgtctgcgtgtgggggtctcccccaagatatgaaataattgcccagtggaaatgagcataaatgcatatttccttgctaagagtcttgtgttttcttccgaagatagtttttagtttcatacaaactcttcccccttgtcaacacatgatgaagcttttaaatacatgggcctaatctgatccttatgatttgcctttgtatcccatttataccataagcatgtttatagccccaaataaagaagtactggtgattctacataatgaaaaatgtactcatttattaaagtttctttgaaatatttgtcctgtttatttatggatacttagagtctaccccatggttgaaaagctgattgtggctaacgctatatcaacattatgtgaaaagaacttaaagaaataagtaatttaaagagataatagaacaatagacatattatcaaggtaaatacagatcattactgttctgtgatattatgtgtggtattttctttcttttctagaacataccaaataattagaagaactctaaaacaagcatttgctgattgcacagtaattctctgtgaacacaggatagaagcaatgctggaatgccaacaatttttggtgagtctttataactttacttaagatctcattgcccttgtaattcttgataacaatctcacatgtgatagttcctgcaaattgcaacaatgtacaagttcttttcaaaaatatgtatcatacagccatccagctttactcaaaatagctgcacaagtttttcactttgatctgagccatgtggtgaggttgaaatatagtaaatctaaaatggcagcatattactaagttatgtttataaataggatatatatactttttgagccctttatttggggaccaagtcatacaaaatactctactgtttaagattttaaaaaaggtccctgtgattctttcaataactaaatgtcccatggatgtggtctgggacaggcctagttgtcttacagtctgatttatggtattaatgacaaagttgagaggcacatttcatttttctagccatgatttgggttcaggtagtacctttctcaaccaccttctcactgttcttaaaaaaactgtcacatggccaggcacagtggcttacatctgtaatcccaatactttgggaggctgaggtggggggattacttgaggccaggaattcaagaccagcccaggcaacatagtgaggccccatctgtctttattaaaacaaaacaaaactgtcacagcttctttcaagtgatgtttacaaattccctatggtttagtcacaaggaagttctgaggatgatgtatcacgtcatttctgttcaggcttttgagcctcctggaggtaaatggtttccttactgaaggcttgttattaccatgattatcactaagcttgaagtaacaaattaggggggcagactcacaacctcttgccctgccatggacaagttcaagaatctaagtaaagtcctctattgtctgatcttggatttgctcaacctgaacaagccaaggaggtgtattaaactcaggcacatcctgaccaatttggaattcttaagcttcagatcactgtggaagaggctcaactctttatggtgctgtagacttacgctcattttctaggtaatttataagggacctaatattttgttttcaaagcaacttcagttctactaaacctccctgaagaatcttccagctgctgagtagaaaatcacaactaatttcacagatggtagaacctccttagagcaaaaggacacagcagttaaatgtgacatacctgattgttcaaaatgcaaggctctggacattgcattctttgacttttattttcctttgagcctgtgccagtttctgtccctgctctggtctgacctgccttctgtcccagatctcactaacagccatttccctaggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttagagagcagcataaatgttgacatgggacatttgctcatggaattggagctcgtgggacagtcacctcatggaattggagctcgtggaacagttacctctgcctcagaaaacaaggatgaattaagtttttttttaaaaaagaaacatttggtaaggggaattgaggacactgatatgggtcttgataaatggcttcctggcaatagtcaaattgtgtgaaaggtacttcaaatccttgaagatttaccacttgtgttttgcaagccagattttcctgaaaacccttgccatgtgctagtaattggaaaggcagctctaaatgtcaatcagcctagttgatcagcttattgtctagtgaaactcgttaatttgtagtgttggagaagaactgaaatcatacttcttagggttatgattaagtaatgataactggaaacttcagcggtttatataagcttgtattcctttttctctcctctccccatgatgtttagaaacacaactatattgtttgctaagcattccaactatctcatttccaagcaagtattagaataccacaggaaccacaagactgcacatcaaaatatgccccattcaacatctagtgagcagtcaggaaagagaacttccagatcctggaaatcagggttagtattgtccaggtctaccaaaaatctcaatatttcagataatcacaatacatcccttacctgggaaagggctgttataatctttcacaggggacaggatggttcccttgatgaagaagttgatatgccttttcccaactccagaaagtgacaagctcacagacctttgaactagagtttagctggaaaagtatgttagtgcaaattgtcacaggacagcccttctttccacagaagctccaggtagagggtgtgtaagtagataggccatgggcactgtgggtagacacacatgaagtccaagcatttagatgtataggttgatggtggtatgttttcaggctagatgtatgtacttcatgctgtctacactaagagagaatgagagacacactgaagaagcaccaatcatgaattagttttatatgcttctgttttataattttgtgaagcaaaattttttctctaggaaatatttattttaataatgtttcaaacatatataacaatgctgtattttaaaagaatgattatgaattacatttgtataaaataatttttatatttgaaatattgactttttatggcactagtatttctatgaaatattatgttaaaactgggacaggggagaacctagggtgatattaaccaggggccatgaatcaccttttggtctggagggaagccttggggctgatgcagttgttgcccacagctgtatgattcccagccagcacagcctcttagatgcagttctgaagaagatggtaccaccagtctgactgtttccatcaagggtacactgccttctcaactccaaactgactcttaagaagactgcattatatttattactgtaagaaaatatcacttgtcaataaaatccatacatttgtgtgaaa

SEQ ID NO: 2 - RNA, 6070 nt, Homo sapiens
  source 1..6070: mol_type = mRNA
gtagtaggtctttggcattaggagcttgagcccagacggccctagcagggaccccagcgcccgagagaccatgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgattgaaaatatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatggtgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgcatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctatacgaaaattttccattgtgcaaaagactcccttacaaatgaatggcatcgaagaggattctgatgagcctttagagagaaggctgtccttagtaccagattctgagcagggagaggcgatactgcctcgcatcagcgtgatcagcactggccccacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcttttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttagagagcagcataaatgttgacatgggacatttgctcatggaattggagctcgtgggacagtcacctcatggaattggagctcgtggaacagttacctctgcctcagaaaacaaggatgaattaagtttttttttaaaaaagaaacatttggtaaggggaattgaggacactgatatgggtcttgataaatggcttcctggcaatagtcaaattgtgtgaaaggtacttcaaatccttgaagatttaccacttgtgttttgcaagccagattttcctgaaaacccttgccatgtgctagtaattggaaaggcagctctaaatgtcaatcagcctagttgatcagcttattgtctagtgaaactcgttaatttgtagtgttggagaagaactgaaatcatacttcttagggttatgattaagtaatgataactggaaacttcagcggtttatataagcttgtattcctttttctctcctctccccatgatgtttagaaacacaactatattgtttgctaagcattccaactatctcatttccaagcaagtattagaataccacaggaaccacaagactgcacatcaaaatatgccccattcaacatctagtgagcagtcaggaaagagaacttccagatcctggaaatcagggttagtattgtccaggtctaccaaaaatctcaatatttcagataatcacaatacatcccttacctgggaaagggctgttataatctttcacaggggacaggatggttcccttgatgaagaagttgatatgccttttcccaactccagaaagtgacaagctcacagacctttgaactagagtttagctggaaaagtatgttagtgcaaattgtcacaggacagcccttctttccacagaagctccaggtagagggtgtgtaagtagataggccatgggcactgtgggtagacacacatgaagtccaagcatttagatgtataggttgatggtggtatgttttcaggctagatgtatgtacttcatgctgtctacactaagagagaatgagagacacactgaagaagcaccaatcatgaattagttttatatgcttctgttttataattttgtgaagcaaaattttttctctaggaaatatttattttaataatgtttcaaacatatataacaatgctgtattttaaaagaatgattatgaattacatttgtataaaataatttttatatttgaaatattgactttttatggcactagtatttctatgaaatattatgttaaaactgggacaggggagaacctagggtgatattaaccaggggccatgaatcaccttttggtctggagggaagccttggggctgatgcagttgttgcccacagctgtatgattcccagccagcacagcctcttagatgcagttctgaagaagatggtaccaccagtctgactgtttccatcaagggtacactgccttctcaactccaaactgactcttaagaagactgcattatatttattactgtaagaaaatatcacttgtcaataaaatccatacatttgtgtgaaa

SEQ ID NO: 3 - DNA, 249 bp, Homo sapiens
  source 1..249: mol_type = genomic DNA
atgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagg

SEQ ID NO: 4 - DNA, 156 bp, Homo sapiens
  source 1..156: mol_type = genomic DNA
gtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacag

SEQ ID NO: 5 - DNA, 14667 bp, Homo sapiens
  source 1..14667: mol_type = genomic DNA
gtgagatttgaacactgcttgctttgttagactgtgttcagtaagtgaatcccagtagcctgaagcaatgtgttagcagaatctatttgtaacattattattgtacagtagaatcaatattaaacacacatgttttattatatggagtcattatttttaatatgaaatttaatttgcagagtcctgaacctatataatgggtttattttaaatgtgattgtacttgcagaatatctaattaattgctaggttaataactaaagaagccattaaataaatcaaaattgtaacatgttttagatttcccatcttgaaaatgtcttccaaaaatatcttattgctgactccatctattgtcttaaattttatctaagttccattctgccaaacaagtgatactttttttctagcttttttcagtttgtttgttttgtttttctttgaagttttaattcagacatagattattttttcccagttatttactatatttattaagcatgagtaattgacattattttgaaatccttcttatggatcccagcactgggctgaacacatagaaggaacttaatatatactgatttctggaattgattcttggagacagggatggtcattatccatatacttcaggctccataaacatatttcttaattgccttcaaatccctattctggactgctctataaatctagacaagagtattatatattttgattgatattttttagataaaataaaagggagctgaaaactgaattgcaaactgaattttaaaactttatctctctgtggttaattgcaaacacagatacaaaaatatagagagagatacagttagtaaagatgttaggtcaccgttactaacactgacatagaaacagttttgctcatgagtttcagaatatatgagtttgattttgcccatggattttagaatatttgataaacatttaatgcattgtacaaattctgtgaaaacatatatataggatgtgcgaaaagtccctgtgtatcatgtgaaatggcttaaaacagaacaccataggtattcatatcagtgaataccataggtagctgaaagtgttttttcctggggtcgccaagatgaatgccaaaagtgatatcattattataaacaatagccagaataggttggtataaacctggtagaaagccttgataaattgactttctctcctcctgacatcctgccacccctttgctttgctgatgctcatttgtccactaaattaaactcaagcaagccctagtaaagtaatagaatttgtggagtcctcattagtataggaagtttccctgatgtgagattagtaattagagatgtagcaaaatgagaaagaagtaatatgcttagatatttcattttctctgaacctgtatatacaaaataggccatgcgtgttcagtaactattcactgcaaggcactctctaggtactttgggggaattggaaattactcacataaggctatggattgtgccatttgtcaaaagacaaaatgacaacaaatttagtttaaagacctcagtcagctttattttctattctagatttggacagtccttcatttcacaaattggagtaagtgttccaataagttgagcaaaggagcttggctttatagacccaaaaaaagggccaaaggaagcagaaacaaagaacaataagagaattggtcatttcaaagttacttttcttgaaaggtggggacaaggagacagaataatagaaaagtcactgattggttaacattggattaagaattaaaacagaggaaactttaagattgaagtttgaaactgacttgtttgggaaatcaggctgtcttctttcttgatttcttagaaggccggataacaactgagttttgctttggtgaacatgggtgactccatttttacttttagtctggtctgttgaggcctcgtgagagagcttaatctaaaacaatgacttcctataatttttgtttgacacatccaaagagggactctaatatttattgagagcttatcatatcttaagtactgtttaaacacttttatttgctattacatttgatcttattataactctaaaggcagaaatgattgcttttattttccacaatggaggaaactgaggttcaattaagtgagtaaggaagcagggatcttaaacccagataccattgctcctctttaaaggtggaagaacagaaaacatggggcaggggaagagagaaagtttctgtcccaggacatgataatctaaaagggaaaacgtaagatccactgaaacctgaggcagatttattgtggcaataacaaagcttaagtttcacagaccttcatttgcctgagccaactttgaaggccatgtatctaattttgtttttataattctataatctttattcttgaaaagagccctccctccaaatttacaagctttgggcccccaaaatccttgaaatgcccttgaataagagatatccaggtaaatgctatgggaattcagaggaggaagcagttagtatcagttggcggagagttaggctattaagagaaggttttatataggaagtggcatttagaatgaagctttgagaactgagctgtgtatttgaacaagtaaaggtggtgttgcagaattttgctccttagttctattaaaaacccgggttcttgtcacatgatccggaaaatttaggcacacagatacattgaagcatgagtagagcaggattttattgggcaaaaaggaaaaaaagaaaactcagcaaatcgagatggagtcttgctcacagattgaatcccaggccaccacaaaggaactgaagagatcgggcttctcccctgcataaggtgcaaattccccatggctccacccacttccccttagtgtgcatgtggggctccagtccacggtgggcatgcccagacaagccttgggcaggttccctcatctgtgcaaaagcatctgatgtaaacacttgaggggtggttcggagattctctgggacccttttattttcttatctgcctaggcatttggctgtctcagtgggtgggaaagggtgctccaggcaaagggcataacatgaggcaaagggcatgcacagaaaacagtgactggttcagtcaggttgggggatgccaaaggaagtaatgggagacaagattggagcaagatagataagagattgtggattttttttcttttttatctatataaatacagagacagggtctcactatgttgcccaggctggtctcaaactcctggcctcaagtgatcctcccacctcatcctcccaaagtgctaggattacaggcatgaggcactgtgcccaacctccaattttggattttgagagctaaagcaatatagtcgaaaactcagataatccaggtagattttgctattaggtgctatttggttcctggtacagagctaaaacccttggaatttcctaagtgataagagctacaggagcatcttttgttatatgtttccccccctagttcctgaaatagctctagagaaatacaggtgaataacatcctttgttattcatatcaagcccctatcaaccataccccagtttctatttatgaagtggcttttgggaagtccctaaagacaggagtggggaaaggctggttgtcagggggatgggttgaaactttcatcttccccccttgacctccagggagggatgagtggctgaaaattgtgtaaaatcaacaatggccagtgatttaatcaaccatgcctatgtaatgaagccacccgataagccttaactggaactttttggagagcctccaggctggtgaagacattgaggtgctcagaaggtggtattccagagagagcacagaatctctgttccccttcccacattcattttgctatgcatctctcccatctggctgttcttgagaggtatccgtttataataaactggtaacctagtaagtaaactgttaccctgagttctgtgagccattctagcaaattatcaaacctaaagagttcatggatacgtgcaatttacagatgcacagtcagaagcacagatgacaatctgggcttgccattggcatttgaagtgtgttgggaggcagtcttacaggaatgagcccttatcctgtggggtctatgctaataacagacagttgtcagcattgcttggtgtcgaaaacccacattgttggtgtcagaagtattgtcagtaggatagggaaaacagtttgttttctttttttagtggtctttggtcatctttaagagcagggcttctcaaagtgtggtccttgaaccagcatcacctgtaccacgtaagaacttatgagaaatgttcattcttgggccccaacaaagaattaaaaattctgagggtgtgaacggggtctgagtttcagcacaacttcccgaccatgctgatgcattcttgcccaagcatgaaagccctcccttgtttaagaaggccattagggccgggtgtggtggctcatgcttgtaatcgagcactttgagaggacatagtgggaggatcacttgagccctggagttctagacaagcctgggcaacatggcaaaatgctgtctccacaaaaatcacaaaaattaggtgggcgtgtgttgtgtgcctataggcccagctacttaggagactgaggcaggaggatcgcttgagcccaggagattaaggctgcagcgagctgtgatggcaccactacagcctggatgacagagtgagacactgtctcaaaaaaaaaaaagaaaaagaaaaagaaaaaagaaaggaaaatgaaaaagaacgccattaggtataaaggagcaatggtaaaagaccagttgcaaaaggttagggaatgggtggttactgaaataagaagctatgtagaacactagtgttggtggcaggaagtagaaagcaagagcactgctctgtgggggatggtcatagcaaatgcaatatggaggcatttgcctctgcactgaggagaaaactatcttttccaagataggaggaaaggagataagtggaattaaagagaacctttgagcacagagttgggaaactgaaggtatttgtgttgtgctccctcaatcttttaattcaactataagctaaacccatgaaacttgagtagtttcagttatctgacttttttcttctcttttgatacagtgttggctattctgggtcttttgcctctctttatgtacttaagaatcagtttgccaatgtatgcaaaataactggctgggattttgattgtgattggcttgaatctatagatggagttgggaaggactgacatcttgacaatgttgaagcttcctattcatcattatgaaatatttctccatttgtttgattctttgatttcttttatcagaatttagttttcctcatatagtcttttaaaatattttgttatattttgttcaagtattttgtttttgaggaatgccaatgtaaatggtattgtgattttaatttcaaattccaatttttcattgctgttatataggaaaatgattttttttgcatgttagccttatatctttcaactttgctataatcaattattgatagtttcaaggattttttggtcaattattttgaatcttctacatagattatcatcatctgaacttagttttatttcttccttcccaatctgtatacctttatctccttttcttatttcattagctaggacttccagtatgatgttgaaagtagtggtgagaggggatatcttggtcttgttcttgatcttagtgggaaaacttcaagtttcttatcattaagtatgattttagctggagggtttttgtagaagttttttttttttaagttgaagaagtctccttctatttttagtttgctgatttttaaaaagaatcaggaatgggtgttaaattttgtgaaatgcttttctgcaactattgatttgagcactttatttttcttctttggcttgttgatgtgaagtacattaattgatttttgaatgctgaatcaaccttttgtacctgagattaatcccgtttggttgtggtatataattatttgtatacatgttgagttcgatttgctaatactttttgagaatttttgcattggtgttcatgaaaaaatattggtgtgtagttttttgtgacatctttatctgcttatggttttaaggtaatgctggcctcatagcatgagttagggagtatttcctctacttttacatttgagaagagattgcagagaattagtaaaattcctactttaaatattttgtggaattcaccagtgaacccatctggacctggtgctttctgttttggaaggtcattaattattttaaaatagatataggcctattcagattacctattttttctcatgcgagttttagcagattgtctttcaaggaattggtctatttcatttaggttatcaaatatgtcaacgtagagttattcatagtattcttttattatccttttaatgtgcaagggatctgtagtgatgtccccttttttgttttattgatattagcaatttgtgtcacatcttttattttgctttgttagccaggctagagatatctctatttttgatgtttttgatgaaccaactttttgttttattgattttctctgttgatttcgtgatttcaatttcatgatttttaaattatgcttacatttgatttaatttgatcttcttttgctagttatccaaggtggaagcttatattgttaagatccttttgcattcttatgcattcaatgatgtaaatttccctctaagcactgctttttctgcatctcacaaatattcatgagttgtattttcatgttcatttagtttgaaatatttttaaatttctcttgatatttctcttttgacccatgtgttacttagaagtgtgttgtttaatcaccatttttaaaaattttctagctatctttctgttattgatttctagtttaattccattgtggtctgagagcatatattgtataattttaatttttataaaatttgttaaggtgtgatttatggcccagaatgtggtctatcttggtgaatgttccatgtaagctttggaagactgtgtattctgctatatttgaatgaggtagtctatagacatcaattatgtccagttgattgatggtgctgttgaattcaactatgtccttactgattttccacctgctagatctgtccattctttgcagagggacactgaagtctccaactctagtagtgaatattctatttcttgttacagttttatcaacttctgcttcatgtcttttgatgctttgttgctagaaacatacacatgaagaattggtatgtcttttggagcatgacccatttatcctcatataatgcccctcattatttcctcgccctgatgtctgttctctctgaaagaaatatagcctctccaggtctcttttggttggtgttaaaatgacttaactttctttatcccccttacttttagtttatatgtggttttaaatttaaagtgggtttcttgtagacagcaaatagttcagagttgtttttcgatccactttgacaatctttgtcttttaattggtatatttggactattgatattttaagtgattattgatatagttagataaacatctactatatttattactgttttctgtctgttacactacttgttctttgtttatatttttattgtctactctttttctttccattgtggttttaatcgagcattttatatgtttccattttcttttcttagcatagtaattcttctttaaaaaaacattttttagtggttgcccctagagtttgcaatatacatttacaactaatctaagtccattttcaaataatactaaataatttcatgtgtagtgcaagtaccttttaataataaaacactcccagttccaccttccagtctcttgtattatagctataatttagttcacttacatatatgggtatacctaagtatatacattatcatatttatgattgaatatattgatgaaattattttgaaaaaactgttatcgttaaatcaattaagagtaagaaaaatagttctaattttattataaaatgaaataccttcatttattcattctctaatacactttctttctttatgtagatccaagtttctgacctgtataattttccttttctctcttcagcttctttgaacatttcttaccagccagacctactgacaacaattttccccaatttttgtttgtctgatagagactttatttcttcttgacttttgaagaataattccacagggcacagaactctagattggtgatttcttcccctcaaacccttaaatatttcattccactgccttcttgcttgcattgtttctgagaagttagatataattcttatctttgcctttctataggtaagatgttttttcctctggcttctatcaagattttttctttatgaacatgatatgcctttctttttgaacatgatatgcctttctttttgaacatgatatgcctttgtgtcggattttttttggcattattctgcttggttttctctgagtttcttggatatgtggtatggtatctgacactaatttggaaaaattctcagtcattattgcttcaaatatttcttctgttcttttttttcctttattctccttctggtattcccattacatgtatgttacagtttttgtagtcatcccgctgttttggatattctgtttttttcagtttttttttccttcgcatttcagtgttggaagtttctattgacatattctcaacctcagagattctttcttcagctgtgttcagtctaccaatgagtccatcaaaggcattttacatttttattacagaatttttgacctatagaatttcttttgattccatctttgaatctccatttctcttctgcttttcatctgttcttgcatgttgcctactttttccatgaaaacctttagctttttttttttttctttttgaggtggagtctcactgttgcccaggctggagtgcagtggtgtgatcttggctcactgcaacctctgcctcctgggttcaagtgattctcctcctcagcctcccaagtagctgggattacaggtgcctgccaccatgcctgagtaatttttgtatttttagtagagatggggttttatcatgttggccaggcgggtcttgaactcctaacctcaagtgatctgcccaccttagcctcccaaattgctgggattataggtgtgagccaccatgccctgcctttagcatgttaatcatagttgttttaaattcctgatctgttaattccaacatccctgtcatatctgactgtggttctgatgcttgctctgtgttttcaaatggtgtttttttttttttgccttttagtaagccttgtaattttttattgaaaggtggacatgatgtgctgggtaaaaggaactgtagtaaataggcctttagtaatgtactggtaggtgtagcagagggtgagggaagtattctgtagtcctatgattaggttttagtcttttagtgagcctgtgcgcctgcagcttggaagcacttgtgaagtgttttttcaccccttttggtgggacatagtgactagtgtgagcgggagttgagtatttcccttcccctaggtcagttaggctctgaaaaaaccctgataggttaggcatggtaaaatagtctcttttgagggcaggcattgttataagaatagaatgctctggggccaggtgcggtggctcacgcctgtaatccccgcactttgggaggctaaggcaggtggatcacctgaggtcaggagttcgagaccagcctggccaacatggtgaaaccccgtctctactaaaaatacaaaaatcagccaggtgtggtggcacacacctataatcccagctactcaggaggctgaggcaggagaactgcttgaacccagtaagtggaggttacagtgacccaagattgtgccactgcagtctagtctgggtgacagagcaagactccgtctcaaaaaaaaaagaatgctctggcatatttgaaaatggttacttttccctttttttctctgatcttcactgtgagaacctggtaagcatcctataggcaaaattcataaaagtatagaagtcggccagtgacttggacccacttggaattttcttgctctcacatcatgcacactgaatctccagcaatttttcacttacagtttaggttttcctaccctactactggttctctcagaggtttctgcttattggtttctgttttgtaagttgtgattctctgtacctaactgcctgtctcccattttggggggcagtggtttgccctgtgacctcacttctctgacagatctaagaaaagttgtttatttttcagtgtgctctgctttttacttgttacgatgaagccaaccactttcagaatttctacaaaccagatcagaatctggaagtcctgtttttttattttttttatccctttgtttagcatgttacctatcttaacacattttaaataagtgaatgcatagcttatatctacttctaggttatatgcttccttagaataggaattgattcttaaaatgtcgttctgctcacgcctgtaattccagcactttgggaggccaaggcaggcggatcacttggggtcaggagttcaagaccagcctggtcaacatggtaaaaccctgtgcctgcaaaaaatacaaaaattagctgggcatggtggtggccatctgtaatcccagctactagggaagctaaggcatgagaatcacttgaacctgggaggtggaggttgcagtgagctgagatcgcgccactgcactccagcctgggtgacaagagcaaaactccatctcataaataaataaataaataaataaataaataataaaaataaaaaaataaaataaaacaaaaattttattctgagcagtctctgaagaatataaattctactgccttgcctttagaacttataacagcatctcgcaaactatcacaagatgctccaaacatacttcttatgtgctgaattaagaagtcaactcaaatttagtatactagtaatatttttggatatcccaaaacactgccagctcagctttaggctgcccttcttgggggggaaaaaagcagttgaaatttaggacttaagtgggcatctcgtttaatttttaatggatttctatgttgttggttatggtgaagaggtgaaaagaataaatattctgtgcagaaaaattattcagtcttcatgtgaaaacactttgtccatagcaattactttatgaaaaagatgtggtattactttctttgctcttaactgagacctttaatttaaagaacctatactttacaagtttttattttcaatgcatgaaaaatgtagcagctatttcacaacctttacttttaaaatccatttttctttttaatctcaaatagttttttcttaaaaccttttgactttttatctaaattgtaatagccagagcaccttcccacaactagaatatctcatcctttttgtcttttctttttcctctcaaaatgcctactgggaacttaatttggagtcagattcttcatgataaatctggacttaatcaaaattcctcatatggtatattgtatatatcacagtactggatagtcctctgattaaatagatatttgatagtactttaaggtctatacttttggatgaacttaactgctttctccatttgtagtctcttgaaaatacagaaatttcagaaataatttataagaatatcaaggattcaaatcatatcagcacaaacacctaaatacttgtttgctttgttaaacacatatcccattttctatcttgataaacattggtgtaaagtagttgaatcattcagtgggtataagcagcatattctcaatactatgtttcattaataattaatagagatatatgaacacataaaagattcaattataatcaccttgtggatctaaatttcagttgacttgtcatcttgatttctggagaccacaaggtaatgaaaaataattacaagagtcttccatctgttgcagtattaaaatggcgagtaagacaccctgaaaggaaatgttctattcatggtacaatgcaattacagctagcaccaaattcaacactgtttaactttcaacatattattttgatttatcttgatccaacattctcagggaggaggtgcattgaagttattagaaaacactgacttagatttagggtatgtcttaaaagcttatttgcgggaagtactctagccttattcaacagatcactgagaagcctggaaaaacaaatcccggaaactaattattatgtgccagttatataaacaagaagactttgttgggtacaaaccagtgattccttgcctttgaaaaatgtgtcagatatcatgcattaccagcagttcaatgatataaggaaaccagagtaatagctaaaacctttaaagctaaaccaaagatttacaaattgcctcttcatccagtctttcccaacctaaaaactgagttctctaaaaattttagtatttttttctgaagaaaagggaacatggacatttatctaatcctcattagaaatctgactaatgataacaaggatttagacctcaagcacttcttaccaaaattcttgatatgaccttatagcaaattactttcacctgttgaactttcctttcttttattcccctgtacctcacctgcactgggcatattcaagttgcttatacaacactttactattgtgttagaaaaatcatgacacatgatgaatgtgtttgtgcaacatgagctgattcataaatgaaaatgtgcattgaaattccacaatattttaaaattaggagtttatctagcaattgaacaaaattgattaaatccattatttgttagatcagctaaattacataagttcattcatctgctcataaatccatccattcttccatctggctatcccttagtcaattcaaataaatatttatggggcactttgggtaagccaggtgctaagaattcaatgcaaaacaagatagactcccctgtccttgttgaacttatatttttggtacaaacaaaagcaataatcaagaaaaaataaaaaaagtactgattgtgattaataatatgaagaaattcaacagagtattgtacttaacatttgattgatctgattttctcagttgtctgagaacaaacatttgtgaaaatctcattgtagagttcttacgatggatagggggtcaactgtgtcattattgcttatcagcttatcccaaagacctagtttattaccagattgcaaatagtgttcaataaattattcttattaagggttgttatgtactctaaaacatttattgtggtcccttcactggttctggtttacaaacttacttttctatgatgacatagtatagaaattgagagtgaatatttagaagttcatttttattatatatttttgaagtattgatatgtagtgaattagaaatttaaaaagaaaacaaaactgtccttcactacagattgaaaagcattatactaaaagaccatttgctcagttatagtatataaaggccaaatgacttaaaaacaaattatgtaaggagaaggaaacaaccatttattcagtgccactaactgtcagccagttttttcagtggtcagttaatgactgcagtagtgttctaccttgctcaaagcaccctcctcaagttctggcatctaagctgacatcagaacacagagttggggctctctgtgggtcacctctagcacttgatctcctcatgcagtgcatggtgctctcacgtctatgctatgttcttatggtctttaggtaacaagaataattttctttcttttccttactatacattttgctttctgaaattcccttctcgccaatccaggtgaatgtcagaatgtgatttgacaactgtccaaagtactcattcactgaggagtggtaaggccttcgcccaacctgccttctctgggaatatactgctgcctgaacatatcattgtttattgccaggcttgaacttcaccaaattaatttattagggtcaacatctaaatattagaactatttcagattaatttttaagtcgtatccactttgggtactagatcaaattgcaggtctctgcttctggcttgagcctatgtttagagatgatgtgcatgaagacactctttgcttttcctttatgcaaaatgggcattttcaatctttttgtcattagtaaaggtcagtgataaaggaagtctgcatcaggggtccaattccttatggccagtttctctattctgttccaaggttgtttgtctccatatatcaacattggtcaggattgaaagtgtgcaacaaggtttgaatgaataagtgaaaatcttccactggtgacaggataaaatattccaatggtttttattgaagtacaatactgaattatgtttatggcatggtacctatatgtcacagaagtgatcccatcacttttaccttatag

SEQ ID NO: 6 - RNA, 249 nt, Homo sapiens
  source 1..249: mol_type = mRNA
atgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagg

SEQ ID NO: 7 - RNA, 156 nt, Homo sapiens
  source 1..156: mol_type = mRNA
gtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacag

SEQ ID NO: 8 - RNA, 14667 nt, Homo sapiens
  source 1..14667: mol_type = mRNA
gtgagatttgaacactgcttgctttgttagactgtgttcagtaagtgaatcccagtagcctgaagcaatgtgttagcagaatctatttgtaacattattattgtacagtagaatcaatattaaacacacatgttttattatatggagtcattatttttaatatgaaatttaatttgcagagtcctgaacctatataatgggtttattttaaatgtgattgtacttgcagaatatctaattaattgctaggttaataactaaagaagccattaaataaatcaaaattgtaacatgttttagatttcccatcttgaaaatgtcttccaaaaatatcttattgctgactccatctattgtcttaaattttatctaagttccattctgccaaacaagtgatactttttttctagcttttttcagtttgtttgttttgtttttctttgaagttttaattcagacatagattattttttcccagttatttactatatttattaagcatgagtaattgacattattttgaaatccttcttatggatcccagcactgggctgaacacatagaaggaacttaatatatactgatttctggaattgattcttggagacagggatggtcattatccatatacttcaggctccataaacatatttcttaattgccttcaaatccctattctggactgctctataaatctagacaagagtattatatattttgattgatattttttagataaaataaaagggagctgaaaactgaattgcaaactgaattttaaaactttatctctctgtggttaattgcaaacacagatacaaaaatatagagagagatacagttagtaaagatgttaggtcaccgttactaacactgacatagaaacagttttgctcatgagtttcagaatatatgagtttgattttgcccatggattttagaatatttgataaacatttaatgcattgtacaaattctgtgaaaacatatatataggatgtgcgaaaagtccctgtgtatcatgtgaaatggcttaaaacagaacaccataggtattcatatcagtgaataccataggtagctgaaagtgttttttcctggggtcgccaagatgaatgccaaaagtgatatcattattataaacaatagccagaataggttggtataaacctggtagaaagccttgataaattgactttctctcctcctgacatcctgccacccctttgctttgctgatgctcatttgtccactaaattaaactcaagcaagccctagtaaagtaatagaatttgtggagtcctcattagtataggaagtttccctgatgtgagattagtaattagagatgtagcaaaatgagaaagaagtaatatgcttagatatttcattttctctgaacctgtatatacaaaataggccatgcgtgttcagtaactattcactgcaaggcactctctaggtactttgggggaattggaaattactcacataaggctatggattgtgccatttgtcaaaagacaaaatgacaacaaatttagtttaaagacctcagtcagctttattttctattctagatttggacagtccttcatttcacaaattggagtaagtgttccaataagttgagcaaaggagcttggctttatagacccaaaaaaagggccaaaggaagcagaaacaaagaacaataagagaattggtcatttcaaagttacttttcttgaaaggtggggacaaggagacagaataatagaaaagtcactgattggttaacattggattaagaattaaaacagaggaaactttaagattgaagtttgaaactgacttgtttgggaaatcaggctgtcttctttcttgatttcttagaaggccggataacaactgagttttgctttggtgaacatgggtgactccatttttacttttagtctggtctgttgaggcctcgtgagagagcttaatctaaaacaatgacttcctataatttttgtttgacacatccaaagagggactctaatatttattgagagcttatcatatcttaagtactgtttaaacacttttatttgctattacatttgatcttattataactctaaaggcagaaatgattgcttttattttccacaatggaggaaactgaggttcaattaagtgagtaaggaagcagggatcttaaacccagataccattgctcctctttaaaggtggaagaacagaaaacatggggcaggggaagagagaaagtttctgtcccaggacatgataatctaaaagggaaaacgtaagatccactgaaacctgaggcagatttattgtggcaataacaaagcttaagtttcacagaccttcatttgcctgagccaactttgaaggccatgtatctaattttgtttttataattctataatctttattcttgaaaagagccctccctccaaatttacaagctttgggcccccaaaatccttgaaatgcccttgaataagagatatccaggtaaatgctatgggaattcagaggaggaagcagttagtatcagttggcggagagttaggctattaagagaaggttttatataggaagtggcatttagaatgaagctttgagaactgagctgtgtatttgaacaagtaaaggtggtgttgcagaattttgctccttagttctattaaaaacccgggttcttgtcacatgatccggaaaatttaggcacacagatacattgaagcatgagtagagcaggattttattgggcaaaaaggaaaaaaagaaaactcagcaaatcgagatggagtcttgctcacagattgaatcccaggccaccacaaaggaactgaagagatcgggcttctcccctgcataaggtgcaaattccccatggctccacccacttccccttagtgtgcatgtggggctccagtccacggtgggcatgcccagacaagccttgggcaggttccctcatctgtgcaaaagcatctgatgtaaacacttgaggggtggttcggagattctctgggacccttttattttcttatctgcctaggcatttggctgtctcagtgggtgggaaagggtgctccaggcaaagggcataacatgaggcaaagggcatgcacagaaaacagtgactggttcagtcaggttgggggatgccaaaggaagtaatgggagacaagattggagcaagatagataagagattgtggattttttttcttttttatctatataaatacagagacagggtctcactatgttgcccaggctggtctcaaactcctggcctcaagtgatcctcccacctcatcctcccaaagtgctaggattacaggcatgaggcactgtgcccaacctccaattttggattttgagagctaaagcaatatagtcgaaaactcagataatccaggtagattttgctattaggtgctatttggttcctggtacagagctaaaacccttggaatttcctaagtgataagagctacaggagcatcttttgttatatgtttccccccctagttcctgaaatagctctagagaaatacaggtgaataacatcctttgttattcatatcaagcccctatcaaccataccccagtttctatttatgaagtggcttttgggaagtccctaaagacaggagtggggaaaggctggttgtcagggggatgggttgaaactttcatcttccccccttgacctccagggagggatgagtggctgaaaattgtgtaaaatcaacaatggccagtgatttaatcaaccatgcctatgtaatgaagccacccgataagccttaactggaactttttggagagcctccaggctggtgaagacattgaggtgctcagaaggtggtattccagagagagcacagaatctctgttccccttcccacattcattttgctatgcatctctcccatctggctgttcttgagaggtatccgtttataataaactggtaacctagtaagtaaactgttaccctgagttctgtgagccattctagcaaattatcaaacctaaagagttcatggatacgtgcaatttacagatgcacagtcagaagcacagatgacaatctgggcttgccattggcatttgaagtgtgttgggaggcagtcttacaggaatgagcccttatcctgtggggtctatgctaataacagacagttgtcagcattgcttggtgtcgaaaacccacattgttggtgtcagaagtattgtcagtaggatagggaaaacagtttgttttctttttttagtggtctttggtcatctttaagagcagggcttctcaaagtgtggtccttgaaccagcatcacctgtaccacgtaagaacttatgagaaatgttcattcttgggccccaacaaagaattaaaaattctgagggtgtgaacggggtctgagtttcagcacaacttcccgaccatgctgatgcattcttgcccaagcatgaaagccctcccttgtttaagaaggccattagggccgggtgtggtggctcatgcttgtaatcgagcactttgagaggacatagtgggaggatcacttgagccctggagttctagacaagcctgggcaacatggcaaaatgctgtctccacaaaaatcacaaaaattaggtgggcgtgtgttgtgtgcctataggcccagctacttaggagactgaggcaggaggatcgcttgagcccaggagattaaggctgcagcgagctgtgatggcaccactacagcctggatgacagagtgagacactgtctcaaaaaaaaaaaagaaaaagaaaaagaaaaaagaaaggaaaatgaaaaagaacgccattaggtataaaggagcaatggtaaaagaccagttgcaaaaggttagggaatgggtggttactgaaataagaagctatgtagaacactagtgttggtggcaggaagtagaaagcaagagcactgctctgtgggggatggtcatagcaaatgcaatatggaggcatttgcctctgcactgaggagaaaactatcttttccaagataggaggaaaggagataagtggaattaaagagaacctttgagcacagagttgggaaactgaaggtatttgtgttgtgctccctcaatcttttaattcaactataagctaaacccatgaaacttgagtagtttcagttatctgacttttttcttctcttttgatacagtgttggctattctgggtcttttgcctctctttatgtacttaagaatcagtttgccaatgtatgcaaaataactggctgggattttgattgtgattggcttgaatctatagatggagttgggaaggactgacatcttgacaatgttgaagcttcctattcatcattatgaaatatttctccatttgtttgattctttgatttcttttatcagaatttagttttcctcatatagtcttttaaaatattttgttatattttgttcaagtattttgtttttgaggaatgccaatgtaaatggtattgtgattttaatttcaaattccaatttttcattgctgttatataggaaaatgattttttttgcatgttagccttatatctttcaactttgctataatcaattattgatagtttcaaggattttttggtcaattattttgaatcttctacatagattatcatcatctgaacttagttttatttcttccttcccaatctgtatacctttatctccttttcttatttcattagctaggacttccagtatgatgttgaaagtagtggtgagaggggatatcttggtcttgttcttgatcttagtgggaaaacttcaagtttcttatcattaagtatgattttagctggagggtttttgtagaagttttttttttttaagttgaagaagtctccttctatttttagtttgctgatttttaaaaagaatcaggaatgggtgttaaattttgtgaaatgcttttctgcaactattgatttgagcactttatttttcttctttggcttgttgatgtgaagtacattaattgatttttgaatgctgaatcaaccttttgtacctgagattaatcccgtttggttgtggtatataattatttgtatacatgttgagttcgatttgctaatactttttgagaatttttgcattggtgttcatgaaaaaatattggtgtgtagttttttgtgacatctttatctgcttatggttttaaggtaatgctggcctcatagcatgagttagggagtatttcctctacttttacatttgagaagagattgcagagaattagtaaaattcctactttaaatattttgtggaattcaccagtgaacccatctggacctggtgctttctgttttggaaggtcattaattattttaaaatagatataggcctattcagattacctattttttctcatgcgagttttagcagattgtctttcaaggaattggtctatttcatttaggttatcaaatatgtcaacgtagagttattcatagtattcttttattatccttttaatgtgcaagggatctgtagtgatgtccccttttttgttttattgatattagcaatttgtgtcacatcttttattttgctttgttagccaggctagagatatctctatttttgatgtttttgatgaaccaactttttgttttattgattttctctgttgatttcgtgatttcaatttcatgatttttaaattatgcttacatttgatttaatttgatcttcttttgctagttatccaaggtggaagcttatattgttaagatccttttgcattcttatgcattcaatgatgtaaatttccctctaagcactgctttttctgcatctcacaaatattcatgagttgtattttcatgttcatttagtttgaaatatttttaaatttctcttgatatttctcttttgacccatgtgttacttagaagtgtgttgtttaatcaccatttttaaaaattttctagctatctttctgttattgatttctagtttaattccattgtggtctgagagcatatattgtataattttaatttttataaaatttgttaaggtgtgatttatggcccagaatgtggtctatcttggtgaatgttccatgtaagctttggaagactgtgtattctgctatatttgaatgaggtagtctatagacatcaattatgtccagttgattgatggtgctgttgaattcaactatgtccttactgattttccacctgctagatctgtccattctttgcagagggacactgaagtctccaactctagtagtgaatattctatttcttgttacagttttatcaacttctgcttcatgtcttttgatgctttgttgctagaaacatacacatgaagaattggtatgtcttttggagcatgacccatttatcctcatataatgcccctcattatttcctcgccctgatgtctgttctctctgaaagaaatatagcctctccaggtctcttttggttggtgttaaaatgacttaactttctttatcccccttacttttagtttatatgtggttttaaatttaaagtgggtttcttgtagacagcaaatagttcagagttgtttttcgatccactttgacaatctttgtcttttaattggtatatttggactattgatattttaagtgattattgatatagttagataaacatctactatatttattactgttttctgtctgttacactacttgttctttgtttatatttttattgtctactctttttctttccattgtggttttaatcgagcattttatatgtttccattttcttttcttagcatagtaattcttctttaaaaaaacattttttagtggttgcccctagagtttgcaatatacatttacaactaatctaagtccattttcaaataatactaaataatttcatgtgtagtgcaagtaccttttaataataaaacactcccagttccaccttccagtctcttgtattatagctataatttagttcacttacatatatgggtatacctaagtatatacattatcatatttatgattgaatatattgatgaaattattttgaaaaaactgttatcgttaaatcaattaagagtaagaaaaatagttctaattttattataaaatgaaataccttcatttattcattctctaatacactttctttctttatgtagatccaagtttctgacctgtataattttccttttctctcttcagcttctttgaacatttcttaccagccagacctactgacaacaattttccccaatttttgtttgtctgatagagactttatttcttcttgacttttgaagaataattccacagggcacagaactctagattggtgatttcttcccctcaaacccttaaatatttcattccactgccttcttgcttgcattgtttctgagaagttagatataattcttatctttgcctttctataggtaagatgttttttcctctggcttctatcaagattttttctttatgaacatgatatgcctttctttttgaacatgatatgcctttctttttgaacatgatatgcctttgtgtcggattttttttggcattattctgcttggttttctctgagtttcttggatatgtggtatggtatctgacactaatttggaaaaattctcagtcattattgcttcaaatatttcttctgttcttttttttcctttattctccttctggtattcccattacatgtatgttacagtttttgtagtcatcccgctgttttggatattctgtttttttcagtttttttttccttcgcatttcagtgttggaagtttctattgacatattctcaacctcagagattctttcttcagctgtgttcagtctaccaatgagtccatcaaaggcattttacatttttattacagaatttttgacctatagaatttcttttgattccatctttgaatctccatttctcttctgcttttcatctgttcttgcatgttgcctactttttccatgaaaacctttagctttttttttttttctttttgaggtggagtctcactgttgcccaggctggagtgcagtggtgtgatcttggctcactgcaacctctgcctcctgggttcaagtgattctcctcctcagcctcccaagtagctgggattacaggtgcctgccaccatgcctgagtaatttttgtatttttagtagagatggggttttatcatgttggccaggcgggtcttgaactcctaacctcaagtgatctgcccaccttagcctcccaaattgctgggattataggtgtgagccaccatgccctgcctttagcatgttaatcatagttgttttaaattcctgatctgttaattccaacatccctgtcatatctgactgtggttctgatgcttgctctgtgttttcaaatggtgtttttttttttttgccttttagtaagccttgtaattttttattgaaaggtggacatgatgtgctgggtaaaaggaactgtagtaaataggcctttagtaatgtactggtaggtgtagcagagggtgagggaagtattctgtagtcctatgattaggttttagtcttttagtgagcctgtgcgcctgcagcttggaagcacttgtgaagtgttttttcaccccttttggtgggacatagtgactagtgtgagcgggagttgagtatttcccttcccctaggtcagttaggctctgaaaaaaccctgataggttaggcatggtaaaatagtctcttttgagggcaggcattgttataagaatagaatgctctggggccaggtgcggtggctcacgcctgtaatccccgcactttgggaggctaaggcaggtggatcacctgaggtcaggagttcgagaccagcctggccaacatggtgaaaccccgtctctactaaaaatacaaaaatcagccaggtgtggtggcacacacctataatcccagctactcaggaggctgaggcaggagaactgcttgaacccagtaagtggaggttacagtgacccaagattgtgccactgcagtctagtctgggtgacagagcaagactccgtctcaaaaaaaaaagaatgctctggcatatttgaaaatggttacttttccctttttttctctgatcttcactgtgagaacctggtaagcatcctataggcaaaattcataaaagtatagaagtcggccagtgacttggacccacttggaattttcttgctctcacatcatgcacactgaatctccagcaatttttcacttacagtttaggttttcctaccctactactggttctctcagaggtttctgcttattggtttctgttttgtaagttgtgattctctgtacctaactgcctgtctcccattttggggggcagtggtttgccctgtgacctcacttctctgacagatctaagaaaagttgtttatttttcagtgtgctctgctttttacttgttacgatgaagccaaccactttcagaatttctacaaaccagatcagaatctggaagtcctgtttttttattttttttatccctttgtttagcatgttacctatcttaacacattttaaataagtgaatgcatagcttatatctacttctaggttatatgcttccttagaataggaattgattcttaaaatgtcgttctgctcacgcctgtaattccagcactttgggaggccaaggcaggcggatcacttggggtcaggagttcaagaccagcctggtcaacatggtaaaaccctgtgcctgcaaaaaatacaaaaattagctgggcatggtggtggccatctgtaatcccagctactagggaagctaaggcatgagaatcacttgaacctgggaggtggaggttgcagtgagctgagatcgcgccactgcactccagcctgggtgacaagagcaaaactccatctcataaataaataaataaataaataaataaataataaaaataaaaaaataaaataaaacaaaaattttattctgagcagtctctgaagaatataaattctactgccttgcctttagaacttataacagcatctcgcaaactatcacaagatgctccaaacatacttcttatgtgctgaattaagaagtcaactcaaatttagtatactagtaatatttttggatatcccaaaacactgccagctcagctttaggctgcccttcttgggggggaaaaaagcagttgaaatttaggacttaagtgggcatctcgtttaatttttaatggatttctatgttgttggttatggtgaagaggtgaaaagaataaatattctgtgcagaaaaattattcagtcttcatgtgaaaacactttgtccatagcaattactttatgaaaaagatgtggtattactttctttgctcttaactgagacctttaatttaaagaacctatactttacaagtttttattttcaatgcatgaaaaatgtagcagctatttcacaacctttacttttaaaatccatttttctttttaatctcaaatagttttttcttaaaaccttttgactttttatctaaattgtaatagccagagcaccttcccacaactagaatatctcatcctttttgtcttttctttttcctctcaaaatgcctactgggaacttaatttggagtcagattcttcatgataaatctggacttaatcaaaattcctcatatggtatattgtatatatcacagtactggatagtcctctgattaaatagatatttgatagtactttaaggtctatacttttggatgaacttaactgctttctccatttgtagtctcttgaaaatacagaaatttcagaaataatttataagaatatcaaggattcaaatcatatcagcacaaacacctaaatacttgtttgctttgttaaacacatatcccattttctatcttgataaacattggtgtaaagtagttgaatcattcagtgggtataagcagcatattctcaatactatgtttcattaataattaatagagatatatgaacacataaaagattcaattataatcaccttgtggatctaaatttcagttgacttgtcatcttgatttctggagaccacaaggtaatgaaaaataattacaagagtcttccatctgttgcagtattaaaatggcgagtaagacaccctgaaaggaaatgttctattcatggtacaatgcaattacagctagcaccaaattcaacactgtttaactttcaacatattattttgatttatcttgatccaacattctcagggaggaggtgcattgaagttattagaaaacactgacttagatttagggtatgtcttaaaagcttatttgcgggaagtactctagccttattcaacagatcactgagaagcctggaaaaacaaatcccggaaactaattattatgtgccagttatataaacaagaagactttgttgggtacaaaccagtgattccttgcctttgaaaaatgtgtcagatatcatgcattaccagcagttcaatgatataaggaaaccagagtaatagctaaaacctttaaagctaaaccaaagatttacaaattgcctcttcatccagtctttcccaacctaaaaactgagttctctaaaaattttagtatttttttctgaagaaaagggaacatggacatttatctaatcctcattagaaatctgactaatgataacaaggatttagacctcaagcacttcttaccaaaattcttgatatgaccttatagcaaattactttcacctgttgaactttcctttcttttattcccctgtacctcacctgcactgggcatattcaagttgcttatacaacactttactattgtgttagaaaaatcatgacacatgatgaatgtgtttgtgcaacatgagctgattcataaatgaaaatgtgcattgaaattccacaatattttaaaattaggagtttatctagcaattgaacaaaattgattaaatccattatttgttagatcagctaaattacataagttcattcatctgctcataaatccatccattcttccatctggctatcccttagtcaattcaaataaatatttatggggcactttgggtaagccaggtgctaagaattcaatgcaaaacaagatagactcccctgtccttgttgaacttatatttttggtacaaacaaaagcaataatcaagaaaaaataaaaaaagtactgattgtgattaataatatgaagaaattcaacagagtattgtacttaacatttgattgatctgattttctcagttgtctgagaacaaacatttgtgaaaatctcattgtagagttcttacgatggatagggggtcaactgtgtcattattgcttatcagcttatcccaaagacctagtttattaccagattgcaaatagtgttcaataaattattcttattaagggttgttatgtactctaaaacatttattgtggtcccttcactggttctggtttacaaacttacttttctatgatgacatagtatagaaattgagagtgaatatttagaagttcatttttattatatatttttgaagtattgatatgtagtgaattagaaatttaaaaagaaaacaaaactgtccttcactacagattgaaaagcattatactaaaagaccatttgctcagttatagtatataaaggccaaatgacttaaaaacaaattatgtaaggagaaggaaacaaccatttattcagtgccactaactgtcagccagttttttcagtggtcagttaatgactgcagtagtgttctaccttgctcaaagcaccctcctcaagttctggcatctaagctgacatcagaacacagagttggggctctctgtgggtcacctctagcacttgatctcctcatgcagtgcatggtgctctcacgtctatgctatgttcttatggtctttaggtaacaagaataattttctttcttttccttactatacattttgctttctgaaattcccttctcgccaatccaggtgaatgtcagaatgtgatttgacaactgtccaaagtactcattcactgaggagtggtaaggccttcgcccaacctgccttctctgggaatatactgctgcctgaacatatcattgtttattgccaggcttgaacttcaccaaattaatttattagggtcaacatctaaatattagaactatttcagattaatttttaagtcgtatccactttgggtactagatcaaattgcaggtctctgcttctggcttgagcctatgtttagagatgatgtgcatgaagacactctttgcttttcctttatgcaaaatgggcattttcaatctttttgtcattagtaaaggtcagtgataaaggaagtctgcatcaggggtccaattccttatggccagtttctctattctgttccaaggttgtttgtctccatatatcaacattggtcaggattgaaagtgtgcaacaaggtttgaatgaataagtgaaaatcttccactggtgacaggataaaatattccaatggtttttattgaagtacaatactgaattatgtttatggcatggtacctatatgtcacagaagtgatcccatcacttttaccttatag

SEQ ID NO: 9 - DNA, 22 bp, synthetic construct
  source 1..22: mol_type = other DNA
cagagggtgagatttgaacact

SEQ ID NO: 10 - DNA, 22 bp, synthetic construct
  source 1..22: mol_type = other DNA
agagggtgagatttgaacactg

SEQ ID NO: 11 - DNA, 22 bp, synthetic construct
  source 1..22: mol_type = other DNA
gagggtgagatttgaacactgc

SEQ ID NO: 12 - DNA, 22 bp, synthetic construct
  source 1..22: mol_type = other DNA
agggtgagatttgaacactgct

SEQ ID NO: 13 - DNA, 22 bp, synthetic construct
  source 1..22: mol_type = other DNA
gggtgagatttgaacactgctt

SEQ ID NO: 14 - DNA, 22 bp, synthetic construct
  source 1..22: mol_type = other DNA
ggtgagatttgaacactgcttg

SEQ ID NO: 15 - DNA, 22 bp, synthetic construct
  source 1..22: mol_type = other DNA
gtgagatttgaacactgcttgc

SEQ ID NO: 16 - DNA, 22 bp, synthetic construct
  source 1..22: mol_type = other DNA
tgagatttgaacactgcttgct

SEQ ID NO: 17 - DNA, 22 bp, synthetic construct
  source 1..22: mol_type = other DNA
gagatttgaacactgcttgctt

SEQ ID NO: 18 - DNA, 22 bp, synthetic construct
  source 1..22: mol_type = other DNA
agatttgaacactgcttgcttt

SEQ ID NO: 19 - DNA, 22 bp, synthetic construct
  source 1..22: mol_type = other DNA
gatttgaacactgcttgctttg

SEQ ID NO: 20 - DNA, 22 bp, synthetic construct
  source 1..22: mol_type = other DNA
catttccttctcaataagtcct

SEQ ID NO: 21 - DNA, 22 bp, synthetic construct
  source 1..22: mol_type = other DNA
ccttctcaataagtcctggcca

SEQ ID NO: 22 - DNA, 22 bp, synthetic construct
  source 1..22: mol_type = other DNA
tcaataagtcctggccagaggg

SEQ ID NO: 23 - DNA, 22 bp, synthetic construct
  source 1..22: mol_type = other DNA
aagtcctggccagagggtgaga

SEQ ID NO: 24 - DNA, 22 bp, synthetic construct
  source 1..22: mol_type = other DNA
ctggccagagggtgagatttga

SEQ ID NO: 25 - DNA, 22 bp, synthetic construct
  source 1..22: mol_type = other DNA
gaacactgcttgctttgttaga

SEQ ID NO: 26 - DNA, 22 bp, synthetic construct
  source 1..22: mol_type = other DNA
ctgcttgctttgttagactgtg

SEQ ID NO: 27 - DNA, 22 bp, synthetic construct
  source 1..22: mol_type = other DNA
tcacttttaccttataggtggg

SEQ ID NO: 28 - DNA, 22 bp, synthetic construct
  source 1..22: mol_type = other DNA
atcacttttaccttataggtgg

SEQ ID NO: 29 - DNA, 22 bp, synthetic construct
  source 1..22: mol_type = other DNA
catcacttttaccttataggtg

SEQ ID NO: 30 - DNA, 22 bp, synthetic construct
  source 1..22: mol_type = other DNA
ccatcacttttaccttataggt

SEQ ID NO: 31 - DNA, 22 bp, synthetic construct
  source 1..22: mol_type = other DNA
cccatcacttttaccttatagg

SEQ ID NO: 32 - DNA, 22 bp, synthetic construct
  source 1..22: mol_type = other DNA
tcccatcacttttaccttatag

SEQ ID NO: 33 - DNA, 22 bp, synthetic construct
  source 1..22: mol_type = other DNA
atcccatcacttttaccttata

SEQ ID NO: 34 - DNA, 22 bp, synthetic construct
  source 1..22: mol_type = other DNA
gatcccatcacttttaccttat

SEQ ID NO: 35 - DNA, 22 bp, synthetic construct
  source 1..22: mol_type = other DNA
tgatcccatcacttttacctta

SEQ ID NO: 36 - DNA, 22 bp, synthetic construct
  source 1..22: mol_type = other DNA
gtgatcccatcacttttacctt

SEQ ID NO: 37 - DNA, 22 bp, synthetic construct
  source 1..22: mol_type = other DNA
agtgttcaaatctcaccctctg

SEQ ID NO: 38 - DNA, 22 bp, synthetic construct
  source 1..22: mol_type = other DNA
cagtgttcaaatctcaccctct

SEQ ID NO: 39 - DNA, 22 bp, synthetic construct
  source 1..22: mol_type = other DNA
gcagtgttcaaatctcaccctc

SEQ ID NO: 40 - DNA, 22 bp, synthetic construct
  source 1..22: mol_type = other DNA
agcagtgttcaaatctcaccct

SEQ ID NO: 41 - DNA, 22 bp, synthetic construct
  source 1..22: mol_type = other DNA
aagcagtgttcaaatctcaccc

SEQ ID NO: 42 - DNA, 22 bp, synthetic construct
  source 1..22: mol_type = other DNA
caagcagtgttcaaatctcacc

SEQ ID NO: 43 - DNA, 22 bp, synthetic construct
  source 1..22: mol_type = other DNA
gcaagcagtgttcaaatctcac

SEQ ID NO: 44 - DNA, 22 bp, synthetic construct
  source 1..22: mol_type = other DNA
agcaagcagtgttcaaatctca

SEQ ID NO: 45 - DNA, 22 bp, synthetic construct
  source 1..22: mol_type = other DNA
aagcaagcagtgttcaaatctc

SEQ ID NO: 46 - DNA, 22 bp, synthetic construct
  source 1..22: mol_type = other DNA
aaagcaagcagtgttcaaatct

SEQ ID NO: 47 - DNA, 22 bp, synthetic construct
  source 1..22: mol_type = other DNA
caaagcaagcagtgttcaaatc

SEQ ID NO: 48 - DNA, 22 bp, synthetic construct
  source 1..22: mol_type = other DNA
aggacttattgagaaggaaatg

SEQ ID NO: 49 - DNA, 22 bp, synthetic construct
  source 1..22: mol_type = other DNA
tggccaggacttattgagaagg

SEQ ID NO: 50 - DNA, 22 bp, synthetic construct
  source 1..22: mol_type = other DNA
ccctctggccaggacttattga

SEQ ID NO: 51 - DNA, 22 bp, synthetic construct
  source 1..22: mol_type = other DNA
tctcaccctctggccaggactt

SEQ ID NO: 52 - DNA, 22 bp, synthetic construct
  source 1..22: mol_type = other DNA
tcaaatctcaccctctggccag

SEQ ID NO: 53 - DNA, 22 bp, synthetic construct
  source 1..22: mol_type = other DNA
tctaacaaagcaagcagtgttc

SEQ ID NO: 54 - DNA, 22 bp, synthetic construct
  source 1..22: mol_type = other DNA
cacagtctaacaaagcaagcag

SEQ ID NO: 55 - DNA, 22 bp, synthetic construct
  source 1..22: mol_type = other DNA
cccacctataaggtaaaagtga

SEQ ID NO: 56 - DNA, 22 bp, synthetic construct
  source 1..22: mol_type = other DNA
ccacctataaggtaaaagtgat

SEQ ID NO: 57 - DNA, 22 bp, synthetic construct
  source 1..22: mol_type = other DNA
cacctataaggtaaaagtgatg

SEQ ID NO: 58 - DNA, 22 bp, synthetic construct
  source 1..22: mol_type = other DNA
acctataaggtaaaagtgatgg

SEQ ID NO: 59 - DNA, 22 bp, synthetic construct
  source 1..22: mol_type = other DNA
cctataaggtaaaagtgatggg

SEQ ID NO: 60 - DNA, 22 bp, synthetic construct
  source 1..22: mol_type = other DNA
ctataaggtaaaagtgatggga

SEQ ID NO: 61 - DNA, 22 bp, synthetic construct
  source 1..22: mol_type = other DNA
tataaggtaaaagtgatgggat

SEQ ID NO: 62 - DNA, 22 bp, synthetic construct
  source 1..22: mol_type = other DNA
ataaggtaaaagtgatgggatc

SEQ ID NO: 63 - DNA, 22 bp, synthetic construct
  source 1..22: mol_type = other DNA
taaggtaaaagtgatgggatca

SEQ ID NO: 64 - DNA, 22 bp, synthetic construct
  source 1..22: mol_type = other DNA
aaggtaaaagtgatgggatcac

SEQ ID NO: 65 - RNA, 1557 nt, Homo sapiens
  source 1..1557: mol_type = mRNA
agagcagcataaatgttgacatgggacatttgctcatggaattggagctcgtgggacagtcacctcatggaattggagctcgtggaacagttacctctgcctcagaaaacaaggatgaattaagtttttttttaaaaaagaaacatttggtaaggggaattgaggacactgatatgggtcttgataaatggcttcctggcaatagtcaaattgtgtgaaaggtacttcaaatccttgaagatttaccacttgtgttttgcaagccagattttcctgaaaacccttgccatgtgctagtaattggaaaggcagctctaaatgtcaatcagcctagttgatcagcttattgtctagtgaaactcgttaatttgtagtgttggagaagaactgaaatcatacttcttagggttatgattaagtaatgataactggaaacttcagcggtttatataagcttgtattcctttttctctcctctccccatgatgtttagaaacacaactatattgtttgctaagcattccaactatctcatttccaagcaagtattagaataccacaggaaccacaagactgcacatcaaaatatgccccattcaacatctagtgagcagtcaggaaagagaacttccagatcctggaaatcagggttagtattgtccaggtctaccaaaaatctcaatatttcagataatcacaatacatcccttacctgggaaagggctgttataatctttcacaggggacaggatggttcccttgatgaagaagttgatatgccttttcccaactccagaaagtgacaagctcacagacctttgaactagagtttagctggaaaagtatgttagtgcaaattgtcacaggacagcccttctttccacagaagctccaggtagagggtgtgtaagtagataggccatgggcactgtgggtagacacacatgaagtccaagcatttagatgtataggttgatggtggtatgttttcaggctagatgtatgtacttcatgctgtctacactaagagagaatgagagacacactgaagaagcaccaatcatgaattagttttatatgcttctgttttataattttgtgaagcaaaattttttctctaggaaatatttattttaataatgtttcaaacatatataacaatgctgtattttaaaagaatgattatgaattacatttgtataaaataatttttatatttgaaatattgactttttatggcactagtatttctatgaaatattatgttaaaactgggacaggggagaacctagggtgatattaaccaggggccatgaatcaccttttggtctggagggaagccttggggctgatgcagttgttgcccacagctgtatgattcccagccagcacagcctcttagatgcagttctgaagaagatggtaccaccagtctgactgtttccatcaagggtacactgccttctcaactccaaactgactcttaagaagactgcattatatttattactgtaagaaaatatcacttgtcaataaaatccatacatttgtgtgaaa

SEQ ID NO: 66 - DNA, 22 bp, synthetic construct
  source 1..22: mol_type = other DNA
  modified_base 1..2: mod_base = OTHER; note = 2-methoxyethoxy ribonucleotide
  modified_base 3: mod_base = OTHER; note = 2-methoxyethoxy thymidine ribonucleotide
  modified_base 4: mod_base = OTHER; note = 2-methoxyethoxy ribonucleotide
  modified_base 5..6: mod_base = OTHER; note = 2-methoxyethoxy thymidine ribonucleotide
  modified_base 7..10: mod_base = OTHER; note = 2-methoxyethoxy ribonucleotide
  modified_base 11: mod_base = OTHER; note = 2-methoxyethoxy thymidine ribonucleotide
  modified_base 12: mod_base = OTHER; note = 2-methoxyethoxy ribonucleotide
  modified_base 13: mod_base = OTHER; note = 2-methoxyethoxy thymidine ribonucleotide
  modified_base 14..18: mod_base = OTHER; note = 2-methoxyethoxy ribonucleotide
  modified_base 19: mod_base = OTHER; note = 2-methoxyethoxy thymidine ribonucleotide
  modified_base 20: mod_base = OTHER; note = 2-methoxyethoxy ribonucleotide
  modified_base 21: mod_base = OTHER; note = 2-methoxyethoxy thymidine ribonucleotide
  modified_base 22: mod_base = OTHER; note = 2-methoxyethoxy ribonucleotide
  modified_base 1..22: mod_base = OTHER; note = Phosphorothioate linkage
agtgttcaaatctcaccctctg

SEQ ID NO: 67 - DNA, 22 bp, synthetic construct
  source 1..22: mol_type = other DNA
  modified_base 1..3: mod_base = OTHER; note = 2-methoxyethoxy ribonucleotide
  modified_base 4: mod_base = OTHER; note = 2-methoxyethoxy thymidine ribonucleotide
  modified_base 5: mod_base = OTHER; note = 2-methoxyethoxy ribonucleotide
  modified_base 6..7: mod_base = OTHER; note = 2-methoxyethoxy thymidine ribonucleotide
  modified_base 8..11: mod_base = OTHER; note = 2-methoxyethoxy ribonucleotide
  modified_base 12: mod_base = OTHER; note = 2-methoxyethoxy thymidine ribonucleotide
  modified_base 13: mod_base = OTHER; note = 2-methoxyethoxy ribonucleotide
  modified_base 14: mod_base = OTHER; note = 2-methoxyethoxy thymidine ribonucleotide
  modified_base 15..19: mod_base = OTHER; note = 2-methoxyethoxy ribonucleotide
  modified_base 20: mod_base = OTHER; note = 2-methoxyethoxy thymidine ribonucleotide
  modified_base 21: mod_base = OTHER; note = 2-methoxyethoxy ribonucleotide
  modified_base 22: mod_base = OTHER; note = 2-methoxyethoxy thymidine ribonucleotide
  modified_base 1..22: mod_base = OTHER; note = Phosphorothioate linkage
cagtgttcaaatctcaccctct

SEQ ID NO: 68 - DNA, 22 bp, synthetic construct
  source 1..22: mol_type = other DNA
  modified_base 1..4: mod_base = OTHER; note = 2-methoxyethoxy ribonucleotide
  modified_base 5: mod_base = OTHER; note = 2-methoxyethoxy thymidine ribonucleotide
  modified_base 6: mod_base = OTHER; note = 2-methoxyethoxy ribonucleotide
  modified_base 7..8: mod_base = OTHER; note = 2-methoxyethoxy thymidine ribonucleotide
  modified_base 9..12: mod_base = OTHER; note = 2-methoxyethoxy ribonucleotide
  modified_base 13: mod_base = OTHER; note = 2-methoxyethoxy thymidine ribonucleotide
  modified_base 14: mod_base = OTHER; note = 2-methoxyethoxy ribonucleotide
  modified_base 15: mod_base = OTHER; note = 2-methoxyethoxy thymidine ribonucleotide
  modified_base 16..20: mod_base = OTHER; note = 2-methoxyethoxy ribonucleotide
  modified_base 21: mod_base = OTHER; note = 2-methoxyethoxy thymidine ribonucleotide
  modified_base 22: mod_base = OTHER; note = 2-methoxyethoxy ribonucleotide
  modified_base 1..22: mod_base = OTHER; note = Phosphorothioate linkage
gcagtgttcaaatctcaccctc

SEQ ID NO: 69 - DNA, 22 bp, synthetic construct
  source 1..22: mol_type = other DNA
  modified_base 1..5: mod_base = OTHER; note = 2-methoxyethoxy ribonucleotide
  modified_base 6: mod_base = OTHER; note = 2-methoxyethoxy thymidine ribonucleotide
  modified_base 7: mod_base = OTHER; note = 2-methoxyethoxy ribonucleotide
  modified_base 8..9: mod_base = OTHER; note = 2-methoxyethoxy thymidine ribonucleotide
  modified_base 10..13: mod_base = OTHER; note = 2-methoxyethoxy ribonucleotide
  modified_base 14: mod_base = OTHER; note = 2-methoxyethoxy thymidine ribonucleotide
  modified_base 15: mod_base = OTHER; note = 2-methoxyethoxy ribonucleotide
  modified_base 16: mod_base = OTHER; note = 2-methoxyethoxy thymidine ribonucleotide
  modified_base 17..21: mod_base = OTHER; note = 2-methoxyethoxy ribonucleotide
  modified_base 22: mod_base = OTHER; note = 2-methoxyethoxy thymidine ribonucleotide
  modified_base 1..22: mod_base = OTHER; note = Phosphorothioate linkage
agcagtgttcaaatctcaccct

SEQ ID NO: 70 - DNA, 22 bp, synthetic construct
  source 1..22: mol_type = other DNA
  modified_base 1..6: mod_base = OTHER; note = 2-methoxyethoxy ribonucleotide
  modified_base 7: mod_base = OTHER; note = 2-methoxyethoxy thymidine ribonucleotide
  modified_base 8: mod_base = OTHER; note = 2-methoxyethoxy ribonucleotide
  modified_base 9..10: mod_base = OTHER; note = 2-methoxyethoxy thymidine ribonucleotide
  modified_base 11..14: mod_base = OTHER; note = 2-methoxyethoxy ribonucleotide
  modified_base 15: mod_base = OTHER; note = 2-methoxyethoxy thymidine ribonucleotide
  modified_base 16: mod_base = OTHER; note = 2-methoxyethoxy ribonucleotide
  modified_base 17: mod_base = OTHER; note = 2-methoxyethoxy thymidine ribonucleotide
  modified_base 18..22: mod_base = OTHER; note = 2-methoxyethoxy ribonucleotide
  modified_base 1..22: mod_base = OTHER; note = Phosphorothioate linkage
aagcagtgttcaaatctcaccc

SEQ ID NO: 71 - DNA, 22 bp, synthetic construct
  source 1..22: mol_type = other DNA
  modified_base 1..7: mod_base = OTHER; note = 2-methoxyethoxy ribonucleotide
  modified_base 8: mod_base = OTHER; note = 2-methoxyethoxy thymidine ribonucleotide
  modified_base 9: mod_base = OTHER; note = 2-methoxyethoxy ribonucleotide
  modified_base 10..11: mod_base = OTHER; note = 2-methoxyethoxy thymidine ribonucleotide
  modified_base 12..15: mod_base = OTHER; note = 2-methoxyethoxy ribonucleotide
  modified_base 16: mod_base = OTHER; note = 2-methoxyethoxy thymidine ribonucleotide
  modified_base 17: mod_base = OTHER; note = 2-methoxyethoxy ribonucleotide
  modified_base 18: mod_base = OTHER; note = 2-methoxyethoxy thymidine ribonucleotide
  modified_base 19..22: mod_base = OTHER; note = 2-methoxyethoxy ribonucleotide
  modified_base 1..22: mod_base = OTHER; note = Phosphorothioate linkage
caagcagtgttcaaatctcacc

SEQ ID NO: 72 - DNA, 22 bp, synthetic construct
  source 1..22: mol_type = other DNA
  modified_base 1..8: mod_base = OTHER; note = 2-methoxyethoxy ribonucleotide
  modified_base 9: mod_base = OTHER; note = 2-methoxyethoxy thymidine ribonucleotide
  modified_base 10: mod_base = OTHER; note = 2-methoxyethoxy ribonucleotide
  modified_base 11..12: mod_base = OTHER; note = 2-methoxyethoxy thymidine ribonucleotide
  modified_base 13..16: mod_base = OTHER; note = 2-methoxyethoxy ribonucleotide
  modified_base 17: mod_base = OTHER; note = 2-methoxyethoxy thymidine ribonucleotide
  modified_base 18: mod_base = OTHER; note = 2-methoxyethoxy ribonucleotide
  modified_base 19: mod_base = OTHER; note = 2-methoxyethoxy thymidine ribonucleotide
  modified_base 20..22: mod_base = OTHER; note = 2-methoxyethoxy ribonucleotide
  modified_base 1..22: mod_base = OTHER; note = Phosphorothioate linkage
gcaagcagtgttcaaatctcac

SEQ ID NO: 73 - DNA, 22 bp, synthetic construct
  source 1..22: mol_type = other DNA
  modified_base 1..9: mod_base = OTHER; note = 2-methoxyethoxy ribonucleotide
  modified_base 10: mod_base = OTHER; note = 2-methoxyethoxy thymidine ribonucleotide
  modified_base 11: mod_base = OTHER; note = 2-methoxyethoxy ribonucleotide
  modified_base 12..13: mod_base = OTHER; note = 2-methoxyethoxy thymidine ribonucleotide
  modified_base 14..17: mod_base = OTHER; note = 2-methoxyethoxy ribonucleotide
  modified_base 18: mod_base = OTHER; note = 2-methoxyethoxy thymidine ribonucleotide
  modified_base 19: mod_base = OTHER; note = 2-methoxyethoxy ribonucleotide
  modified_base 20: mod_base = OTHER; note = 2-methoxyethoxy thymidine ribonucleotide
  modified_base 21..22: mod_base = OTHER; note = 2-methoxyethoxy ribonucleotide
  modified_base 1..22: mod_base = OTHER; note = Phosphorothioate linkage
agcaagcagtgttcaaatctca

SEQ ID NO: 74 - DNA, 22 bp, synthetic construct
  source 1..22: mol_type = other DNA
  modified_base 1..10: mod_base = OTHER; note = 2-methoxyethoxy ribonucleotide
  modified_base 11: mod_base = OTHER; note = 2-methoxyethoxy thymidine ribonucleotide
  modified_base 12: mod_base = OTHER; note = 2-methoxyethoxy ribonucleotide
  modified_base 13..14: mod_base = OTHER; note = 2-methoxyethoxy thymidine ribonucleotide
  modified_base 15..18: mod_base = OTHER; note = 2-methoxyethoxy ribonucleotide
  modified_base 19: mod_base = OTHER; note = 2-methoxyethoxy thymidine ribonucleotide
  modified_base 20: mod_base = OTHER; note = 2-methoxyethoxy ribonucleotide
  modified_base 21: mod_base = OTHER; note = 2-methoxyethoxy thymidine ribonucleotide
  modified_base 22: mod_base = OTHER; note = 2-methoxyethoxy ribonucleotide
  modified_base 1..22: mod_base = OTHER; note = Phosphorothioate linkage
aagcaagcagtgttcaaatctc

SEQ ID NO: 75 - DNA, 22 bp, synthetic construct
  source 1..22: mol_type = other DNA
  modified_base 1..11: mod_base = OTHER; note = 2-methoxyethoxy ribonucleotide
  modified_base 12: mod_base = OTHER; note = 2-methoxyethoxy thymidine ribonucleotide
  modified_base 13: mod_base = OTHER; note = 2-methoxyethoxy ribonucleotide
  modified_base 14..15: mod_base = OTHER; note = 2-methoxyethoxy thymidine ribonucleotide
  modified_base 16..19: mod_base = OTHER; note = 2-methoxyethoxy ribonucleotide
  modified_base 20: mod_base = OTHER; note = 2-methoxyethoxy thymidine ribonucleotide
  modified_base 21: mod_base = OTHER; note = 2-methoxyethoxy ribonucleotide
  modified_base 22: mod_base = OTHER; note = 2-methoxyethoxy thymidine ribonucleotide
  modified_base 1..22: mod_base = OTHER; note = Phosphorothioate linkage
aaagcaagcagtgttcaaatct

SEQ ID NO: 76 - DNA, 22 bp, synthetic construct
  source 1..22: mol_type = other DNA
  modified_base 1..12: mod_base = OTHER; note = 2-methoxyethoxy ribonucleotide
  modified_base 13: mod_base = OTHER; note = 2-methoxyethoxy thymidine ribonucleotide
  modified_base 14: mod_base = OTHER; note = 2-methoxyethoxy ribonucleotide
  modified_base 15..16: mod_base = OTHER; note = 2-methoxyethoxy thymidine ribonucleotide
  modified_base 17..20: mod_base = OTHER; note = 2-methoxyethoxy ribonucleotide
  modified_base 21: mod_base = OTHER; note = 2-methoxyethoxy thymidine ribonucleotide
  modified_base 22: mod_base = OTHER; note = 2-methoxyethoxy ribonucleotide
  modified_base 1..22: mod_base = OTHER; note = Phosphorothioate linkage
caaagcaagcagtgttcaaatc

SEQ ID NO: 77 - DNA, 22 bp, synthetic construct
  source 1..22: mol_type = other DNA
  modified_base 1..5: mod_base = OTHER; note = 2-methoxyethoxy ribonucleotide
  modified_base 6..7: mod_base = OTHER; note = 2-methoxyethoxy thymidine ribonucleotide
  modified_base 8: mod_base = OTHER; note = 2-methoxyethoxy ribonucleotide
  modified_base 9..10: mod_base = OTHER; note = 2-methoxyethoxy thymidine ribonucleotide
  modified_base 11..20: mod_base = OTHER; note = 2-methoxyethoxy ribonucleotide
  modified_base 21: mod_base = OTHER; note = 2-methoxyethoxy thymidine ribonucleotide
  modified_base 22: mod_base = OTHER; note = 2-methoxyethoxy ribonucleotide
  modified_base 1..22: mod_base = OTHER; note = Phosphorothioate linkage
aggacttattgagaaggaaatg

SEQ ID NO: 78 - DNA, 22 bp, synthetic construct
  source 1..22: mol_type = other DNA
  modified_base 1: mod_base = OTHER; note = 2-methoxyethoxy thymidine ribonucleotide
  modified_base 2..10: mod_base = OTHER; note = 2-methoxyethoxy ribonucleotide
  modified_base 11..12: mod_base = OTHER; note = 2-methoxyethoxy thymidine ribonucleotide
  modified_base 13: mod_base = OTHER; note = 2-methoxyethoxy ribonucleotide
  modified_base 14..15: mod_base = OTHER; note = 2-methoxyethoxy thymidine ribonucleotide
  modified_base 16..22: mod_base = OTHER; note = 2-methoxyethoxy ribonucleotide
  modified_base 1..22: mod_base = OTHER; note = Phosphorothioate linkage
tggccaggacttattgagaagg

SEQ ID NO: 79 - DNA, 22 bp, synthetic construct
  source 1..22: mol_type = other DNA
  modified_base 1..3: mod_base = OTHER; note = 2-methoxyethoxy ribonucleotide
  modified_base 4: mod_base = OTHER; note = 2-methoxyethoxy thymidine ribonucleotide
  modified_base 5: mod_base = OTHER; note = 2-methoxyethoxy ribonucleotide
  modified_base 6: mod_base = OTHER; note = 2-methoxyethoxy thymidine ribonucleotide
  modified_base 7..15: mod_base = OTHER; note = 2-methoxyethoxy ribonucleotide
  modified_base 16..17: mod_base = OTHER; note = 2-methoxyethoxy thymidine ribonucleotide
  modified_base 18: mod_base = OTHER; note = 2-methoxyethoxy ribonucleotide
  modified_base 19..20: mod_base = OTHER; note = 2-methoxyethoxy thymidine ribonucleotide
  modified_base 21..22: mod_base = OTHER; note = 2-methoxyethoxy ribonucleotide
  modified_base 1..22: mod_base = OTHER; note = Phosphorothioate linkage
ccctctggccaggacttattga

SEQ ID NO: 80 - DNA, 22 bp, synthetic construct
  source 1..22: mol_type = other DNA
  modified_base 1: mod_base = OTHER; note = 2-methoxyethoxy thymidine ribonucleotide
  modified_base 2: mod_base = OTHER; note = 2-methoxyethoxy ribonucleotide
  modified_base 3: mod_base = OTHER; note = 2-methoxyethoxy thymidine ribonucleotide
  modified_base 4..8: mod_base = OTHER; note = 2-methoxyethoxy ribonucleotide
  modified_base 9: mod_base = OTHER; note = 2-methoxyethoxy thymidine ribonucleotide
  modified_base 10: mod_base = OTHER; note = 2-methoxyethoxy ribonucleotide
  modified_base 11: mod_base = OTHER; note = 2-methoxyethoxy thymidine ribonucleotide
  modified_base 12..20: mod_base = OTHER; note = 2-methoxyethoxy ribonucleotide
  modified_base 21..22: mod_base = OTHER; note = 2-methoxyethoxy thymidine ribonucleotide
  modified_base 1..22: mod_base = OTHER; note = Phosphorothioate linkage
tctcaccctctggccaggactt

SEQ ID NO: 81 - DNA, 22 bp, synthetic construct
  source 1..22: mol_type = other DNA
  modified_base 1: mod_base = OTHER; note = 2-methoxyethoxy thymidine ribonucleotide
  modified_base 2..5: mod_base = OTHER; note = 2-methoxyethoxy ribonucleotide
  modified_base 6: mod_base = OTHER; note = 2-methoxyethoxy thymidine ribonucleotide
  modified_base 7: mod_base = OTHER; note = 2-methoxyethoxy ribonucleotide
  modified_base 8: mod_base = OTHER; note = 2-methoxyethoxy thymidine ribonucleotide
  modified_base 9..13: mod_base = OTHER; note = 2-methoxyethoxy ribonucleotide
  modified_base 14: mod_base = OTHER; note = 2-methoxyethoxy thymidine ribonucleotide
  modified_base 15: mod_base = OTHER; note = 2-methoxyethoxy ribonucleotide
  modified_base 16: mod_base = OTHER; note = 2-methoxyethoxy thymidine ribonucleotide
  modified_base 17..22: mod_base = OTHER; note = 2-methoxyethoxy ribonucleotide
  modified_base 1..22: mod_base = OTHER; note = Phosphorothioate linkage
tcaaatctcaccctctggccag

SEQ ID NO: 82 - DNA, 22 bp, synthetic construct
  source 1..22: mol_type = other DNA
  modified_base 1: mod_base = OTHER; note = 2-methoxyethoxy thymidine ribonucleotide
  modified_base 2: mod_base = OTHER; note = 2-methoxyethoxy ribonucleotide
  modified_base 3: mod_base = OTHER; note = 2-methoxyethoxy thymidine ribonucleotide
  modified_base 4..17: mod_base = OTHER; note = 2-methoxyethoxy ribonucleotide
  modified_base 18: mod_base = OTHER; note = 2-methoxyethoxy thymidine ribonucleotide
  modified_base 19: mod_base = OTHER; note = 2-methoxyethoxy ribonucleotide
  modified_base 20..21: mod_base = OTHER; note = 2-methoxyethoxy thymidine ribonucleotide
  modified_base 22: mod_base = OTHER; note = 2-methoxyethoxy ribonucleotide
  modified_base 1..22: mod_base = OTHER; note = Phosphorothioate linkage
tctaacaaagcaagcagtgttc

SEQ ID NO: 83 - DNA, 22 bp, synthetic construct
  source 1..22: mol_type = other DNA
  modified_base 1..5: mod_base = OTHER; note = 2-methoxyethoxy ribonucleotide
  modified_base 6: mod_base = OTHER; note = 2-methoxyethoxy thymidine ribonucleotide
  modified_base 7: mod_base = OTHER; note = 2-methoxyethoxy ribonucleotide
  modified_base 8: mod_base = OTHER; note = 2-methoxyethoxy thymidine ribonucleotide
  modified_base 9..22: mod_base = OTHER; note = 2-methoxyethoxy ribonucleotide
  modified_base 1..22: mod_base = OTHER; note = Phosphorothioate linkage
cacagtctaacaaagcaagcag

SEQ ID NO: 84 - DNA, 22 bp, synthetic construct
  source 1..22: mol_type = other DNA
  modified_base 1..6: mod_base = OTHER; note = 2-methoxyethoxy ribonucleotide
  modified_base 7: mod_base = OTHER; note = 2-methoxyethoxy thymidine ribonucleotide
  modified_base 8: mod_base = OTHER; note = 2-methoxyethoxy ribonucleotide
  modified_base 9: mod_base = OTHER; note = 2-methoxyethoxy thymidine ribonucleotide
  modified_base 10..13: mod_base = OTHER; note = 2-methoxyethoxy ribonucleotide
  modified_base 14: mod_base = OTHER; note = 2-methoxyethoxy thymidine ribonucleotide
  modified_base 15..19: mod_base = OTHER; note = 2-methoxyethoxy ribonucleotide
  modified_base 20: mod_base = OTHER; note = 2-methoxyethoxy thymidine ribonucleotide
  modified_base 21..22: mod_base = OTHER; note = 2-methoxyethoxy ribonucleotide
  modified_base 1..22: mod_base = OTHER; note = Phosphorothioate linkage
cccacctataaggtaaaagtga

SEQ ID NO: 85 - DNA, 22 bp, synthetic construct
  source 1..22: mol_type = other DNA
  modified_base 1..5: mod_base = OTHER; note = 2-methoxyethoxy ribonucleotide
  modified_base 6: mod_base = OTHER; note = 2-methoxyethoxy thymidine ribonucleotide
  modified_base 7: mod_base = OTHER; note = 2-methoxyethoxy ribonucleotide
  modified_base 8: mod_base = OTHER; note = 2-methoxyethoxy thymidine ribonucleotide
  modified_base 9..12: mod_base = OTHER; note = 2-methoxyethoxy ribonucleotide
  modified_base 13: mod_base = OTHER; note = 2-methoxyethoxy thymidine ribonucleotide
  modified_base 14..18: mod_base = OTHER; note = 2-methoxyethoxy ribonucleotide
  modified_base 19: mod_base = OTHER; note = 2-methoxyethoxy thymidine ribonucleotide
  modified_base 20..21: mod_base = OTHER; note = 2-methoxyethoxy ribonucleotide
  modified_base 22: mod_base = OTHER; note = 2-methoxyethoxy thymidine ribonucleotide
  modified_base 1..22: mod_base = OTHER; note = Phosphorothioate linkage
ccacctataaggtaaaagtgat

SEQ ID NO: 86 - DNA, 22 bp, synthetic construct
  source 1..22: mol_type = other DNA
  modified_base 1..4: mod_base = OTHER; note = 2-methoxyethoxy ribonucleotide
  modified_base 5: mod_base = OTHER; note = 2-methoxyethoxy thymidine ribonucleotide
  modified_base 6: mod_base = OTHER; note = 2-methoxyethoxy ribonucleotide
  modified_base 7: mod_base = OTHER; note = 2-methoxyethoxy thymidine ribonucleotide
  modified_base 8..11: mod_base = OTHER; note = 2-methoxyethoxy ribonucleotide
  modified_base 12: mod_base = OTHER; note = 2-methoxyethoxy thymidine ribonucleotide
  modified_base 13..17: mod_base = OTHER; note = 2-methoxyethoxy ribonucleotide
  modified_base 18: mod_base = OTHER; note = 2-methoxyethoxy thymidine ribonucleotide
  modified_base 19..20: mod_base = OTHER; note = 2-methoxyethoxy ribonucleotide
  modified_base 21: mod_base = OTHER; note = 2-methoxyethoxy thymidine ribonucleotide
  modified_base 22: mod_base = OTHER; note = 2-methoxyethoxy ribonucleotide
  modified_base 1..22: mod_base = OTHER; note = Phosphorothioate linkage
cacctataaggtaaaagtgatg

SEQ ID NO: 87 - DNA, 22 bp, synthetic construct
  source 1..22: mol_type = other DNA
  modified_base 1..3: mod_base = OTHER; note = 2-methoxyethoxy ribonucleotide
  modified_base 4: mod_base = OTHER; note = 2-methoxyethoxy thymidine ribonucleotide
  modified_base 5: mod_base = OTHER; note = 2-methoxyethoxy ribonucleotide
  modified_base 6: mod_base = OTHER; note = 2-methoxyethoxy thymidine ribonucleotide
  modified_base 7..10: mod_base = OTHER; note = 2-methoxyethoxy ribonucleotide
  modified_base 11: mod_base = OTHER; note = 2-methoxyethoxy thymidine ribonucleotide
  modified_base 12..16: mod_base = OTHER; note = 2-methoxyethoxy ribonucleotide
  modified_base 17: mod_base = OTHER; note = 2-methoxyethoxy thymidine ribonucleotide
  modified_base 18..19: mod_base = OTHER; note = 2-methoxyethoxy ribonucleotide
  modified_base 20: mod_base = OTHER; note = 2-methoxyethoxy thymidine ribonucleotide
  modified_base 21..22: mod_base = OTHER; note = 2-methoxyethoxy ribonucleotide
  modified_base 1..22: mod_base = OTHER; note = Phosphorothioate linkage
acctataaggtaaaagtgatgg

SEQ ID NO: 88 - DNA, 22 bp, synthetic construct
  source 1..22: mol_type = other DNA
  modified_base 1..2: mod_base = OTHER; note = 2-methoxyethoxy ribonucleotide
  modified_base 3: mod_base = OTHER; note = 2-methoxyethoxy thymidine ribonucleotide
  modified_base 4: mod_base = OTHER; note = 2-methoxyethoxy ribonucleotide
  modified_base 5: mod_base = OTHER; note = 2-methoxyethoxy thymidine ribonucleotide
  modified_base 6..9: mod_base = OTHER; note = 2-methoxyethoxy ribonucleotide
  modified_base 10: mod_base = OTHER; note = 2-methoxyethoxy thymidine ribonucleotide
  modified_base 11..15: mod_base = OTHER; note = 2-methoxyethoxy ribonucleotide
  modified_base 16: mod_base = OTHER; note = 2-methoxyethoxy thymidine ribonucleotide
  modified_base 17..18: mod_base = OTHER; note = 2-methoxyethoxy ribonucleotide
  modified_base 19: mod_base = OTHER; note = 2-methoxyethoxy thymidine ribonucleotide
  modified_base 20..22: mod_base = OTHER; note = 2-methoxyethoxy ribonucleotide
  modified_base 1..22: mod_base = OTHER; note = Phosphorothioate linkage
cctataaggtaaaagtgatggg

SEQ ID NO: 89 - DNA, 22 bp, synthetic construct
  source 1..22: mol_type = other DNA
  modified_base 1: mod_base = OTHER; note = 2-methoxyethoxy ribonucleotide
  modified_base 2: mod_base = OTHER; note = 2-methoxyethoxy thymidine ribonucleotide
  modified_base 3: mod_base = OTHER; note = 2-methoxyethoxy ribonucleotide
  modified_base 4: mod_base = OTHER; note = 2-methoxyethoxy thymidine ribonucleotide
  modified_base 5..8: mod_base = OTHER; note = 2-methoxyethoxy ribonucleotide
  modified_base 9: mod_base = OTHER; note = 2-methoxyethoxy thymidine ribonucleotide
  modified_base 10..14: mod_base = OTHER; note = 2-methoxyethoxy ribonucleotide
  modified_base 15: mod_base = OTHER; note = 2-methoxyethoxy thymidine ribonucleotide
  modified_base 16..17: mod_base = OTHER; note = 2-methoxyethoxy ribonucleotide
  modified_base 18: mod_base = OTHER; note = 2-methoxyethoxy thymidine ribonucleotide
  modified_base 19..22: mod_base = OTHER; note = 2-methoxyethoxy ribonucleotide
  modified_base 1..22: mod_base = OTHER; note = Phosphorothioate linkage
ctataaggtaaaagtgatggga

SEQ ID NO: 90 - DNA, 22 bp, synthetic construct
  source 1..22: mol_type = other DNA
  modified_base 1: mod_base = OTHER; note = 2-methoxyethoxy thymidine ribonucleotide
  modified_base 2: mod_base = OTHER; note = 2-methoxyethoxy ribonucleotide
  modified_base 3: mod_base = OTHER; note = 2-methoxyethoxy thymidine ribonucleotide
  modified_base 4..7: mod_base = OTHER; note = 2-methoxyethoxy ribonucleotide
  modified_base 8: mod_base = OTHER; note = 2-methoxyethoxy thymidine ribonucleotide
  modified_base 9..13: mod_base = OTHER; note = 2-methoxyethoxy ribonucleotide
  modified_base 14: mod_base = OTHER; note = 2-methoxyethoxy thymidine ribonucleotide
  modified_base 15..16: mod_base = OTHER; note = 2-methoxyethoxy ribonucleotide
  modified_base 17: mod_base = OTHER; note = 2-methoxyethoxy thymidine ribonucleotide
  modified_base 18..21: mod_base = OTHER; note = 2-methoxyethoxy ribonucleotide
  modified_base 22: mod_base = OTHER; note = 2-methoxyethoxy thymidine ribonucleotide
  modified_base 1..22: mod_base = OTHER; note = Phosphorothioate linkage
tataaggtaaaagtgatgggat

SEQ ID NO: 91 - DNA, 22 bp, synthetic construct
  source 1..22: mol_type = other DNA
  modified_base 1: mod_base = OTHER; note = 2-methoxyethoxy ribonucleotide
  modified_base 2: mod_base = OTHER; note = 2-methoxyethoxy thymidine ribonucleotide
  modified_base 3..6: mod_base = OTHER; note = 2-methoxyethoxy ribonucleotide
  modified_base 7: mod_base = OTHER; note = 2-methoxyethoxy thymidine ribonucleotide
  modified_base 8..12: mod_base = OTHER; note = 2-methoxyethoxy ribonucleotide
  modified_base 13: mod_base = OTHER; note = 2-methoxyethoxy thymidine ribonucleotide
  modified_base 14..15: mod_base = OTHER; note = 2-methoxyethoxy ribonucleotide
  modified_base 16: mod_base = OTHER; note = 2-methoxyethoxy thymidine ribonucleotide
  modified_base 17..20: mod_base = OTHER; note = 2-methoxyethoxy ribonucleotide
  modified_base 21: mod_base = OTHER; note = 2-methoxyethoxy thymidine ribonucleotide
  modified_base 22: mod_base = OTHER; note = 2-methoxyethoxy ribonucleotide
  modified_base 1..22: mod_base = OTHER; note = Phosphorothioate linkage
ataaggtaaaagtgatgggatc

SEQ ID NO: 92 - DNA, 22 bp, synthetic construct
  source 1..22: mol_type = other DNA
  modified_base 1: mod_base = OTHER; note = 2-methoxyethoxy thymidine ribonucleotide
  modified_base 2..5: mod_base = OTHER; note = 2-methoxyethoxy ribonucleotide
  modified_base 6: mod_base = OTHER; note = 2-methoxyethoxy thymidine ribonucleotide
  modified_base 7..11: mod_base = OTHER; note = 2-methoxyethoxy ribonucleotide
  modified_base 12: mod_base = OTHER; note = 2-methoxyethoxy thymidine ribonucleotide
  modified_base 13..14: mod_base = OTHER; note = 2-methoxyethoxy ribonucleotide
  modified_base 15: mod_base = OTHER; note = 2-methoxyethoxy thymidine ribonucleotide
  modified_base 16..19: mod_base = OTHER; note = 2-methoxyethoxy ribonucleotide
  modified_base 20: mod_base = OTHER; note = 2-methoxyethoxy thymidine ribonucleotide
  modified_base 21..22: mod_base = OTHER; note = 2-methoxyethoxy ribonucleotide
  modified_base 1..22: mod_base = OTHER; note = Phosphorothioate linkage
taaggtaaaagtgatgggatca

SEQ ID NO: 93 - DNA, 22 bp, synthetic construct
  source 1..22: mol_type = other DNA
  modified_base 1..4: mod_base = OTHER; note = 2-methoxyethoxy ribonucleotide
  modified_base 5: mod_base = OTHER; note = 2-methoxyethoxy thymidine ribonucleotide
  modified_base 6..10: mod_base = OTHER; note = 2-methoxyethoxy ribonucleotide
  modified_base 11: mod_base = OTHER; note = 2-methoxyethoxy thymidine ribonucleotide
  modified_base 12..13: mod_base = OTHER; note = 2-methoxyethoxy ribonucleotide
  modified_base 14: mod_base = OTHER; note = 2-methoxyethoxy thymidine ribonucleotide
  modified_base 15..18: mod_base = OTHER; note = 2-methoxyethoxy ribonucleotide
  modified_base 19: mod_base = OTHER; note = 2-methoxyethoxy thymidine ribonucleotide
  modified_base 20..22: mod_base = OTHER; note = 2-methoxyethoxy ribonucleotide
  modified_base 1..22: mod_base = OTHER; note = Phosphorothioate linkage
aaggtaaaagtgatgggatcac

SEQ ID NO: 94 - RNA, 20 nt, synthetic construct
  source 1..20: mol_type = other RNA
tgctcagttatagtatataa

SEQ ID NO: 95 - RNA, 20 nt, synthetic construct
  source 1..20: mol_type = other RNA
ttagttatctgtttaaacta

SEQ ID NO: 96 - DNA, 20 bp, synthetic construct
  source 1..20: mol_type = other DNA
gactcccctgtccttgttga

SEQ ID NO: 97 - DNA, 23 bp, synthetic construct
  source 1..23: mol_type = other DNA
gatcccactcctaggtccttcga

SEQ ID NO: 98 - DNA, 23 bp, synthetic construct
  source 1..23: mol_type = other DNA
agccagcacagcctcttagatgc

SEQ ID NO: 99 - protein, 9 aa, synthetic construct
VRFEHCLLC

(19) United States
(12) Patent Application Publication
(10) Pub. No.: US 2026/0125686 A1
(43) Pub. Date: May 7, 2026

(54) COMPOSITIONS AND METHODS FOR MODULATION OF CFTR

(71) Applicant: Cystic Fibrosis Foundation, Bethesda, MD (US)

(72) Inventors: Normand ALLAIRE, Bethesda, MD (US); Matthew ARMSTRONG, Bethesda, MD (US); Jae Seok YOON, Bethesda, MD (US)

(21) Appl. No.: 19/117,420

(22) PCT Filed: Oct. 2, 2023
(86) PCT No.: PCT/US23/75747

§ 371 Date: Apr. 1, 2025

Related U.S. Application Data
(60) Provisional application No. 63/412,771, filed on Oct. 3, 2022.

(51) Int. Cl. C12N 15/113

(52) U.S. Cl. CPC C12N 15/1138; C12N 2310/14; C12N 2320/33

(57) ABSTRACT

In some aspects, provided herein are compositions, methods, and kits relating to an agent modulates expression of a CFTR protein. An agent provide herein can modify the CFTR gene or modulate process of the CFTR pre-mRNA. In some embodiments, the compositions, methods, and kits provided herein are applicable for treatment of cystic fibrosis.

### CROSS-REFERENCE

This application claims the benefit of U.S. Provisional Application No. 63/412,771, filed Oct. 3, 2022, which application is incorporated herein by reference.

### BACKGROUND

Cystic fibrosis (CF) is the most common life-shortening autosomal recessive disease among populations of Northern European descent, with a frequency of 1 in 2000 to 3000 live births. Despite progress in the treatment of CF, there is no cure. Cystic fibrosis can be caused by pathogenic mutations in the CFTR gene, which comprises 250 kilobases of genomic sequence that encodes an epithelial cell protein that is composed of 1480 amino acids in its mature state.

Premature Termination Codons (PTC) mutations in the CFTR gene represent the largest class of cystic fibrosis (CF) causing mutations for which there is no therapy. PTC mutations can result in a poorly functional or non-functional protein product and can trigger a dramatic reduction of CFTR mRNA template via Nonsense Mediated mRNA Decay (NMD). Certain C-terminal truncated CFTR protein can retain some chloride channel function in the cells. Therefore, without wishing to be bound by a certain theory, inhibition of NMD to increase truncated protein expression is an attractive approach for treating diseases or conditions caused by PTC mutations near the 3′ end of CFTR. However, given the critical role of NMD as a global quality control mechanism of the cell, a CFTR-specific mechanism to evade NMD is desirable.

### SUMMARY

In some aspects, the present disclosure provides a method of modulating expression of a CFTR gene in a cell, comprising contacting an agent or a vector encoding the agent to the cell, wherein the cell comprises a pre-mRNA, wherein the pre-mRNA is transcribed from the CFTR gene and comprises a first intron that comprises an alternative polyadenylation site, and wherein the agent modifies the CFTR gene or modulates processing of the pre-mRNA, thereby increasing a level of a processed mRNA that is processed from the pre-mRNA and that lacks nucleic acid sequence of the pre-mRNA downstream of the first intron. In some embodiments, the cell is a human cell, and the CFTR gene is a human gene. In some embodiments, the first intron is Intron 22. In some embodiments, first intron is located at a region from GRCh38.p14/hg38: chr7: 117,627,771 to GRCh38.p14/hg38: chr7: 117,642,437. In some embodiments, the agent removes from genome of the cell nucleic acid sequence of the CFTR gene that is downstream of the first intron. In some embodiments, the nucleic acid sequence that is removed from the genome is located from GRCh38.p14/hg38: chr7: 117,642,438 to GRCh38.p14/hg38: chr7: 117,668,665. In some embodiments, the agent comprises gene editing agents based on CRISPR/Cas9, TALEN, Zinc Finger, or any combination thereof. In some embodiments, the agent comprises a pair of guide RNAs, and wherein the pair of guide RNAs comprise the sequences of SEQ ID NOs: 94 and 95, respectively. In some embodiments, the agent removes from the pre-mRNA the nucleic acid sequence of the pre-mRNA downstream of the first intron. In some embodiments, the nucleic acid sequence that is removed from the pre-mRNA is located from GRCh38.p14/hg38: chr7: 117,642,438 to GRCh38.p14/hg38: chr7: 117,668,665. In some embodiments, the agent suppresses splicing out of the first intron from the pre-mRNA.

In some aspects, the present disclosure provides a method of modulating expression of a CFTR gene in a cell, comprising contacting an agent or a vector encoding the agent to the cell, wherein the cell comprises a pre-mRNA that is transcribed from the CFTR gene and comprises a first intron that comprises an alternative polyadenylation site, and wherein the agent suppresses splicing out of the first intron from the pre-mRNA during splicing of the pre-mRNA in the cell. In some embodiments, the cell is a human cell, and the CFTR gene is a human gene. In some embodiments, first intron is Intron 22. In some embodiments, the first intron is located at a region from GRCh38.p14/hg38: chr7: 117,627,771 to GRCh38.p14/hg38: chr7: 117,642,437. In some embodiments, the agent increases a level of a processed mRNA that is processed from the pre-mRNA and that lacks nucleic acid sequence of the pre-mRNA downstream of the first intron. In some embodiments, the nucleic acid sequence of the pre-mRNA downstream of the first intron is located at a region from GRCh38.p14/hg38: chr7: 117,642,438 to GRCh38.p14/hg38: chr7: 117,668,665.

In some embodiments, the level of the processed mRNA is increased in the cell by at least about 1.5, at least about 2, at least about 3, at least about 4, at least about 5, at least about 6, at least about 7, at least about 8, at least about 9, at least about 10, at least about 11, at least about 12, at least about 13, at least about 14, at least about 15, or at least about 20 times as compared to a corresponding cell that is not contacted with the agent or the vector encoding the agent. In some embodiments, the level of the processed mRNA is increased in the cell by at least about 10 times as compared to a corresponding cell that is not contacted with the agent or the vector encoding the agent.

In some embodiments, the processed mRNA comprises, in a 5′ to 3′ order, 22 exons, an intronic sequence encoding nine amino acids, a stop codon, and an alternative 3′ untranslated region. In some embodiments, the intronic sequence encoding nine amino acids is transcribed from genomic sequence located from GRCh38.p14/hg38: chr7: 117,627,771 to GRCh38.p14/hg38: chr7: 117,627,797. In some embodiments, the processed mRNA is polyadenylated at an alternative polyadenylation site in a nucleic acid sequence transcribed from genomic sequence located between GRCh38.p14/hg38: chr7: 117,627,771 and GRCh38.p14/hg38: chr7: 117,642,437.

In some embodiments, the agent increases a level of a truncated CTFR protein in the cell, which lacks amino acid sequence expressed from exonic sequences of the CFTR gene downstream of the first intron. In some embodiments, the level of the truncated CFTR protein in the cell is increased by at least about 1.5, at least about 2, at least about 3, at least about 4, at least about 5, at least about 6, at least about 7, at least about 8, at least about 9, at least about 10, at least about 11, at least about 12, at least about 13, at least about 14, at least about 15, or at least about 20 times as compared to a corresponding cell that is not contacted with the agent or the vector encoding the agent. In some embodiments, the truncated CFTR protein in the cell has a chloride channel conductivity that is less than 100%, less than 90%, less than 80%, less than 70%, less than 60%, less than 50%, less than 40%, less than 30%, less than 20%, less than 10%, or less than 5% of a chloride channel conductivity of a wildtype CFTR protein.

In some embodiments, the methods provided herein further comprises contacting the cell with a second agent. In some embodiments, the second agent comprises a modulator of CFTR protein that potentiates chloride conductance of CFTR in the cells. In some embodiments, the second agent comprises ivacaftor, lumacaftor, tezacaftor, elexacaftor, a combination of lumacaftor and ivacaftor, a combination of tezacaftor and ivacaftor, or a combination of elexacaftor, ivacaftor, and tezacaftor. In some embodiments, the second agent improves the chloride channel conductivity of the truncated CFTR protein by at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300%, at least 350%, at least 400%, at least 450%, at least 500%, at least 550%, at least 600%, at least 650%, at least 700%, at least 750%, at least 800%, at least at least 850%, at least 900%, at least 950%, at least 1000%. In some embodiments, the second agent restores the chloride channel conductivity of the truncated CFTR protein to about at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more of the chloride channel conductivity of wildtype CFTR protein.

In some embodiments, the agent: (a) binds to a 5′ splice site of the first intron; (b) binds to a 3′ splice site of the first intron; (c) binds to a branch point for the 3′ splice site of the first intron; or (d) interferes with a splicing factor that is involved in splicing out of the first intron.

In some embodiments, the agent comprises a polynucleotide sequence that is at least 80% complementary to at least 8 contiguous nucleic acids of a sequence selected from the group consisting of SEQ ID NOs: 6-36. In some embodiments, the agent comprises a polynucleotide sequence that is at least 80% complementary to at least 10, 12, 14, or 16 contiguous nucleic acids of a sequence selected from the group consisting of SEQ ID NOs: 6-36. In some embodiments, the agent comprises a polynucleotide sequence that is at least 80% complementary to at least 18 contiguous nucleic acids of a sequence selected from the group consisting of SEQ ID NOs: 6-36. In some embodiments, the agent comprises a polynucleotide sequence that is 100% complementary to a sequence selected from the group consisting of SEQ ID NOs: 6-36. In some embodiments, the agent comprises a polynucleotide sequence with at least 80% identity to a sequence selected from the group consisting of SEQ ID NOs: 37-64. In some embodiments, the agent comprises a polynucleotide sequence that comprises at least 8 contiguous nucleotides of a sequence selected from the group consisting of SEQ ID NOs: 37-64.

In some aspects, the present disclosure provides a method comprising contacting an agent or a vector encoding the agent to the cell, wherein the agent comprises a polynucleotide sequence that is at least 80% complementary to a sequence selected from the group consisting of SEQ ID NOs: 6-36.

In some aspects, the present disclosure provides a method comprising contacting an agent or a vector encoding the agent to the cell, wherein the agent comprises a polynucleotide sequence with at least 80% identity to a sequence selected from the group consisting of SEQ ID NOs: 37-64.

In some aspects, the present disclosure provides a method comprising contacting an agent or a vector encoding the agent to the cell, wherein the agent comprises at least 8 contiguous nucleotides of a sequence selected from the group consisting of SEQ ID NOs: 37-64.

In some embodiments, the polynucleotide sequence of the agent has at least 90% identity to a sequence selected from the group consisting of SEQ ID NOs: 37-64. In some embodiments, the polynucleotide sequence of the agent has 100% identity to a sequence selected from the group consisting of SEQ ID NOs: 37-64. In some embodiments, the polynucleotide sequence of the agent comprises at least 10, 12, 14, or 16 contiguous nucleotides of a sequence selected from the group consisting of SEQ ID NOs: 37-64. In some embodiments, the polynucleotide sequence of the agent comprises at least 18 contiguous nucleotides of a sequence selected from the group consisting of SEQ ID NOs: 37-64. In some embodiments, the polynucleotide sequence of the agent comprises at least 20 contiguous nucleotides of a sequence selected from the group consisting of SEQ ID NOs: 37-64.

In some embodiments, the agent is an antisense oligomer. In some embodiments, the antisense oligomer comprises a backbone modification, a modified sugar moiety or a combination thereof. In some embodiments, the antisense oligomer comprises a phosphorothioate linkage or a phosphorodiamidate linkage. In some embodiments, each internucleotide linkage of the antisense oligomer is a phosphorothioate linkage. In some embodiments, the antisense oligomer comprises a phosphorodiamidate morpholino, a locked nucleic acid, a peptide nucleic acid, a 2′-O-methyl moiety, a 2′-Fluoro moiety, a 2′-O-methoxyethyl moiety, or a 2′-NMA moiety. In some embodiments, the antisense oligomer comprises at least one modified sugar moiety. In some embodiments, each nucleotide of the antisense oligomer comprises a modified sugar moiety. In some embodiments, each nucleotide of the antisense oligomer comprises a 2′-O-methoxyethyl moiety. In some embodiments, the antisense oligomer comprises at least one modified nucleobase. In some embodiments, wherein the antisense oligomer comprises hypoxanthine, xanthine, 7-methylguanine, 5, 6-dihydrouracil, 5-methylcytosine, or 5-hydroxymethoylcytosine.

In some embodiments, the antisense oligomer is from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 12 to 50 nucleobases, 12 to 40 nucleobases, 12 to 35 nucleobases, 12 to 30 nucleobases, 12 to 25 nucleobases, 12 to 20 nucleobases, 12 to 15 nucleobases, 15 to 50 nucleobases, 15 to 40 nucleobases, 15 to 35 nucleobases, 15 to 30 nucleobases, 15 to 25 nucleobases, 15 to 20 nucleobases, 15 to 19 nucleobases, 15 to 18 nucleobases, 15 to 16 nucleobases, 16 to 20 nucleobases, 16 to 19 nucleobases, 16 to 18 nucleobases, 17 to 20 nucleobases, 17 to 19 nucleobases, or 18 to 20 nucleobases in length. In some embodiments, the antisense oligomer comprises the sequence set forth in any one of SEQ ID NOs: 66-93.

In some embodiments, the method comprises contacting to the cell the vector, and wherein the vector comprises a viral vector encoding the agent. In some embodiments, the viral vector comprises an adenoviral vector, adeno-associated viral (AAV) vector, lentiviral vector, Herpes Simplex Virus (HSV) viral vector, or retroviral vector.

In some embodiments, the CFTR gene comprises a mutation downstream the first intron. In some embodiments, the mutation downstream of the first intron is a nonsense mutation. In some embodiments, the CFTR gene comprises a mutation that leads to presence of an in-frame premature termination codon that is downstream of the first intron. In some embodiments, at least one allele of the CFTR gene in the cell is a variant selected from the group consisting of: c.3717G>A, c.3717+4A>G, c.3717+5G>A, c.3717+40A>G, c.3718-2477C>T, c.3718-1G>A, c.3718-3T>G, c.3719T>G, c.3731G>A, c.3737C>T, c.3744delA, c.3745G>A, c.3747delG, c.3752G>A, c.3761T>G, c.3763T>C, c.3764C>A, c.3773dupT, c.3806T>A, c.3808delG, c.3808G>A, c.3822G>A, c.3846G>A, c. [3846G>A;3848G>T], c.3848G>T, c.3872A>G, c.3873G>C, c.3873+1G>A, c. (3873+1_3874-1)_(3963+1_3964-1) del, c.3873+2T>C, 3876delA, c.3883_3886delATTT, c.3883delA, c.3889dupT, c.3891 dupT, c.3908delA, c.3909C>G, c.3929G>A, c.3937C>T, c. (3963+1_3964-1)_(*1_?) del, c.3964-78_4242+577del, c.3971T>C, c.3988C>T, c.4004T>C, c.4036_4042del, c.4046G>A, c.4077_4080delTGTTinsAA, c.4086dupT, c.4097T>A, c.4111G>T, c.4124A>C, c.4127_4131delTGGAT, c.4144C>T, c.4147dupA, c.4197_4198delCT, c.4231C>T, c.4234C>T, c.4242+1G>T, c.4242+1G>A, c.4251delA, c.4300_4301dup, c.4364C>G, c.4426C>T, and c.4439T>C.

In some aspects, the present disclosure provides a composition comprising an agent or a vector encoding the agent, wherein the agent, when present in a human cell that comprises a pre-mRNA, the pre-mRNA being transcribed from the CFTR gene and comprising a first intron that comprises an alternative polyadenylation site, modifies the CFTR gene or modulates processing of the pre-mRNA, thereby increasing a level of a processed mRNA that is processed from the pre-mRNA and that lacks nucleic acid sequence of the pre-mRNA downstream of the first intron.

In some embodiments, the cell is a human cell, and the CFTR gene is a human gene. In some embodiments, the first intron is Intron 22. In some embodiments, the first intron is located at a region from GRCh38.p14/hg38: chr7: 117,627,771 to GRCh38.p14/hg38: chr7: 117,642,437. In some embodiments, the agent removes from genome of the cell nucleic acid sequence of the CFTR gene that is downstream of the first intron. In some embodiments, the removed nucleic acid sequence is located from GRCh38.p14/hg38: chr7: 117,642,438 to GRCh38.p14/hg38: chr7: 117,668,665. In some embodiments, the agent comprises gene editing agents based on CRISPR/Cas9, TALEN, Zinc Finger, or any combination thereof. In some embodiments, the agent comprises a pair of guide RNAs, and wherein the pair of guide RNAs comprise the sequences of SEQ ID NOs: 94 and 95, respectively. In some embodiments, the agent removes from the pre-mRNA the nucleic acid sequence of the pre-mRNA downstream of the first intron. In some embodiments, the removed nucleic acid sequence is located from GRCh38.p14/hg38: chr7: 117,642,438 to GRCh38.p14/hg38: chr7: 117,668,665. In some embodiments, the agent suppresses splicing out of the first intron.

In some aspects, the present disclosure provides a composition comprising an agent or a vector encoding the agent, wherein the agent, when present in a cell that comprises a pre-mRNA, the pre-mRNA being transcribed from the CFTR gene and comprising a first intron that comprises an alternative polyadenylation site, suppresses splicing out of the first intron. In some embodiments, the cell is a human cell, and the CFTR gene is a human gene. In some embodiments, the first intron is Intron 22. In some embodiments, the first intron is located at a region from GRCh38.p14/hg38: chr7: 117,627,771 to GRCh38.p14/hg38: chr7: 117,642,437. In some embodiments, the agent increases a level of a processed mRNA that is processed from the pre-mRNA and that lacks nucleic acid sequence of the pre-mRNA downstream of the first intron. In some embodiments, the nucleic acid sequence of the pre-mRNA downstream of the first intron is located at a region from GRCh38.p14/hg38: chr7: 117,642,438 to GRCh38.p14/hg38: chr7: 117,668,665.

In some embodiments, the level of the processed mRNA is increased in the cell by at least about 1.5, at least about 2, at least about 3, at least about 4, at least about 5, at least about 6, at least about 7, at least about 8, at least about 9, at least about 10, at least about 11, at least about 12, at least about 13, at least about 14, at least about 15, or at least about 20 times as compared to a corresponding cell that is not contacted with the agent or the vector encoding the agent. In some embodiments, the level of the processed mRNA is increased in the cell by at least about 10 times as compared to a corresponding cell that is not contacted with the agent or the vector encoding the agent. In some embodiments, the processed mRNA comprises, in a 5′ to 3′ order, 22 exons, an intronic sequence encoding nine amino acids, a stop codon, and an alternative 3′ untranslated region. In some embodiments, the intronic sequence encoding nine amino acids is transcribed from genomic sequence located from GRCh38.p14/hg38: chr7: 117,627,771 to GRCh38.p14/hg38: chr7: 117,627,797. In some embodiments, the processed mRNA is polyadenylated at an alternative polyadenylation site in a nucleic acid sequence transcribed from genomic sequence located between GRCh38.p14/hg38: chr7: 117,627,771 and GRCh38.p14/hg38: chr7: 117,642,437. In some embodiments, the agent increases a level of a truncated CTFR protein in the cell, which lacks amino acid sequence expressed from exonic sequences of the CFTR gene downstream of the first intron. In some embodiments, the level of the truncated CFTR protein in the cell is increased by at least about 1.5, at least about 2, at least about 3, at least about 4, at least about 5, at least about 6, at least about 7, at least about 8, at least about 9, at least about 10, at least about 11, at least about 12, at least about 13, at least about 14, at least about 15, or at least about 20 times as compared to a corresponding cell that is not contacted with the agent or the vector encoding the agent. In some embodiments, the truncated CFTR protein in the cell has a chloride channel conductivity that is less than 100%, less than 90%, less than 80%, less than 70%, less than 60%, less than 50%, less than 40%, less than 30%, less than 20%, less than 10%, or less than 5% of a chloride channel conductivity of a wildtype CFTR protein.

In some embodiments, the composition provided herein further comprises a second agent. In some embodiments, the second agent comprises a modulator of CFTR protein that potentiates chloride conductance of CFTR in the cells. In some embodiments, the second agent comprises ivacaftor, lumacaftor, tezacaftor, elexacaftor, a combination of lumacaftor and ivacaftor, a combination of tezacaftor and ivacaftor, or a combination of elexacaftor, ivacaftor, and tezacaftor. In some embodiments, the second agent improves the chloride channel conductivity of the truncated CFTR protein by at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300%, at least 350%, at least 400%, at least 450%, at least 500%, at least 550%, at least 600%, at least 650%, at least 700%, at least 750%, at least 800%, at least at least 850%, at least 900%, at least 950%, at least 1000%. In some embodiments, the second agent restores the chloride channel conductivity of the truncated CFTR protein to about at least 5%, at least 10%, at least 15%, at least 20%, at least 25%, at least 30%, at least 35%, at least 40%, least 45%, at least 50%, at least 55%, at least 60%, least 65%, at least 70%, at least 75%, at least 80%, at least 85%, at least 90%, at least 95% or more of the chloride channel conductivity of wildtype CFTR protein.

In some embodiments, the agent: (a) binds to a 5′ splice site of the first intron; (b) binds to a 3′ splice site of the first intron; (c) binds to a branch point for the 3′ splice site of the first intron; or (d) interferes with a splicing factor that is involved in splicing out of the first intron.

In some embodiments, the agent comprises a polynucleotide sequence that is at least 80% complementary to at least 8 contiguous nucleic acids of a sequence selected from the group consisting of SEQ ID NOs: 6-36. In some embodiments, the agent comprises a polynucleotide sequence that is at least 80% complementary to at least 10, 12, 14, or 16 contiguous nucleic acids of a sequence selected from the group consisting of SEQ ID NOs: 6-36. In some embodiments, the agent comprises a polynucleotide sequence that is at least 80% complementary to at least 18 contiguous nucleic acids of a sequence selected from the group consisting of SEQ ID NOs: 6-36.

In some embodiments, the agent comprises a polynucleotide sequence that is 100% complementary to a sequence selected from the group consisting of SEQ ID NOs: 6-36. In some embodiments, agent comprises a polynucleotide sequence with at least 80% identity to a sequence selected from the group consisting of SEQ ID NOs: 37-64. In some embodiments, the agent comprises a polynucleotide sequence that comprises at least 8 contiguous nucleotides of a sequence selected from the group consisting of SEQ ID NOs: 37-64.

In some aspects, the present disclosure provides a composition comprising an agent or a vector encoding the agent, wherein the agent comprises a polynucleotide sequence that is at least 80% complementary to a sequence selected from the group consisting of SEQ ID NOs: 6-36.

In some aspects, the present disclosure provides a composition comprising an agent or a vector encoding the agent, wherein the agent comprises a polynucleotide sequence with at least 80% identity to a sequence selected from the group consisting of SEQ ID NOs: 37-64.

In some aspects, the present disclosure provides a composition comprising an agent or a vector encoding the agent, wherein the agent comprises at least 8 contiguous nucleotides of a sequence selected from the group consisting of SEQ ID NOs: 37-64.

In some embodiments, the polynucleotide sequence of the agent has at least 90% identity to a sequence selected from the group consisting of SEQ ID NOs: 37-64. In some embodiments, the polynucleotide sequence of the agent has 100% identity to a sequence selected from the group consisting of SEQ ID NOs: 37-64. In some embodiments, the polynucleotide sequence of the agent comprises at least 10, 12, 14, or 16 contiguous nucleotides of a sequence selected from the group consisting of SEQ ID NOs: 37-64. In some embodiments, the polynucleotide sequence of the agent comprises at least 18 contiguous nucleotides of a sequence selected from the group consisting of SEQ ID NOs: 37-64. In some embodiments, the polynucleotide sequence of the agent comprises at least 20 contiguous nucleotides of a sequence selected from the group consisting of SEQ ID NOs: 37-64. In some embodiments, the polynucleotide sequence of the agent is at least 80% complementary to at least 8 contiguous nucleic acids of a sequence selected from the group consisting of SEQ ID NOs: 6-36. In some embodiments, the polynucleotide sequence of the agent is at least 80% complementary to at least 10, 12, 14, or 16 contiguous nucleic acids of a sequence selected from the group consisting of SEQ ID NOs: 6-36. In some embodiments, the polynucleotide sequence of the agent is at least 80% complementary to at least 18 contiguous nucleic acids of a sequence selected from the group consisting of SEQ ID NOs: 6-36. In some embodiments, the polynucleotide sequence of the agent is 100% complementary to a sequence selected from the group consisting of SEQ ID NOs: 6-36. In some embodiments, the agent is an antisense oligomer. In some embodiments, the antisense oligomer comprises a backbone modification, a modified sugar moiety or a combination thereof. In some embodiments, the antisense oligomer comprises a phosphorothioate linkage or a phosphorodiamidate linkage. In some embodiments, each internucleotide linkage of the antisense oligomer is a phosphorothioate linkage. In some embodiments, the antisense oligomer comprises a phosphorodiamidate morpholino, a locked nucleic acid, a peptide nucleic acid, a 2′-O-methyl moiety, a 2′-Fluoro moiety, a 2′-O-methoxyethyl moiety, or a 2′-NMA moiety. In some embodiments, the antisense oligomer comprises at least one modified sugar moiety. In some embodiments, each nucleotide of the antisense oligomer comprises a modified sugar moiety. In some embodiments, each nucleotide of the antisense oligomer comprises a 2′-O-methoxyethyl moiety. In some embodiments, the antisense oligomer comprises at least one modified nucleobase. In some embodiments, the antisense oligomer comprises hypoxanthine, xanthine, 7-methylguanine, 5, 6-dihydrouracil, 5-methylcytosine, or 5-hydroxymethoylcytosine.

In some embodiments, the antisense oligomer is from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 12 to 50 nucleobases, 12 to 40 nucleobases, 12 to 35 nucleobases, 12 to 30 nucleobases, 12 to 25 nucleobases, 12 to 20 nucleobases, 12 to 15 nucleobases, 15 to 50 nucleobases, 15 to 40 nucleobases, 15 to 35 nucleobases, 15 to 30 nucleobases, 15 to 25 nucleobases, 15 to 20 nucleobases, 15 to 19 nucleobases, 15 to 18 nucleobases, 15 to 16 nucleobases, 16 to 20 nucleobases, 16 to 19 nucleobases, 16 to 18 nucleobases, 17 to 20 nucleobases, 17 to 19 nucleobases, or 18 to 20 nucleobases in length. In some embodiments, the antisense oligomer comprises the sequence set forth in any one of SEQ ID NOs: 66-93. In some embodiments, the composition comprises the vector, and wherein the vector comprises a viral vector encoding the agent. In some embodiments, the viral vector comprises an adenoviral vector, adeno-associated viral (AAV) vector, lentiviral vector, Herpes Simplex Virus (HSV) viral vector, or retroviral vector.

In some aspects, the present disclosure provides a method of treating a subject in need thereof, comprising contacting cells of the subject with the composition provided herein. In some embodiments, the cells are ex vivo. In some embodiments, the cells are in vivo. In some embodiments, the method comprises administering the composition to the subject via intranasal administration, intratracheal administration, intravenous injection, intraperitoneal injection, intramuscular injection, intrathecal injection, subcutaneous injection, oral administration, synovial injection, intracerebroventricular injection, intravitreal administration, subretinal injection, topical application, or implantation. In some embodiments, the method comprises administering the composition to the subject via respiratory route. In some embodiments, the method further comprises administering to the subject a second agent. In some embodiments, the second agent comprises a modulator of CFTR protein that potentiates chloride conductance of CFTR in the cells. In some embodiments, the second agent comprises ivacaftor, lumacaftor, tezacaftor, elexacaftor, a combination of lumacaftor and ivacaftor, a combination of tezacaftor and ivacaftor, or a combination of elexacaftor, ivacaftor, and tezacaftor. In some embodiments, the second agent comprises a mucolytic agent for airway clearance, optionally wherein the mucolytic agent for airway clearance is selected from the group consisting of: acetylcysteine, ambroxol, bromhexine, carbocisteine, erdosteine, mecysteine, dornase alfa, hypertonic saline, and mannitol. In some embodiments, the second agent comprises a bronchodilator, optionally wherein the bronchodilator is albuterol. In some embodiments, the second agent comprises an immunosuppressive agent. In some embodiments, the immunosuppressive agent is a corticosteroid selected from the group consisting of beclomethasone, budesonide, budesonide, formoterol, ciclesonide, fluticasone, salmeterol, umeclidinium, vilanterol, mometasone, or mometasone, prednisone, and methylprednisolone, and pharmaceutically acceptable salts thereof. In some embodiments, the immunosuppressive agent is a non-steroidal immunosuppressive agent selected from the group consisting of polyclonal anti-lymphocyte antibodies, monoclonal anti-lymphocyte antibodies, interleukin-2 (IL-2) receptor antagonists, calcineurin inhibitors, cell cycle inhibitors, mammalian target of rapamycin (mTOR) inhibitors, methotrexate, cyclophosphamide, an anthracycline, a taxane.

In some embodiments, the method treats a disease or condition caused by a mutation in CFTR gene in the subject. In some embodiments, the CFTR gene comprises a mutation downstream the first intron. In some embodiments, the mutation downstream of the first intron is a nonsense mutation. In some embodiments, the CFTR gene comprises a mutation that leads to presence of an in-frame premature termination codon that is downstream of the first intron. In some embodiments, a least one allele of the CFTR gene in the cells of the subject is a variant selected from the group consisting of: c.3717G>A, c.3717+4A>G, c.3717+5G>A, c.3717+40A>G, c.3718-2477C>T, c.3718-1G>A, c.3718-3T>G, c.3719T>G, c.3731G>A, c.3737C>T, c.3744delA, c.3745G>A, c.3747delG, c.3752G>A, c.3761T>G, c.3763T>C, c.3764C>A, c.3773dupT, c.3806T>A, c.3808delG, c.3808G>A, c.3822G>A, c.3846G>A, c. [3846G>A;3848G>T], c.3848G>T, c.3872A>G, c.3873G>C, c.3873+1G>A, c. (3873+1_3874-1)_(3963+1_3964-1) del, c.3873+2T>C, c.3883_3886delATTT, c.3883delA, c.3889dupT, c.3891dupT, c.3908delA, c.3909C>G, c.3929G>A, c.3937C>T, c. (3963+1_3964-1)_(*1_?) del, c.3964-78_4242+577del, c.3971T>C, c.3988C>T, c.4004T>C, c.4036_4042del, c.4046G>A, c.4077_4080delTGTTinsAA, c.4086dupT, c.4097T>A, c.4111G>T, c.4124A>C, c.4127_4131delTGGAT, c.4144C>T, c.4147dupA, c.4197_4198delCT, c.4231C>T, c.4234C>T, c.4242+1G>T, c.4242+1G>A, c.4251delA, c.4300_4301dup, c.4364C>G, c.4426C>T, and c.4439T>C. In some embodiments, the method ameliorates or prevents one or more symptoms associated with cystic fibrosis.

In some aspects, the present disclosure provides a pharmaceutical composition, comprising: (a) a pharmaceutically acceptable excipient or carrier; and (b) the composition provided in this disclosure. In some embodiments, the pharmaceutical composition is formulated for intranasal administration, intratracheal administration, intravenous injection, intraperitoneal injection, intramuscular injection, intrathecal injection, subcutaneous injection, oral administration, synovial injection, intracerebroventricular injection, intravitreal administration, subretinal injection, topical application, or implantation. In some embodiments, the pharmaceutical composition is formulated for administration via respiratory route. In some embodiments, the pharmaceutical composition further comprises a second therapeutic agent. In some embodiments, the second therapeutic agent comprises a modulator of CFTR protein that potentiates chloride conductance of CFTR in the cells. In some embodiments, the second agent comprises ivacaftor, lumacaftor, tezacaftor, elexacaftor, a combination of lumacaftor and ivacaftor, a combination of tezacaftor and ivacaftor, or a combination of elexacaftor, ivacaftor, and tezacaftor. In some embodiments, the second agent comprises a mucolytic agent for airway clearance, optionally wherein the mucolytic agent for airway clearance is selected from the group consisting of: acetylcysteine, ambroxol, bromhexine, carbocisteine, erdosteine, mecysteine, dornase alfa, hypertonic saline, and mannitol. In some embodiments, the second agent comprises a bronchodilator, optionally wherein the bronchodilator is albuterol. In some embodiments, the second agent comprises an immunosuppressive agent. In some embodiments, the immunosuppressive agent is a corticosteroid selected from the group consisting of beclomethasone, budesonide, budesonide, formoterol, ciclesonide, fluticasone, salmeterol, umeclidinium, vilanterol, mometasone, or mometasone, prednisone, and methylprednisolone, and pharmaceutically acceptable salts thereof. In some embodiments, the immunosuppressive agent is a non-steroidal immunosuppressive agent selected from the group consisting of polyclonal anti-lymphocyte antibodies, monoclonal anti-lymphocyte antibodies, interleukin-2 (IL-2) receptor antagonists, calcineurin inhibitors, cell cycle inhibitors, mammalian target of rapamycin (mTOR) inhibitors, methotrexate, cyclophosphamide, an anthracycline, a taxane.

In some aspects, the present disclosure provides a kit comprising: (a) the composition provided herein or the pharmaceutical composition provided herein; and (b) instructions for use of the composition or the pharmaceutical composition.

In some aspects, the present disclosure provides a kit comprising: (a) the composition provided herein or the pharmaceutical composition provided herein; and (b) a second therapeutic agent. In some embodiments, the second therapeutic agent comprises a modulator of CFTR protein that potentiates chloride conductance of CFTR in the cells. In some embodiments, the second agent comprises ivacaftor, lumacaftor, tezacaftor, elexacaftor, a combination of lumacaftor and ivacaftor, a combination of tezacaftor and ivacaftor, or a combination of elexacaftor, ivacaftor, and tezacaftor. In some embodiments, the second agent comprises a mucolytic agent for airway clearance, optionally wherein the mucolytic agent for airway clearance is selected from the group consisting of: acetylcysteine, ambroxol, bromhexine, carbocisteine, erdosteine, mecysteine, dornase alfa, hypertonic saline, and mannitol. In some embodiments, the second agent comprises a bronchodilator, optionally wherein the bronchodilator is albuterol. In some embodiments, the second agent comprises an immunosuppressive agent. In some embodiments, the immunosuppressive agent is a corticosteroid selected from the group consisting of beclomethasone, budesonide, budesonide, formoterol, ciclesonide, fluticasone, salmeterol, umeclidinium, vilanterol, mometasone, or mometasone, prednisone, and methylprednisolone, and pharmaceutically acceptable salts thereof. In some embodiments, the immunosuppressive agent is a non-steroidal immunosuppressive agent selected from the group consisting of polyclonal anti-lymphocyte antibodies, monoclonal anti-lymphocyte antibodies, interleukin-2 (IL-2) receptor antagonists, calcineurin inhibitors, cell cycle inhibitors, mammalian target of rapamycin (mTOR) inhibitors, methotrexate, cyclophosphamide, an anthracycline, a taxane. In some embodiments, the kit further comprises instructions for use of the composition or the pharmaceutical composition, and instructions for use of the second therapeutic agent.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1B depict a schematic representation of W1282X CFTR mRNA processing and the therapeutic strategy of promoting Exon 22 truncated mRNA (E22 trunc mRNA) level to modulate expression of eligible CFTR variants. As depicted in FIG. 1A, nascent W1282X CFTR mRNA can be processed to two types of mature transcripts. One transcript, Exon 22 truncated mRNA, which arises from usage of Intron 22 alternative polyadenylation (ApA) sites, can escape from NMD, and lacks the premature termination codon (PTC)-containing Exon 23. Another transcript, FL W1282X CFTR mRNA transcript, which contains a PTC in Exon 23 and an ordinary poly A tail after the 3′ UTR in exon 27, can be sensitive to NMD. Without wishing to be bound by a certain theory, as depicted in FIG. 1B, suppressing splicing out of Intron 22 using the method according to some embodiments of the present disclosure can increase the level of Exon 22 truncated mRNA transcript and thus Exon 22 CFTR protein expression. FIG. 1C lists 13 CFTR variants residing in Exons 23-27 that result in PTCs, including the CF-causing variants and the variants that exhibit varying clinical consequences. These CFTR variants, at the time of this application, are not approved for treatment with Trikafta.

FIGS. 2A-2D depict the discovery of 3′ truncated transcripts. FIG. 2A depicts the discovery of 3′ truncated transcripts in 16HBEge-W1282X and 16HBEge-R1162X cells, indicated by the low sequence coverage of 3′-end exons of CFTR transcripts (e.g., Exons 23-27). All sequence counts were normalized to wildtype 16HBE cells, and sequence coverage data for CFTR exon 7 to exon 27 regions were shown. Sequencing results from 16HBEge-Y122X cell line showed similar coverage profile as wildtype cells as no splicing liability was expected. Both R553X and G542X showed a reduction of coverage in exon 12, which is consistent with previous experiments. FIG. 2B depicts exemplary sequences of the truncated 16HBEge-W1282X CFTR transcripts, which terminated within Intron 22 with extension of ˜140 bp into the intron on the 3′ end. The 16HBEge-W1282X transcripts ended with a series of non-aligned adenosine nucleotides. Consistent with this finding, a consensus ApA (alternative polyadenylation) motif was identified within Intron 22 near the abovementioned site of polyadenylation, indicating ApA usage. FIG. 2C depicts IGV screen capture of CFTR 3′ RACE reads from 16HBE14o- cells aligned to Hg38 reference genome. Expanded view of IGV screen capture showing locations of putative ApA hexanucleotide and CA cleavage sites in black boxes. FIG. 2D depicts the CFTR genomic locus with intron 22ApA. Introns not shown to scale. E22 trunc mRNA representation showing alternative 3′UTR and poly A tail. E22 trunc protein representation showing additional 9 alternative amino acids and missing NBD2.

FIG. 3 is a table showing expression levels of E22 trunc mRNA and full length CFTR mRNA in wild type and W1282X airway and intestinal cells.

FIG. 4 depicts the predicted effect of Exon 22 truncation on the CFTR protein. The truncated mRNA sequences were approximately 3857 base pairs (bp) long, including the first 22 exons and ˜140 bp of Intron 22 sequence. The first 27 bp of the intronic 22 sequence encodes 9 amino acids in-frame, followed by a stop codon and an alternative 3′ UTR (untranslated region). Thus, truncated CFTR protein resulted from the 3′ truncated Exon 22 mRNA-ApA can contain the first 1239 amino acids of a WT CFTR and additional few (˜9) amino acids encoded by the intronic sequence within Intron 22.

FIG. 5 depicts a graph plotting the percent Exon 22 truncated mRNA transcripts normalized to full length CFTR transcripts (“FL CFTR”). Higher fractions of Exon 22 truncated mRNA over FL CFTR were observed in 16HBEge-W1282X and R1162X cells, as compared to that in wildtype cells (WT), and the addition of SMG-1 inhibitor significantly reduced the said fraction in cells, indicating NMD escape in transcript processing.

FIG. 6A depicts the schematic representation of the RNA stability study. Briefly, percent of mRNA remaining was tracked using droplet digital PCR (ddPCR) after Actinomycin D administration in a mutant cell line, W1282X-I22-SAd, which was generated by disrupting the splice acceptor site of Intron 22. The W1282X-I22-SAd cells produced the following three CFTR transcripts: the Exon 22 truncated mRNA, full-length CFTR mRNA containing the PTC (“FL-W1282X transcript”), and the Exon 23-skipped CFTR mRNA. FIG. 6B upper panel depicts a graph plotting the mRNA level of the said three transcripts in the W1282X-I22-SAd cells at steady state. Exon 22 truncated mRNA transcripts were found to be present at a level over four times higher than those in the W1282X parental line. FIG. 6B lower panel depicts exemplary results from the RNA stability study. The amount of each CFTR mRNA species assessed at the first time point (t=0) was set to 1 (100%), and the percentage of the remaining mRNA was measured by ddPCR. The mRNA half-life was calculated from the exponential decay. The t_{1/2} of Exon 22 truncated mRNA was estimated to be about 4.24 hours, significantly longer than that of the FL-W1282X transcript (<<<2 hours). Exon 23-skipped mRNA transcript exhibited a longer half-life (t_{1/2}=10.61 hours), equivalent to the wildtype CFTR transcript.

FIGS. 7A-7B depict exemplary results from the transepithelial chloride conductance assay (TECC-24 assay) of transiently expressed CFTR variants under Trikafta treatment. FIG. 7A depicts results from the TECC-24 assay for the Exon 22 truncated and F508del CFTR variants under Trikafta treatment. Compared with the untreated group, F508del CFTR function was enhanced in vitro by addition of Trikafta, as indicated by an increase of area under curve (AUC) of CFTR-mediated chloride current. Similarly, Exon 22 truncated CFTR function was also enhanced by Trikafta. It was observed that Trikafta recovered F508del CFTR function to about 35% of that of wildtype and enhanced Exon 22 truncated CFTR function to about 15% of that of wildtype. FIG. 7B depicts results from the TECC-24 assay for FRT cells overexpressing the Exon 22 trunc cDNA.

FIG. 8 depicts the schematic representation of the Δ23-27 (or Del23-27) gene edited model, 16HBE14o- cells with genomic deletion of the regions spanning from 5′ portion of Intron 22 to post-3′UTR. The resulting cell genome contains Exons 1-22 followed by ˜13.5 kb of Intron 22, and then intergenic region upstream of CTTNBP2.

FIG. 9 depicts results from sequencing three exemplary Δ23-27 gene edited model cell lines: 2-H07, 3-B09, and 3-D01. Exon 22 truncated CFTR mRNA transcripts (black bars) from three Del23-27 clonal lines and 16HBE14o- cells were assayed using ddPCR. Full length CFTR was measured from 16HBE14o- cells (grey bar). All three Del23-27 cell lines contained significantly higher levels of Exon 22 truncated CFTR mRNA compared to the parental 16HBE14o- line, with ˜12 times the amount of that in parental cells. The levels of Exon 22 truncated CFTR mRNA in all three Del23-27 cell lines were greater than the level of full-length WT CFTR transcript in parental 16HBE14o- cells.

FIG. 10 depicts exemplary Western blot results of truncated forms of the CFTR proteins detected from Del23-27 clonal lines (2-H07, 3-B09, 3-D01) using the α-CFTR UNC596 antibody (raised against epitope: 1204-1211 aa in Ex22). Reactivity against Exon 22 was detected in 2 bands (Band C and B) in all Del23-27 cells. Seven leftmost lanes: Western blot analysis of 2-H07, 3-B09, 3-D01 and Δ23-27 gene edited (+/−) VX-445/VX-661 and 16HBE14o-

parental line. Six rightmost lanes: PNGaseF treated (de-glycosylated) W1282X, Exon 22 trunc, cDNA overexpression control from HEK293 cells and Δ23-27 2-H07 and 16HBE14o- parental line were diluted to produce equivalent band intensity. ACTB and Na/K-ATPase loading controls were greyed out for PNGaseF treated samples. Na/K-ATPase was blotted as a loading control. VX-445/VX-661 (3/3 μM) treatment did not significantly affect the level of truncated CFTR protein.

FIGS. 11A-11C depict exemplary results from TECC-24 assay conducted in various cell types treated with DMSO (vehicle) or pretreatment with VX-445/VX-661 (3/3 μM) for 48 hours. FIG. 11A shows results of WT16HBE140 cells. In the presence of VX-445/VX-661, Del23-27 CFTR protein function was restored by VX-770, shown as chloride conductance induced by VX-770 and inhibited by CFTR (inh)-172. All samples treated in assay with VX-770 (1 μM). As shown in FIG. 11B, CFTR function in the three Del23-27 cell lines 2-H07, 3-B09, and 3-D01 was restored to ˜25%, ˜17% and ˜18% of WT function, respectively, much higher than that of F508del potentiated by VX-809/VX-770 (˜5% of WT). FIG. 11C depicts TECC-24 Ieq Assay results of 16HBE14o- and CFF-16HBEge-W1282X and dose escalation of CFF-16HBEge-W1282X-A23-27-2H07 (+/−) VX-445/VX-661 3/3 μM.

FIG. 12A depicts a schematic representation of ASO sequence design. Steric blocking ASOs were designed via a 10 “step” 1-nucleotide “walk” tiled scheme to target the Intron 22 donor site (black) or acceptor site (gray bars). FIG. 12B depicts two graphs showing increased expression of the Exon 22 truncated CFTR mRNA in 16HBE14o- WT cells after administration of exemplary ASOs targeting the Intron 22 donor (ASO SD, mid) or acceptor (ASO SA, lower). SD10 (black bar upper) and SA8 (black bar lower) increased the percentage of Exon 22 truncated CFTR mRNA to ˜37% and ˜25% of the amount of FL CFTR mRNA in WT cells, respectively. Scrambled ASOs, off-target ASO (ASO targeting CEP290 mRNA), and untreated cells were included in the experiment as controls.

FIGS. 13A-13B are bar graphs depicting the changes in Exon 22 truncated CFTR mRNA after treating 16HBEge-W1282X cells with various doses of exemplary ASO(s), with and without Trikafta treatment. FIG. 13A shows that both SA08 and SD10 ASOs individually modulated the processing of CFTR mRNA and increased the amount of Exon 22 truncated CFTR mRNA. Combination treatment with two ASOs together further increased the amount of Exon 22 truncated CFTR mRNA. FIG. 13B depicts effect of exemplary ASO(s) at various doses. SD-10 and SA-08 ASOs were administered at up to 100 μM and up to 10 μM, respectively.

FIG. 14 depicts a Western Blot analysis of de-glycosylated Exon 22 truncated CFTR protein expression in 16HBEge-W1282X cells treated with exemplary ASO(s) alone or in combination (e.g., SD10; SD10 and SA08), in the presence of drug vehicle or correctors VX-445/661. The expression of Exon 22 truncated CFTR protein was increased by both treatment with SD10 alone and treatment with SD10/SA08 combination, while treatment with correctors VX-445/VX-661 did not significantly affect the expression of Exon 22 truncated CFTR protein.

FIGS. 15A-15C depict the effect of ASO(s) in 16HBEge-W1282X cells. FIG. 15A depicts exemplary chloride conductance traces from TECC-24 assay conducted with ASOs and Trikafta administration in 16HBEge-W1282X cells. Treatment with exemplary ASOs, SD10 and SA8, induced a much larger chloride channel conductance in the presence of Trikafta (VX-445/VX-661/VX-770). FIG. 15B depicts enhanced chloride conductance AUC of 16HBEge-W1282X cells normalized to WT CFTR (percent of WT CFTR AUC) after treatment with exemplary ASO(s), with or without Trikafta treatment. All ASO treatments increased the percent of WT CFTR AUC compared to vehicle alone. In presence of Trikafta, ASO(s) further enhanced the percent of WT CFTR AUC in a dose-dependent fashion. Combination of two ASOs increased the percent of WT AUC larger than either ASO alone. The largest effect was observed in the 2 μM SA8/10 μM SD10 group with Trikafta treatment, enhancing the chloride conductance AUC of 16HBEge-W1282X cells to ˜13.4% of WT. FIG. 15C depicts the TECC-24 assay results for various ASOs doses. CFF-16HBEge W1282X cells were treated with DMSO (vehicle), SD-10 or SA-08 alone or in combination for 48 hours, with or without pretreatment with VX-445/VX-661 (3/3 μM) for 48 hours. All samples treated in assay with VX-770 (3 μM). Combination of two ASOs increased chloride conductance more than either ASO alone. The largest effect was observed in the 10 μM SA8/100 μM SD10 group with Trikafta treatment.

FIG. 16 is a bar graph showing Exon 22 truncated CFTR mRNA level after treating primary W1282X/HBE with exemplary ASO(s) for 2 week or for 3 weeks. In both long-term treatment groups, the expression of Exon 22 truncated CFTR mRNA increased dramatically compared to no ASO treatment.

FIG. 17 depicts representative I_{eq} traces of chloride conductance from TECC-24 assay. Transepithelial electrical resistance (TEER) were consistent in all groups of the experiment and no toxicity was observed with repeated treatments. Stronger chloride conductance (I_{eq}) traces were observed after treatment with exemplary ASO(s) for 2 week and for 3 weeks.

FIG. 18 is a bar graph that shows long-term ASO(s) treatment for 2 weeks and 3 weeks increased normalized chloride conductance AUC measured in primary W1282X HBE cells and improved the function of the Exon22 truncated CFTR protein.

FIG. 19 is a bar graph depicting the changes in Exon 22 truncated CFTR mRNA after treating fully differentiated primary W1282X^{+/+} HBE. The cells were used in TECC-24 assay with 48-hour treatment using DMSO (vehicle) or SD-10 or SA-08 alone or in combination, with or without VX-445/VX-661 (3/3 μM) pretreatment for 48 hr. All samples treated in assay with VX-770 (1 μM). Treatment with SD-10 (100 μM) and SA-08 (10 μM) resulted in the highest amount of E22 trunc mRNA.

FIGS. 20A-20B depict CFTR protein expression in the primary W1282X^{+/+} HBE cells after treatments with ASO(s) and drug. FIG. 20A is a Western blot analysis from fully differentiated HBE W1282X+/+ at air-liquid interphase (ALI) post TECC-24 assay as described above. CFTR UNC596 and Beta Actin (ACTB) were used to detect CFTR and as loading controls respectively. FIG. 20B is a bar graph of CFTR B and C normalized to (−) ASO control (−) VX-445/VX-661 (3/3 μM). The ASO(s) administered increased the expression of E22 trunc CFTR protein.

FIG. 21 depicts results from TECC-24 assay from fully differentiated HBE W1282X+/+ at ALI with 48-hour treatments of DMSO (vehicle) or SD-10 or SA-08, alone or in combination, with or without VX-445/VX-661 (3/3 μM) pretreatment for 48 hours. All samples treated in assay with VX-770 (1 μM). Data represented as % WT 16HBE14o- (ratio of AUC/min W1282X (Fsk+VX-770)/WT (Fsk). ASO treatments significantly restored the chloride conductance of mutant CFTR.

### DETAILED DESCRIPTION

In some aspects, the present disclosure relates to compositions, methods, and kits involving an agent that modulates expression of a CFTR gene. The agent provided herein can modify a CFTR gene or modulates processing of a pre-mRNA that is transcribed from CFTR gene (CFTR pre-mRNA).

In some cases, the agent increases production of Exon 22 truncated CFTR mRNA, and thus expression of a C-terminal truncated CFTR protein. C-terminal truncated CFTR protein produced by translation of Exon 22 truncated CFTR mRNA can have partial function as compared to a wildtype CFTR protein, for instance, having chloride channel conductivity that is less than 100% (e.g., up to 90%, 80%, 70%, 60%, 50%, 40%, 30%, 20%, 10%, or 5%) of the chloride channel conductivity of a wildtype CFTR protein. In some aspects, the present disclosure relates to compositions, methods, and kits involving promotion of expression of C-terminal truncated CFTR protein produced by translation of Exon 22 truncated CFTR mRNA in a cell and restoration of CFTR protein function in the cell by concurrent or subsequent treatment of the cell with a CFTR corrector or potentiator, e.g., an agent that potentiates chloride channel conductivity of CFTR protein.

In some cases, the compositions, methods, and kits provided herein involve agents (e.g., antisense oligomers (ASOs)) that can modulate splicing events around Intron 22 of CFTR pre-mRNA in a cell, thus increasing level of Exon 22 truncated CFTR mRNA in the cell. In some cases, the compositions, methods, and kits provided herein involve agents (e.g., antisense oligomers (ASOs)) that can promote alternative polyadenylation (ApA) usage within Intron 22 of CFTR pre-mRNA in a cell, thus increasing level of Exon 22 truncated CFTR mRNA in the cell. In various embodiments, level of C-terminal truncated CFTR proteins can be increased using the methods of the disclosure to treat disease and conditions associated with one or more genetic mutations in CFTR gene located downstream (in the 3′ direction) of Intron 22.

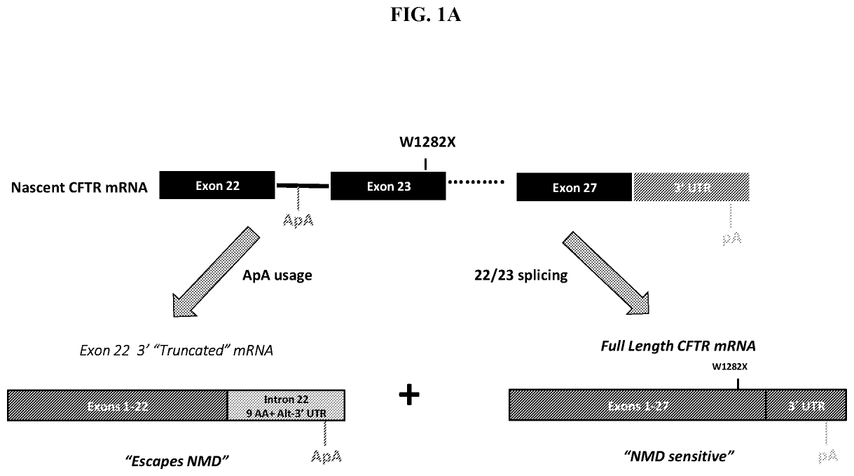

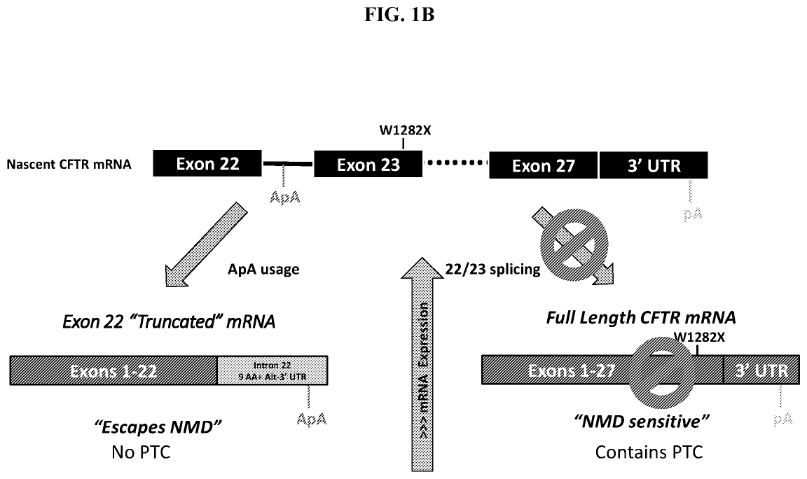

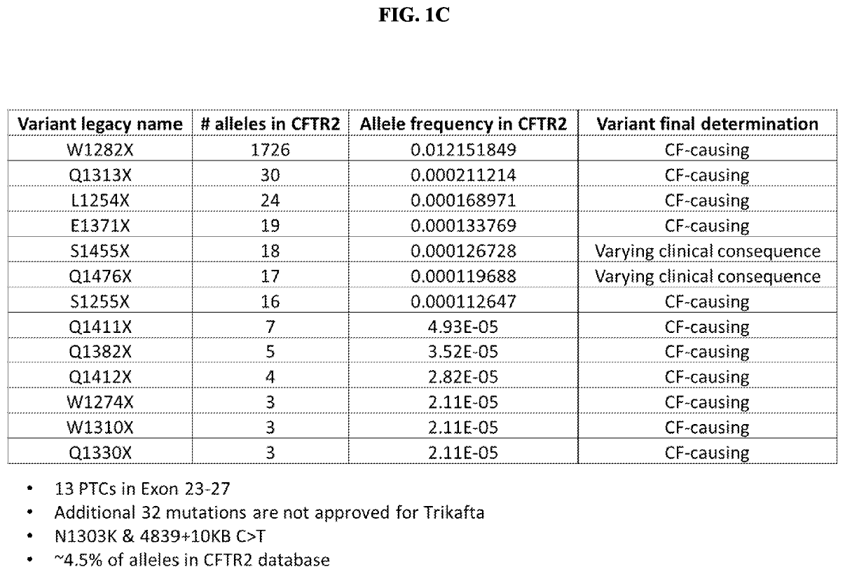

As depicted in FIG. 1A, in some cases, nascent pre-mRNA transcripts of some mutated CFTR (e.g., W1282X CFTR mRNA) can be processed into two types of mature mRNA transcripts. One transcript, full-length CFTR mRNA transcript, contains a PTC in Exon 23 caused by the genetic mutation, for instance, caused by W1282X in this case. The full-length, PTC-containing CFTR mRNA is polyadenylated at an ordinary polyadenylation site within the 3′ untranslated region (3′ UTR) of the CFTR pre-mRNA and can be sensitive to NMD due to the presence of the PTC in Exon 23. Another transcript, Exon 22 truncated CFTR mRNA transcript lacks exons downstream of Intron 22 and is polyadenylated at one of the alternatively polyadenylation sites within Intron 22. Exon 22 truncated CFTR mRNA transcripts lacks the PTC-containing Exon 23 and can escape from NMD in the cell. The C-terminal truncated CFTR protein produced from Exon 22 truncated CFTR mRNA transcript can be modulated by CFTR correctors and potentiators such as Trikafta. Without wishing to be bound by a certain theory, as depicted in FIG. 1B, blocking Exons 22-23 splicing using the methods according to some embodiments of the present disclosure can increase the level of Exon 22 truncated CFTR mRNA transcript, and thus, can increase the expression of the C-terminal truncated CFTR protein, e.g., the Exon 22 truncated CFTR protein.

In some cases, the method provided herein modulates expression of a CFTR gene in a cell. The method can comprise contacting an agent or a vector encoding the agent to the cell, wherein the cell comprises a pre-mRNA, wherein the pre-mRNA is transcribed from the CFTR gene and comprises a first intron that comprises an alternative polyadenylation site, and wherein the agent modifies the CFTR gene or modulates processing of the pre-mRNA, thereby increasing a level of a processed mRNA that is processed from the pre-mRNA and that lacks nucleic acid sequence of the pre-mRNA downstream of the first intron.

In some cases, the method comprises contacting an agent or a vector encoding the agent to the cell, wherein the cell comprises a pre-mRNA that is transcribed from the CFTR gene and comprises a first intron that comprises an alternative polyadenylation site, and wherein the agent suppresses splicing out of the first intron from the pre-mRNA during splicing of the pre-mRNA in the cell.

In some cases, the method comprises contacting an agent or a vector encoding the agent to the cell, wherein the agent comprises a polynucleotide sequence that is at least 80% complementary to a sequence selected from the group consisting of SEQ ID NOs: 6-36.

In some cases, the method comprises contacting an agent or a vector encoding the agent to the cell, wherein the agent comprises a polynucleotide sequence with at least 80% identity to a sequence selected from the group consisting of SEQ ID NOs: 37-64.

In some cases, the method comprises contacting an agent or a vector encoding the agent to the cell, wherein the agent comprises at least 8 contiguous nucleotides of a sequence selected from the group consisting of SEQ ID NOs: 37-64.

In some cases, the method comprises contacting an agent or a vector encoding the agent to the cell, wherein the agent comprises a polynucleotide that comprises the sequence set forth in any one of SEQ ID NOs: 66-93. In some cases, the method comprises contacting an agent or a vector encoding the agent to the cell, wherein the agent comprises a polynucleotide that consists of the sequence set forth in any one of SEQ ID NOs: 66-93.

In some cases, the method provided herein comprises an agent or a vector encoding the agent, wherein the agent, when present in a human cell that comprises a pre-mRNA, the pre-mRNA being transcribed from the CFTR gene and comprising a first intron that comprises an alternative polyadenylation site, modifies the CFTR gene or modulates processing of the pre-mRNA, thereby increasing a level of a processed mRNA that is processed from the pre-mRNA and that lacks nucleic acid sequence of the pre-mRNA downstream of the first intron.

In some cases, the composition comprises an agent or a vector encoding the agent, wherein the agent, when present in a cell that comprises a pre-mRNA, the pre-mRNA being transcribed from the CFTR gene and comprising a first intron that comprises an alternative polyadenylation site, suppresses splicing out of the first intron.

In some cases, the composition comprises an agent or a vector encoding the agent, wherein the agent comprises a polynucleotide sequence that is at least 80% complementary to a sequence selected from the group consisting of SEQ ID NOs: 6-36.

In some cases, the composition comprises an agent or a vector encoding the agent, wherein the agent comprises a polynucleotide sequence with at least 80% identity to a sequence selected from the group consisting of SEQ ID NOs: 37-64.

In some cases, the composition comprises an agent or a vector encoding the agent, wherein the agent comprises at least 8 contiguous nucleotides of a sequence selected from the group consisting of SEQ ID NOs: 37-64.

In some cases, the composition comprises an agent or a vector encoding the agent, wherein the agent comprises a polynucleotide that comprises the sequence set forth in any one of SEQ ID NOs: 66-93. In some cases, the composition comprises an agent or a vector encoding the agent, wherein the agent comprises a polynucleotide that consists of the sequence set forth in any one of SEQ ID NOs: 66-93.

In some cases, the method provided herein treats a subject in need thereof. The method of treatment provided herein can comprise contacting cells of the subject with the composition provided herein. In some cases, the method comprises concurrent or subsequent administration of a second therapeutic agent to the subject.

In some cases, a pharmaceutical composition provided herein comprise a pharmaceutically acceptable excipient or carrier; and the composition provided herein.

In some cases, a kit provided herein comprises the composition or the pharmaceutical composition disclosed herein, and instructions for use of the composition or the pharmaceutical composition. In some cases, a kit provided herein comprises the composition or the pharmaceutical composition disclosed herein, and a second therapeutic agent.

### CFTR

CFTR gene can encode cystic fibrosis transmembrane conductance regulator (CFTR protein), which is a member of the ATP-binding cassette (ABC) transporter superfamily. ABC proteins can transport various molecules across extra- and intra-cellular membranes. Generally, ABC genes are divided into seven distinct subfamilies (ABC1, MDR/TAP, MRP, ALD, OABP, GCN20, White). CFTR protein is a member of the MRP subfamily that can be involved in multi-drug resistance. CFTR protein can function as a chloride channel and control the regulation of other transport pathways. In some cases, mutations in CFTR gene are associated with the autosomal recessive disorders, such as cystic fibrosis and congenital bilateral aplasia of the vas deferens. Alternatively spliced CFTR transcript variants have been described, many of which can result from mutations in this gene.

Human (Homo sapiens) CFTR gene ((Gene ID: 1080) is located on chromosome 7, and can be defined by chromosomal coordinates GRCh38.p14/hg38: chr7: 117,480,025 to GRCh38.p14/hg38: chr7: 117,668,665. CFTR is also designated as Ensembl:ENSG00000001626; MIM:602421; AllianceGenome:HGNC:1884. CFTR is also known as: CF; MRP7; ABC35; ABCC7; CFTR/MRP; TNR-CFTR; dJ76005.1. The CFTR transcript has 27 exons and is designated as NCBI Reference Sequence NM_000492.4; ACCESSION: NM_000492, and Ensembl: ENST00000003084.11. Human CFTR protein is designated as NCBI Reference Sequence: NP 000483.3.

The CFTR protein can form a cell membrane-spanning chloride channel whose function can be regulated by phosphorylation mediated by cAMP-dependent phosphokinases. Phosphorylation of CFTR expressed on the membrane of a cell in the presence of adenosine triphosphate (ATP) can trigger channel opening to allow outflux of chloride ions from the cell through the channel formed by the CFTR protein, about 10 chloride ions every minute. Certain CFTR gene mutations can lead to generation of defective CFTR proteins that cannot be processed normally by the endoplasmic reticulum for effective transport to the cell membrane. The few mutated CFTR protein molecules that do reach the cell membrane can be dysfunctional and thus cannot carry out chloride ion transportation, leading to accumulation of chloride ions and associated water molecules in epithelial cells and lack of hydration of extracellular mucus and secretions.

CFTR mutations can be categorized according to the abnormalities they can lead to, including dysfunctional protein translation, cell processing, or CFTR channel gating. Missense (single amino acid substitution) mutations account for 38.74% of CFTR mutants, frameshift (insertion or deletion) mutations account for 16.25%, splicing (incorrect intron splicing) mutations account for 10.93%, and nonsense (early termination codon) mutations account for 8.41% of all known CFTR mutations detected worldwide. Mutations of the CFTR gene can fall into six different classes that roughly correspond to specific types of CFTR dysfunction. In general, mutations in classes I to III can cause more severe disease than those in classes IV to VI. Clinical manifestations of CF caused by any combination of mutations can vary, perhaps due to effects of gene modifiers. For example, genotype-phenotype correlations are weak for CF associated with pulmonary disease but can be stronger for CF types associated with pancreatic insufficiency. Characterization of mutations can be useful to guide initial therapy for some patients, as several new therapies have been recently developed that target CF disease caused by specific classes of CFTR mutations.

There can be five different types of CFTR mutations. Class I mutations: defective protein production. This type of defect can be caused by nonsense, frameshift, or splice-site mutations, leading to premature termination of messenger RNA (mRNA) transcripts and absence of full length CFTR protein. Non-limiting examples include G542X, W1282X, R553X, 621+G>T, and 1717-1G>A. Class II mutations: defective protein processing. This class of mutation can cause abnormal post-translational processing of the CFTR protein, which prevents the protein from trafficking to the correct cellular location, as exemplified by the F508del mutation that is present in a homozygous state in approximately 50% of CF patients and in at least a heterozygous state in 90% of CF patients. Class III mutations: defective regulation. These mutations cause diminished channel activity even when ATP levels are adequate. Many mutations can alter NBF ATP-binding regions (designated NBD1 and NBD2), whereby some mutants retain varying degrees of sensitivity to nucleotide binding. The mutation giving rise to CFTR substitution G551D, which can abolish ATP binding, is the most common class III mutation in Caucasian populations. Meanwhile, other CFTR mutations within the region encoding the CFTR R domain can also fall into this category. Class IV mutations: defective conduction. CTFR protein carrying these mutations is produced and transported correctly to the cell surface. However, the rate of ion flow and the duration of channel opening can be reduced as compared to normal CFTR protein even though chloride currents are generated in response to CAMP stimulation. A mutation that induces a CFTR protein amino acid substitution (R117H) is the most common class IV mutation in Caucasian populations. Class V mutations: reduced amounts of functional CFTR protein. It includes several mutations that can alter mRNA stability and other types of mutations that can alter stability of the mature CFTR protein (with the latter sometimes classified separately into an additional class, class VI). Class VI mutations: decreased CFTR stability. This class can cause substantial plasma membrane instability and includes Phe508del when rescued by most correctors (rPhe508del).

In some embodiments, the function of various CFTR proteins can be assessed by assays (e.g., electrophysiology assays) known to a person skilled in the art. Exemplary electrophysiology assays can include transepithelial chloride conductance assay (e.g., TECC-24 assay). In some embodiments, the function of various CFTR proteins can be assessed by comparing the area under curve (AUC) of the induced chloride conductance obtained in the electrophysiology assays. The AUC of various mutant cell lines (e.g., 16HBEge-W1282X cells), with or without treatment, can be normalized to the AUC obtained from cells expressing WT CFTR protein and expressed as either a percentage of WT CFTR (% WT CFTR) or a ratio to WT CFTR (variant/WT CFTR). Many cell lines can be used to assess the function of CFTR proteins. Variations of human bronchial epithelial (HBE) cell lines including wildtype, mutants, primary and immortalized cell lines can be used in the electrophysiology assays to assess CFTR channel function.

In some cases, the function of a C-terminal truncated CFTR protein is less than 100% of that of a WT CFTR protein. For instance, the function of a C-terminal truncated CFTR proteins can be up to 95%, up to 90%, up to 85%, up to 80%, up to 70%, up to 60%, up to 50%, up to 40%, up to 30%, up to 20%, up to 10%, up to 5%, up to 2%, or up to 1% of that of a WT CFTR protein, as assessed by an assay (e.g., an electrophysiology assay) known to a person skilled in the art.

In the classic form of CF, a patient can demonstrate clinical disease in one or more organ systems (as described below) and can have elevated sweat chloride (>60 mmol/L). Most of these patients can have disease manifestations in multiple organ systems (pancreas, upper and lower respiratory tract, and male reproductive tract). A CFTR-related disorder (CFTR-RD) can refer to a clinical disease limited to only one organ system associated with some evidence of CFTR dysfunction that does not meet full genetic or functional criteria for a CF diagnosis. CFTR-RDs comprise disorders of the lungs, such as disseminated bronchiectasis. CFTR-RDs comprise disorders of the gastrointestinal tract, including CF-related pancreatic insufficiency, CF-related pancreatitis, CF-related diabetes, CF-related liver disease and gallbladder disease. CFTR-RDs comprise disorders of the reproductive tract, including congenital bilateral absence of the vas deferens (CBAVD). Clinical manifestations can include isolated obstructive azoospermia, chronic sinusitis, chronic pancreatitis, or pulmonary disease in adulthood. In cystic fibrosis, deranged transport of chloride and other CFTR-affected ions, such as sodium and bicarbonate, can lead to thick, viscous secretions in the lungs, pancreas, liver, intestine, or reproductive tract, or any combination thereof. Alternatively, or additionally, deranged transport of chloride and other CFTR-affected ions can lead to increased salt content in sweat gland secretions. Symptoms of CF can vary in different age groups of individuals. Infants and children can present respiratory symptoms, meconium ileus, failure to thrive, or any combination thereof. Typical respiratory manifestations of CF can include a persistent productive cough, hyperinflation of the lung fields on chest radiograph, and pulmonary function tests that are consistent with obstructive airway disease. Other clinical manifestations of pulmonary disease can include microorganism infection, bronchiectasis, airway hyperreactivity, allergic bronchopulmonary aspergillosis, obstructive sleep apnea, and pulmonary hypertension. Patients presenting with CF later in life are more likely to have atypical symptoms, including sinus disease, pancreatic disease, infertility, musculoskeletal disorders, recurrent venous thrombosis, anemia, electrolyte abnormalities, nephrolithiasis, and aquagenic wrinkling.

### Processing of CFTR Pre-mRNA

In some aspects, provided herein are compositions, methods, and kits involving an agent that modulates processing of a pre-mRNA that encodes CFTR protein (CFTR pre-mRNA) and that comprises a first intron that comprises an alternative polyadenylation site. Processing of a pre-mRNA in a cell can comprise splicing of the pre-mRNA followed by polyadenylation of the resulting mRNA transcript. The terms “mature mRNA,” “fully-spliced mRNA,” and “processed mRNA” are used interchangeably herein to describe a fully processed mRNA that is processed from a pre-mRNA. In some cases, the agent provided herein suppresses splicing out of the first intron (e.g., Intron 22) of the CFTR pre-mRNA. Alternatively, or additionally, the agent provided herein promotes polyadenylation of CFTR mRNA transcript at an alternative polyadenylation site within the first intron. In some cases, the CFTR pre-mRNA comprises a mutation that can result in the presence of a PTC downstream of Exon 22 when the pre-mRNA is processed into a mature CFTR mRNA transcript, which can result in NMD of the processed CFTR mRNA transcript.

Splicing and Nonsense-Mediated mRNA Decay

Intervening sequences or introns are removed by a large and highly dynamic RNA-protein complex termed the spliceosome, which orchestrates complex interactions between primary transcripts, small nuclear RNAs (snRNAs) and a large number of proteins. Spliceosomes assemble ad hoc on each intron in an ordered manner, starting with recognition of the 5′ splice site (5′ss) by U1 snRNA or the 3′ splice site (3′ss) by the U2 pathway, which involves binding of the U2 auxiliary factor (U2AF) to the 3′ss region to facilitate U2 binding to the branch point sequence (BPS). U2AF is a stable heterodimer composed of a U2AF2-encoded 65-kD subunit (U2AF65), which binds the polypyrimidine tract (PPT), and a U2AF1-encoded 35-kD subunit (U2AF35), which interacts with highly conserved AG dinucleotides at 3′ss and stabilizes U2AF65 binding. In addition to the BPS/PPT unit and 3′ss/5′ss, accurate splicing requires auxiliary sequences or structures that activate or repress splice site recognition, known as intronic or exonic splicing enhancers or silencers. These elements allow genuine splice sites to be recognized among a vast excess of cryptic or pseudo-sites in the genome of higher eukaryotes, which have the same sequences but outnumber authentic sites by an order of magnitude. Although they often have a regulatory function, the exact mechanisms of their activation or repression are poorly understood.

The decision of whether to splice or not to splice can be typically modeled as a stochastic rather than deterministic process, such that even the most defined splicing signals can sometimes splice incorrectly. However, under normal conditions, pre-mRNA splicing proceeds at surprisingly high fidelity. This is attributed in part to the activity of adjacent cis-acting auxiliary exonic and intronic splicing regulatory elements (ESRs or ISRs). Typically, these functional elements are classified as either exonic or intronic splicing enhancers (ESEs or ISEs) or silencers (ESSs or ISSs) based on their ability to stimulate or inhibit splicing, respectively. Although there is now evidence that some auxiliary cis-acting elements may act by influencing the kinetics of spliceosome assembly, such as the arrangement of the complex between U1 snRNP and the 5′ss, it seems very likely that many elements function in concert with trans-acting RNA-binding proteins (RBPs). For example, the serine- and arginine-rich family of RBPs (SR proteins) is a conserved family of proteins that have a key role in defining exons. SR proteins promote exon recognition by recruiting components of the pre-spliceosome to adjacent splice sites or by antagonizing the effects of ESSs in the vicinity. The repressive effects of ESSs can be mediated by members of the heterogeneous nuclear ribonucleoprotein (hnRNP) family and can alter recruitment of core splicing factors to adjacent splice sites. In addition to their roles in splicing regulation, silencer elements are suggested to have a role in repression of pseudo-exons, sets of decoy intronic splice sites with the typical spacing of an exon but without a functional open reading frame. ESEs and ESSs, in cooperation with their cognate trans-acting RBPs, represent important components in a set of splicing controls that specify how, where and when mRNAs are assembled from their precursors.

The sequences marking the exon-intron boundaries are degenerate signals of varying strengths that can occur at high frequency within human genes. In multi-exon genes, different pairs of splice sites can be linked together in many different combinations, creating a diverse array of transcripts from a single gene. This is commonly referred to as alternative pre-mRNA splicing. Although most mRNA isoforms produced by alternative splicing can be exported from the nucleus and translated into functional polypeptides, different mRNA isoforms from a single gene can vary greatly in their translation efficiency. Those mRNA isoforms with premature termination codons (PTCs) at least 50 bp upstream of an exon junction complex are likely to be targeted for degradation by the nonsense-mediated mRNA decay (NMD) pathway.

Mutations in traditional (BPS/PPT/3′ss/5′ss) and auxiliary splicing motifs can cause aberrant splicing, such as exon skipping or cryptic (or pseudo-) exon inclusion or splice-site activation and contribute significantly to human morbidity and mortality. Both aberrant and alternative splicing patterns can be influenced by natural DNA variants in exons and introns.

Given that exon-intron boundaries can occur at any of the three positions of a codon, it is clear that only a subset of alternative splicing events can maintain the canonical open reading frame. For example, only exons that are evenly divisible by 3 can be skipped or included in the mRNA without any alteration of reading frame. Splicing events that do not have compatible phases will induce a frameshift. Unless reversed by downstream events, frameshifts can certainly lead to one or more PTCs, probably resulting in subsequent degradation by NMD. NMD is a translation-coupled mechanism that eliminates mRNAs containing PTCs. NMD can function as a surveillance pathway that exists in all eukaryotes. NMD can reduce errors in gene expression by eliminating mRNA transcripts that contain premature stop codons. Translation of these aberrant mRNAs could, in some cases, lead to deleterious gain-of-function or dominant-negative activity of the resulting proteins. NMD targets not only transcripts with PTCs but also a broad array of mRNA isoforms expressed from many endogenous genes, suggesting that NMD is a master regulator that drives both fine and coarse adjustments in steady-state RNA levels in the cell.

### Alternative Polyadenylation

In some aspects, provided herein is a composition that modifies alternative polyadenylation during processing of CFTR pre-mRNA into a mature mRNA.

Alternative polyadenylation (APA) is an RNA-processing mechanism that can generate distinct 3′ termini on mRNAs and other RNA polymerase II transcripts. It is widespread across all eukaryotic species and is recognized as a major mechanism of gene regulation. APA can exhibit tissue specificity and can be important for cell proliferation and differentiation. The roles of APA can cover a range of diverse cellular processes, including mRNA metabolism, protein, diversification, and protein localization, and can be generally in gene regulation. The molecular mechanisms underlying APA comprise variation in the concentration of core processing factors and RNA-binding proteins, as well as transcription-based regulation.

### Modulation of Processing of CFTR Pre-mRNA

In some aspects, provided herein are methods, compositions, and kits relating to an agent that modulates processing of a CFTR pre-mRNA transcript into a mature CFTR mRNA transcript. The CFTR pre-mRNA can comprise a first intron that comprises an alternative polyadenylation site. In some cases, the agent provided herein modulates processing of the CFTR pre-mRNA, and thus increases a level of a processed mRNA that is processed from the pre-mRNA and that lacks nucleic acid sequence of the pre-mRNA downstream of the first intron that comprises an alternative polyadenylation site. In some cases, the first intron is Intron 22, such as an intron located from GRCh38.p14/hg38: chr7: 117,627,771 to GRCh38.p14/hg38: chr7: 117,642,437.

In some cases, the agent provided herein removes from the pre-mRNA the nucleic acid sequence of the pre-mRNA downstream of the first intron, such as the nucleic acid sequence located downstream of Intron 22 of CFTR pre-mRNA. In some cases, the removed nucleic acid sequence is located from GRCh38.p14/hg38: chr7: 117,642,438 to GRCh38.p14/hg38: chr7: 117,668,665.

In some cases, the agent provided herein suppresses splicing out of the first intron from CFTR pre-mRNA, for instance, by suppressing splicing at 5′ splice site, 3′ splice site, or both, of the first intron. In some cases, the agent provided herein suppresses splicing out of Intron 22 from CFTR pre-mRNA. For instance, the agent provided herein suppresses splicing at 5′ splice site, 3′ splice site, or both of Intron 22 of CFTR pre-mRNA.

Without wishing to be bound by a certain theory, suppression of splicing out of Intron 22 from CFTR pre-mRNA can offer opportunity for polyadenylation of the mRNA transcript at one of the alternative polyadenylation sites located within Intron 22, which can then lead to truncation of the mature mRNA transcript, for instance, generation of Exon 22 truncated CFTR mRNA (CFTR mRNA that does not have exonic sequences downstream of Exon 22).

Alternatively, or additionally, without wishing to be bound by a certain theory, suppression of splicing out of Intron 22 from CFTR pre-mRNA can suppress splicing at one or more splice sites downstream of Intron 22, which can then lead to truncation of the mature mRNA transcript, e.g., generation of Exon 22 truncated CFTR mRNA transcript.

In some cases, upon processing, Exon 22 truncated CFTR mRNA transcript, which is a processed mRNA that is processed from the CFTR pre-mRNA, comprises, in a 5′ to 3′ order, 22 exons, an intronic sequence encoding nine amino acids, a stop codon, and an alternative 3′ untranslated region. The 22 exons can be canonical Exon 1 to Exon 22 of CFTR pre-mRNA. The intronic sequence encoding nine amino acids can be a nucleic acid sequence located within Intron 22 of CFTR pre-mRNA. In some cases, the intronic sequence encoding nine amino acids is transcribed from genomic sequence located from GRCh38.p14/hg38: chr7: 117,627,771 to GRCh38.p14/hg38: chr7: 117,627,797. In some cases, the processed mRNA that does not have nucleic acid sequence of the CFTR pre-mRNA downstream of the first intron, e.g., Exon 22 truncated CFTR mRNA, is polyadenylated at an alternative polyadenylation site within the first intron, e.g., an alternative polyadenylation site in a nucleic acid sequence transcribed from genomic sequence located between GRCh38.p14/hg38: chr7: 117,627,771 and GRCh38.p14/hg38: chr7: 117,642,437.

In some cases, the agent provided herein binds to a 5′ splice site of the first intron (e.g., Intron 22) of CFTR pre-mRNA. In some cases, the agent provided herein binds to a 3′ splice site of the first intron (e.g., Intron 22) of CFTR pre-mRNA. In some cases, the agent provided herein binds to a branch point for the 3′ splice site of the first intron (e.g., Intron 22) of CFTR pre-mRNA. In some cases, the agent provided herein interferes with a splicing factor that is involved in splicing out of the first intron (e.g., Intron 22) of CFTR pre-mRNA. In some cases, the agent interferes with a splicing factor that is involved in splicing out of the first intron (e.g., Intron 22) of CFTR pre-mRNA by binding to: (a) a 5′ splice site of the first intron, (b) a 3′ splice site of the first intron, (c) a branch point for the 3′ splice site of the first intron, or (d) any combination thereof. In some cases, the agent provided herein binds to a targeted portion located within the first intron (e.g., Intron 22) of CFTR pre-mRNA. In some cases, the agent provided herein binds to a targeted portion located within an exon immediately preceding the first intron (e.g., Exon 22) of CFTR pre-mRNA. In some cases, the agent provided herein binds to a targeted portion located within an exon immediately following the first intron (e.g., Exon 23) of CFTR pre-mRNA. In some cases, the agent provided herein binds to a targeted portion located at a junction between the first intron (e.g., Intron) 22 and an exon immediately preceding the first intron, or an exon immediately following the first intron, e.g., Exon22-Intron 22 junction or Intron 22-Exon 23 junction.

As provided herein, an agent of the present disclosure, upon contacted with a cell, can increase a level of the processed mRNA that does not have nucleic acid sequence of the CFTR pre-mRNA downstream of a first intron that comprises an alternative polyadenylation site (e.g., Intron 22) in the cell. For instance, the level of the processed mRNA that does not have nucleic acid sequence of the CFTR pre-mRNA downstream of the first intron is increased in the cell by at least about 1.5, at least about 2, at least about 3, at least about 4, at least about 5, at least about 6, at least about 7, at least about 8, at least about 9, at least about 10, at least about 11, at least about 12, at least about 13, at least about 14, at least about 15, or at least about 20 times as compared to a corresponding cell that is not contacted with the agent. In some cases, the level of the processed mRNA is increased in the cell by at least about 10 times as compared to a corresponding cell that is not contacted with the agent.

As provided herein, an agent of the present disclosure, upon contact with a cell, can increase a level of a truncated CTFR protein in the cell that is translated from a processed mRNA that does not have nucleic acid sequence of the CFTR pre-mRNA downstream of a first intron that comprises an alternative polyadenylation site (e.g., Intron 22), which lacks amino acid sequence expressed from exonic sequences of the CFTR gene downstream of the first intron. In some cases, the level of the truncated CFTR protein in the cell is increased by at least about 1.5, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 25, at least about 30, at least about 40, at least about 50, at least about 60, at least about 70, at least about 80, at least about 90, at least about 100, at least about 120, at least about 150, at least about 180, at least about 200, at least about 250, at least about 300, at least about 400, at least about 500, at least about 750, or at least about 1000 times as compared to a corresponding cell that is not contacted with the agent. In some cases, the level of the truncated CFTR protein in the cell is increased by at least about 10 times as compared to a corresponding cell that is not contacted with the agent.

In some cases, the level of the truncated CFTR protein in the cell is increased by about 1.5, about 2, about 3, about 4, about 5, about 6, about 7, about 8, about 9, about 10, about 11, about 12, about 13, about 14, about 15, about 16, about 17, about 18, about 19, about 20, about 25, about 30, about 40, about 50, about 60, about 70, about 80, about 90, about 100, about 120, about 150, about 180, about 200, about 250, about 300, about 400, about 500, about 750, or about 1000 times as compared to a corresponding cell that is not contacted with the agent. In some cases, the level of the truncated CFTR protein in the cell is increased by about 10 times as compared to a corresponding cell that is not contacted with the agent.

In some cases, the truncated CFTR protein in the cell has a chloride channel conductivity that is less than 100%, less than 90%, less than 80%, less than 70%, less than 60%, less than 50%, less than 40%, less than 30%, less than 20%, less than 10%, or less than 5% of a chloride channel conductivity of a wildtype CFTR protein.

In some cases, the chloride channel conductivity of the truncated CFTR protein is increased by a modulator of CFTR protein that potentiates chloride conductance of CFTR in the cells. In some cases, the modulator of CFTR protein comprises ivacaftor, lumacaftor, tezacaftor, elexacaftor, a combination of lumacaftor and ivacaftor, a combination of tezacaftor and ivacaftor, or a combination of elexacaftor, ivacaftor, and tezacaftor.

In some cases, the modulator of CFTR protein increases the chloride channel conductivity of the truncated CFTR protein by at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300%, at least 350%, at least 400%, at least 450%, at least 500%, at least 550%, at least 600%, at least 650%, at least 700%, at least 750%, at least 800%, at least at least 850%, at least 900%, at least 950%, at least 1000%. In some cases, the modulator of CFTR protein increases the chloride channel conductivity of the truncated CFTR protein by about 1.5, about 2, about 3, about 4, about 5, about 6, about 7, about 8, about 9, about 10, about 11, about 12, about 13, about 14, about 15, about 16, about 17, about 18, about 19, about 20, about 25, about 30, about 40, about 50, about 60, about 70, about 80, about 90, about 100, about 120, about 150, about 180, about 200, about 250, about 300, about 400, about 500, about 750, or about 1000 times.

In some cases, the modulator of CFTR protein restores the chloride channel conductivity of the truncated CFTR protein to about at least 5%, at least 10%, at least 15%, at least 20%, at least 25%, at least 30%, at least 35%, at least 40%, least 45%, at least 50%, at least 55%, at least 60%, least 65%, at least 70%, at least 75%, at least 80%, at least 85%, at least 90%, at least 95% or more of the chloride channel conductivity of wildtype CFTR protein.

### Target Nucleic Acids and Agents

In some aspects, provided herein are compositions and methods relating to an agent (e.g., antisense oligomer) or a vector encoding an agent that targets a targeted portion of a nucleic acid encoding a CFTR protein, such as a CFTR pre-mRNA or a CFTR gene in a cell.

In some cases, the agent provided herein binds to a targeted portion located within the first intron (e.g., Intron 22) of CFTR pre-mRNA. In some cases, the agent provided herein binds to a targeted portion located within an exon immediately preceding the first intron (e.g., Exon 22) of CFTR pre-mRNA. In some cases, the agent provided herein binds to a targeted portion located within an exon immediately following the first intron (e.g., Exon 23) of CFTR pre-mRNA. In some cases, the agent provided herein binds to a targeted portion located at a junction between the first intron (e.g., Intron) 22 and an exon immediately preceding the first intron, or an exon immediately following the first intron, e.g., Exon22-Intron 22 junction or Intron 22-Exon 23 junction.

Where reference is made to a polynucleic acid polymer sequence, the skilled person will understand that one or more substitutions may be tolerated, optionally two substitutions may be tolerated in the sequence, such that it maintains the ability to hybridize to the target sequence; or where the substitution is in a target sequence, the ability to be recognized as the target sequence. References to sequence identity may be determined by BLAST sequence alignment using standard/default parameters. For example, the sequence may have 99% identity and still function according to the present disclosure. In other embodiments, the sequence may have 98% identity and still function according to the present disclosure. In another embodiment, the sequence may have 95% identity and still function according to the present disclosure. In another embodiment, the sequence may have 90% identity and still function according to the present disclosure.

Polynucleotide sequence, including oligomers, such as oligonucleotides, are “complementary” to one another when hybridization occurs in an antiparallel configuration between two single-stranded polynucleotides. A double-stranded polynucleotide can be “complementary” to another polynucleotide if hybridization can occur between one of the strands of the first polynucleotide and the second. Complementarity (the degree to which one polynucleotide is complementary with another) is quantifiable in terms of the proportion (e.g., the percentage) of bases in opposing strands that are expected to form hydrogen bonds with each other, according to generally accepted base-pairing rules. The sequence of a polynucleotide sequence, such as an antisense oligomer (ASO), need not be 100% complementary to that of its target nucleic acid to hybridize. In certain embodiments, agents (e.g., ASOs) comprise at least 70%, at least 75%, at least 80%, at least 85%, at least 90%, at least 95%, at least 96%, at least 97%, at least 98%, or at least 99% sequence complementarity to a target region within the target nucleic acid sequence to which they are targeted. For example, an ASO in which 18 of 20 nucleobases of the oligomeric compound are complementary to a target region, and would therefore specifically hybridize, would represent 90 percent complementarity. In this example, the remaining non-complementary nucleobases may be clustered together or interspersed with complementary nucleobases and need not be contiguous to each other or to complementary nucleobases. Percent complementarity of an agent (such as an ASO) with a region of a target nucleic acid can be determined routinely using BLAST programs (basic local alignment search tools) and PowerBLAST programs known in the art (Altschul, et al., J. Mol. Biol., 1990, 215, 403-410; Zhang and Madden, Genome Res., 1997, 7, 649-656, of which entire content is incorporated herein by reference).

In some cases, the target nucleic acid is a portion of the sequence of SEQ ID NO: 1 or 2. In some cases, the target nucleic acid has at least 80%, 85%, 90%, 91%, 92%, 93%, 94%, 95%, 96%, 97%, 98%, or 99%, or 100% sequence identity to SEQ ID NO: 1 or 2. In some cases, the target nucleic acid has about 80%, 85%, 90%, 91%, 92%, 93%, 94%, 95%, 96%, 97%, 98%, 99%, or 100% sequence identity to SEQ ID NO: 1 or 2. In some cases, the target nucleic acid is a portion of the sequence of SEQ ID NO: 1 or 2. In some cases, the target nucleic acid has a sequence that has at least 80%, 85%, 90%, 91%, 92%, 93%, 94%, 95%, 96%, 97%, 98%, or 99%, or 100% sequence identity to at least 8 contiguous nucleic acids of SEQ ID NO: 1 or 2. In some cases, the target nucleic acid has a sequence that has at least 80%, 85%, 90%, 91%, 92%, 93%, 94%, 95%, 96%, 97%, 98%, or 99%, or 100% sequence identity to at least 8 contiguous nucleic acids of SEQ ID NO: 1 or 2. In some cases, the target nucleic acid has a sequence that has at least 80%, 85%, 90%, 91%, 92%, 93%, 94%, 95%, 96%, 97%, 98%, or 99%, or 100% sequence identity to at least 10, 12, 14, or 16 contiguous nucleic acids of SEQ ID NO: 1 or 2. In some cases, the target nucleic acid has a sequence that has at least 80%, 85%, 90%, 91%, 92%, 93%, 94%, 95%, 96%, 97%, 98%, or 99%, or 100% sequence identity to at least 18 contiguous nucleic acids of SEQ ID NO: 1 or 2.

In some cases, the target nucleic acid is a portion of Exon 22 of CFTR pre-mRNA. In some cases, the target nucleic acid has at least 80%, 85%, 90%, 91%, 92%, 93%, 94%, 95%, 96%, 97%, 98%, or 99%, or 100% sequence identity to SEQ ID NO: 6. In some cases, the target nucleic acid has about 80%, 85%, 90%, 91%, 92%, 93%, 94%, 95%, 96%, 97%, 98%, 99%, or 100% sequence identity to SEQ ID NO: 6. In some cases, the target nucleic acid is a portion of the sequence of SEQ ID NO: 6. In some cases, the target nucleic acid has a sequence that has at least 80%, 85%, 90%, 91%, 92%, 93%, 94%, 95%, 96%, 97%, 98%, or 99%, or 100% sequence identity to at least 8 contiguous nucleic acids of SEQ ID NO: 6. In some cases, the target nucleic acid has a sequence that has at least 80%, 85%, 90%, 91%, 92%, 93%, 94%, 95%, 96%, 97%, 98%, or 99%, or 100% sequence identity to at least 8 contiguous nucleic acids of SEQ ID NO: 6. In some cases, the target nucleic acid has a sequence that has at least 80%, 85%, 90%, 91%, 92%, 93%, 94%, 95%, 96%, 97%, 98%, or 99%, or 100% sequence identity to at least 10, 12, 14, or 16 contiguous nucleic acids of SEQ ID NO: 6. In some cases, the target nucleic acid has a sequence that has at least 80%, 85%, 90%, 91%, 92%, 93%, 94%, 95%, 96%, 97%, 98%, or 99%, or 100% sequence identity to at least 18 contiguous nucleic acids of SEQ ID NO: 6.

In some cases, the target nucleic acid is a portion of Exon 23 of CFTR pre-mRNA. In some cases, the target nucleic acid has at least 80%, 85%, 90%, 91%, 92%, 93%, 94%, 95%, 96%, 97%, 98%, or 99%, or 100% sequence identity to SEQ ID NO: 7. In some cases, the target nucleic acid has about 80%, 85%, 90%, 91%, 92%, 93%, 94%, 95%, 96%, 97%, 98%, 99%, or 100% sequence identity to SEQ ID NO: 7. In some cases, the target nucleic acid is a portion of the sequence of SEQ ID NO: 7. In some cases, the target nucleic acid has a sequence that has at least 80%, 85%, 90%, 91%, 92%, 93%, 94%, 95%, 96%, 97%, 98%, or 99%, or 100% sequence identity to at least 8 contiguous nucleic acids of SEQ ID NO: 7. In some cases, the target nucleic acid has a sequence that has at least 80%, 85%, 90%, 91%, 92%, 93%, 94%, 95%, 96%, 97%, 98%, or 99%, or 100% sequence identity to at least 8 contiguous nucleic acids of SEQ ID NO: 7. In some cases, the target nucleic acid has a sequence that has at least 80%, 85%, 90%, 12, 14, or 16 contiguous nucleic acids of SEQ ID NO: 7. In some cases, the target nucleic acid has a sequence that has at least 80%, 85%, 90%, 91%, 92%, 93%, 94%, 95%, 96%, 97%, 98%, or 99%, or 100% sequence identity to at least 18 contiguous nucleic acids of SEQ ID NO: 7.

In some cases, the target nucleic acid is a portion of Intron 22 of CFTR pre-mRNA. In some cases, the target nucleic acid has at least 80%, 85%, 90%, 91%, 92%, 93%, 94%, 95%, 96%, 97%, 98%, or 99%, or 100% sequence identity to SEQ ID NO: 8. In some cases, the target nucleic acid has about 80%, 85%, 90%, 91%, 92%, 93%, 94%, 95%, 96%, 97%, 98%, 99%, or 100% sequence identity to SEQ ID NO: 8. In some cases, the target nucleic acid is a portion of the sequence of SEQ ID NO: 8. In some cases, the target nucleic acid has a sequence that has at least 80%, 85%, 90%, 91%, 92%, 93%, 94%, 95%, 96%, 97%, 98%, or 99%, or 100% sequence identity to at least 8 contiguous nucleic acids of SEQ ID NO: 8. In some cases, the target nucleic acid has a sequence that has at least 80%, 85%, 90%, 91%, 92%, 93%, 94%, 95%, 96%, 97%, 98%, or 99%, or 100% sequence identity to at least 8 contiguous nucleic acids of SEQ ID NO: 8. In some cases, the target nucleic acid has a sequence that has at least 80%, 85%, 90%, 91%, 92%, 93%, 94%, 95%, 96%, 97%, 98%, or 99%, or 100% sequence identity to at least 10, 12, 14, or 16 contiguous nucleic acids of SEQ ID NO: 8. In some cases, the target nucleic acid has a sequence that has at least 80%, 85%, 90%, 91%, 92%, 93%, 94%, 95%, 96%, 97%, 98%, or 99%, or 100% sequence identity to at least 18 contiguous nucleic acids of SEQ ID NO: 8.

In some cases, the target nucleic acid comprises an exon-intron junction of CFTR pre-mRNA. In some cases, the target nucleic acid comprises junction between Exon 22 and Intron 22 of CFTR pre-mRNA. In some cases, the target nucleic acid comprises junction between Intron 22 and Exon 23 of CFTR pre-mRNA.

In some cases, the target nucleic acid is a portion of Exon 22 of CFTR gene. In some cases, the target nucleic acid has at least 80%, 85%, 90%, 91%, 92%, 93%, 94%, 95%, 96%, 97%, 98%, or 99%, or 100% sequence identity to SEQ ID NO: 3. In some cases, the target nucleic acid has about 80%, 85%, 90%, 91%, 92%, 93%, 94%, 95%, 96%, 97%, 98%, 99%, or 100% sequence identity to SEQ ID NO: 3. In some cases, the target nucleic acid is a portion of the sequence of SEQ ID NO: 3. In some cases, the target nucleic acid has a sequence that has at least 80%, 85%, 90%, 91%, 92%, 93%, 94%, 95%, 96%, 97%, 98%, or 99%, or 100% sequence identity to at least 8 contiguous nucleic acids of SEQ ID NO: 3. In some cases, the target nucleic acid has a sequence that has at least 80%, 85%, 90%, 91%, 92%, 93%, 94%, 95%, 96%, 97%, 98%, or 99%, or 100% sequence identity to at least 8 contiguous nucleic acids of SEQ ID NO: 3. In some cases, the target nucleic acid has a sequence that has at least 80%, 85%, 90%, 12, 14, or 16 contiguous nucleic acids of SEQ ID NO: 3. In some cases, the target nucleic acid has a sequence that has at least 80%, 85%, 90%, 91%, 92%, 93%, 94%, 95%, 96%, 97%, 98%, or 99%, or 100% sequence identity to at least 18 contiguous nucleic acids of SEQ ID NO: 3.

In some cases, the target nucleic acid is a portion of Exon 23 of CFTR gene. In some cases, the target nucleic acid has at least 80%, 85%, 90%, 91%, 92%, 93%, 94%, 95%, 96%, 97%, 98%, or 99%, or 100% sequence identity to SEQ ID NO: 4. In some cases, the target nucleic acid has about 80%, 85%, 90%, 91%, 92%, 93%, 94%, 95%, 96%, 97%, 98%, 99%, or 100% sequence identity to SEQ ID NO: 4. In some cases, the target nucleic acid is a portion of the sequence of SEQ ID NO: 4. In some cases, the target nucleic acid has a sequence that has at least 80%, 85%, 90%, 91%, 92%, 93%, 94%, 95%, 96%, 97%, 98%, or 99%, or 100% sequence identity to at least 8 contiguous nucleic acids of SEQ ID NO: 4. In some cases, the target nucleic acid has a sequence that has at least 80%, 85%, 90%, 91%, 92%, 93%, 94%, 95%, 96%, 97%, 98%, or 99%, or 100% sequence identity to at least 8 contiguous nucleic acids of SEQ ID NO: 4. In some cases, the target nucleic acid has a sequence that has at least 80%, 85%, 90%, 91%, 92%, 93%, 94%, 95%, 96%, 97%, 98%, or 99%, or 100% sequence identity to at least 10, 12, 14, or 16 contiguous nucleic acids of SEQ ID NO: 4. In some cases, the target nucleic acid has a sequence that has at least 80%, 85%, 90%, 91%, 92%, 93%, 94%, 95%, 96%, 97%, 98%, or 99%, or 100% sequence identity to at least 18 contiguous nucleic acids of SEQ ID NO: 4.

In some cases, the target nucleic acid is a portion of Intron 22 of CFTR gene. In some cases, the target nucleic acid has at least 80%, 85%, 90%, 91%, 92%, 93%, 94%, 95%, 96%, 97%, 98%, or 99%, or 100% sequence identity to SEQ ID NO: 5. In some cases, the target nucleic acid has about 80%, 85%, 90%, 91%, 92%, 93%, 94%, 95%, 96%, 97%, 98%, 99%, or 100% sequence identity to SEQ ID NO: 5. In some cases, the target nucleic acid is a portion of the sequence of SEQ ID NO: 5. In some cases, the target nucleic acid has a sequence that has at least 80%, 85%, 90%, 91%, 92%, 93%, 94%, 95%, 96%, 97%, 98%, or 99%, or 100% sequence identity to at least 8 contiguous nucleic acids of SEQ ID NO: 5. In some cases, the target nucleic acid has a sequence that has at least 80%, 85%, 90%, 91%, 92%, 93%, 94%, 95%, 96%, 97%, 98%, or 99%, or 100% sequence identity to at least 8 contiguous nucleic acids of SEQ ID NO: 5. In some cases, the target nucleic acid has a sequence that has at least 80%, 85%, 90%, 91%, 92%, 93%, 94%, 95%, 96%, 97%, 98%, or 99%, or 100% sequence identity to at least 10, 12, 14, or 16 contiguous nucleic acids of SEQ ID NO: 5. In some cases, the target nucleic acid has a sequence that has at least 80%, 85%, 90%, 91%, 92%, 93%, 94%, 95%, 96%, 97%, 98%, or 99%, or 100% sequence identity to at least 18 contiguous nucleic acids of SEQ ID NO: 5.

In some cases, the target nucleic acid comprises an exon-intron junction of CFTR gene. In some cases, the target nucleic acid comprises junction between Exon 22 and Intron 22 of CFTR gene. In some cases, the target nucleic acid comprises junction between Intron 22 and Exon 23 of CFTR gene.

In some cases, the target nucleic acid has at least 80%, 85%, 90%, 91%, 92%, 93%, 94%, 95%, 96%, 97%, 98%, or 99%, or 100% sequence identity to any one of SEQ ID NOs: 9-36. In some cases, the target nucleic acid has about 80%, 85%, 90%, 91%, 92%, 93%, 94%, 95%, 96%, 97%, 98%, 99%, or 100% sequence identity to any one of SEQ ID NOS: 9-36. In some cases, the target nucleic acid is a portion of the sequence of any one of SEQ ID NOS: 9-36. In some cases, the target nucleic acid has a sequence that has at least 80%, 85%, 90%, 91%, 92%, 93%, 94%, 95%, 96%, 97%, 98%, or 99%, or 100% sequence identity to at least 8 contiguous nucleic acids of any one of SEQ ID NOS: 9-36. In some cases, the target nucleic acid has a sequence that has at least 80%, 85%, 90%, 91%, 92%, 93%, 94%, 95%, 96%, 97%, 98%, or 99%, or 100% sequence identity to at least 8 contiguous nucleic acids of any one of SEQ ID NOS: 9-36. In some cases, the target nucleic acid has a sequence that has at least 80%, 85%, 90%, 91%, 92%, 93%, 94%, 95%, 96%, 97%, 98%, or 99%, or 100% sequence identity to at least 10, 12, 14, or 16 contiguous nucleic acids of any one of SEQ ID NOS: 9-36. In some cases, the target nucleic acid has a sequence that has at least 80%, 85%, 90%, 91%, 92%, 93%, 94%, 95%, 96%, 97%, 98%, or 99%, or 100% sequence identity to at least 18 contiguous nucleic acids of any one of SEQ ID NOS: 9-36.

Agents provided herein can comprise a polynucleotide sequence. The polynucleotide sequence of the agent can be complementary to a target sequence located within CFTR gene or CFTR pre-mRNA.

In some cases, the agent comprises a polynucleotide sequence that is complementary to a portion of Exon 22 of CFTR gene or CFTR pre-mRNA. In some cases, the agent comprises a polynucleotide sequence that is at least 80%, 85%, 90%, 91%, 92%, 93%, 94%, 95%, 96%, 97%, 98%, or 99%, or 100% complementary to SEQ ID NO: 3 or 6. In some cases, the agent comprises a polynucleotide sequence that is about 80%, 85%, 90%, 91%, 92%, 93%, 94%, 95%, 96%, 97%, 98%, 99%, or 100% complementary to SEQ ID NO: 3 or 6. In some cases, the agent comprises a polynucleotide sequence that is complementary to a portion of the sequence of SEQ ID NO: 3 or 6. In some cases, the agent comprises a polynucleotide sequence that is at least 80%, 85%, 90%, 91%, 92%, 93%, 94%, 95%, 96%, 97%, 98%, or 99%, or 100% complementary to at least 8 contiguous nucleic acids of SEQ ID NO: 3 or 6. In some cases, the agent comprises a polynucleotide sequence that is at least 80%, 85%, 90%, 91%, 92%, 93%, 94%, 95%, 96%, 97%, 98%, or 99%, or 100% complementary to at least 8 contiguous nucleic acids of SEQ ID NO: 3 or 6. In some cases, the agent comprises a polynucleotide sequence that is at least 80%, 85%, 90%, 91%, 92%, 93%, 94%, 95%, 96%, 97%, 98%, or 99%, or 100% complementary to at least 10, 12, 14, or 16 contiguous nucleic acids of SEQ ID NO: 3 or 6. In some cases, the agent comprises a polynucleotide sequence that is at least 80%, 85%, 90%, 91%, 92%, 93%, 94%, 95%, 96%, 97%, 98%, or 99%, or 100% complementary to at least 18 contiguous nucleic acids of SEQ ID NO: 3 or 6.

In some cases, the agent comprises a polynucleotide sequence that is complementary to a portion of Exon 23 of CFTR gene or CFTR pre-mRNA. In some cases, the agent comprises a polynucleotide sequence that is at least 80%, 85%, 90%, 91%, 92%, 93%, 94%, 95%, 96%, 97%, 98%, or 99%, or 100% complementary to SEQ ID NO: 4 or 7. In some cases, the agent comprises a polynucleotide sequence that is about 80%, 85%, 90%, 91%, 92%, 93%, 94%, 95%, 96%, 97%, 98%, 99%, or 100% complementary to SEQ ID NO: 4 or 7. In some cases, the agent comprises a polynucleotide sequence that is complementary to a portion of the sequence of SEQ ID NO: 4 or 7. In some cases, the agent comprises a polynucleotide sequence that is at least 80%, 85%, 90%, 91%, 92%, 93%, 94%, 95%, 96%, 97%, 98%, or 99%, or 100% complementary to at least 8 contiguous nucleic acids of SEQ ID NO: 4 or 7. In some cases, the agent comprises a polynucleotide sequence that is at least 80%, 85%, 90%, 91%, 92%, 93%, 94%, 95%, 96%, 97%, 98%, or 99%, or 100% complementary to at least 8 contiguous nucleic acids of SEQ ID NO: 4 or 7. In some cases, the agent comprises a polynucleotide sequence that is at least 80%, 85%, 90%, 91%, 92%, 93%, 94%, 95%, 96%, 97%, 98%, or 99%, or 100% complementary to at least 10, 12, 14, or 16 contiguous nucleic acids of SEQ ID NO: 4 or 7. In some cases, the agent comprises a polynucleotide sequence that is at least 80%, 85%, 90%, 91%, 92%, 93%, 94%, 95%, 96%, 97%, 98%, or 99%, or 100% complementary to at least 18 contiguous nucleic acids of SEQ ID NO: 4 or 7.

In some cases, the agent comprises a polynucleotide sequence that is complementary to a portion of Intron 22 of CFTR gene or CFTR pre-mRNA. In some cases, the agent comprises a polynucleotide sequence that is at least 80%, 85%, 90%, 91%, 92%, 93%, 94%, 95%, 96%, 97%, 98%, or 99%, or 100% complementary to SEQ ID NO: 5 or 8. In some cases, the agent comprises a polynucleotide sequence that is about 80%, 85%, 90%, 91%, 92%, 93%, 94%, 95%, 96%, 97%, 98%, 99%, or 100% complementary to SEQ ID NO: 5 or 8. In some cases, the agent comprises a polynucleotide sequence that is complementary to a portion of the sequence of SEQ ID NO: 5 or 8. In some cases, the agent comprises a polynucleotide sequence that is at least 80%, 85%, 90%, 91%, 92%, 93%, 94%, 95%, 96%, 97%, 98%, or 99%, or 100% complementary to at least 8 contiguous nucleic acids of SEQ ID NO: 5 or 8. In some cases, the agent comprises a polynucleotide sequence that is at least 80%, 85%, 90%, 91%, 92%, 93%, 94%, 95%, 96%, 97%, 98%, or 99%, or 100% complementary to at least 8 contiguous nucleic acids of SEQ ID NO: 5 or 8. In some cases, the agent comprises a polynucleotide sequence that is at least 80%, 85%, 90%, 91%, 92%, 93%, 94%, 95%, 96%, 97%, 98%, or 99%, or 100% complementary to at least 10, 12, 14, or 16 contiguous nucleic acids of SEQ ID NO: 5 or 8. In some cases, the agent comprises a polynucleotide sequence that is at least 80%, 85%, 90%, 91%, 92%, 93%, 94%, 95%, 96%, 97%, 98%, or 99%, or 100% complementary to at least 18 contiguous nucleic acids of SEQ ID NO: 5 or 8.

In some cases, the agent comprises a polynucleotide sequence that is complementary to an exon-intron junction of CFTR gene or CFTR pre-mRNA. In some cases, the agent comprises a polynucleotide sequence that is complementary to junction between Exon 22 and Intron 22 of CFTR gene or CFTR pre-mRNA. In some cases, the agent comprises a polynucleotide sequence that is complementary to junction between Intron 22 and Exon 23 of CFTR gene or CFTR pre-mRNA.

The agent provided herein can comprise a polynucleotide sequence that is at least 80%, 85%, 90%, 91%, 92%, 93%, 94%, 95%, 96%, 97%, 98%, or 99%, or 100% sequence identity to any one of SEQ ID NOs: 9-36. In some cases, the agent comprises a polynucleotide sequence that is about 80%, 85%, 90%, 91%, 92%, 93%, 94%, 95%, 96%, 97%, 98%, 99%, or 100% complementary to any one of SEQ ID NOS: 9-36. In some cases, the agent comprises a polynucleotide sequence that is complementary to a portion of the sequence of any one of SEQ ID NOS: 9-36. In some cases, the agent comprises a polynucleotide sequence that is at least 80%, 85%, 90%, 91%, 92%, 93%, 94%, 95%, 96%, 97%, 98%, or 99%, or 100% complementary to at least 8 contiguous nucleic acids of any one of SEQ ID NOS: 9-36. In some cases, the agent comprises a polynucleotide sequence that is at least 80%, 85%, 90%, 91%, 92%, 93%, 94%, 95%, 96%, 97%, 98%, or 99%, or 100% complementary to at least 8 contiguous nucleic acids of any one of SEQ ID NOS: 9-36. In some cases, the agent comprises a polynucleotide sequence that is at least 80%, 85%, 90%, 91%, 92%, 93%, 94%, 95%, 96%, 97%, 98%, or 99%, or 100% complementary to at least 10, 12, 14, or 16 contiguous nucleic acids of any one of SEQ ID NOS: 9-36. In some cases, the agent comprises a polynucleotide sequence that is at least 80%, 85%, 90%, 91%, 92%, 93%, 94%, 95%, 96%, 97%, 98%, or 99%, or 100% complementary to at least 18 contiguous nucleic acids of any one of SEQ ID NOS: 9-36.

In some cases, the agent comprises a polynucleotide sequence that has at least 80%, 85%, 90%, 91%, 92%, 93%, 94%, 95%, 96%, 97%, 98%, or 99%, or 100% sequence identity to any one of SEQ ID NOs: 37-64. In some cases, the agent comprises a polynucleotide sequence that has about 80%, 85%, 90%, 91%, 92%, 93%, 94%, 95%, 96%, 97%, 98%, 99%, or 100% sequence identity to any one of SEQ ID NOS: 37-64. In some cases, the agent comprises a polynucleotide sequence that has a sequence that has at least 80%, 85%, 90%, 91%, 92%, 93%, 94%, 95%, 96%, 97%, 98%, or 99%, or 100% sequence identity to at least 8 contiguous nucleic acids of any one of SEQ ID NOS: 37-64. In some cases, the agent comprises a polynucleotide sequence that has a sequence that has at least 80%, 85%, 90%, 91%, 92%, 93%, 94%, 95%, 96%, 97%, 98%, or 99%, or 100% sequence identity to at least 8 contiguous nucleic acids of any one of SEQ ID NOS: 37-64. In some cases, the agent comprises a polynucleotide sequence that has a sequence that has at least 80%, 85%, 90%, 91%, 92%, 93%, 94%, 95%, 96%, 97%, 98%, or 99%, or 100% sequence identity to at least 10, 12, 14, or 16 contiguous nucleic acids of any one of SEQ ID NOS: 37-64. In some cases, the agent comprises a polynucleotide sequence that has a sequence that has at least 80%, 85%, 90%, 91%, 92%, 93%, 94%, 95%, 96%, 97%, 98%, or 99%, or 100% sequence identity to at least 18 contiguous nucleic acids of any one of SEQ ID NOS: 37-64.

In some cases, the agent provided herein target a nucleic acid encoding a CFTR protein in a cell. In some cases, the nucleic acid encoding a CFTR protein in the cell (e.g., CFTR gene or CFTR pre-mRNA) is a variant, e.g., has one or more mutations as compared to a wildtype nucleic acid (e.g., wildtype CFTR gene or wildtype CFTR pre-mRNA) encoding a wildtype CFTR protein. In some cases, the CFTR variant has one or more mutations at a location downstream of Intron 22 of CFTR gene or pre-mRNA. In some cases, the CFTR variant has one or more mutations such as c.3717G>A, c.3717+4A>G, c.3717+5G>A, c.3717+40A>G, c.3718-2477C>T, c.3718-1G>A, c.3718-3T>G, c.3719T>G, c.3731G>A, c.3737C>T, c.3744delA, c.3745G>A, c.3747delG, c.3752G>A, c.3761T>G, c.3763T>C, c.3764C>A, c.3773dupT, c.3806T>A, c.3808delG, c.3808G>A, c.3822G>A, c.3846G>A, c. [3846G>A;3848G>T], c.3848G>T, c.3872A>G, c.3873G>C, c.3873+1G>A, c. (3873+1_3874-1)_(3963+1_3964-1) del, c.3873+2T>C, 3876delA, c.3883_3886delATTT, c.3883delA, c.3889dupT, c.3891dupT, c.3908delA, c.3909C>G, c.3929G>A, c.3937C>T, c. (3963+1 3964-1)_(*1_?) del, c.3964-78_4242+577del, c.3971T>C, c.3988C>T, c.4004T>C, c.4036_4042del, c.4046G>A, c.4077_4080delTGTTinsAA, c.4086dupT, c.4097T>A, c.4111G>T, c.4124A>C, c.4127_4131delTGGAT, c.4144C>T, c.4147dupA, c.4197_4198delCT, c.4231C>T, c.4234C>T, c.4242+1G>T, c.4242+1G>A, c.4251delA, c.4300_4301dup, c.4364C>G, c.4426C>T, or c.4439T>C.

In some cases, at least one allele of CFTR gene in the cell, into which the agent provided herein is introduced, is a variant, for instance, having one or more mutations at a location downstream of Intron 22 of CFTR gene or pre-mRNA. In some cases, at least one allele of CFTR gene in the cell, into which the agent provided herein is introduced, has one or more mutations such as c.3717G>A, c.3717+4A>G, c.3717+5G>A, c.3717+40A>G, c.3718-2477C>T, c.3718-1G>A, c.3718-3T>G, c.3719T>G, c.3731G>A, c.3737C>T, c.3744delA, c.3745G>A, c.3747delG, c.3752G>A, c.3761T>G, c.3763T>C, c.3764C>A, c.3773dupT, c.3806T>A, c.3808delG, c.3808G>A, c.3822G>A, c.3846G>A, c. [3846G>A;3848G>T], c.3848G>T, c.3872A>G, c.3873G>C, c.3873+1G>A, c. (3873+1_3874-1)_(3963+1_3964-1) del, c.3873+2T>C, 3876delA, c.3883_3886delATTT, c.3883delA, c.3889dupT, c.3891dupT, c.3908delA, c.3909C>G, c.3929G>A, c.3937C>T, c. (3963+1_3964-1)_(*1_?) del, c.3964-78_4242+577del, c.3971T>C, c.3988C>T, c.4004T>C, c.4036_4042del, c.4046G>A, c.4077_4080delTGTTinsAA, c.4086dupT, c.4097T>A, c.4111G>T, c.4124A>C, c.4127_4131delTGGAT, c.4144C>T, c.4147dupA, c.4197_4198delCT, c.4231C>T, c.4234C>T, c.4242+1G>T, c.4242+1G>A, c.4251delA, c.4300_4301dup, c.4364C>G, c.4426C>T, or c.4439T>C.

### Therapeutic Agents

### ASO (Antisense Oligomers)

Provided herein is a composition comprising an antisense oligomer that modulates the processing of a CFTR pre-mRNA, e.g., suppressing splicing out of an intron (e.g., Intron 22) by binding to a targeted portion of a CFTR pre-mRNA. As used herein, the terms “ASO” and “antisense oligomer” are used interchangeably and refer to an oligomer such as a polynucleotide, comprising nucleobases that hybridizes to a target nucleic acid (e.g., a CFTR pre-mRNA) sequence by Watson-Crick base pairing or wobble base pairing (G-U). The ASO may have exact sequence complementary to the target sequence or near complementarity (e.g., sufficient complementarity to bind the target sequence and enhancing splicing at a splice site). ASOs are designed so that they bind (hybridize) to a target nucleic acid (e.g., a targeted portion of a pre-mRNA transcript) and remain hybridized under physiological conditions. Typically, if they hybridize to a site other than the intended (targeted) nucleic acid sequence, they hybridize to a limited number of sequences that are not a target nucleic acid (to a few sites other than a target nucleic acid). Design of an ASO can take into consideration the occurrence of the nucleic acid sequence of the targeted portion of the pre-mRNA transcript or a sufficiently similar nucleic acid sequence in other locations in the genome or cellular pre-mRNA or transcriptome, such that the likelihood the ASO will bind other sites and cause “off-target” effects is limited. Any antisense oligomers known in the art, for example in PCT Application No. PCT/US2014/054151, published as WO 2015/035091, titled “Reducing Nonsense-Mediated mRNA Decay,” incorporated by reference herein, can be used to practice the methods described herein.

In some embodiments, ASOs “specifically hybridize” to or are “specific” to a target nucleic acid or a targeted portion of a CFTR pre-mRNA. Typically, such hybridization occurs with a Tm substantially greater than 37° C., preferably at least 50° C., and typically between 60° C., to approximately 90° C. Such hybridization preferably corresponds to stringent hybridization conditions. At a given ionic strength and pH, the Tm is the temperature at which 50% of a target sequence hybridizes to a complementary oligonucleotide.

An ASO need not hybridize to all nucleobases in a target sequence and the nucleobases to which it does hybridize may be contiguous or noncontiguous. ASOs may hybridize over one or more segments of a pre-mRNA transcript, such that intervening or adjacent segments are not involved in the hybridization event (e.g., a loop structure or hairpin structure may be formed). In certain embodiments, an ASO hybridizes to noncontiguous nucleobases in a target pre-mRNA transcript. For example, an ASO can hybridize to nucleobases in a pre-mRNA transcript that are separated by one or more nucleobase(s) to which the ASO does not hybridize.

The ASOs described herein comprise nucleobases that are complementary to nucleobases present in a targeted portion of a CFTR pre-mRNA. The term ASO embodies oligonucleotides and any other oligomeric molecule that comprises nucleobases capable of hybridizing to a complementary nucleobase on a target mRNA but does not comprise a sugar moiety, such as a peptide nucleic acid (PNA). The ASOs may comprise naturally occurring nucleotides, nucleotide analogs, modified nucleotides, or any combination of two or three of the preceding. The term “naturally occurring nucleotides” includes deoxyribonucleotides and ribonucleotides. The term “modified nucleotides” includes nucleotides with modified or substituted sugar groups and/or having a modified backbone. In some embodiments, all the nucleotides of the ASO are modified nucleotides. Chemical modifications of ASOs or components of ASOs that are compatible with the methods and compositions described herein will be evident to one of skill in the art and can be found, for example, in U.S. Pat. No. 8,258,109 B2, U.S. Pat. No. 5,656,612, U.S. Patent Publication No. 2012/0190728, and Dias and Stein, Mol. Cancer Ther. 2002, 347-355, herein incorporated by reference in their entirety.

One or more nucleobases of an ASO may be any naturally occurring, unmodified nucleobase such as adenine, guanine, cytosine, thymine, and uracil, or any synthetic or modified nucleobase that is sufficiently similar to an unmodified nucleobase such that it is capable of hydrogen bonding with a nucleobase present on a target pre-mRNA. Examples of modified nucleobases include, without limitation, hypoxanthine, xanthine, 7-methylguanine, 5, 6-dihydrouracil, 5-methylcytosine, and 5-hydroxymethoylcytosine.

The ASOs described herein also comprise a backbone structure that connects the components of an oligomer. The term “backbone structure” and “oligomer linkages” may be used interchangeably and refer to the connection between monomers of the ASO. In naturally occurring oligonucleotides, the backbone comprises a 3′-5′ phosphodiester linkage connecting sugar moieties of the oligomer. The backbone structure or oligomer linkages of the ASOs described herein may include (but are not limited to) phosphorothioate, phosphorodithioate, phosphoroselenoate, phosphorodiselenoate, phosphoroanilothioate, phosphoraniladate, phosphoramidate, and the like. See, e.g., LaPlanche, et al., Nucleic Acids Res. 14:9081 (1986); Stec, et al., J. Am. Chem. Soc. 106:6077 (1984), Stein, et al., Nucleic Acids Res. 16:3209 (1988), Zon, et al., Anti-Cancer Drug Design 6:539 (1991); Zon, et al., Oligonucleotides and Analogues: A Practical Approach, pp. 87-108 (F. Eckstein, Ed., Oxford University Press, Oxford England (1991)); Stec, et al., U.S. Pat. No. 5,151,510; Uhlmann and Peyman, Chemical Reviews 90:543 (1990), of which entire content is incorporated herein by reference. In some embodiments, the backbone structure of the ASO does not contain phosphorous but rather contains peptide bonds, for example in a peptide nucleic acid (PNA), or linking groups including carbamate, amides, and linear and cyclic hydrocarbon groups. In some embodiments, the backbone modification is a phosphorothioate linkage. In some embodiments, the backbone modification is a phosphoramidate linkage.

In embodiments, the stereochemistry at each of the phosphorus internucleotide linkages of the ASO backbone is random. In embodiments, the stereochemistry at each of the phosphorus internucleotide linkages of the ASO backbone is controlled and is not random. For example, US2014/0194610, “Methods for the Synthesis of Functionalized Nucleic Acids,” incorporated herein by reference, describes methods for independently selecting the handedness of chirality at each phosphorous atom in a nucleic acid oligomer. In embodiments, an ASO used in the methods of the invention, including, but not limited to, any of the ASOs set forth herein in Tables 3A-3B, comprises an ASO having phosphorus internucleotide linkages that are not random. In embodiments, a composition used in the methods of the invention comprises a pure diastereomeric ASO. In embodiments, a composition used in the methods of the invention comprises an ASO that has diastereomeric purity of at least about 90%, at least about 91%, at least about 92%, at least about 93%, at least about 94%, at least about 95%, at least about 96%, at least about 97%, at least about 98%, at least about 99%, about 100%, about 90% to about 100%, about 91% to about 100%, about 92% to about 100%, about 93% to about 100%, about 94% to about 100%, about 95% to about 100%, about 96% to about 100%, about 97% to about 100%, about 98% to about 100%, or about 99% to about 100%.

In some cases, the antisense oligomer is at least 80%, 85%, 90%, 91%, 92%, 93%, 94%, 95%, 96%, 97%, 98%, or 99%, or 100% complementary to the sequence of any one of SEQ ID NOs: 9-36. In some cases, the antisense oligomer is about 80%, 85%, 90%, 91%, 92%, 93%, 94%, 95%, 96%, 97%, 98%, 99%, or 100% complementary to the sequence of any one of SEQ ID NOS: 7-36. In some cases, the antisense oligomer is complementary to a portion of the sequence of any one of SEQ ID NOS: 9-36. In some cases, the antisense oligomer has a sequence that is at least 80%, 85%, 90%, 91%, 92%, 93%, 94%, 95%, 96%, 97%, 98%, or 99%, or 100% complementary to at least 8 contiguous nucleic acids of any one of SEQ ID NOS: 9-36. In some cases, the antisense oligomer has a sequence that has at least 80%, 85%, 90%, 91%, 92%, 93%, 94%, 95%, 96%, 97%, 98%, or 99%, or 100% complementary to at least 8 contiguous nucleic acids of any one of SEQ ID NOS: 9-36. In some cases, the antisense oligomer has a sequence that has at least 80%, 85%, 90%, 91%, 92%, 93%, 94%, 95%, 96%, 97%, 98%, or 99%, or 100% complementary to at least 10, 12, 14, or 16 contiguous nucleic acids of any one of SEQ ID NOS: 9-36. In some cases, the antisense oligomer has a sequence that has at least 80%, 85%, 90%, 91%, 92%, 93%, 94%, 95%, 96%, 97%, 98%, or 99%, or 100% complementary to at least 18 contiguous nucleic acids of any one of SEQ ID NOS: 9-36.

In some cases, the antisense oligomer has at least 80%, 85%, 90%, 91%, 92%, 93%, 94%, 95%, 96%, 97%, 98%, or 99%, or 100% sequence identity to any one of SEQ ID NOs: 37-64. In some cases, the antisense oligomer has about 80%, 85%, 90%, 91%, 92%, 93%, 94%, 95%, 96%, 97%, 98%, 99%, or 100% sequence identity to any one of SEQ ID NOS: 37-64. In some cases, the antisense oligomer has a sequence that has at least 80%, 85%, 90%, 91%, 92%, 93%, 94%, 95%, 96%, 97%, 98%, or 99%, or 100% sequence identity to at least 8 contiguous nucleic acids of any one of SEQ ID NOS: 37-64. In some cases, the antisense oligomer has a sequence that has at least 80%, 85%, 90%, 91%, 92%, 93%, 94%, 95%, 96%, 97%, 98%, or 99%, or 100% sequence identity to at least 8 contiguous nucleic acids of any one of SEQ ID NOS: 37-64. In some cases, the antisense oligomer has a sequence that has at least 80%, 85%, 90%, 91%, 92%, 93%, 94%, 95%, 96%, 97%, 98%, or 99%, or 100% sequence identity to at least 10, 12, 14, or 16 contiguous nucleic acids of any one of SEQ ID NOS: 37-64. In some cases, the antisense oligomer has a sequence that has at least 80%, 85%, 90%, 91%, 92%, 93%, 94%, 95%, 96%, 97%, 98%, or 99%, or 100% sequence identity to at least 18 contiguous nucleic acids of any one of SEQ ID NOS: 37-64.

In embodiments, the ASO has a nonrandom mixture of Rp and Sp configurations at its phosphorus internucleotide linkages. For example, it has been suggested that a mix of Rp and Sp is required in antisense oligonucleotides or antisense oligomers to achieve a balance between good activity and nuclease stability (Wan, et al., 2014, “Synthesis, biophysical properties and biological activity of second-generation antisense oligonucleotides containing chiral phosphorothioate linkages,” Nucleic Acids Research 42 (22): 13456-13468, incorporated herein by reference). In embodiments, an ASO used in the methods of the invention, including, but not limited to, any of the ASOs set forth herein in SEQ ID NOs: 37-64, comprises about 5-100% Rp, at least about 5% Rp, at least about 10% Rp, at least about 15% Rp, at least about 20% Rp, at least about 25% Rp, at least about 30% Rp, at least about 35% Rp, at least about 40% Rp, at least about 45% Rp, at least about 50% Rp, at least about 55% Rp, at least about 60% Rp, at least about 65% Rp, at least about 70% Rp, at least about 75% Rp, at least about 80% Rp, at least about 85% Rp, at least about 90% Rp, or at least about 95% Rp, with the remainder Sp, or about 100% Rp. In embodiments, an ASO used in the methods of the invention, including, but not limited to, any of the ASOs set forth herein in SEQ ID NOs: 37-64, comprises about 10% to about 100% Rp, about 15% to about 100% Rp, about 20% to about 100% Rp, about 25% to about 100% Rp, about 30% to about 100% Rp, about 35% to about 100% Rp, about 40% to about 100% Rp, about 45% to about 100% Rp, about 50% to about 100% Rp, about 55% to about 100% Rp, about 60% to about 100% Rp, about 65% to about 100% Rp, about 70% to about 100% Rp, about 75% to about 100% Rp, about 80% to about 100% Rp, about 85% to about 100% Rp, about 90% to about 100% Rp, or about 95% to about 100% Rp, about 20% to about 80% Rp, about 25% to about 75% Rp, about 30% to about 70% Rp, about 40% to about 60% Rp, or about 45% to about 55% Rp, with the remainder Sp.

In embodiments, an ASO used in the methods of the invention, including, but not limited to, any of the ASOs set forth herein in SEQ ID NOs: 37-64, comprises about 5-100% Sp, at least about 5% Sp, at least about 10% Sp, at least about 15% Sp, at least about 20% Sp, at least about 25% Sp, at least about 30% Sp, at least about 35% Sp, at least about 40% Sp, at least about 45% Sp, at least about 50% Sp, at least about 55% Sp, at least about 60% Sp, at least about 65% Sp, at least about 70% Sp, at least about 75% Sp, at least about 80% Sp, at least about 85% Sp, at least about 90% Sp, or at least about 95% Sp, with the remainder Rp, or about 100% Sp. In embodiments, an ASO used in the methods of the invention, including, but not limited to, any of the ASOs set forth herein in SEQ ID NOs: 37-64, comprises about 10% to about 100% Sp, about 15% to about 100% Sp, about 20% to about 100% Sp, about 25% to about 100% Sp, about 30% to about 100% Sp, about 35% to about 100% Sp, about 40% to about 100% Sp, about 45% to about 100% Sp, about 50% to about 100% Sp, about 55% to about 100% Sp, about 60% to about 100% Sp, about 65% to about 100% Sp, about 70% to about 100% Sp, about 75% to about 100% Sp, about 80% to about 100% Sp, about 85% to about 100% Sp, about 90% to about 100% Sp, or about 95% to about 100% Sp, about 20% to about 80% Sp, about 25% to about 75% Sp, about 30% to about 70% Sp, about 40% to about 60% Sp, or about 45% to about 55% Sp, with the remainder Rp.

Any of the ASOs described herein may contain a sugar moiety that comprises ribose or deoxyribose, as present in naturally occurring nucleotides, or a modified sugar moiety or sugar analog, including a morpholine ring. Non-limiting examples of modified sugar moieties include 2′ substitutions such as 2′-O-methyl (2′-O-Me), 2′-O-methoxyethyl (2′MOE), 2′-O-aminoethyl, 2′-O—N-methyl-acetamide (2′-NMA), 2′ F; N3′->P5′ phosphoramidate, 2′ dimethylaminooxyethoxy, 2′ dimethylaminoethoxyethoxy, 2′-guanidinidium, 2′-O-guanidinium ethyl, carbamate modified sugars, and bicyclic modified sugars. In some embodiments, the sugar moiety modification is selected from 2′-O-Me, 2′-NMA, 2′F, and 2′MOE. In some embodiments, the sugar moiety modification is an extra bridge bond, such as in a locked nucleic acid (LNA). In some embodiments the sugar analog contains a morpholine ring, such as phosphorodiamidate morpholino (PMO). In some embodiments, the sugar moiety comprises a ribofuransyl or 2′ deoxyribofuransyl modification. In some embodiments, the sugar moiety comprises 2′4′-constrained 2′O-methyloxyethyl (cMOE) modifications. In some embodiments, the sugar moiety comprises cEt 2′, 4′ constrained 2′-O ethyl BNA modifications. In some embodiments, the sugar moiety comprises tricycloDNA (tcDNA) modifications. In some embodiments, the sugar moiety comprises ethylene nucleic acid (ENA) modifications. In some embodiments, the sugar moiety comprises MCE modifications. Modifications are known in the art and described in the literature, e.g., by Jarver, et al., 2014, Nucleic Acid Therapeutics 24 (1): 37-47, incorporated by reference for this purpose herein. “A Chemical View of Oligonucleotides for Exon Skipping and Related Drug Applications,” Nucleic Acid Therapeutics 24 (1): 37-47, incorporated by reference for this purpose herein.

In some embodiments, each monomer of the ASO is modified in the same way, for example each linkage of the backbone of the ASO comprises a phosphorothioate linkage or each ribose sugar moiety comprises a 2′O-methyl modification. Such modifications that are present on each of the monomer components of an ASO are referred to as “uniform modifications.” In some examples, a combination of different modifications may be desired, for example, an ASO may comprise a combination of phosphorodiamidate linkages and sugar moieties comprising morpholine rings (morpholinos). Combinations of different modifications to an ASO are referred to as “mixed modifications” or “mixed chemistries.”

In some embodiments, the ASO comprises one or more backbone modifications. In some embodiments, the ASO comprises one or more sugar moiety modification. In some embodiments, the ASO comprises one or more backbone modifications and one or more sugar moiety modifications. In some embodiments, the ASO comprises a 2′MOE modification and a phosphorothioate backbone. In some embodiments, the ASO comprises a phosphorodiamidate morpholino (PMO). In some embodiments, the ASO comprises a peptide nucleic acid (PNA). Any of the ASOs or any component of an ASO (e.g., a nucleobase, sugar moiety, backbone) described herein may be modified in order to achieve desired properties or activities of the ASO or reduce undesired properties or activities of the ASO. For example, an ASO or one or more components of any ASO may be modified to enhance binding affinity to a target sequence on a pre-mRNA transcript; reduce binding to any non-target sequence; reduce degradation by cellular nucleases (i.e., RNase H); improve uptake of the ASO into a cell and/or into the nucleus of a cell; alter the pharmacokinetics or pharmacodynamics of the ASO; and/or modulate the half-life of the ASO.

In some embodiments, the ASOs are comprised of 2′-O-(2-methoxyethyl) (MOE) phosphorothioate-modified nucleotides. ASOs comprised of such nucleotides are especially well-suited to the methods disclosed herein; oligomers having such modifications have been shown to have significantly enhanced resistance to nuclease degradation and increased bioavailability, making them suitable, for example, for oral delivery in some embodiments described herein. See e.g., Geary, et al., J Pharmacol Exp Ther. 2001; 296(3):890-7; Geary, et al., J Pharmacol Exp Ther. 2001; 296(3):898-904, of which entire content is incorporated herein by reference.

In some cases, the antisense oligomer comprises the sequence set forth in any one of SEQ ID NOs: 66-93. In some cases, the antisense oligomer consists of the sequence set forth in any one of SEQ ID NOs: 66-93.

Methods of synthesizing ASOs will be known to one of skill in the art. Alternatively or in addition, ASOs may be obtained from a commercial source.

Unless specified otherwise, the left-hand end of single-stranded nucleic acid (e.g., pre-mRNA transcript, oligonucleotide, ASO, etc.) sequences is the 5′ end and the left-hand direction of single or double-stranded nucleic acid sequences is referred to as the 5′ direction. Similarly, the right-hand end or direction of a nucleic acid sequence (single or double stranded) is the 3′ end or direction. Generally, a region or sequence that is 5′ to a reference point in a nucleic acid is referred to as “upstream,” and a region or sequence that is 3′ to a reference point in a nucleic acid is referred to as “downstream.” Generally, the 5′ direction or end of an mRNA is where the initiation or start codon is located, while the 3′ end or direction is where the termination codon is located. In some aspects, nucleotides that are upstream of a reference point in a nucleic acid may be designated by a negative number, while nucleotides that are downstream of a reference point may be designated by a positive number. For example, a reference point (e.g., an exon-exon junction in mRNA) may be designated as the “zero” site, and a nucleotide that is directly adjacent and upstream of the reference point is designated “minus one,” e.g., “−1,” while a nucleotide that is directly adjacent and downstream of the reference point is designated “plus one,” e.g., “+1.”

In some embodiments, the ASOs are complementary to (and bind to) a targeted portion of a CFTR pre-mRNA that is downstream (in the 3′ direction) of the 5′ splice site of Intron 22 (e.g., intron located from GRCh38.p14/hg38: chr7: 117,627,771 to GRCh38.p14/hg38: chr7: 117,642,437) in a CFTR pre-mRNA (e.g., the direction designated by positive numbers relative to the 5′ splice site). In some embodiments, the ASOs are complementary to a targeted portion of the CFTR pre-mRNA that is within the region about +1 to about +500 relative to the 5′ splice site of Intron 22 (e.g., intron located from GRCh38.p14/hg38: chr7: 117,627,771 to GRCh38.p14/hg38: chr7: 117,642,437). In some embodiments, the ASOs may be complementary to a targeted portion of a CFTR pre-mRNA that is within the region between nucleotides +6 and +496 relative to the 5′ splice site of Intron 22 (e.g., intron located from GRCh38.p14/hg38: chr7: 117,627,771 to GRCh38.p14/hg38: chr7: 117,642,437). In some aspects, the ASOs are complementary to a targeted portion that is within the region about +1 to about +500, about +1 to about +490, about +1 to about +480, about +1 to about +470, about +1 to about +460, about +1 to about +450, about +1 to about +440, about +1 to about +430, about +1 to about +420, about +1 to about +410, about +1 to about +400, about +1 to about +390, about +1 to about +380, about +1 to about +370, about +1 to about +360, about +1 to about +350, about +1 to about +340, about +1 to about +330, about +1 to about +320, about +1 to about +310, about +1 to about +300, about +1 to about +290, about +1 to about +280, about +1 to about +270, about +1 to about +260, about +1 to about +250, about +1 to about +240, about +1 to about +230, about +1 to about +220, about +1 to about +210, about +1 to about +200, about +1 to about +190, about +1 to about +180, about +1 to about +170, about +1 to about +160, about +1 to about +150, about +1 to about +140, about +1 to about +130, about +1 to about +120, about +1 to about +110, about +1 to about +100, about +1 to about +90, about +1 to about +80, about +1 to about +70, about +1 to about +60, about +1 to about +50, about +1 to about +40, about +1 to about +30, or about +1 to about +20 relative to 5′ splice site of Intron 22 (e.g., intron located from GRCh38.p14/hg38: chr7: 117,627,771 to GRCh38.p14/hg38: chr7: 117,642,437). In some aspects, the ASOs are complementary to a targeted portion that is within the region from about +1 to about +100, from about +100 to about +200, from about +200 to about +300, from about +300 to about +400, or from about +400 to about +500 relative to 5′ splice site of Intron 22 (e.g., intron located from GRCh38.p14/hg38: chr7: 117,627,771 to GRCh38.p14/hg38: chr7: 117,642,437).

In some embodiments, the ASOs are complementary to (and bind to) a targeted portion of a CFTR pre-mRNA that is upstream (in the 5′ direction) of the 5′ splice site of Intron 22 (e.g., intron located from GRCh38.p14/hg38: chr7: 117,627,771 to GRCh38.p14/hg38: chr7: 117,642,437). In some embodiments, the ASOs are complementary to a targeted portion of the CFTR pre-mRNA that is within the region about −4 to about −270 relative to the 5′ splice site of Intron 22 (e.g., intron located from GRCh38.p14/hg38: chr7: 117,627,771 to GRCh38.p14/hg38: chr7: 117,642,437). In some embodiments, the ASOs may be complementary to a targeted portion of a CFTR pre-mRNA that is within the region between nucleotides-1 and -264 relative to the 5′ splice site of Intron 22 (e.g., intron located from GRCh38.p14/hg38: chr7: 117,627,771 to GRCh38.p14/hg38: chr7: 117,642,437). In some aspects, the ASOs are complementary to a targeted portion that is within the region about −1 to about −270, about −1 to about −260, about −1 to about −250, about −1 to about −240, about −1 to about −230, about −1 to about −220, about −1 to about −210, about −1 to about −200, about −1 to about −190, about −1 to about −180, about −1 to about −170, about −1 to about −160, about −1 to about −150, about −1 to about −140, about −1 to about −130, about −1 to about −120, about −1 to about −110, about −1 to about −100, about −1 to about −90, about −1 to about −80, about −1 to about −70, about −1 to about −60, about −1 to about-50, about −1 to about −40, about −1 to about −30, or about −1 to about −20 relative to 5′ splice site of Intron 22 (e.g., intron located from GRCh38.p14/hg38: chr7: 117,627,771 to GRCh38.p14/hg38: chr7: 117,642,437). In some aspects, the ASOs are complementary to a targeted portion that is within the region from about −1 to about −50, from about −50 to about-100, from about −100 to about −150, from about −150 to about −200, or from about −200 to about −250 relative to 5′ splice site of Intron 22 (e.g., intron located from GRCh38.p14/hg38: chr7: 117,627,771 to GRCh38.p14/hg38: chr7: 117,642,437).

In some embodiments, the ASOs are complementary to a targeted region of a CFTR pre-mRNA that is upstream (in the 5′ direction) of the 3′ splice site of Intron 22 (e.g., intron located from GRCh38.p14/hg38: chr7: 117,627,771 to GRCh38.p14/hg38: chr7: 117,642,437). In some embodiments, the ASOs are complementary to a targeted portion of the CFTR pre-mRNA that is within the region about −1 to about −500 relative to the 3′ splice site of Intron 22 (e.g., intron located from GRCh38.p14/hg38: chr7: 117,627,771 to GRCh38.p14/hg38: chr7: 117,642,437). In some embodiments, the ASOs are complementary to a targeted portion of the CFTR pre-mRNA that is within the region-1 to -496 relative to the 3′ splice site of Intron 22 (e.g., intron located from GRCh38.p14/hg38: chr7: 117,627,771 to GRCh38.p14/hg38: chr7: 117,642,437). In some aspects, the ASOs are complementary to a targeted portion that is within the region about −1 to about −500, about −1 to about −490, about −1 to about −480, about −1 to about −470, about −1 to about −460, about −1 to about −450, about −1 to about −440, about −1 to about −430, about −1 to about −420, about −1 to about −410, about −1 to about −400, about −1 to about −390, about −1 to about −380, about −1 to about −370, about −1 to about −360, about −1 to about −350, about −1 to about −340, about −1 to about −330, about −1 to about −320, about −1 to about −310, about −1 to about −300, about −1 to about −290, about −1 to about −280, about −1 to about −270, about −1 to about −260, about −1 to about −250, about −1 to about −240, about −1 to about −230, about −1 to about −220, about −1 to about −210, about −1 to about −200, about −1 to about −190, about −1 to about −180, about −1 to about −170, about −1 to about −160, about −1 to about −150, about −1 to about −140, about −1 to about −130, about −1 to about −120, about −1 to about −110, about −1 to about −100, about −1 to about −90, about −1 to about −80, about −1 to about −70, about −1 to about −60, about −1 to about −50, about −1 to about −40, or about −1 to about −30 relative to 3′ splice site of Intron 22 (e.g., intron located from GRCh38.p14/hg38: chr7: 117,627,771 to GRCh38.p14/hg38: chr7: 117,642,437) In some aspects, the ASOs are complementary to a targeted portion that is within the region from about −1 to about −100, from about −100 to about −200, from about −200 to about −300, from about −300 to about −400, or from about −400 to about −500 relative to 3′ splice site of Intron 22 (e.g., intron located from GRCh38.p14/hg38: chr7: 117,627,771 to GRCh38.p14/hg38: chr7: 117,642,437).

In some embodiments, the ASOs are complementary to a targeted region of a CFTR pre-mRNA that is downstream (in the 3′ direction) of the 3′ splice site of Intron 22 (e.g., intron located from GRCh38.p14/hg38: chr7: 117,627,771 to GRCh38.p14/hg38: chr7: 117,642,437). In some embodiments, the ASOs are complementary to a targeted portion of the CFTR pre-mRNA that is within the region of about +1 to about +100 relative to the 3′ splice site of Intron 22 (e.g., intron located from GRCh38.p14/hg38: chr7: 117,627,771 to GRCh38.p14/hg38: chr7: 117,642,437). In some aspects, the ASOs are complementary to a targeted portion that is within the region about +1 to about +90, about +1 to about +80, about +1 to about +70, about +1 to about +60, about +1 to about +50, about +1 to about +40, about +1 to about +30, about +1 to about +20, or about +1 to about +10 relative to 3′ splice site of Intron 22 (e.g., intron located from GRCh38.p14/hg38: chr7: 117,627,771 to GRCh38.p14/hg38: chr7: 117,642,437).

In some embodiments, the targeted portion of the CFTR pre-mRNA is within the region +100 relative to the 5′ splice site of Intron 22 (e.g., intron located from GRCh38.p14/hg38: chr7: 117,627,771 to GRCh38.p14/hg38: chr7: 117,642,437) to -100 relative to the 3′ splice site of Intron 22 (e.g., intron located from GRCh38.p14/hg38: chr7: 117,627,771 to GRCh38.p14/hg38: chr7: 117,642,437). In some embodiments, the targeted portion of the CFTR pre-mRNA is within Intron 22 (e.g., intron located from GRCh38.p14/hg38: chr7: 117,627,771 to GRCh38.p14/hg38: chr7: 117,642,437). In some embodiments, the targeted portion of the (TR pre-mRNA comprises an exon and intron boundary.

The ASOs may be of any length suitable for specific binding and effective enhancement of splicing. In some embodiments, the ASOs consist of 8 to 50 nucleobases. For example, the ASO may be 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, or 50 nucleobases in length. In some embodiments, the ASOs consist of more than 50 nucleobases. In some embodiments, the ASO is from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 9 to 50 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 50 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 50 nucleobases, 12 to 40 nucleobases, 12 to 35 nucleobases, 12 to 30 nucleobases, 12 to 25 nucleobases, 12 to 20 nucleobases, 12 to 15 nucleobases, 13 to 50 nucleobases, 13 to 40 nucleobases, 13 to 35 nucleobases, 13 to 30 nucleobases, 13 to 25 nucleobases, 13 to 20 nucleobases, 14 to 50 nucleobases, 14 to 40 nucleobases, 14 to 35 nucleobases, 14 to 30 nucleobases, 14 to 25 nucleobases, 14 to 20 nucleobases, 15 to 50 nucleobases, 15 to 40 nucleobases, 15 to 35 nucleobases, 15 to 30 nucleobases, 15 to 25 nucleobases, 15 to 20 nucleobases, 20 to 50 nucleobases, 20 to 40 nucleobases, 20 to 35 nucleobases, 20 to 30 nucleobases, 20 to 25 nucleobases, 25 to 50 nucleobases, 25 to 40 nucleobases, 25 to 35 nucleobases, or 25 to 30 nucleobases in length. In some embodiments, the ASOs are 18 nucleotides in length. In some embodiments, the ASOs are 15 nucleotides in length. In some embodiments, the ASOs are 25 nucleotides in length.

In some embodiments, two or more ASOs with different chemistries but complementary to the same targeted portion of the CFTR pre-mRNA are used. In some embodiments, two or more ASOs that are complementary to different targeted portions of the CFTR pre-mRNA are used.

In embodiments, the antisense oligonucleotides, or antisense oligomers of the disclosure are chemically linked to one or more moieties or conjugates, e.g., a targeting moiety or other conjugate that enhances the activity or cellular uptake of the oligonucleotide. Such moieties include, but are not limited to, a lipid moiety, e.g., as a cholesterol moiety, a cholesteryl moiety, an aliphatic chain, e.g., dodecandiol or undecyl residues, a polyamine, or a polyethylene glycol chain, or adamantane acetic acid. Oligonucleotides comprising lipophilic moieties and preparation methods have been described in the published literature. In some embodiments, the antisense oligonucleotide or antisense oligomer is conjugated with a moiety including, but not limited to, an abasic nucleotide, an aptamer, a polyether, a polyamine, a polyamide, a peptide, a polypeptide (e.g., antibody), a carbohydrate, e.g., N-acetylgalactosamine (GalNAc), N-Ac-Glucosamine (GluNAc), or mannose (e.g., mannose-6-phosphate), a lipid, or a polyhydrocarbon compound. Conjugates can be linked to one or more of any nucleotides comprising the antisense oligonucleotide or antisense oligomer at any of several positions on the sugar, base, or phosphate group, as understood in the art and described in the literature, e.g., using a linker. Linkers can include a bivalent or trivalent branched linker. In embodiments, the conjugate is attached to the 3′ end of the antisense oligonucleotide or antisense oligomer. Methods of preparing oligonucleotide conjugates are described, e.g., in U.S. Pat. No. 8,450,467, “Carbohydrate conjugates as delivery agents for oligonucleotides,” incorporated by reference herein. In some embodiments, the antisense oligonucleotides, or antisense oligomers of the disclosure are conjugated non-covalently with a moiety comprises a nanoparticle for nucleic acid drug delivery that is known to a person skilled in the art. Examples of strategies that can be applied to enhance delivery of antisense oligonucleotide or antisens oligomer of the present disclosure are described, e.g., in Roberts et al., Advances in oligonucleotide drug delivery. Nat Rev Drug Discov 19, 673-694 (2020), which is incorporated herein by reference in its entirety.

In some embodiments, the nucleic acid to be targeted by an ASO is a CFTR pre-mRNA expressed in a cell, such as a eukaryotic cell. In some embodiments, the term “cell” may refer to a population of cells. In some embodiments, the cell is in a subject. In some embodiments, the cell is isolated from a subject. In some embodiments, the cell is ex vivo. In some embodiments, the cell is a condition or disease-relevant cell or a cell line. In some embodiments, the cell is in vitro (e.g., in cell culture).

In some embodiments, the ASO is the salt of a nucleotide. In some embodiments, the ASO is the salt of a nucleotide, fully phosphorothioate-linked oligonucleotide. In some embodiments, the ASO is the salt of a nucleotide in which the salt binds to the phosphate-link. In some embodiments, the ASO is the salt of a nucleotide, fully phosphorothioate-linked oligonucleotide in which the salt binds to the phosphate-link. In some embodiments, the ASO is the sodium salt of a nucleotide. In some embodiments, the ASO is the sodium salt of a nucleotide, fully phosphorothioate-linked oligonucleotide. In some embodiments, the ASO is the sodium salt of a nucleotide in which the sodium salt binds to the phosphate-link. In some embodiments, the ASO is the sodium salt of a nucleotide, fully phosphorothioate-linked oligonucleotide in which the sodium salt binds to the phosphate-link. In some embodiments, the ASO is the potassium salt of a nucleotide. In some embodiments, the ASO is the potassium salt of a nucleotide, fully phosphorothioate-linked oligonucleotide. In some embodiments, the ASO is the potassium salt of a nucleotide in which the potassium salt binds to the phosphate-link. In some embodiments, the ASO is the potassium salt of a nucleotide, fully phosphorothioate-linked oligonucleotide in which the potassium salt binds to the phosphate-link.

In some embodiment, the ASO is the undecasodium salt of a 12-nucleotide (12-mer). In some embodiment, the ASO is the dodecasodium salt of a 13-nucleotide (13-mer). In some embodiment, the ASO is the tridecasodium salt of a 14-nucleotide (14-mer). In some embodiment, the ASO is the tetradecasodium salt of a 15-nucleotide (15-mer). In some embodiment, the ASO is the pentadecasodium salt of a 16-nucleotide (16-mer). In some embodiment, the ASO is the hexadecasodium salt of a 17-nucleotide (17-mer). In some embodiment, the ASO is the heptadecasodium salt of an 18-nucleotide (18-mer). In some embodiment, the ASO is the octadecasodium salt of a 19-nucleotide (19-mer). In some embodiment, the ASO is the nonadecasodium salt of a 20-nucleotide (20-mer). In some embodiment, the ASO is the icosasodium salt of a 21-nucleotide (21-mer). In some embodiment, the ASO is the henicosasodium salt of a 22-nucleotide (22-mer). In some embodiment, the ASO is the docosasodium salt of a 23-nucleotide (23-mer). In some embodiment, the ASO is the tricosasodium salt of a 24-nucleotide (24-mer). In some embodiment, the ASO is the tetracosasodium salt of a 25-nucleotide (25-mer). In some embodiment, the ASO is the pentacosasodium salt of a 26-nucleotide (26-mer). In some embodiment, the ASO is the hexacosasodium salt of a 27-nucleotide (27-mer). In some embodiment, the ASO is the heptacosasodium salt of a 28-nucleotide (28-mer). In some embodiment, the ASO is the octacosasodium salt of a 29-nucleotide (29-mer). In some embodiment, the ASO is the nonacosasodium salt of a 30-nucleotide (30-mer). In some embodiment, the ASO is the triacontasodium salt of a 31-nucleotide (31-mer). In some embodiment, the ASO is the hentriacontasodium salt of a 32-nucleotide (32-mer). In some embodiment, the ASO is the dotriacontasodium salt of a 33-nucleotide (33-mer). In some embodiment, the ASO is the tritriacontasodium salt of a 34-nucleotide (34-mer). In some embodiment, the ASO is the tetratriacontasodium salt of a 35-nucleotide (35-mer). In some embodiment, the ASO is the pentatriacontasodium salt of a 36-nucleotide (36-mer). In some embodiment, the ASO is the hexatriacontasodium salt of a 37-nucleotide (37-mer). In some embodiment, the ASO is the heptatriacontasodium salt of a 38-nucleotide (38-mer). In some embodiment, the ASO is the octatriacontasodium salt of a 39-nucleotide (39-mer). In some embodiment, the ASO is the nonatriacontasodium salt of a 40-nucleotide (40-mer). In some embodiment, the ASO is the tetracontasodium salt of a 41-nucleotide (41-mer). In some embodiment, the ASO is the hentetracontasodium salt of a 42-nucleotide (42-mer). In some embodiment, the ASO is the dotetracontasodium salt of a 43-nucleotide (43-mer). In some embodiment, the ASO is the tritetracontasodium salt of a 44-nucleotide (44-mer). In some embodiment, the ASO is the tetratetracontasodium salt of a 45-nucleotide (45-mer). In some embodiment, the ASO is the pentatetracontasodium salt of a 46-nucleotide (46-mer). In some embodiment, the ASO is the hexatetracontasodium salt of a 47-nucleotide (47-mer). In some embodiment, the ASO is the heptatetracontasodium salt of a 48-nucleotide (48-mer). In some embodiment, the ASO is the octatetracontasodium salt of a 49-nucleotide (49-mer). In some embodiment, the ASO is the nonatetracontasodium salt of a 50-nucleotide (50-mer). In some embodiment, the ASO is the pentacontasodium salt of a 51-nucleotide (51-mer).

In some embodiment, the ASO is the undecapotassium salt of a 12-nucleotide (12-mer). In some embodiment, the ASO is the dodecapotassium salt of a 13-nucleotide (13-mer). In some embodiment, the ASO is the tridecapotassium salt of a 14-nucleotide (14-mer). In some embodiment, the ASO is the tetradecapotassium salt of a 15-nucleotide (15-mer). In some embodiment, the ASO is the pentadecapotassium salt of a 16-nucleotide (16-mer). In some embodiment, the ASO is the hexadecapotassium salt of a 17-nucleotide (17-mer). In some embodiment, the ASO is the heptadecapotassium salt of a 18-nucleotide (18-mer). In some embodiment, the ASO is the octadecapotassium salt of a 19-nucleotide (19-mer). In some embodiment, the ASO is the nonadecapotassium salt of a 20-nucleotide (20-mer). In some embodiment, the ASO is the icosapotassium salt of a 21-nucleotide (21-mer). In some embodiment, the ASO is the henicosapotassium salt of a 22-nucleotide (22-mer). In some embodiment, the ASO is the docosapotassium salt of a 23-nucleotide (23-mer). In some embodiment, the ASO is the tricosapotassium salt of a 24-nucleotide (24-mer). In some embodiment, the ASO is the tetracosapotassium salt of a 25-nucleotide (25-mer). In some embodiment, the ASO is the pentacosapotassium salt of a 26-nucleotide (26-mer). In some embodiment, the ASO is the hexacosapotassium salt of a 27-nucleotide (27-mer). In some embodiment, the ASO is the heptacosapotassium salt of a 28-nucleotide (28-mer). In some embodiment, the ASO is the octacosapotassium salt of a 29-nucleotide (29-mer). In some embodiment, the ASO is the nonacosapotassium salt of a 30-nucleotide (30-mer). In some embodiment, the ASO is the triacontapotassium salt of a 31-nucleotide (31-mer). In some embodiment, the ASO is the hentriacontapotassium salt of a 32-nucleotide (32-mer). In some embodiment, the ASO is the dotriacontapotassium salt of a 33-nucleotide (33-mer). In some embodiment, the ASO is the tritriacontapotassium salt of a 34-nucleotide (34-mer). In some embodiment, the ASO is the tetratriacontapotassium salt of a 35-nucleotide (35-mer). In some embodiment, the ASO is the pentatriacontapotassium salt of a 36-nucleotide (36-mer). In some embodiment, the ASO is the hexatriacontapotassium salt of a 37-nucleotide (37-mer). In some embodiment, the ASO is the heptatriacontapotassium salt of a 38-nucleotide (38-mer). In some embodiment, the ASO is the octatriacontapotassium salt of a 39-nucleotide (39-mer). In some embodiment, the ASO is the nonatriacontapotassium salt of a 40-nucleotide (40-mer). In some embodiment, the ASO is the tetracontapotassium salt of a 41-nucleotide (41-mer). In some embodiment, the ASO is the hentetracontapotassium salt of a 42-nucleotide (42-mer). In some embodiment, the ASO is the dotetracontapotassium salt of a 43-nucleotide (43-mer). In some embodiment, the ASO is the tritetracontapotassium salt of a 44-nucleotide (44-mer). In some embodiment, the ASO is the tetratetracontapotassium salt of a 45-nucleotide (45-mer). In some embodiment, the ASO is the pentatetracontapotassium salt of a 46-nucleotide (46-mer). In some embodiment, the ASO is the hexatetracontapotassium salt of a 47-nucleotide (47-mer). In some embodiment, the ASO is the heptatetracontapotassium salt of a 48-nucleotide (48-mer). In some embodiment, the ASO is the octatetracontapotassium salt of a 49-nucleotide (49-mer). In some embodiment, the ASO is the nonatetracontapotassium salt of a 50-nucleotide (50-mer). In some embodiment, the ASO is the pentacontapotassium salt of a 51-nucleotide (51-mer).

### Vectors

In some aspects, provided herein is a vector encoding an agent provided herein. The vector can express an agent that comprises a polynucleotide sequence disclosed herein. For instance, the vector can be a viral vector expressing a polynucleotide sequence that binds to a targeted portion of a gene or a pre-mRNA the encodes the target peptide sequence (e.g., CFTR gene or CFTR pre-mRNA that encodes a CFTR protein). The methods provided herein can be adapted to contacting a vector that encodes an agent, e.g., an oligonucleotide, to a cell, so that the agent binds to a gene or a pre-mRNA in the cell, and thus the agent can modulate the gene or the processing of the pre-mRNA. In some cases, the vector encoding the agent comprises plasmid DNA, viral vectors, and bacterial vectors. In some cases, the viral vector comprises an adenoviral vector, adeno-associated viral (AAV) vector, lentiviral vector, Herpes Simplex Virus (HSV) viral vector, retroviral vector, a parvovirus-based vector, or any applicable viral vector.

### Gene Editing

In some aspects, provided herein is an agent that can be used for gene editing.

In some cases, an agent comprises or forms part of a gene editing tool that is configured to modify CFTR gene. For instance, the agent can remove nucleic acid sequence of the CFTR gene that is downstream of a first intron that comprises an alternative adenylation site (e.g., Intron 22). In some cases, the nucleic acid sequence removed by the agent from CFTR gene is located from GRCh38.p14/hg38: chr7: 117,642,438 to GRCh38.p14/hg38: chr7: 117,668,665. As such, a level of a processed mRNA that encodes a CFTR protein and that lacks nucleic acid sequence downstream of the first intron is increased.

In some cases, a gene editing tool comprises vector, e.g., viral vector, for gene editing based on CRISPR-Cas9, TALEN, Zinc Finger, or other applicable technologies.

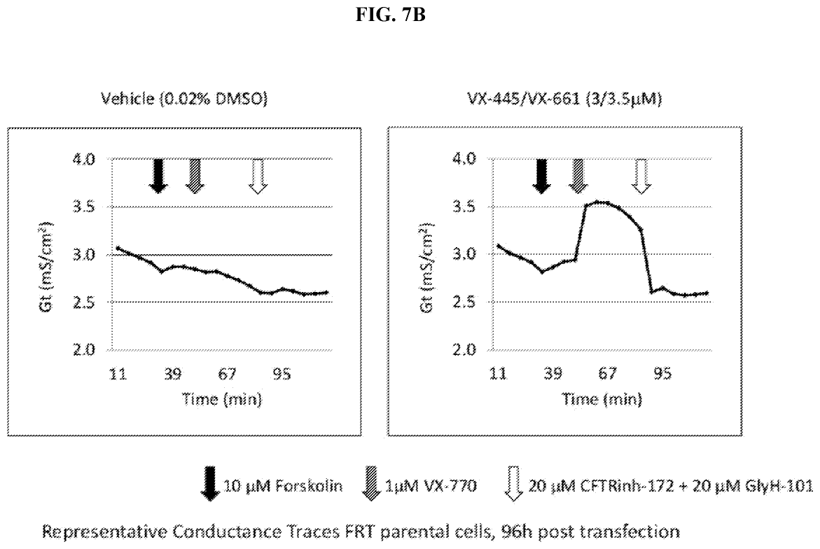

In some cases, the agent for gene editing provided herein comprises a polynucleotide sequence complementary to a target nucleic acid located within CFTR gene. For instance, the agent can comprise a guide RNA complementary to a target nucleic acid located within CFTR gene. As depicted in FIG. 8, in some embodiments, the guide RNA targets a genomic region of CFTR gene 5′ to Exon 23. In some embodiments, the guide RNA targets a genomic region within Intron 22 of the CFTR gene. In some embodiments, the guide RNA targets a genomic region of CFTR gene 3′ downstream to Exon 27. In some embodiments, the guide RNA targets a genomic region downstream of the 3′UTR of the CFTR gene. In some cases, the agent for gene editing comprises more than one guide RNAs. In some cases, the agent for gene editing comprises at least 2 guide RNAs. In some cases, the guide RNA comprises the sequences of SEQ ID NO: 94. In some cases, the guide RNA comprises the sequences of SEQ ID NO: 95. In some cases, the agent for gene editing comprises a pair of guide RNAs, wherein the pair of guide RNAs comprise the sequences of SEQ ID NOs: 94 and 95, respectively.

In some cases, the agent for gene editing comprises a polynucleotide sequence that is complementary to a portion of Exon 23 of CFTR gene. In some cases, the agent comprises a polynucleotide sequence that is at least 80%, 85%, 90%, 91%, 92%, 93%, 94%, 95%, 96%, 97%, 98%, or 99%, or 100% complementary to SEQ ID NO: 4. In some cases, the agent comprises a polynucleotide sequence that is about 80%, 85%, 90%, 91%, 92%, 93%, 94%, 95%, 96%, 97%, 98%, 99%, or 100% complementary to SEQ ID NO: 4. In some cases, the agent comprises a polynucleotide sequence that is complementary to a portion of the sequence of SEQ ID NO: 4. In some cases, the agent comprises a polynucleotide sequence that is at least 80%, 85%, 90%, 91%, 92%, 93%, 94%, 95%, 96%, 97%, 98%, or 99%, or 100% complementary to at least 8 contiguous nucleic acids of SEQ ID NO: 4. In some cases, the agent comprises a polynucleotide sequence that is at least 80%, 85%, 90%, 91%, 92%, 93%, 94%, 95%, 96%, 97%, 98%, or 99%, or 100% complementary to at least 8 contiguous nucleic acids of SEQ ID NO: 4. In some cases, the agent comprises a polynucleotide sequence that is at least 80%, 85%, 90%, 91%, 92%, 93%, 94%, 95%, 96%, 97%, 98%, or 99%, or 100% complementary to at least 10, 12, 14, or 16 contiguous nucleic acids of SEQ ID NO: 4. In some cases, the agent comprises a polynucleotide sequence that is at least 80%, 85%, 90%, 91%, 92%, 93%, 94%, 95%, 96%, 97%, 98%, or 99%, or 100% complementary to at least 18 contiguous nucleic acids of SEQ ID NO: 4.

In some cases, the agent for gene editing comprises a polynucleotide sequence that is complementary to a portion of Exon 22 of CFTR gene. In some cases, the agent comprises a polynucleotide sequence that is at least 80%, 85%, 90%, 91%, 92%, 93%, 94%, 95%, 96%, 97%, 98%, or 99%, or 100% complementary to SEQ ID NO: 3. In some cases, the agent comprises a polynucleotide sequence that is about 80%, 85%, 90%, 91%, 92%, 93%, 94%, 95%, 96%, 97%, 98%, 99%, or 100% complementary to SEQ ID NO: 3. In some cases, the agent comprises a polynucleotide sequence that is complementary to a portion of the sequence of SEQ ID NO: 3. In some cases, the agent comprises a polynucleotide sequence that is at least 80%, 85%, 90%, 91%, 92%, 93%, 94%, 95%, 96%, 97%, 98%, or 99%, or 100% complementary to at least 8 contiguous nucleic acids of SEQ ID NO: 3. In some cases, the agent comprises a polynucleotide sequence that is at least 80%, 85%, 90%, 91%, 92%, 93%, 94%, 95%, 96%, 97%, 98%, or 99%, or 100% complementary to at least 8 contiguous nucleic acids of SEQ ID NO: 3. In some cases, the agent comprises a polynucleotide sequence that is at least 80%, 85%, 90%, 91%, 92%, 93%, 94%, 95%, 96%, 97%, 98%, or 99%, or 100% complementary to at least 10, 12, 14, or 16 contiguous nucleic acids of SEQ ID NO: 3. In some cases, the agent comprises a polynucleotide sequence that is at least 80%, 85%, 90%, 91%, 92%, 93%, 94%, 95%, 96%, 97%, 98%, or 99%, or 100% complementary to at least 18 contiguous nucleic acids of SEQ ID NO: 3.

In some cases, the agent for gene editing comprises a polynucleotide sequence that is complementary to a portion of Intron 22 of CFTR gene. In some cases, the agent comprises a polynucleotide sequence that is at least 80%, 85%, 90%, 91%, 92%, 93%, 94%, 95%, 96%, 97%, 98%, or 99%, or 100% complementary to SEQ ID NO: 5. In some cases, the agent comprises a polynucleotide sequence that is about 80%, 85%, 90%, 91%, 92%, 93%, 94%, 95%, 96%, 97%, 98%, 99%, or 100% complementary to SEQ ID NO: 5. In some cases, the agent comprises a polynucleotide sequence that is complementary to a portion of the sequence of SEQ ID NO: 5. In some cases, the agent comprises a polynucleotide sequence that is at least 80%, 85%, 90%, 91%, 92%, 93%, 94%, 95%, 96%, 97%, 98%, or 99%, or 100% complementary to at least 8 contiguous nucleic acids of SEQ ID NO: 5. In some cases, the agent comprises a polynucleotide sequence that is at least 80%, 85%, 90%, 91%, 92%, 93%, 94%, 95%, 96%, 97%, 98%, or 99%, or 100% complementary to at least 8 contiguous nucleic acids of SEQ ID NO: 5. In some cases, the agent comprises a polynucleotide sequence that is at least 80%, 85%, 90%, 91%, 92%, 93%, 94%, 95%, 96%, 97%, 98%, or 99%, or 100% complementary to at least 10, 12, 14, or 16 contiguous nucleic acids of SEQ ID NO: 5. In some cases, the agent comprises a polynucleotide sequence that is at least 80%, 85%, 90%, 91%, 92%, 93%, 94%, 95%, 96%, 97%, 98%, or 99%, or 100% complementary to at least 18 contiguous nucleic acids of SEQ ID NO: 5.

In some cases, the agent for gene editing further comprises a polynucleotide sequence that is complementary to a portion of 3′ UTR of CFTR gene. In some cases, the agent comprises a polynucleotide sequence that is at least 80%, 85%, 90%, 91%, 92%, 93%, 94%, 95%, 96%, 97%, 98%, or 99%, or 100% complementary to SEQ ID NO: 65. In some cases, the agent comprises a polynucleotide sequence that is about 80%, 85%, 90%, 91%, 92%, 93%, 94%, 95%, 96%, 97%, 98%, 99%, or 100% complementary to SEQ ID NO: 65. In some cases, the agent comprises a polynucleotide sequence that is complementary to a portion of the sequence of SEQ ID NO: 65. In some cases, the agent comprises a polynucleotide sequence that is at least 80%, 85%, 90%, 91%, 92%, 93%, 94%, 95%, 96%, 97%, 98%, or 99%, or 100% complementary to at least 8 contiguous nucleic acids of SEQ ID NO: 65. In some cases, the agent comprises a polynucleotide sequence that is at least 80%, 85%, 90%, 91%, 92%, 93%, 94%, 95%, 96%, 97%, 98%, or 99%, or 100% complementary to at least 8 contiguous nucleic acids of SEQ ID NO: 65. In some cases, the agent comprises a polynucleotide sequence that is at least 80%, 85%, 90%, 91%, 92%, 93%, 94%, 95%, 96%, 97%, 98%, or 99%, or 100% complementary to at least 10, 12, 14, or 16 contiguous nucleic acids of SEQ ID NO: 65. In some cases, the agent comprises a polynucleotide sequence that is at least 80%, 85%, 90%, 91%, 92%, 93%, 94%, 95%, 96%, 97%, 98%, or 99%, or 100% complementary to at least 18 contiguous nucleic acids of SEQ ID NO: 65.

### Treatment of Subjects

Any of the compositions provided herein may be administered to an individual. “Individual” may be used interchangeably with “subject” or “patient.” An individual may be a mammal, for example a human or animal such as a non-human primate, a rodent, a rabbit, a rat, a mouse, a horse, a donkey, a goat, a cat, a dog, a cow, a pig, or a sheep. In embodiments, the individual is a human. In embodiments, the individual is a fetus, an embryo, or a child. In other embodiments, the individual may be another eukaryotic organism, such as a plant. In some embodiments, the compositions provided herein are administered to a cell ex vivo.

In some embodiments, the compositions provided herein are administered to an individual as a method of treating a disease or disorder. In some embodiments, the individual has a genetic disease, such as any of the diseases described herein. In some embodiments, the individual is at risk of having a disease, such as any of the diseases described herein. In some embodiments, the individual is at increased risk of having a disease or disorder caused by insufficient amount of a protein or insufficient activity of a protein. If an individual is “at an increased risk” of having a disease or disorder caused insufficient amount of a protein or insufficient activity of a protein, the method involves preventative or prophylactic treatment. For example, an individual may be at an increased risk of having such a disease or disorder because of family history of the disease. Typically, individuals at an increased risk of having such a disease or disorder benefit from prophylactic treatment (e.g., by preventing or delaying the onset or progression of the disease or disorder). In embodiments, a fetus is treated in utero, e.g., by administering the ASO composition to the fetus directly or indirectly (e.g., via the mother).

In some cases, a therapeutic agent is an agent provided herein. For instance, a therapeutic agent can comprise an antisense oligomer. In some cases, a therapeutic agent comprises a vector, e.g., a viral vector, expressing an oligonucleotide that binds to the targeted region of a pre-mRNA the encodes the target peptide sequence (e.g., CFTR pre-mRNA that encodes CFTR protein). The methods provided herein can be adapted to contacting a vector that encodes an agent, e.g., an oligonucleotide, to a cell, so that the agent binds to a pre-mRNA in the cell and modulates the processing of the pre-mRNA. In some cases, the viral vector comprises an adenoviral vector, adeno-associated viral (AAV) vector, lentiviral vector, Herpes Simplex Virus (HSV) viral vector, retroviral vector, a parvovirus-based vector, or any applicable viral vector. In some cases, a therapeutic agent comprises a gene editing tool that is configured to modify a gene encoding the target peptide sequence such that a gene region that encodes the inefficient translation region is deleted. In some cases, a gene editing tool comprises vector, e.g., viral vector, for gene editing based on CRISPR-Cas9, TALEN, Zinc Finger, or other applicable technologies.

Suitable routes for administration of agents of the present disclosure may vary depending on cell type to which delivery of the agents is desired. Multiple tissues and organs are affected by cystic fibrosis, with the lungs being the most significantly affected tissue. The agents of the present disclosure may be administered to patients parenterally, for example, via intranasal administration, intratracheal administration, intravenous injection, intraperitoneal injection, intramuscular injection, intrathecal injection, subcutaneous injection, oral administration, synovial injection, intracerebroventricular injection, intravitreal administration, subretinal injection, topical application, or implantation.

In some embodiments, subjects treated using the methods and compositions are evaluated for improvement in condition using any methods known and described in the art.

Any of the compositions provided herein may be administered to an individual (e.g., a human subject) to treat one or more symptoms associated with cystic fibrosis or related complications. In some cases, any of the composition provided herein can be administered to an individual to reduce a risk of developing cystic fibrosis or developing one or more symptoms associated with cystic fibrosis or related complications. In some cases, the one or more symptoms ameliorated or prevented by the subject composition or method of the present disclosure include isolated obstructive azoospermia, chronic sinusitis, chronic pancreatitis, pulmonary dysfunction, thick, viscous secretions in the lungs, pancreas, liver, intestine, and reproductive tract, elevated sweat chloride, and increased salt content in sweat gland secretions. In some cases, the one or more symptoms ameliorated or prevented by the subject composition or method of the present disclosure include respiratory and/or pulmonary symptoms, such as persistent productive cough, hyperinflation of the lung fields on chest radiograph, microorganism infection, bronchiectasis, airway hyperreactivity, allergic bronchopulmonary aspergillosis, obstructive sleep apnea, and pulmonary hypertension. In some cases, the subject composition or method of the present disclosure reduces risk of developing atypical symptoms that can arise in late life of a cystic fibrosis patient, including sinus disease, pancreatic disease, infertility, musculoskeletal disorders, recurrent venous thrombosis, anemia, electrolyte abnormalities, nephrolithiasis, and aquagenic wrinkling.

Any of the compositions provided herein may be administered to an individual to treat a CFTR-related disorder (CFTR-RD). CFTR-RD can comprise one or more clinical entities associated with CFTR dysfunction that may not fulfil diagnostic criteria for CF. CFTR-RDs can be caused by impaired CFTR channel function in multiple affected organs (e.g., lungs, pancreas, liver, reproductive tract), and can also be associated with worse lung function, poorer nutritional status, and chest infections. CFTR-RDs can comprise disorders of the gastrointestinal tract (GI tract), including CF-related pancreatic insufficiency, CF-related pancreatitis, CF-related diabetes, CF-related liver disease and gallbladder disease. CFTR-RDs comprise disorders of the reproductive tract, including congenital bilateral absence of the vas deferens (CBAVD), and disorders of the lungs, including disseminated bronchiectasis.

In some cases, the compositions provided herein can ameliorate a CFTR-RD by restoring the transport of CFTR-associated ions in the lungs and improving lung function. In some cases, the compositions provided herein can address the underlying cause of a CFTR-RD directly by enhancing transport of CFTR-associated ions in affected organs (e.g., lungs, pancreas, liver, reproductive tract). Any of the compositions provided herein may be administered to an individual (e.g., a male human subject) to treat one or more symptoms associated with congenital bilateral absence of the vas deferens (CBAVD). In some cases, the congenital bilateral absence of the vas deferens in the subject is associated with one or more mutations in the CFTR gene. Subjects that suffer from congenital bilateral absence of the vas deferens can have retarded development or underdevelopment of vas deferens (the tubes that carry sperm out of the testes). In some cases, the compositions provided herein promote development of vas deferens in the subject or treat one or more dysfunctions of the vas deferens in the subject.

Where reference is made to increasing partially functional and functional CFTR protein levels, the increase may be clinically significant. The increase may be relative to the level of partially functional and functional CFTR protein in the subject without treatment, or relative to the amount of CFTR protein in a population of similar subjects. It is generally accepted that 5% of wild type CFTR function can be therapeutically relevant, e.g., when a therapeutic intervention restores CFTR function in a patient to a level that is at least 5% of wild type CFTR function, the intervention would be therapeutically effective, e.g., ameliorating, eliminating, or preventing one or more symptoms associated with deficiency in CFTR activity and/or function that the patient suffers. The increase may be at least 10% or more CFTR protein function relative to the average subject, or the subject prior to treatment. The increase may be at least 20% or more CFTR protein relative to the average subject, or the subject prior to treatment. The increase may be at least 40% or more CFTR protein relative to the average subject, or the subject prior to treatment. The increase may be at least 50% or more CFTR protein relative to the average subject, or the subject prior to treatment. The increase may be at least 80% or more CFTR protein relative to the average subject, or the subject prior to treatment. The increase may be at least 100% or more CFTR protein relative to the average subject, or the subject prior to treatment.

The therapy of promoting Exon 22 truncation as described herein can be used for treating many CFTR mutations that are downstream 3′ of the first Exon 22 ApA site. For example, FIG. 1C listed lists 13 mutations that can result in PTC in Exon 23 to 27, all potential candidates for the Exon 22 truncation therapy. Non-limiting exemplary CFTR mutations that the subject compositions, methods, and kits are applicable to include c.3717G>A, c.3717+4A>G, c.3717+5G>A, c.3717+40A>G, c.3718-2477C>T, c.3718-1G>A, c.3718-3T>G, c.3719T>G, c.3731G>A, c.3737C>T, c.3744delA, c.3745G>A, c.3747delG, c.3752G>A, c.3761T>G, c.3763T>C, c.3764C>A, c.3773dupT, c.3806T>A, c.3808delG, c.3808G>A, c.3822G>A, c.3846G>A, c. [3846G>A;3848G>T], c.3848G>T, c.3872A>G, c.3873G>C, c.3873+1G>A, c. (3873+1_3874-1)_(3963+1_3964-1) del, c.3873+2T>C, 3876delA, c.3883_3886delATTT, c.3883delA, c.3889dupT, c.3891dupT, c.3908delA, c.3909C>G, c.3929G>A, c.3937C>T, c. (3963+1_3964-1)_(*1_?) del, c.3964-78_4242+577del, c.3971T>C, c.3988C>T, c.4004T>C, c.4036_4042del, c.4046G>A, c.4077_4080delTGTTinsAA, c.4086dupT, c.4097T>A, c.4111G>T, c.4124A>C, c.4127_4131delTGGAT, c.4144C>T, c.4147dupA, c.4197_4198delCT, c.4231C>T, c.4234C>T, c.4242+1G>T, c.4242+1G>A, c.4251 delA, c.4300_4301dup, c.4364C>G, c.4426C>T, or c.4439T>C.

### Pharmaceutical Composition

Pharmaceutical compositions or formulations comprising the agent, e.g., antisense oligonucleotide, of the described compositions and for use in any of the described methods can be prepared according to conventional techniques well known in the pharmaceutical industry and described in the published literature. In embodiments, a pharmaceutical composition or formulation for treating a subject comprises an effective amount of any antisense oligomer as described herein, or a pharmaceutically acceptable salt, solvate, hydrate or ester thereof. The pharmaceutical formulation comprising an antisense oligomer may further comprise a pharmaceutically acceptable excipient, a diluent, or a carrier.

Pharmaceutically acceptable salts are suitable for use in contact with the tissues of humans and lower animals without undue toxicity, irritation, allergic response, etc., and are commensurate with a reasonable benefit/risk ratio. (See, e.g., S. M. Berge, et al., J. Pharmaceutical Sciences, 66:1-19 (1977), incorporated herein by reference for this purpose. The salts can be prepared in situ during the final isolation and purification of the compounds, or separately by reacting the free base form with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other documented methodologies such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

In some embodiments, the compositions are formulated into any of many possible dosage forms such as, but not limited to, tablets, capsules, gel capsules, liquid syrups, soft gels, suppositories, and enemas. In embodiments, the compositions are formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers. In embodiments, a pharmaceutical formulation or composition of the present disclosure includes, but is not limited to, a solution, emulsion, microemulsion, foam or liposome-containing formulation (e.g., cationic or noncationic liposomes).

The pharmaceutical composition or formulation described herein may comprise one or more penetration enhancers, carriers, excipients or other active or inactive ingredients as appropriate and well known to those of skill in the art or described in the published literature. In some embodiments, liposomes also include sterically stabilized liposomes, e.g., liposomes comprising one or more specialized lipids. These specialized lipids result in liposomes with enhanced circulation lifetimes. In some embodiments, a sterically stabilized liposome comprises one or more glycolipids or is derivatized with one or more hydrophilic polymers, such as a polyethylene glycol (PEG) moiety. In some embodiments, a surfactant is included in the pharmaceutical formulation or compositions. The use of surfactants in drug products, formulations and emulsions is well known in the art. In embodiments, the present disclosure employs a penetration enhancer to promote the efficient delivery of the antisense oligonucleotide, e.g., to aid diffusion across cell membranes and/or enhance the permeability of a lipophilic drug. In some embodiments, the penetration enhancers are a surfactant, fatty acid, bile salt, chelating agent, or non-chelating nonsurfactant. In some embodiments, the pharmaceutical composition or formulation can comprise a lipid nanoparticle. In some embodiments, the pharmaceutical formulation comprises multiple antisense oligonucleotides. In embodiments, the antisense oligonucleotide is administered in combination with another drug or therapeutic agent.

In some cases, the pharmaceutical composition is formulated for any suitable administration method for a cystic fibrosis patient, such as parenteral administration, including intranasal administration, intratracheal administration, intravenous injection, intraperitoneal injection, intramuscular injection, intrathecal injection, subcutaneous injection, oral administration, synovial injection, intracerebroventricular injection, intravitreal administration, subretinal injection, topical application, or implantation. In some cases, intratracheal administration comprises administering pulmonary dosage forms, including forms for administration via a dry powder inhaler, a metered dose inhaler, or a nebulizer either in conventional or nanoparticles and microparticles form.

### Combination Treatment

In some cases, methods provided herein comprise administration of a first therapeutic agent and second therapeutic agent. In some cases, the first agent comprises a therapeutic agent provided herein, and the second agent is another agent that is a known agent for treatment of cystic fibrosis or another diseases or conditions.

In one embodiment, the additional agent is an antibiotic or anti-infective agent (e.g., azithromycin, amoxicillin and clavulanic acid, cloxacillin and dicloxacillin, ticarcillin and clavulanic acid, cephalexin, cefdinir, cefprozil, cefaclor; sulfamethoxazole and trimethoprim, erythromycin/sulfisoxazole, erythromycin, clarithromycin, tetracycline, doxycycline, minocycline, tigecycline, vancomycin, imipenem, meropenem, colistimethate, linezolid, ciprofloxacin, levofloxacin, or a combination thereof.

In one embodiment, the additional agent is one or more CFTR modulators. In some cases, the CFTR modulator can improve the processing and trafficking of one or more variants of CFTR proteins, e.g., lumacaftor, tezacaftor, elexacaftor, or tezacaftor. In some cases, the CFTR modulator can potentiate chloride conductance of one or more variants of CFTR proteins on the cells, e.g., ivacaftor. In some embodiments, the one or more CFTR modulators are a combination of agents that can potentiate chloride conductance and agents that can improve the processing and trafficking of one or more variants of the CFTR proteins, e.g., ivacaftor (KALYDECO®), lumacaftor, lumacaftor/ivacaftor (ORKAMBI®), tezacaftor/ivacaftor (SYMDEKO®), or TRIKAFTA® elexacaftor/ivacaftor/tezacaftor (TRIKAFTA®). In some cases, the CFTR modulators are referred to by the names used in basic research, which can be used interchangeably herein, e.g., elexacaftor/VX-661; tezacaftor/VX-445; ivacaftor/VX-770, lumacaftor/VX-809, or Trikafta/VX-445/VX-661/VX-770.

In one embodiment, the additional agent is a mucolytic agent for airway clearance e.g., acetylcysteine, ambroxol, bromhexine, carbocisteine, erdosteine, mecysteine, dornase alfa, hypertonic saline, or mannitol. In one embodiment, the additional agent is a bronchodilator (e.g., albuterol).

In one embodiment, the additional agent is an immunosuppressive agent includes corticosteroids (e.g., an inhaled corticosteroid (e.g., beclomethasone (QVAR®), budesonide (PULMICORT®), budesonide/formoterol (SYMBICORT®), ciclesonide (ALVESCO®), fluticasone (FLOVENT HFA®), fluticasone propionate (FLOVENT DISKUS®), fluticasone furoate (ARNUITY ELLIPTA®), fluticasone propionate/salmeterol (ADVAIR®), fluticasone furoate/umeclidinium/vilanterol (TRELEGY ELLIPTA®), mometasone furoate (ASMANEX®), or mometasone/formoterol (DULERA®), prednisone, or methylprednisolone). In one embodiment, the additional agent is a non-steroidal immunosuppressive agent, which can be biologics, including polyclonal anti-lymphocyte antibodies (e.g., anti-lymphocyte globulin (ALG) or anti-thymocyte globulin (ATG) antibodies, which may be, for example, horse- or rabbit-derived), monoclonal anti-lymphocyte antibodies (e.g., anti-CD3 antibodies (e.g., muromonab or alemtuzumab) or anti-CD20 antibodies (e.g., rituximab)), interleukin-2 (IL-2) receptor antagonists (e.g., daclizumab or basiliximab). In one embodiment, the additional agent is a non-steroidal immunosuppressive agents, which can be small molecules drugs including calcineurin inhibitors (e.g., cyclosporin A or tacrolimus), cell cycle inhibitors (e.g., azathioprine, mycophenolate mofetil (MMF), or mycophenolic acid (MPA)), mammalian target of rapamycin (mTOR) inhibitors (e.g., sirolimus (rapamycin) or everolimus), methotrexate, cyclophosphamide, an anthracycline (e.g., doxorubicin, idarubicin, aclarubicin, daunorubicin, epirubicin, valrubicin, mitoxantrone, or a combination thereof), a taxane (e.g., TAXOL® (paclitaxel)), and a combination thereof (e.g., a combination of a calcineurin inhibitor, a cell cycle inhibitor, or a corticosteroid).

In one embodiment, the additional agent is a nutritional agent, such as pancrelipase (pancreatic enzyme replacement) (e.g., Pancrease®, Pancreacarb®, or Ultrase®), Creon®, Liprotamase® (formerly Trizytek®), Aquadeks®, and glutathione inhalation.

In some embodiments, the ASOs disclosed in the present disclosure can be used in combination with one or more additional therapeutic agents. In some embodiments, the one or more additional therapeutic agents can comprise a small molecule. For example, the one or more additional therapeutic agents can comprise a small molecule described in USUS20220241206, which is incorporated by reference herein in its entirety. In some embodiments, the one or more additional therapeutic agents comprise an ASO that can be used to inhibit intron splicing out.

In some embodiments, the ASOs disclosed herein can be used in combination with one or more additional nucleic-acid-based therapeutic agents for treating cystic fibrosis. In some cases, the additional therapeutic agents comprise agents for replacement therapy (e.g., gene therapy and RNA replacement), agents for gene editing (e.g., CRISPR-Cas9, TALEN, and Zinc Finger), agents for RNA interference (e.g., siRNA, miRNA), ASOs (e.g., gene silencing ASO, exon-skipping ASO, or read-through ASO), and tRNA (e.g., suppressor tRNA).

### Kit

In other aspects, provide herein is a kit. The kit disclosed herein can comprise the composition or the pharmaceutical composition disclosed herein, and instructions for use of the composition or the pharmaceutical composition. The kit can comprise the composition or the pharmaceutical composition disclosed herein, and a second therapeutic agent (or an additional agent). In some cases, the kit further comprises one or more additional reagents, such as an immunosuppressive agent as described above, or one or more agents for treating cystic fibrosis as described herein. Kits provided herein typically include a label indicating the intended use of the contents of the kit. The term label includes any writing, or recorded material supplied on or with the kit, or which otherwise accompanies the kit.

### Methods of Identifying Additional Asos

Also within the scope of the present disclosure are methods for identifying or determining ASOs that modulates processing of CFTR pre-mRNA transcripts, e.g., splicing of CFTR pre-mRNA and/or alternative polyadenylation of CFTR splicing products. For example, a method can comprise identifying or determining ASOs that processing of CFTR pre-mRNA transcripts, e.g., splicing of CFTR pre-mRNA and/or alternative polyadenylation of CFTR splicing products. ASOs that specifically hybridize to different nucleotides within the targeted portion of the CFTR pre-mRNA can be screened to identify or determine ASOs that promote production of Exon 22 truncated CFTR mRNA transcripts, for instance, ASOs that suppress splicing out of Intron 22 and/or promotes polyadenylation at an alternative polyadenylation site in Intron 22. In some embodiments, the ASO may interfere interaction of one or more splicing factors with CFTR pre-mRNA. Any method known in the art may be used to identify (determine) an ASO that when hybridized to the targeted portion of the processed mRNA results in the desired effect (e.g., increasing level of Exon 22 truncated CFTR mRNA, suppressing splicing out of Intron 22, promoting polyadenylation at an alternative polyadenylation site in Intron 22). An example of a method that may be used is provided below.

A round of screening, referred to as an ASO “walk” may be performed using ASOs that have been designed to hybridize to a targeted portion of a CFTR pre-mRNA transcript, e.g., a CFTR pre-mRNA transcript that comprises a nonsense mutation downstream of Intron 22. For example, the ASOs used in the ASO walk can be tiled every 5 nucleotides from approximately 100 nucleotides upstream of a region of interest (e.g., a targeted portion within Exon 22, Intron 22, and/or Exon 23 of CFTR pre-mRNA) to approximately 100 nucleotides downstream of the region of interest. For example, a first ASO of 15 nucleotides in length may be designed to specifically hybridize to the first 18 nucleotides at the 5′ end of Intron 22 of CFTR pre-mRNA, e.g., +1 to +15 relative to the 5′ end of Intron 22 of CFTR pre-mRNA. A second ASO may be designed to specifically hybridize to nucleotides +6 to +20 relative to the 5′ end of Intron 22 of CFTR pre-mRNA. ASOs are designed as such spanning the targeted portion of the CFTR pre-mRNA transcript. In embodiments, the ASOs can be tiled more closely, e.g., every 1, 2, 3, or 4 nucleotides.

One or more ASOs, or a control ASO (an ASO with a scrambled sequence, sequence that is not expected to hybridize to the target region) are delivered, for example by transfection, into a disease-relevant cell line that expresses CFTR pre-mRNA. The modulation effects of each of the ASOs may be assessed by any method known in the art, for example by assessing the mature CFTR mRNA transcripts level in the cell, or the expression level of CFTR protein encoded by the processed mRNA. Assessment of the mature CFTR mRNA transcripts can be performed by reverse transcriptase (RT)-PCR, for instance, using primers that span across a portion of Exon 23. A reduction or absence of a longer RT-PCR product produced using the primers spanning the region containing the excluded nucleic acid in ASO-treated cells as compared to in control ASO-treated cells indicates that level of the processed mRNA that lacks the nucleic acid sequence downstream of the first intron that comprises the alternative adenylation site has been increased. In some embodiments, splicing out of Intron 22 may be suppressed using the ASOs described herein. The amount of C-terminal truncated CFTR protein that is encoded by the processed mRNA can also be assessed to determine whether each ASO achieved the desired effect (e.g., enhanced partial protein expression). Any method known in the art for assessing and/or quantifying protein production, such as Western blotting, flow cytometry, immunofluorescence microscopy, and ELISA, can be used. In some cases, function of CFTR protein in the cell can also be assessed to screen for desirable ASOs according to some embodiments of the present disclosure. For instance, an increase in chloride channel conductivity can indicate an increase in the level of functional or partially functional CFTR protein in the cell. In some cases, CFTR corrector/potentiator is used for the functional assay.

A second round of screening, referred to as an ASO “micro-walk” may be performed using ASOs that have been designed to hybridize to a target region of a pre-mRNA. The ASOs used in the ASO micro-walk are tiled every 1 nucleotide to further refine the nucleotide acid sequence of the pre-mRNA that when hybridized with an ASO results in modulation of the processing of the pre-mRNA.

Regions defined by ASOs that promote splicing of the target intron are explored in greater detail by means of an ASO “micro-walk”, involving ASOs spaced in 1-nt steps, as well as longer ASOs, typically 18-25 nt.

As described for the ASO walk above, the ASO micro-walk is performed by delivering one or more ASOs, or a control ASO (an ASO with a scrambled sequence, sequence that is not expected to hybridize to the target region), for example by transfection, into a disease-relevant cell line that expresses the target pre-mRNA. Similar approaches as the ones in the ASO walk above can be used to assess the effects of the candidate ASOs during the ASO micro-walk.

ASOs that when hybridized to a region of a pre-mRNA result in modulation of processing of CFTR pre-mRNA and increased production of C-terminal truncated CFTR protein can be tested in vivo using animal models, for example transgenic mouse models in which the full-length human CFTR gene (with or without genetic mutation(s) identified in human cystic fibrosis patients) has been knocked-in or in mouse models of disease. Suitable routes for administration of ASOs may vary depending on the disease and/or the cell types to which delivery of the ASOs is desired. ASOs may be administered, for example, by intranasal administration, intratracheal administration, intravenous injection, intraperitoneal injection, intramuscular injection, intrathecal injection, subcutaneous injection, oral administration, synovial injection, intracerebroventricular injection, intravitreal administration, subretinal injection, topical application, or implantation. Following administration, the cells, tissues, and/or organs of the model animals may be assessed to determine the effect of the ASO treatment by for example evaluating mRNA processing and protein production by methods known in the art and described herein. The animal models may also be any phenotypic or behavioral indication of the disease or disease severity.

### EXAMPLES

The following examples are provided to further illustrate some embodiments of the present disclosure but are not intended to limit the scope of the disclosure; it will be understood by their exemplary nature that other procedures, methodologies, or techniques known to those skilled in the art may alternatively be used.

In this work, a novel alternative approach is described that is based on the use of ASOs, which has therapeutic potential to address subset of CFTR PTC variants that reside in 3′ terminus for which there are no approved treatments. A novel feature of this approach is that it allows for CFTR 3′ PTC specific NMD evasion and does not require global NMD inhibition. This strategy takes advantage of a mechanistic understanding of regulatory events that culminate in 2 fates of intron 22 processing, 1) canonical exon 22/23 splicing that result in intron 22 excision or 2) usage of intron 22 ApA sites that result in a 3′ truncated CFTR mRNA isoform which is termed as E22 trunc here. As depicted in FIG. 1B, inhibition of canonical exon 22/23 splicing may promote intron 22 ApA usage and promote high expression of E22 trunc mRNA via NMD escape for 3′ terminal CFTR PTCs (i.e., R1162X & W1282X), and E22 trunc protein. Additionally, given that CFTR W1281 truncated protein is known to retain partial function when treated with modulators, upregulated E22 trunc protein (1259+9 aa) can retain some degree of chloride conductance and provide benefit to patients with Cystic Fibrosis with 3′ terminal PTCs.

### Methods

Cell culture. Minimum Essential Medium (MEM), Fetal Bovine Serum (FBS), Penicillin/Streptomycin, LHC-8 basal medium, Bovine serum albumin 7.5%, and TrypLE Express were purchased from Gibco. 16HBE cells were grown at 37° C./5% CO2 in MEM supplemented with 10% FBS and 1% Penicillin/Streptomycin. Plates/flasks were coated by incubating a thin layer of coating solution [LHC-8 basal medium, 1.34 μl/ml Bovine serum albumin 7.5%, 10 μl/ml Bovine collagen solution, Type 1 (Advanced BioMatrix), 10 μl/ml Fibronectin from human plasma (Thermo Fisher Scientific)] at 37° C./5% CO2 for 2-3 h followed by thorough removal of coating solution and storage at 4° C. Cells were dissociated with TrypLE Express and centrifuged at 120×g for 5 min before seeding. Fisher Rat Thyroid (FRT) cells and primary hu-man bronchial epithelial cells were cultured as previously described.

Generation of gene edited cell lines. 16HBEge cells were cultured, edited, and cloned as previously described. The following targeting component of the crRNA guide RNAs were used to generate the exon 23 to 3′ UTR deletion clones: 5′-TGCTCAGTTATAGTATATAA-3′ (SEQ ID No: 94) and 5′-TTAGTTATCTGTTTAAACTA-3′ (SEQ ID No: 95). PCR amplification using primers spanning the genomic deletion [5′-gactcccctgtccttgttga-3′ and 5′-GATCCCACTCCTAGGTCCTTCGA-3′] were used to identify clones with the desired deletion and primers at the 3′ junction [5′-AGCCAGCACAGCCTCTTAGATGC-3′ and 5′-GATCCCACTCCTAGGTCCTTCGA-3′] were used to identify clones that were not homozygous for the desired deletion.

Free uptake ASO treatments in 16HBEs. Steric blocking ASOs were purchased from IDT with Phosphorothioate (PS) backbone and 2-O-Methoxyethyl (2′-MOE) modifications at each nucleotide. Lyophilized steric blocking ASOs were resuspended in PBS+Mg+Ca to 1 mM and stored in frozen aliquots to minimize the effects of freeze thaw cycles. 16HBEge and 16HBE14o- lines were seeded at 0.150×10\hat{ }6 cells/well in 24 well plates and grown at 37° C./5% CO2 in MEM supplemented with 10% FBS and 1% Penicil-lin/Streptomycin. After 24 hours, media was refreshed with addition of ASOs and RNA was harvested after 48 hours of treatment.

ddPCR Isoform Quantification. RNA was isolated from treated cells with the Aurum Total RNA Mini Kit. One Step reverse transcriptase and quantitative PCR was performed with the iTaq Universal Probes One Step Kit. All qPCR was run on the CFX Touch Deep Well real-time PCR detection system and analyzed with Bio-Rad CFX Maestro 1.0 software (version 4.0.2325.0418). ddPCR assays were composed of: cDNA or digested genomic DNA, 2×ddPCR Supermix for Probes (no dUTP) (1× final) (Bio-Rad), 40× as-say (1× final), and nuclease free water in 20 μl total reaction volume. cDNA synthesis was performed with the iScript Advanced cDNA Synthesis Kit for RT-qPCR (Bio-Rad). Droplets were generated with QX200 Droplet Digital PCR System (Bio-Rad), cycled in the CFX Touch Deep Well real-time PCR detec-tion system (Bio-Rad), and analyzed with the QX200 Droplet Digital PCR system Droplet Reader. (Bio-Rad). Data was analyzed with the Quantasoft software version 1.7.4. ddPCR probes designed to the junction of exon 25/26 were used as a proxy of full-length WT or W1282X CFTR mRNA and probes de-signed to exon 22/intron 22 junction including the first 140 bp of intron 22 were used as a proxy of ex-on 22 truncated CFTR mRNA.

Western blot analyses. Western blot analyses were performed as described previously, except the loading control, Na+/K+-ATPase, was detected with an antibody from Santa Cruz Biotechnology (sc-48345). 2.8.1.

Electrophysiology. Clonal lines were seeded at a density of 4.5×10⁵ cells cm-2 onto HTS Transwell 24-well filter inserts pre-coated with human collagen type IV. Cells were grown as submerged cultures in MEM (Gibco, 11095) containing 10% FBS and 1% Pen/Strep, and incubated at 37° C. and 5% CO2. Cells were treated from both the basolateral and apical side with fresh medium containing either control (vehicle) or test article for the treatment time indicated in the figure legends. After a total of 7 days, 16HBE cells typically formed electrically tight epithelia with a transepithelial resistance (Rt) of 200-600 Ω·cm2 and CFTR-mediated Cl— equivalent current (Ieq) was determined as described below.

Prior to functional (Ieq) studies, MEM was replaced with fresh HEPES-buffered (pH 7.4) solutions (assay buffer). A driving force for chloride ions was established through application of a basolateral to apical chloride ion gradient (see buffer composition below). Cell plates were mounted onto an automated robotic assay platform and equilibrated at ˜36° C. for 90 min. After equilibration, transepithelial voltage (Vt) and resistance (Rt) were monitored at ˜5 min intervals using a 24-channel transepithelial current clamp amplifier (TECC-24, EP Design, Bertem, Belgium). Electrode potential differences for each pair of Ag/AgCl voltage electrodes were also monitored at 5 min intervals by taking voltage measurements from a control plate with matching buffer solutions and 16HBE cells that were left untreated. Ieq was calculated from values of Vt and Rt using Ohm's law after correcting for series resistance and (electrode) voltage offsets unrelated to Vt. Ieq traces are plotted as mean±SD (n=3). The first 4 data points reflect baseline Ieq currents prior to sequential stimulation of CFTR with forskolin (10 μM) and VX-770/ivacaftor (1 μM). The last six data points were recorded in the presence of CFTR inhibitor CFTRinh-172 (20 μM). Agonists/antagonists were pre-diluted to 10-fold concentrations in assay buffer and add-ed to either the basolateral (forskolin) or apical (forskolin, VX-770/ivacaftor, and CFTRinh-172) side of the membrane (assay plates only). CFTR-mediated changes in Ieq, (i.e., delta forskolin, delta VX-770, delta CFTRinh-172, or the area under the curve (AUC) between forskolin and CFTRinh-172 addition) are used as a measure of functional CFTR surface expression or treatment-related functional rescue of mutant CFTR.

Assay buffer: CFTR-mediated transepithelial currents were recorded using a Cl— concentration gradient. The basolateral solution contained (mM): 137 NaCl, 4 KCl, 1.8 CaCl2, 1 MgCl2, 10 HEPES and D-Glucose, adjusted to pH 7.4 with NaOH/HCl ([CI-] total: 146.6 mM). The apical solution was matched to the basolateral except for (mM): 137 Na-gluconate replaced 137 NaCl ([Cl—] total: 9.6 mM).

### Example 1: Intron 22 Alternative Polyadenylation (ApA) Usage Results in a Naturally Occurring, Lowly Expressed CFTR Exon 22 Truncated CFTR mRNA Isoform (e22 Trunc mRNA) and Truncated Protein (e22 Trunc Protein) and is Insensitive to Nonsense Mediated Decay (NMD)

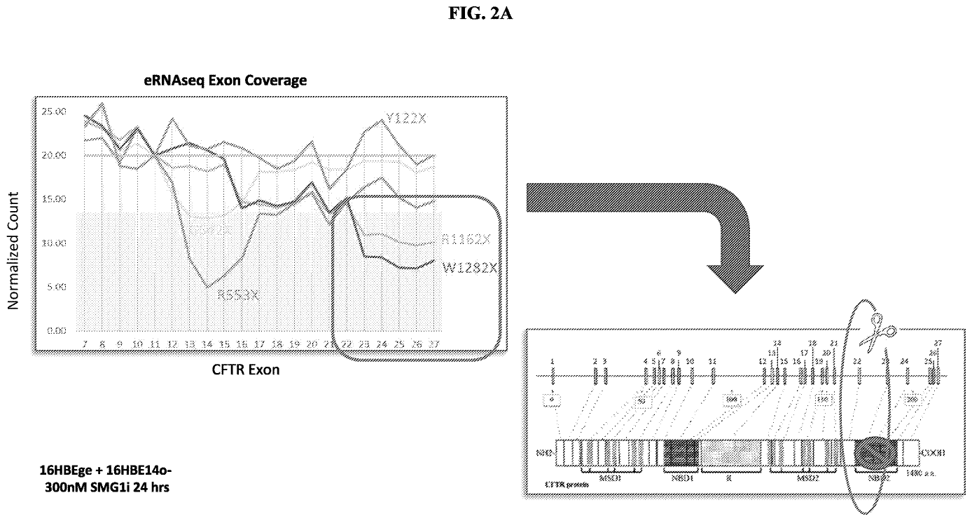

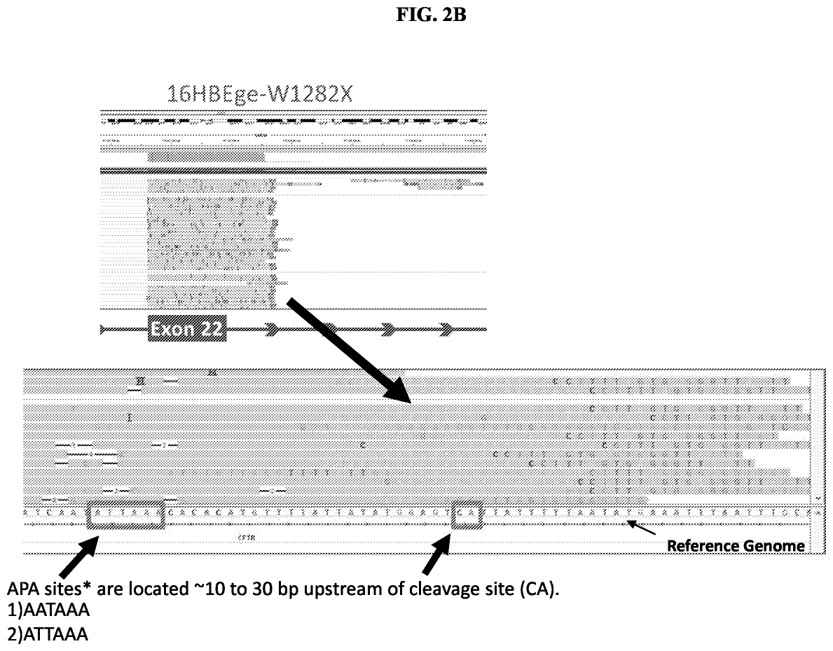

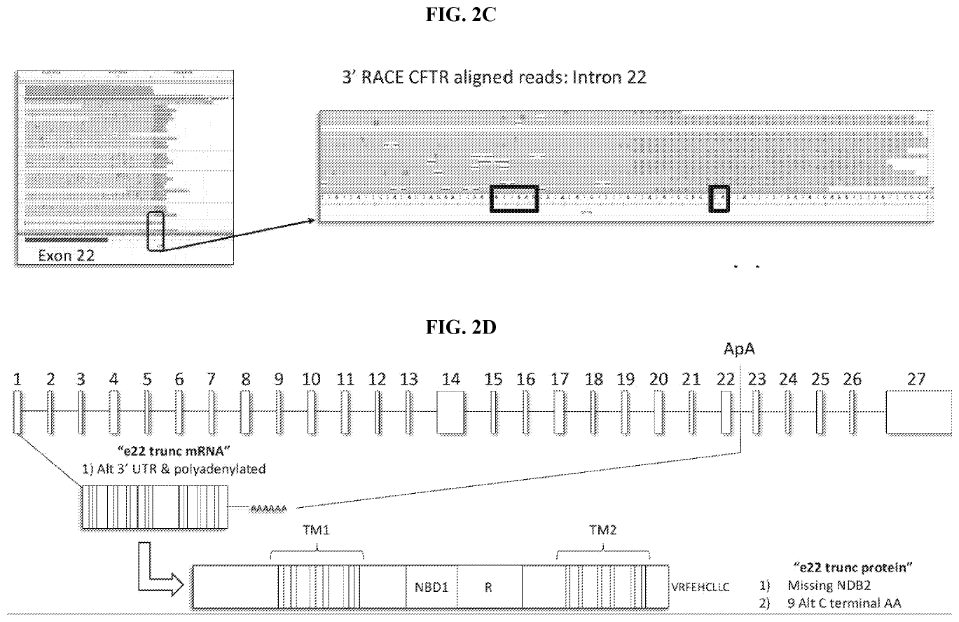

To achieve deep and uniform exon coverage across CFTR mRNA transcripts, enriched RNA sequencing (eRNAseq) was performed using multiple genetically modified 16HBE cell lines (human bronchial epithelial cell lines). As depicted in FIG. 2A, low sequence coverage of 3′-end exons of CFTR mRNA transcripts (e.g., Exons 23-27) was discovered in 16HBEge-W1282X and 16HBEge-R1162X cells, suggesting that W1282X and R1162X repertoires contain 3′-truncated CFTR mRNA transcripts. All sequence counts were normalized to wildtype 16HBE cells, and sequence coverage data for CFTR Exons 7-27 regions were shown in the figure. Sequencing results from 16HBEge-Y122X cell line showed similar coverage profile as wildtype cells as no splicing liability was expected. Both R553X and G542X showed a reduction of coverage in Exon 12, consistent with previous experiments.

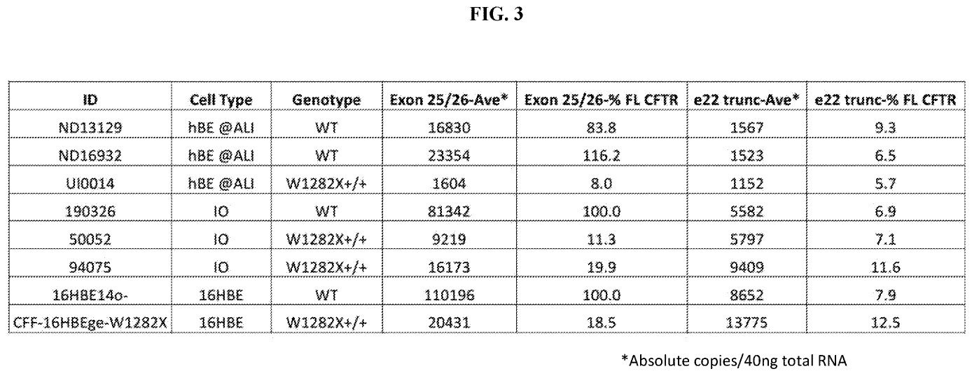

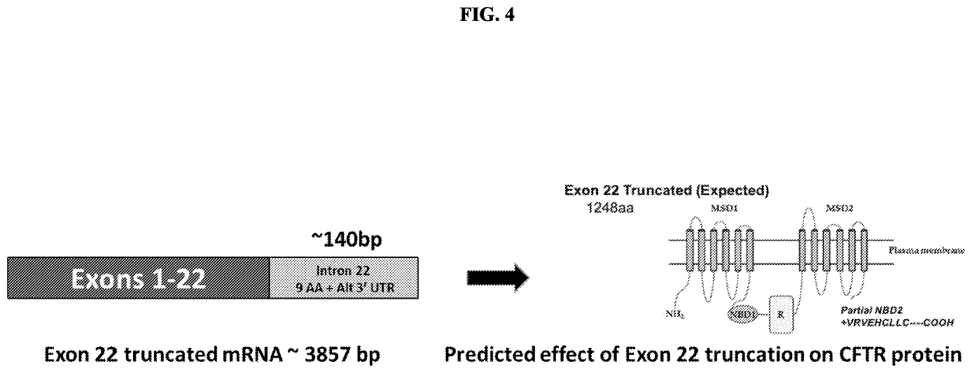

As depicted in FIG. 2B, further sequence analysis revealed that the 16HBEge-W1282X CFTR mRNA transcripts presented with normal 5′ ends but terminated within Intron 22 with extension of ˜140 bp into the intron on the 3′ end. Additionally, as shown in FIG. 3, the 16HBEge-W1282X transcripts terminated with a series of non-aligned adenosine nucleotides, indicating post transcriptional modification of the mRNA. Consistent with this finding, a consensus alternative polyadenylation (ApA) motif was identified within Intron 22 near the abovementioned site of polyadenylation, indicating ApA usage during processing of CFTR pre-mRNA into mature mRNA transcripts in the 16HBEge-W1282X and R1162X cells. Based on the sequence of the 3′-truncated mRNA with alternative polyadenylation discovered in the 16HBEge-W1282X and R1162X cells, it is predicted that a truncated CFTR protein of approximately 1239 amino acids can be translated, as depicted in FIG. 4. The truncated CFTR mRNA sequences were approximately 3857 base pairs (bp) long, including the first 22 exons and ˜140 bp of Intron 22 sequence. The first 27 bp of the intronic 22 sequence encodes 9 amino acids in-frame, followed by a stop codon and an alternative 3′ untranslated region (UTR). Thus, truncated CFTR protein resulted from the Exon 22 truncated mRNA-ApA can contain the first 1239 amino acids of a WT CFTR and additional few (˜9) amino acids encoded by the intronic sequence within Intron 22.

A novel exon 22 truncated mRNA isoform was detected in 16HBE14o- cells using 3′ RACE with a 5′ primer targeting exon 8 coupled with long read sequencing that contained exons 8-22. Exon 22 truncated CFTR transcripts contain 10-30 post-transcriptionally added adenosine residues that align with putative consensus hexanucleotide alternative polyadenylation and dinucleotide cleavage sites indicating usage of intron 22 alternative polyadenylation site (FIG. 2C, top). Exon 22 truncated isoforms were characterized using a CFTR specific cDNA long read isoform sequencing with 5′ UTR and 3′ intron 22 targeting primers and identified a novel exon 22 truncated CFTR transcript containing all 22 exons. These alternatively polyadenylated CFTR mRNAs isoforms that extend into intron 22 are referred to as “e22 trunc” in the description below. E22 trunc mRNAs contain the first 22 CFTR exons followed by ˜140 bp of intron 22 which encode 9 alternative amino acid residues and alternative 3′ UTR (FIG. 2C, bottom). Translation of E22 trunc mRNA results in a truncated CFTR protein that retains transmembrane domains 1 and 2, nucleotide binding domain 1 (NBD1), and R domain, but lacks a complete NBD2 domain and contains 9 alternative intron 22 amino acid residues (VRFEHCLLC) followed by a stop codon.

To test whether E22 trunc mRNA is NMD insensitive, E22 trunc mRNA expression levels were assayed in wild type and the CF relevant W1282X genotype in airway and intestinal tissues. Absolute copies of E22 trunc and full-length mRNA were assessed using RT-ddPCR and quantitative fractions relative to WT full length CFTR were calculated in 16HBE14o- (7.9%), WT IO (6.9%) and WT HBE from 2 donors at ALI (9.3% and 6.5%) (FIG. 3) indicating constitutive low-level E22 trunc mRNA expression. As predicted, E22 trunc mRNA expression was detected in approximately the same absolute levels and low frequency (5.7-12.5% WT full length CFTR) in W1282X airway and intestinal cells as wild type suggesting it is NMD insensitive.

### Example 2: Stable Exon 22 Truncated CFTR mRNA Transcript Levels were Elevated in the 16HBEge-W1282X and R1162X Cells

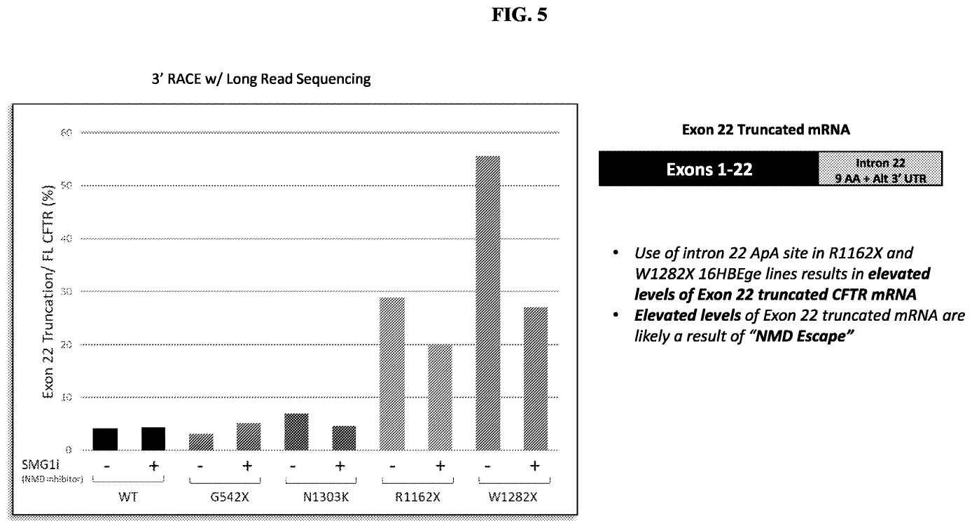

The levels of Exon 22 truncated CFTR mRNA transcripts were elevated at steady state in the 16HBEge-W1282X and R1162X cells, presumably due to non-sense-mediated decay (NMD) escape. To test this hypothesis, the levels of Exon 22 truncated CFTR mRNA transcripts were measured in multiple 16HBE-derived cell lines in the presence of an NMD inhibitor (SMG-1 inhibitor) via 3′ RACE long read sequencing. All sequencing results were normalized to full length CFTR transcripts (“FL CFTR”), and fractions of Exon 22 truncated CFTR mRNA transcripts were compared. As depicted in FIG. 5, higher fractions of Exon 22 truncated CFTR mRNA transcripts over FL CFTR mRNA transcripts were observed in 16HBEge-W1282X and R1162X cells, as compared to that in wildtype (WT) cells, and the addition of SMG-1 inhibitor significantly reduced the fraction in 16HBEge-W1282X and R1162X cells. As expected, low levels of Exon 22 truncated CFTR mRNA transcripts were observed in 16HBEge-G542X and N1303K cells, comparable to WT cells.

These data suggest that NMD escape can contribute to the increased fractions of Exon 22 truncated/FL CFTR mRNA transcripts observed in W1282X and R1162X cells. The presence of premature stop codon (PTC) in a mRNA transcript can usually trigger the degradation of the transcript via NMD. The FL CFTR transcripts produced in both 16HBEge-W1282X and R1162X cells contain premature termination codons in Exon 23 and Exon 22, respectively, and can be degraded through NMD. Upon NMD blockade by the SMG-1 inhibitor, the levels of FL CFTR transcripts were elevated significantly, resulting in reduced fractions of the truncated mRNA Exon 22 transcripts. This observation suggested that the Exon 22 truncated CFTR mRNA transcripts were not as affected by NMD inhibition, indicating NMD escape during the processing of Exon 22 truncated CFTR mRNA transcripts.

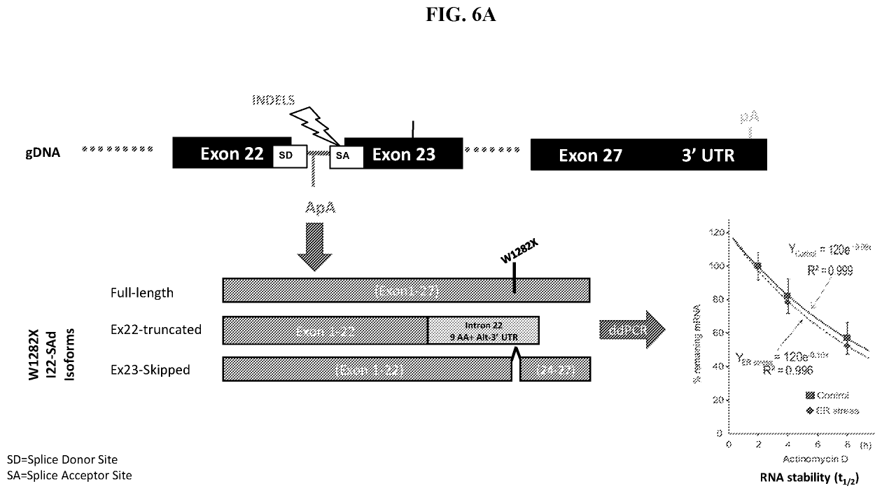

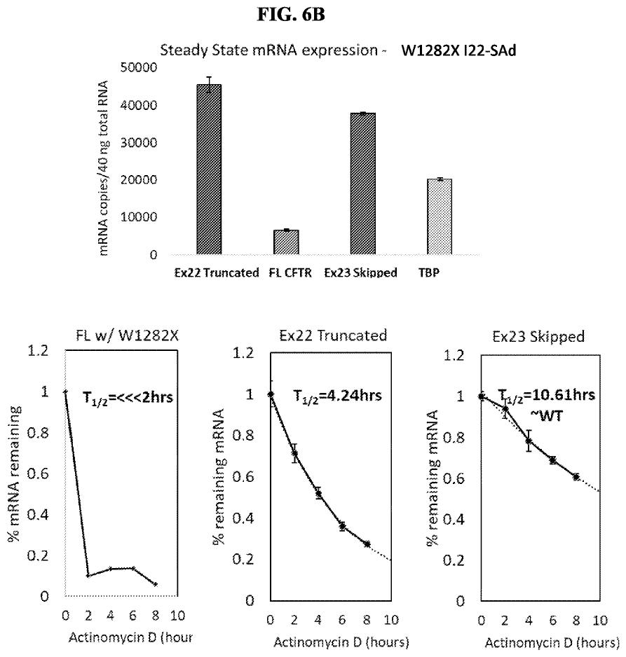

The stability of Exon 22 truncated CFTR mRNA (e.g., E22 trunc mRNA) was quantified. For this purpose, Actinomycin D time courses were employed to block transcription in 16HBE14o- and CFF-16HBEge-W1282X cells and measured % remaining CFTR isoforms (WT FL CFTR, W1282X FL CFTR, and E22 trunc mRNAs) using ddPCR at t0, t2, t4, t6, t8, t10 hours (FIGS. 6A-6B). To measure the half-lives of the Exon 22 truncated CFTR mRNA transcripts, W1282X-I22-SAd cell lines were generated from the 16HBEge-W1282X line using cloning techniques, as depicted in FIG. 6A. Briefly, the splice acceptor site of Intron 22 of 16HBEge-W1282X cells was disrupted in the W1282X-I22-SAd cells. The resulting W1282X-122-SAd cells produced Exon 22 truncated CFTR mRNA transcripts, full-length CFTR mRNA containing the PTC (“FL-W1282X transcript”), and Exon 23 skipped CFTR mRNA. Exon 22 truncated CFTR mRNA transcripts were found to be present in W1282X-I22-SAd cells at a level over four times higher than those in the W1282X parental line. W1282X-I22-SAd cells were treated with Actinomycin D to inhibit transcription, and RNA was harvested at various time points shown in FIG. 6A. Droplet Digital PCR (ddPCR) assays for exon 25/26 junction and Exon22/Intron 22 junction were used to assess full-length and Exon 22 truncated CFTR mRNA, respectively. The amount of each CFTR mRNA species assessed at the first time point (t=0) was set to 1 (100%), and the percentage of the remaining mRNA was measured by ddPCR. The mRNA half-life was calculated from the exponential decay. As shown in FIG. 6B, the t_{1/2} of Exon 22 truncated CFTR mRNA was estimated to be about 4.24 hours, significantly longer than that of the FL-W1282X transcript (<<<2 hours). Exon 23 skipped mRNA transcript exhibited a longer half-life (t_{1/2}=10.61 hours), equivalent to the wildtype CFTR transcript, as depicted in FIG. 6B.

Steady state mRNA expression of Exon 22 truncated CFTR mRNA remained the highest in 16HBEge-W1282X-I22 SAd (SAd=Splice Acceptor disrupted) cells, despite of their relatively shorter t_{1/2} as compared to that of the Exon 23 skipped CFTR transcripts. This observation might be due to a faster rate of generation or mRNA processing of the mature Exon 22 truncated CFTR mRNA compared to that of the mature Exon 23 skipped CFTR transcripts, considering that these two transcripts originated from the same nascent mRNA. Consistent with this finding, it was also observed that the splice donor site of the CFTR Intron 22 was relatively weak, which can contribute to slower processing of the Exon 23 skipped CFTR transcript.

### Example 3: Exon 22 Truncated CFTR Proteins were Responsive to VX-661/445/770 (Trikafta) Potentiation/Correction

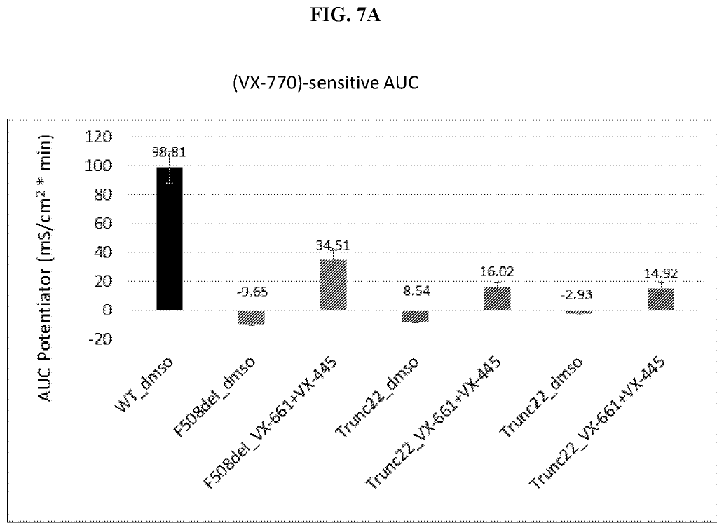

Using the TECC-24 assay, functions of CFTR variants were measured. Briefly, cDNA of wildtype, Exon 22 truncated, and F508del CFTR proteins were expressed in Fischer Rat Thyroid (FRT) cells. Cells expressing Exon 22 truncated CFTR proteins and F508del CFTR proteins were incubated with DMSO or VX661/445 prior to the assay. As depicted in FIG. 7A, as compared with the untreated group, F508del CFTR function was enhanced in vitro by addition of VX770, as indicated by an increase of area under curve (AUC) of CFTR-mediated chloride current. Similarly, Exon 22 truncated CFTR function was also enhanced by Trikafta. It was observed that Trikafta recovered F508del CFTR function to about 35% of that of wildtype and enhanced Exon 22 truncated CFTR function to about 15% of that of wildtype. When treated with Orkambi, a lumacaftor/ivacaftor combination therapy approved by the FDA, F508del CFTR function was restored to ˜68% of that of wildtype.

E22 trunc cDNA was overexpressed in Fischer Rat Thyroid (FRT) cells and subjected it to Ussing chamber analysis 96 hours post transfection (+/−) 48-hour pre-assay treatment with VX445/VX661 (3/3.5 μM) and in assay acute additions of Forskolin (10 μM) and VX-770 (1 μM). As depicted in FIG. 7B, no detectable change in CFTR Cl⁻ current was observed with E22 trunc transfection and DMSO (0.002%) vehicle treatment after addition of VX-770 (1 μM). Conversely, 48 hr pretreatment with VX-445/VX-661 (3/3.5 μM) and acute VX-770 (1 μM) resulted in an increase of 0.42 AUC/min Gt (mS/cm2) CFTR Cl— current that was reduced to pre-VX-770 levels with the addition of the CFTR inhibitors CFTRinh-172 (20 μM)+GlyH-101 (20 μM). These data demonstrate that E22 trunc protein possesses partial CFTR function when treated with modulators.

### Example 4: Deletion of CFTR Exons 23-27 Promoted Intron 22 Alternative Polyadenylation Usage

Δ23-27 (or Del23-27) gene edited model was established using CRISPR gene editing technology as depicted in FIG. 8. This ˜27 kb genomic deletion leaves all 11 putative consensus hexanucleotide alternative polyadenylation and dinucleotide cleavage sites in the remaining 13.5 kb 5′ end of intron 22. Briefly, using a 5′ guide ˜13.5 kilo base pairs (kb) into the Intron 22 and a 3′ guide ˜159 bp immediately downstream of the 3′ UTR of the CFTR gene, regions spanning from 5′ portion of Intron 22 to post-3′UTR were deleted from the genome of 16HBE14o- cells. The resulting cell genome contains Exons 1-22 followed by ˜13.5 kb of Intron 22, and then intergenic region upstream of (TTNBP2, as depicted in FIG. 8. Three exemplary Δ23-27 gene edited model cell lines were generated: 2-H07, 3-B09, and 3-D01.

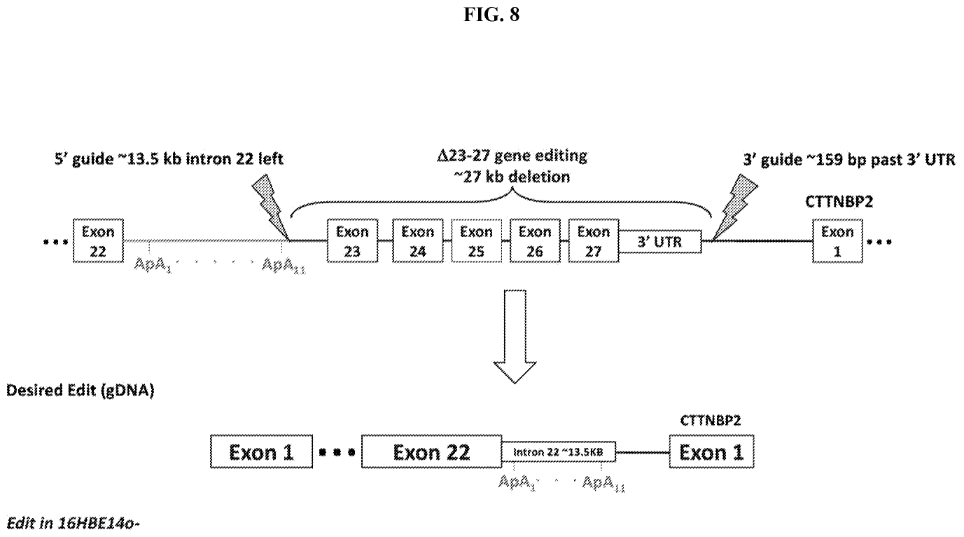

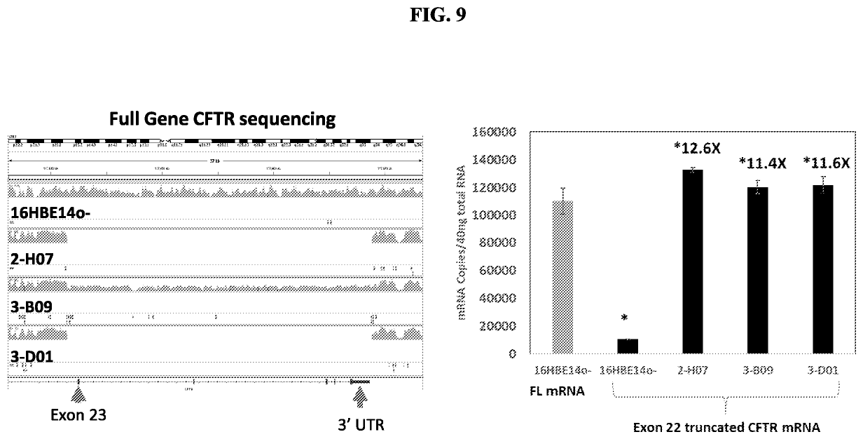

Exon 22 truncated CFTR mRNA transcripts (black bars) from 3 Del23-27 clonal lines (2-H07, 3-B09, 3-D01) and 16HBE14o- cells were assayed using droplet digital PCR (ddPCR) as depicted in FIG. 9. Full length CFTR transcript was measured using ddPCR from 16HBE14o- cells (grey bar). Fold change of Exon 22 truncated CFTR mRNA was calculated for each clone as compared to Exon 22 truncated CFTR mRNA level in 16HBE14o- cells. As depicted in FIG. 9, all three Del23-27 cell lines contained significantly higher levels of Exon 22 truncated CFTR mRNA compared to the parental 16HBE14o- line, about 12 times the amount of that in parental cells. The levels of Exon 22 truncated CFTR mRNA in all three Del23-27 cells were also higher than the level of full-length WT (TR transcript in parental 16HBE14o- cells. Absolute copies of E22 trunc mRNA from the D23-27 clonal lines; 2-H07 (132894 copies/40 ng total RNA+/−1775), 3-B09 (120237 copies/40 ng total RNA+/−4839), and 3-D01 (121891 copies/40 ng total RNA+/−6122) were 12.6×, 11.4×, and 11.6× higher relative to parental levels (10505 copies/40 ng total RNA+/−172) and equivalent to parental full length CFTR mRNA levels (FIG. 3B) suggesting almost complete induction of intron 22 ApA usage.

### Example 5: Del23-27 Cells Expressed Truncated CFTR Proteins, Whose Functions were Significantly Enhanced by Potentiators/Correctors

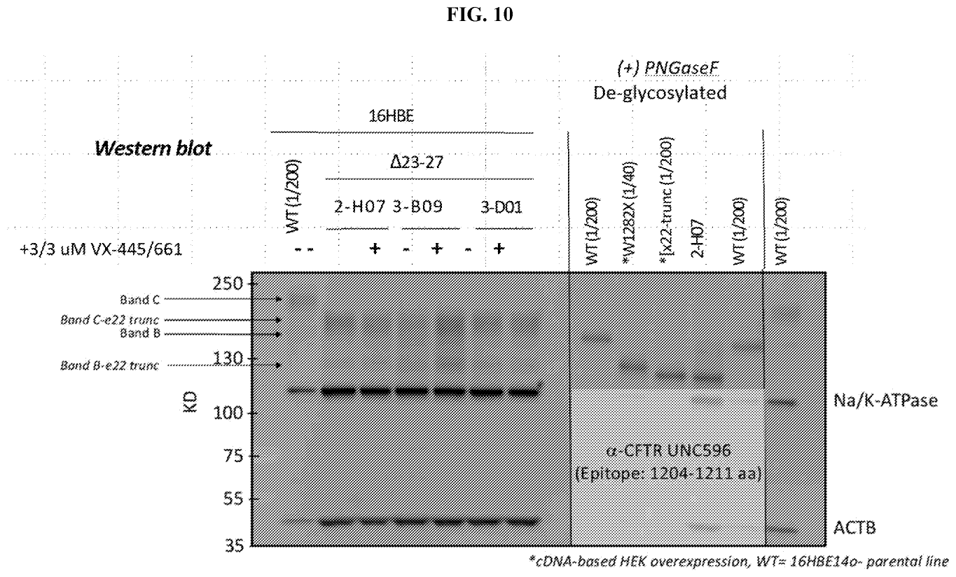

Next, Western blots were used to characterize the effect of forcing intron 22 ApA usage on CFTR protein expression. Three D23-27 clonal lines 2-H07, 3-B09, and 3-D01 and WT 16HBE14o- parental lines were treated with/without 3/3 μM VX-445/VX-661 for 48 hrs. Control lysates from WT (diluted 1:2) and HEK293 E22 trunc cDNA overexpression (diluted 1:100) were run for comparison to full length and truncated Band B and Band C protein respectively. Blots were probed with α-CFTR UNC596 (epitope: 1204-1211 aa; Ex22) to detect both FL and truncated CFTR protein. 2-H07, 3-B09, and 3-D01 lysates resulted in bands at 150 kDa and 110 kDa that align with HEK293 overexpressed E22 trunc Band B and C controls indicating expression of E22 trunc protein. Furthermore, 48 hr treatment with VX-445/VX-661 (3/3 μM) resulted in ˜2× increase in truncated band B and C indicating improved trafficking of the E22 trunc protein with modulators. As depicted in FIG. 10, truncated forms of the CFTR proteins were detected from Del23-27 clonal lines (2-H07, 3-B09, 3-D01) using α-CFTR UNC596 antibody (raised against epitope: 1204-1211 aa in Ex22). Reactivity against Exon 22 was picked up in 2 bands (Band C and B in FIG. 10) in all Del23-27 cells and aligned with bands in the positive control HEK293 overexpressing the Exon 22 truncated CFTR cDNA. Na/K-ATPase were blotted as a loading control. VX-445/VX-661 (3/3 μM) were used to treat the cells, which did not significantly affect the level of truncated CFTR protein as indicated by Band C and B. 16HBE14o- WT were included for comparison as depicted in FIG. 10.

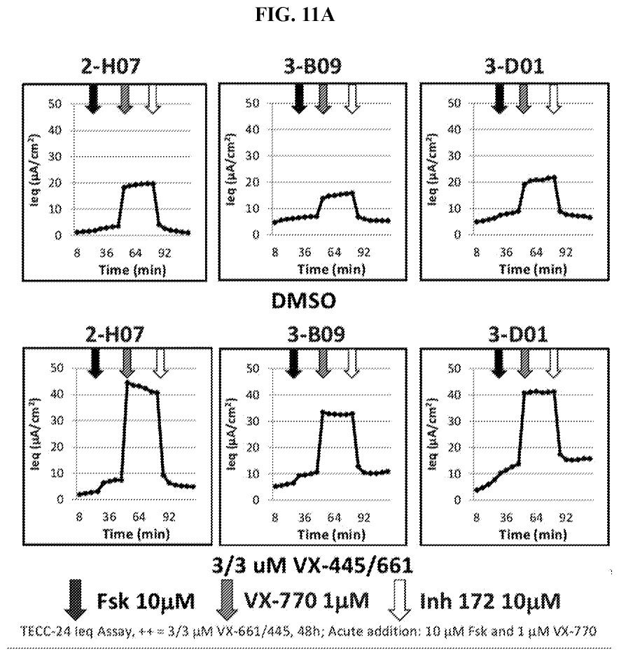

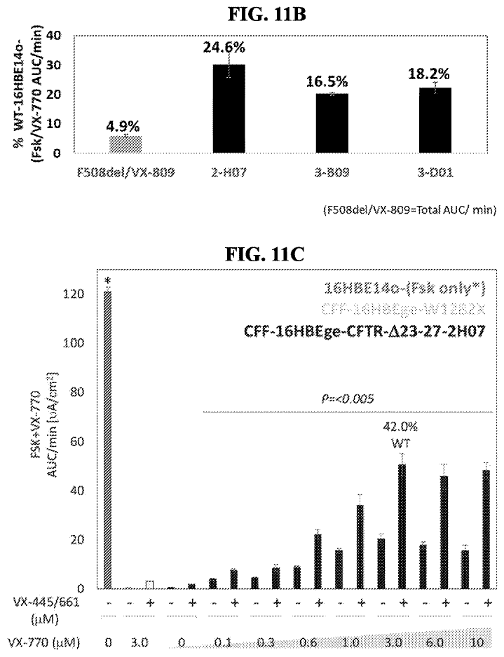

To assess the functional consequence of forcing intron 22 ApA usage, D23-27 clonal lines 2-H07, 3-B09, 3-D01 were subjected to 48 hours treatment of vehicle control (0.002% DMSO) or VX-445/VX-661 (3/3 μM), and acute in assay addition of VX-770 (1 μM) and CFTR function was assessed using the transepithelial current clamp (TECC) conductance assay. Potentiators/correctors Trikafta restored the function of the Del23-27 CFTR protein to a significant fraction of WT function, as shown in FIG. 11A. Briefly, transepithelial chloride conductance assay (TECC-24 assay) was conducted in WT 16HBE14o- (treated with vehicle), 16HBEge-F508del (treated with VX-809), and Del23-27 clones (treated with VX-445/661), using sequential treatment of forskolin, VX-770, and CFTR inhibitor 172. Representative traces from TECC-24 assay are shown in FIG. 11A. In the presence of VX-445/VX-661/VX-770 (Trikafta), Del23-27 CFTR protein function was restored, shown as chloride conductance induced by VX-770 and inhibited by to CFTR (inh)-172. CFTR function can be assayed by comparing the induced chloride conductance area under curve (AUC) of the cell lines utilized. Unlike the E22 trunc cDNA FRT overexpression results, VX-770 treatment alone resulted in an increase in Cl⁻ current. Vehicle treatment of 2-H07, 3-B09, and 3-D01 cells resulted in 11.4+/−2.1 FSK+VX-770 AUC/min [μA/cm²], 6.5+/−0.9 FSK+VX-770 AUC/min [μA/cm2], and 8.3 +/−1.3 FSK+VX-770 AUC/min [μA/cm2] respectively. 48-hour treatment with 3/3 μM VX-445/VX-661 increased Cl— current 27.8+/−3.9 FSK+VX-770 AUC/min [μA/cm2] (2.4×), 19.2 +/−0.5 FSK+VX-770 AUC/min [μA/cm²] (3.0×), and 21.2+/−1.1 FSK+VX-770 AUC/min [μA/cm²] (2.6×), in 2-H07, 3-B09, and 3-D01 cells relative to vehicle treatment respectively.

The AUC of various mutant cell lines (e.g., 16HBEge-W1282X cells), with or without treatment, can be normalized to the AUC obtained from cells expressing WT CFTR protein and expressed as either a percentage of WT CFTR (% WT CFTR) or a ratio to WT CFTR (variant/WT CFTR). As shown in FIG. 11B, CFTR function in the three Del23-27 cell lines 2-H07, 3-B09, and 3-D01 were restored to ˜25%, ˜17% and ˜18% of WT function, respectively, much higher than that of F508del potentiated by VX-809/VX-770 (˜5% of WT).

A dose escalation study was performed with VX-770 (0.1-10 μM) in the 2-H07 clonal line, as shown in FIG. 11C. All concentrations tested resulted in significantly increased CFTR chloride conductance relative to vehicle control (+/−) VX-445/VX-661 (3/3 μM) (p=<0.005, ANOVA). VX-770 treatment resulted a dose dependent increase in Cl⁻ current from 0.1 to 3 μM (+/−) VX-445/VX-661 (3/3 μM) and a maximum CFTR Cl— current of 50.6 FSK+VX-770 AUC/min [μA/cm²] was achieved at 3.0 μM (+) VX-445/VX-661/VX-770 (3/3/3 μM). This level of Cl— current equates to ˜42% WT 16HBE14o- function (FSK only). Example 6: Modulation of CFTR mRNA processing by Intron 22-targeting ASOs in 16HBE14o- WT cells.

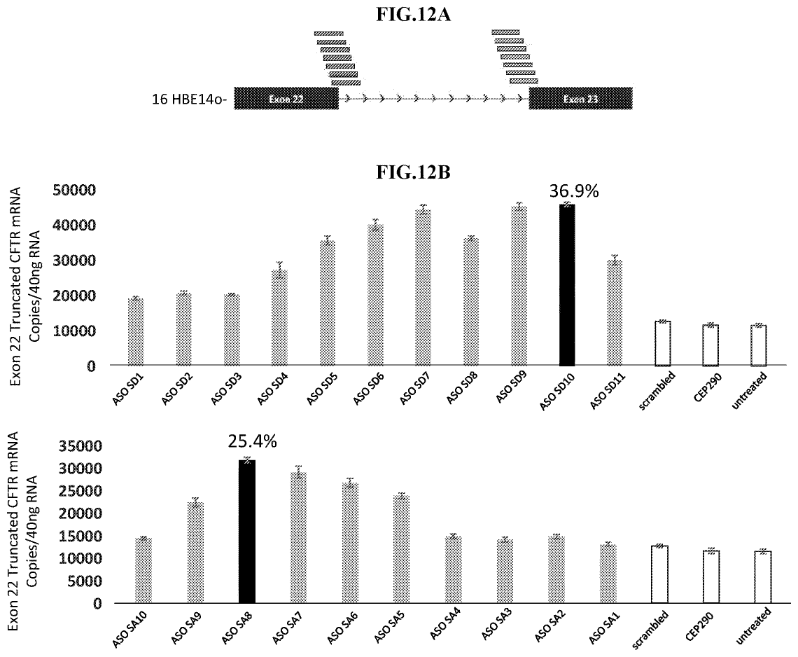

Steric blocking ASOs were designed via a 10 “step” 1-nucleotide “walk” tiled scheme to target Intron 22 donor site (black) or acceptor site (gray bars), as depicted in FIG. 12 A.

16HBE14o- WT were treated with exemplary ASOs according to some embodiments of the present disclosure, and Exon 22 truncated CFTR mRNA was assayed using ddPCR. As shown in FIG. 12B, most candidate ASOs increased Exon 22 truncated CFTR mRNA level in WT 16HBE14o- cells, among which exemplary ASOs, SD10 (black bar upper) induced Exon 22 truncated (′F TR mRNA to a level that is ˜37% of FL CFTR level in WT cells, and SA8 (black bar lower) induced Exon 22 truncated CFTR mRNA to ˜25% of FL CFTR in WT cells. Scrambled ASOs, off-target ASO (ASO targeting CEP290 mRNA), and untreated cells were included in the experiment as controls.

### Example 7: Modulation of CFTR mRNA Processing and Restoration of CFTR Channel Function by Intron 22-Targeting ASOs in 16HBEge-W1282X Cells

16HBEge-W1282X cells were treated with exemplary ASOs according to some embodiments of the present disclosure. In some cases, the exemplary ASOs were administered at various doses alone, in combination with other ASOs at various doses, or co-administered with Trikafta, and a plurality of assays were used to assess the effects of the ASO treatments in 16HBEge-W1282X cells.

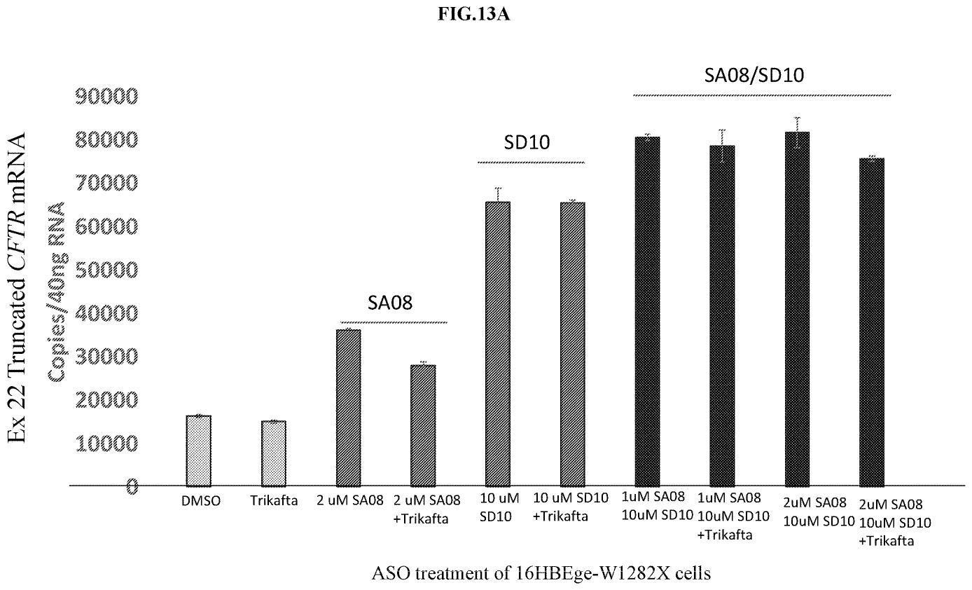

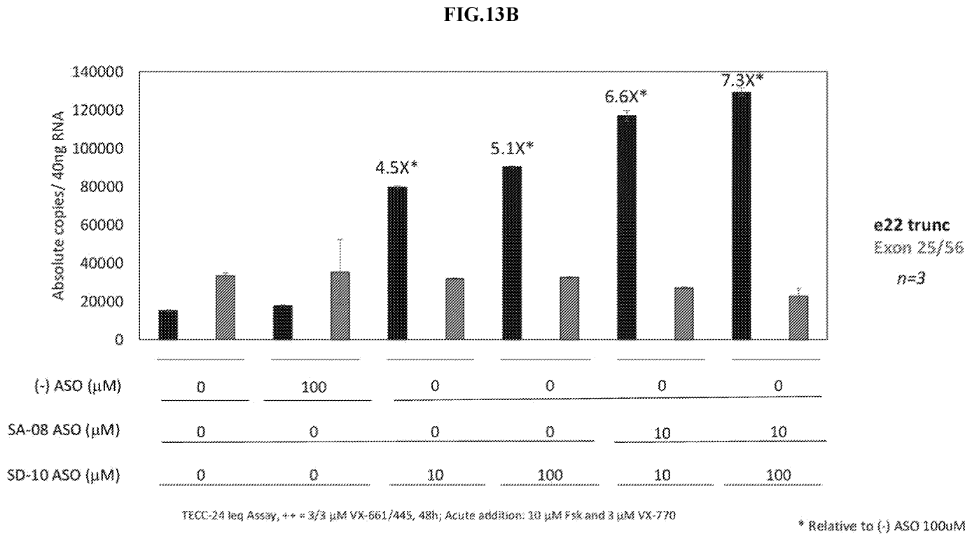

Exon 22 truncated CFTR mRNA was assayed using ddPCR, as depicted in FIGS. 13A-13B. 16HBEge-W1282X cells were treated with SA08 alone, SD10 alone, or SA08 in combination with SD10 at various doses, in the presence of DMSO vehicle control or Trikafta. As depicted in FIG. 13A, Exon 22 truncated CFTR mRNA was increased in all groups treated with exemplary ASO(s) compared to no ASO treatment. Separate treatment of SA08 alone and SD10 alone enhanced the expression of Exon 22 truncated CFTR mRNA by about 100% and by over 200%, from less than 20000 copies per 40 ng RNA to about 30000 copies and about 65000 copies per 40 ng RNA, respectively. The combination of SA08 and SD10 further enhanced the expression of Exon 22 truncated CFTR mRNA to about 80000 copies per 40 ng RNA. In all groups in this experiment, Trikafta treatment did not significantly alter the expression of Exon 22 truncated CFTR mRNA. Full length CFTR W1282X mRNA levels were unchanged in SD-10 & SA-08 or combination treatment compared to untreated or (−) ASO (100 μM) control. (−) ASO (100 μM) E22 trunc mRNA levels were also unchanged compared to untreated cells. As shown in FIG. 13B, SD-10 and SA-08 ASO treatments did significantly increase E22 trunc mRNA levels. SD-10 & SA-08 or combination treatments induced a 4.5×, 5.1×, 6.6×, and 7.3× fold change increase in E22 trunc mRNA levels relative to (−) ASO (100 μM) with SD-10 (10 μM), SD-10 (100 μM), SD-10 (10 μM) & SA-08 (10 μM), and SD-10 (100 μM) & SA-08 (10 μM) respectively.

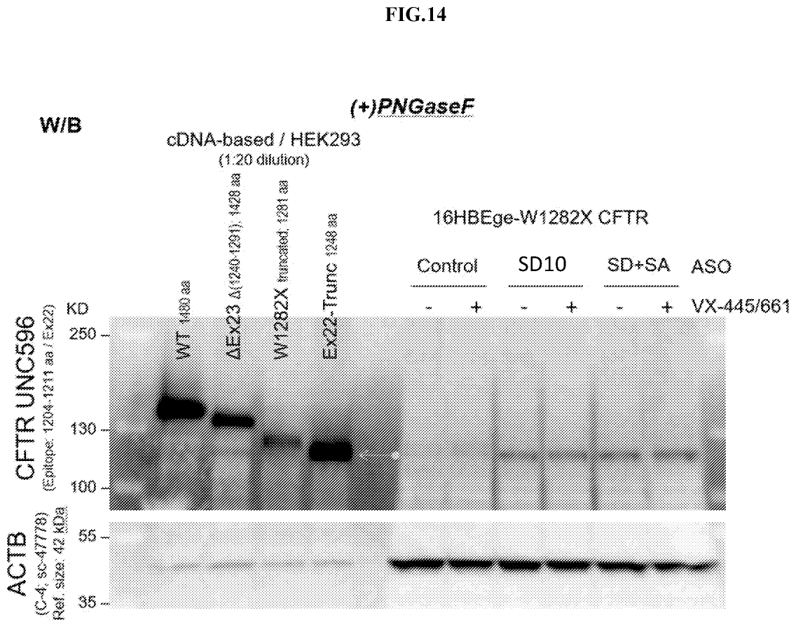

Exon 22 truncated CFTR protein expression was assayed using Western blotting, as depicted in FIG. 14. 16HBEge-W1282X cells were treated with exemplary ASO(s) alone or in combination (e.g., SD10; SD10 and SA08), in the presence of drug vehicle or correctors VX-445/661. Prior to electrophoresis and Western blotting, 16HBEge-W1282X cell extracts were treated with PNGase F to remove glycosylation. cDNA of four CFTR variants were transiently transfected in HEK293 cells, including WT CFTR (1480 aa), DelEx23 CFTR (1428 aa), W1282X CFTR (1281 aa), and Ex22 truncated CFTR (1248 aa). HEK293 cell extracts from these transfections were also treated with PNGase F and used as controls for Western blot. As depicted in FIG. 14, the α-CFTR UNC596 antibody (raised against epitope: 1204-1211 aa in Ex22) recognizes the deglycosylated Exon 22 truncated CFTR protein as one band at about 130 kDa. Western blotting results show that expression of Exon 22 truncated CFTR protein was increased by both treatment with SD10 alone and treatment with SD10 and SA08 combination, while treatment with correctors VX-445/VX-661 did not significantly affect the expression of Exon 22 truncated CFTR protein. Actin (ACTB) was blotted as a loading control, which showed consistent loading of proteins within each group.

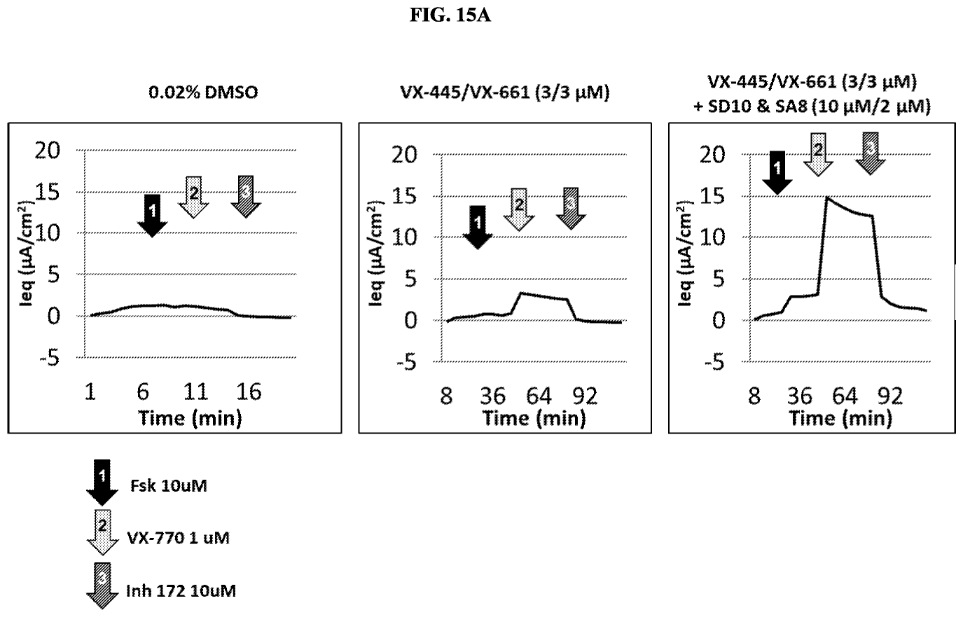

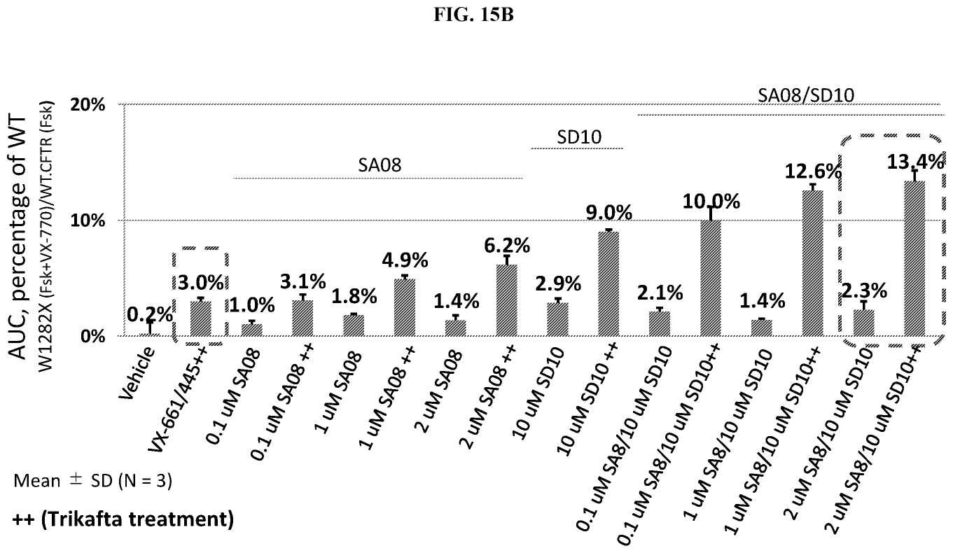

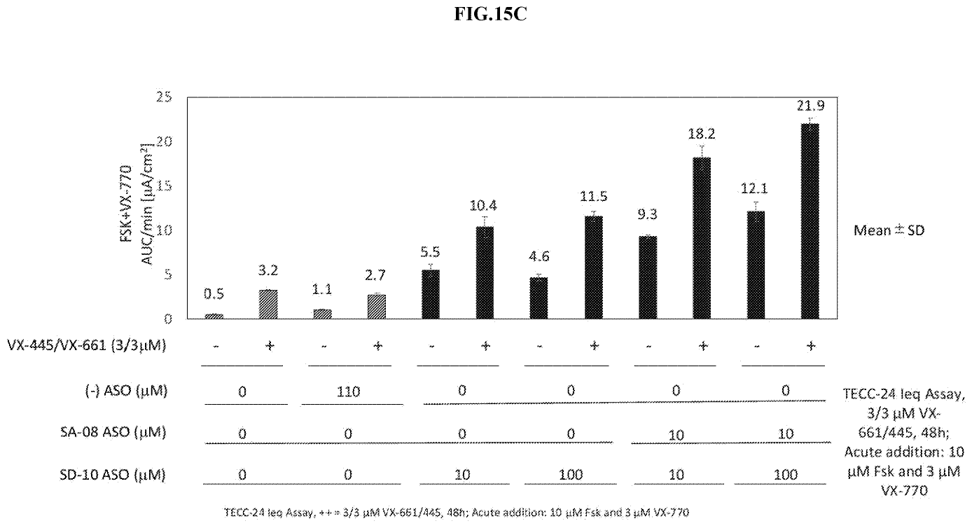

The function of Exon 22 truncated CFTR protein was assayed by the transepithelial chloride conductance assay (TECC-24 assay), as depicted in FIG. 15A and FIG. 15B. 16HBEge-W1282X cells were incubated in 0.02% DMSO (vehicle) or VX445/VX661 prior to the TECC assay. Characteristic CFTR responses to forskolin, potentiator (VX-770), and CFTR inhibition (Inh-172) were recorded in the presence and absence of various doses and combinations of ASO treatments. Representative traces from the TECC-24 assay are shown in FIG. 15A. As shown in the figure, chloride channel conductance was increased by Trikafta treatment, suggesting that Trikafta treatment restored CFTR trafficking to the cell membrane, and induced a chloride channel conductance of the Exon 22 truncated CFTR. ASO treatment further induced a larger chloride channel conductance compared to no ASO treatment.

The function of various CFTR proteins in the cells can be assessed by comparing the area under curve (AUC) of the induced chloride conductance obtained in the electrophysiology assays. The AUC of various mutant cell lines (e.g., 16HBEge-W1282X cells), with or without treatment, can be normalized to the AUC obtained from cells expressing WT CFTR protein and expressed as either a percentage of WT CFTR (% WT CFTR) or a ratio to WT CFTR (variant/WT CFTR). FIG. 15B depicts enhanced chloride conductance AUC of 16HBEge-W1282X cells normalized to WT CFTR (percent of WT CFTR AUC) after treatment with exemplary ASO(s), with or without Trikafta. Briefly, 48 hours prior to the assay, 16HBEge-W1282X cells were treated with either drug vehicle or 3/3 μM VX-661/445. Also administered to the cells at this time were various dosing regiments of ASO(s), including SA08 at 0.1 μM, 1 μM, 2 μM, SD10 at 10 μM, and combinations of SA08/SD10. After the 48-hour incubation, 16HBEge-W1282X cells were administered acute sequential treatment of 10 μM forskolin with, 3 μM VX-770, and 10 μM Inh 172. The combination treatment with VX-661/445 and VX-770 is designated as Trikafta (++) in FIG. 15B. In the control group, 16HBE14o- WT cells were treated with 10 μM forskolin without any other drugs or ASO(s). Trikafta treatment can restore Ex22 truncated CFTR function to ˜3% of WT CFTR function. All ASO treatments increased Ex22 truncated CFTR function to from ˜1% to ˜2.9% of WT, in a dose-dependent fashion. Treating the cells with ASO and Trikafta further enhanced the function of Ex22 truncated CFTR from ˜3.1% to ˜13.4% of WT. In the Trikafta treatment group, 0.1 μM SA08 enhanced the chloride conductance AUC of 16HBEge-W1282X cells to about 3.1% of WT. With increasing doses of SA08, the effect of the ASO increased. 1 μM SA08 enhanced the AUC to ˜4.9% of WT and 2 μM SA08 enhanced the AUC to ˜6.2% of WT. Also, with Trikafta treatment, 10 μM of SD10 enhanced the chloride conductance AUC of 16HBEge-W1282X cells to about 9% of WT, compared to the 2.9% increase in AUC without Trikafta treatment. In presence of Trikafta, the use of two ASOs enhanced the Ex22 truncated CFTR function more than either ASO alone. 0.1 μM SA8/10 μM SD10 ASO treatment with Trikafta increased the chloride conductance AUC of 16HBEge-W1282X cells to about 10% of WT. Similarly, 1 μM SA8/10 μM SD10 ASO treatment with Trikafta increased the AUC to about 12.6% of WT, and 2 μM SA8/10 μM SD10 ASO treatment with Trikafta increased the AUC to about 13.4% of WT.

The TECC-24 assay (n=3) was used to quantify the functional rescue of CFTR chloride current via ASO treatment in CFF-16HBEge-W1282X, as shown in FIG. 15C. Untreated vehicle produced a marginally detectable CFTR Cl— current of 0.5+/−0.09 FSK+VX-770 AUC/min (μA/cm²) that increased to 3.2+/−0.06 FSK+VX-770 AUC/min (μA/cm²) with VX-445/VX-661 (3/3 μM). These data are consistent with previously assayed functional response of CFF-16HBEge-W1282X to VX-445/VX-661/VX-770 (3/3/1 μM). Treatment with (−) ASO (110 μM) was approximately equivalent to vehicle alone (+/−) VX-445/VX-661 (3/3 μM), 2.7+/−0.2 and 2.2+/−0.1 FSK+VX-770 AUC/min (μA/cm²) respectively. All ASO treatments resulted in statistically significant increases in CFTR CI-current compared to (−) ASO control with vehicle and VX-661/445, respectively (p<0.05, ANOVA with Tukey post-hoc test). In all cases, functional response to SD-10, SA-08, or combination ASO treatments were increased with VX-445/VX-661 (3/3 μM) relative to ASO alone. SD-10 (μM), SD-10 (100 μM), SD-10 (10 μM) & SA-08 (10 μM), and SD-10 (100 μM) & SA-08 (10 μM) were 5.5+/−0.7, 4.6+/−0.4, 9.3 +/−0.2, and 12.1+/−1.1 (−) VX-445/VX-661 (3/3 μM) and 10.4+/−1.2, 11.5+/−0.5, 18.2+/−1.3 and 21.9+/−0.7 FSK+VX-770 AUC/min (μA/cm²) (+) VX-445/VX-661 (3/3 μM) respectively. As observed in the mRNA response data, SD-10 (10 μM) & SA-08 (10 μM) resulted in a larger effect than SD-10 (100 μM) treatment.

### Example 8: Modulation of CFTR mRNA Processing and Restoration of CFTR Channel Function by Intron 22-Targeting ASOs in Primary Human Bronchial Epithelial (HBE) Cells

Primary HBE cells were treated with exemplary ASO(s) for 2 weeks or 3 weeks in order to assess the long-term effects of ASO treatments. Briefly, primary HBE cells were extracted from a CF patient carrying the homozygote W1282X″ CFTR variant (primary W1282X^{+/+} HBE) and were allowed to expand and differentiate in vitro according to established protocols that are well known to those skilled in the art, including culturing at an air liquid interface. The cells were then treated with either 10 μM SD10 or 4 μM SA08/10 μM SD10 for 2 weeks or 3 weeks, with retreatment of ASOs every 2 to 3 days. CFTR correctors 3/3 μM VX-661/445 were added to the cells 48 hours prior to the transepithelial chloride conductance assay (TECC-24 assay), which was conducted similarly as described above in Example 7. Benzamil was added to the primary cells prior to forskolin activation to block epithelial sodium channel (ENaC) activity, which can interfere with detection of CFTR chloride channel activity.

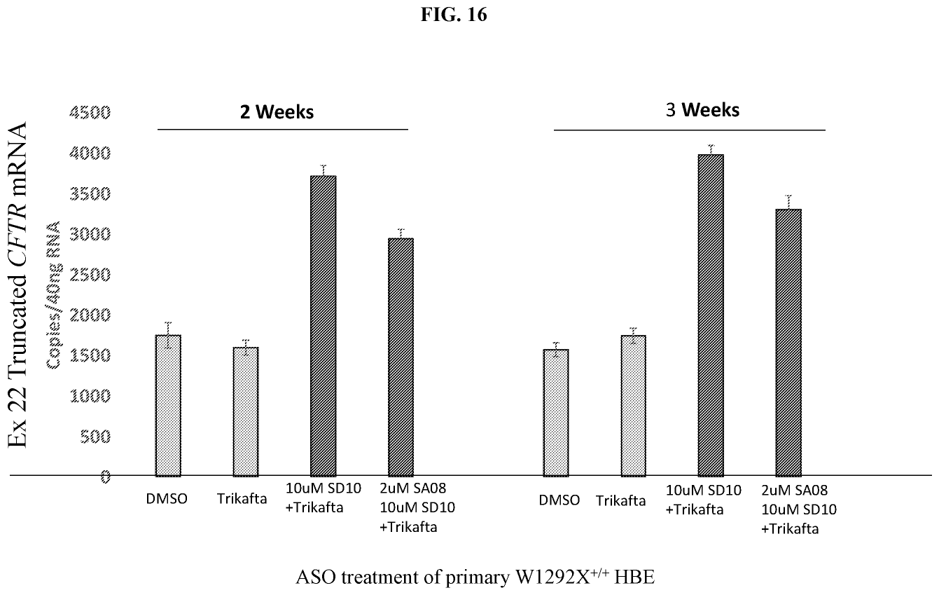

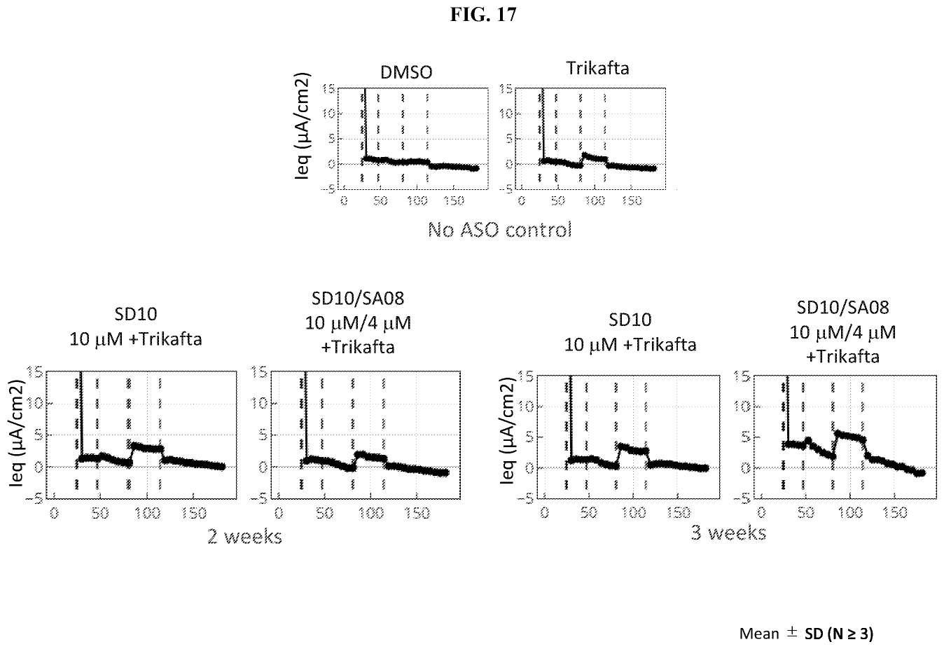

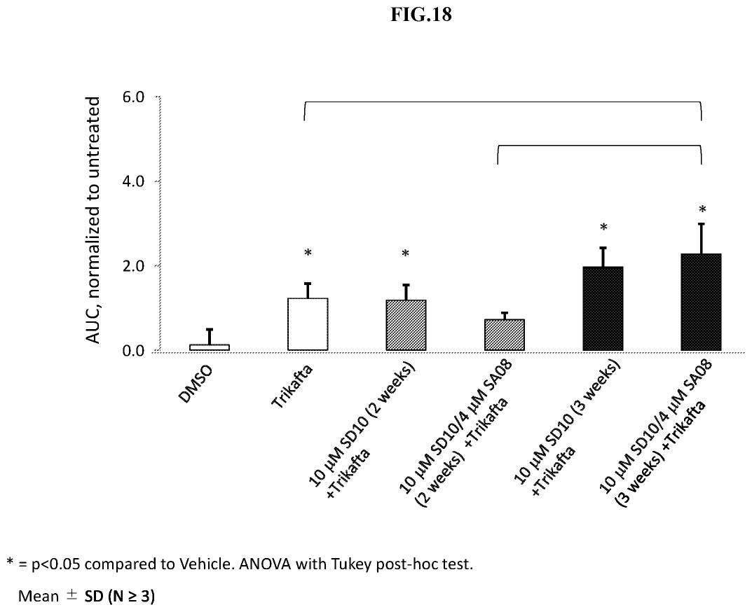

Exon 22 truncated CFTR mRNA was assayed using ddPCR, as depicted in FIG. 16. Treatment with exemplary ASO(s) for 2 week and for 3 weeks both increased the expression of Exon 22 truncated CFTR mRNA in primary W1282X^{+/+} HBE. Representative I_{eq} traces of chloride conductance from TECC-24 assay are depicted in FIG. 17. Transepithelial electrical resistance (TEER) were consistent in all groups of the experiment and no toxicity was observed with repeated treatments. Stronger chloride conductance (I_{eq}) was observed after treatment with exemplary ASO(s) for 2 week and for 3 weeks. Elevated baseline and increased slope of the chloride conductance curves (I_{eq} traces) were observed in the 3-week SD10/SA08 treatment group. FIG. 18 is a bar graph depicting that chloride conductance AUC measured in primary W1282X^{+/+} HBE when normalized to WT cells (percent of WT CFTR AUC) was increased after two or three weeks of ASO treatment. These data suggest that long term treatment of exemplary ASO(s) improved Ex22 truncated CFTR function compared to no treatment.

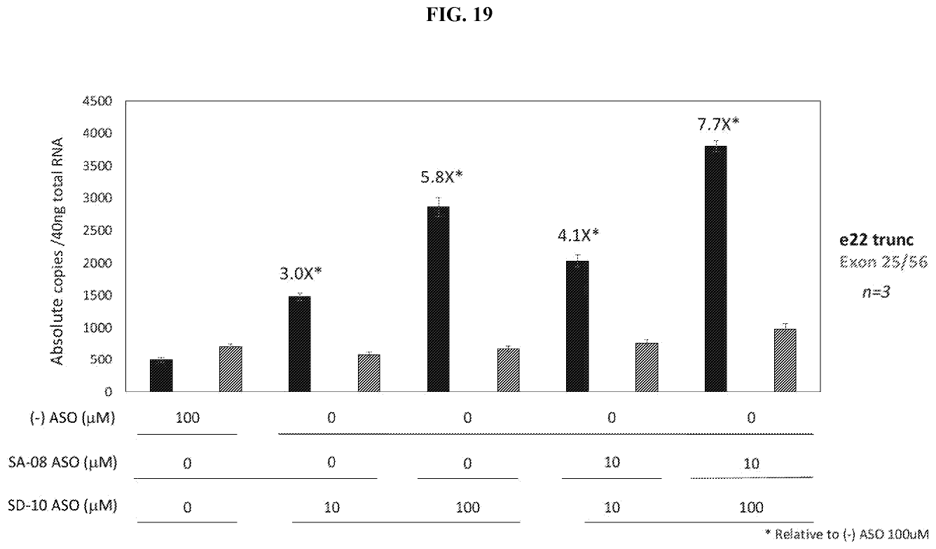

To extend the analysis into a more patient relevant model, the “gold standard” CF model, human bronchiole epithelial (hBE) culture at air/liquid interface (ALI), was employed. Unlike the previous CFF-16HBEgeW1282X cultures, HBEs at ALI form a pseudo epithelium that creates a barrier to free uptake, therefore, to achieve the sufficient upregulation of E22 trunc mRNA, it was found that it was necessary to subject hBE at ALI cultures to long ASOs exposure by starting ASO dosing at the undifferentiated state and refreshing with ASO at each feeding throughout differentiation. Undifferentiated W1282X^{+/+} hBEs (CF290) were taken through 28-day differentiation with ASO(s) refreshments every 2 days at feeding. The ASO(s) were administered/maintained at various doses: SD-10 (10 μM), SD-10 (100 μM), SD-10 (10 μM) & SA-08 (10 μM) combination treatment, SD-10 (100 μM) & SA-08 (10 μM) combination treatment, and (−) ASO TNMD (100 μM). ASO treatments were discontinued 48 hours before RNA harvest on day 21. To evaluate the effect of ASO blockade of exon 22/23 splicing on transcription, E22 trunc and full length CFTR mRNA (25/26 assay) were assessed via ddPCR from cells subjected to functional analysis. As depicted in FIG. 19, CF290 cells treated with (−) ASO control (100 μM) resulted in 495+/−42 and 715+/−36 absolute copies/40 ng total RNA of E22 trunc and full length CFTR mRNA, respectively. SD-10 treatment (10 μM) alone increased E22 trunc 3.0-fold to 1482+/−61 absolute copies/40 ng total RNA and full length CFTR mRNA remained unchanged at 583+/−40 absolute copies/40 ng total RNA. SD-10 (100 μM) alone further increased E22 trunc mRNA 5.8-fold to 2870+/−145 absolute copies/40 ng total RNA and did not change full length CFTR mRNA appreciably, 682+/−40 absolute copies/40 ng total RNA. SD-10 (10 μM) & SA-08 (10 μM) treatment increased E22 trunc mRNA 4.1-fold to 2035 +/−98 absolute copies/40 ng total RNA and again, full length CFTR mRNA remained unchanged at 767+/−56 absolute copies/40 ng total RNA. Lastly, consistent with the CFF-16HBEge-W1282X data, SD-10 (100 μM) & SA-08 (10 μM) treatment resulted in the maximum induction of E22 trunc, 7.7-fold and 3802+/−88 absolute copies/40 ng total RNA. SD-10 (100 μM) & SA-08 (10 UM) treatment did not significantly impact full length CFTR mRNA levels, 983+/−78 absolute copies/40 ng total RNA. As in the CFF-16HBEge W1282X line, exon 22/23 splice blocking ASO treatments upregulated E22 trunc mRNA levels and FL-CFTR-W1282X levels were largely unchanged.

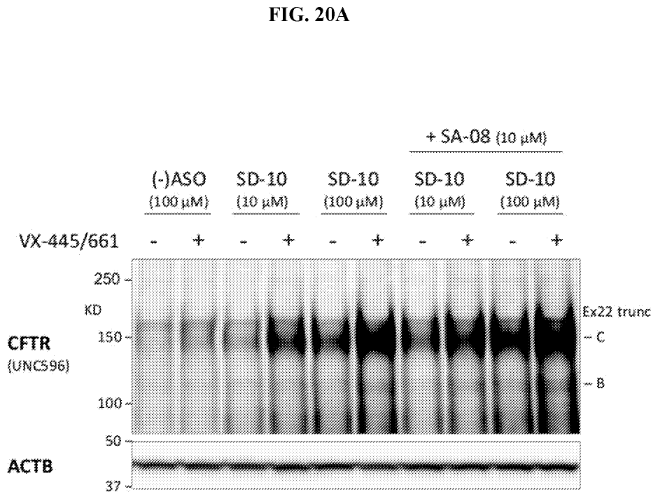

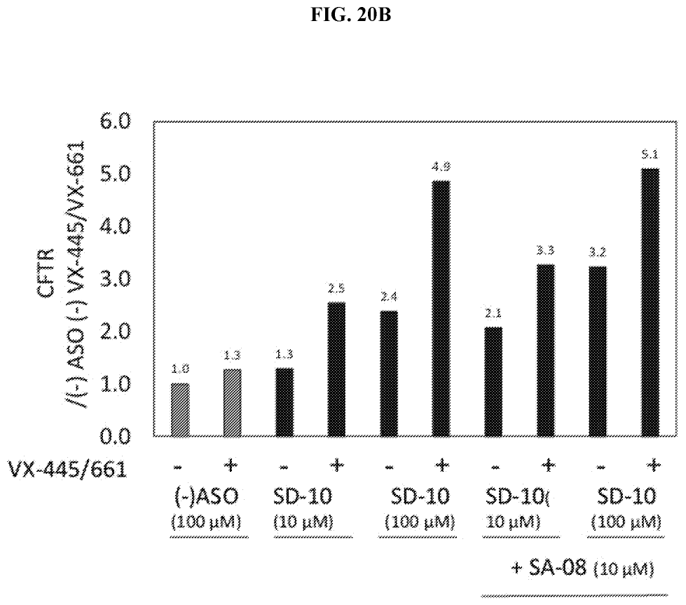

Next, Western blot analysis was used to measure the impact of ASO blockade of exon 22/23 splicing on E22 trunc protein expression (+/−) VX-445/VX-661/VX-770 (3/3/3 μM), as shown in FIG. 20A. Protein lysates were harvested from assay filters used in the TECC-24 functional assessments as above, run on gel and probed with CFTR UNC 596 and ACTB loading control. CFTR mature Band C intensity and ACTB band were quantified using densitometry. Band C intensities were normalized to ACTB and referenced to (−) ASO (100 μM) (−) VX-445/VX-661 (3/3 μM). All ASO blockade treatments increased Band C intensity relative to (−) ASO (−) VX-445/VX-661 (3/3 μM). Furthermore, VX-445/VX-661 increased Band C intensity relative to vehicle control for all ASO treatments. As observed with E22 trunc mRNA copy number, as shown in FIG. 20B, the order of Band C intensity was SD-10 (100 μM) & SA-08 (10 μM)>SD-10 (100 μM)>SD-10 (10 μM) & SA-08 (10 μM)>SD-10 (10 μM), 5.1×, 4.9×, 3.3×, and 2.5× and 3.2×, 2.4×, 2.1×, and 1.3× for VX-445/VX-661 (3/3 μM) (+/−) respectively.

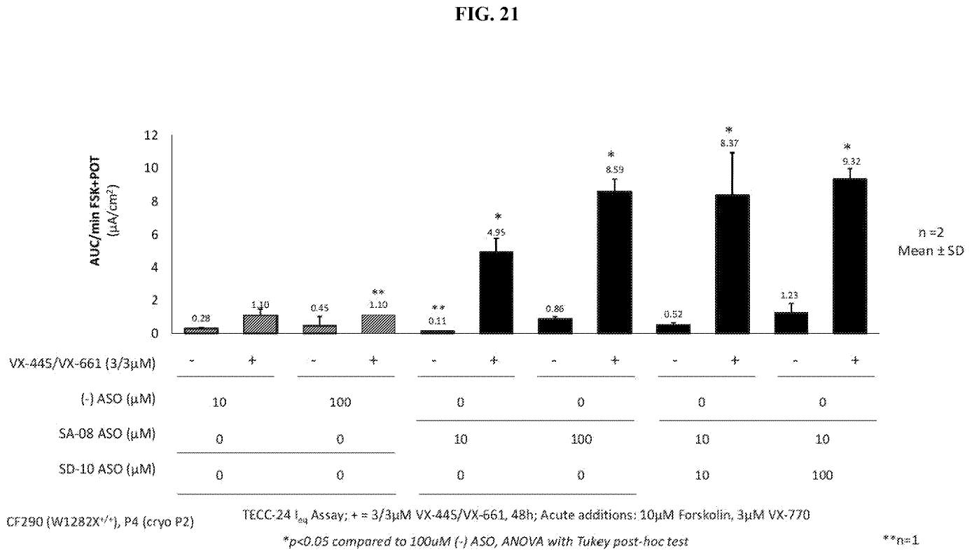

Lastly, to evaluate the functional impact of blockade of exon 22/23 splicing to upregulated E22 trunc mRNA and protein, TECC-24 assay were completed on fully differentiated hBEs W1282X^{+/+} at ALI with SD-10 (10 μM) or SD-10 (10 μM) and SA-08 (4 μM) (+/−) 445/VX-661 (3/3 μM), as shown in FIG. 21. (−) ASO control (10 μM or 100 μM) (n=2) with vehicle (0.002% DMSO) resulted in negligible CFTR Cl⁻ current 0.28+/−0.06 and 0.45+/−0.58 AUC/min FSK+POT (μA/cm²) that was increased marginally with 445/VX-661 (3/3 μM) to 1.1+/−0.37 and 1.1 (n=1) AUC/min FSK+POT (μA/cm²). Unlike the gene edited model and the exon 22/23 splice blocking ASO treatments in CFF-16HBEge-W1282X, ASO treatments in the absence of VX-445/VX-661 (3/3 μM) did not increase CFTR Cl⁻ currents even though increased mRNA and protein levels were observed. After 28 days at ALI treatment, CFTR Cl⁻ currents were significantly increased (p<0.05 compared to vehicle, ANOVA with Tukey post-hoc test) to 4.95+/−0.83, 8.59+/−0.74, 8.37+/−2.55, and 9.32+/−0.64 with SD-10 (10 μM), SD-10 (100 μM), SD-10 (10 μM) & SA-08 (10 μM), and SD-10 (100 μM) & SA-08 (10 μM) respectively with 48 hour VX-445/VX-661 (3/3 μM) chronic treatment.

This data supports that modulation of intron 22 ApA usage has therapeutic potential for patients with Cystic Fibrosis with 3′ terminal PTCs. There is reasonable evidence based on existing clinical and correlated in vitro data that achieving 10% WT response in an in vitro functional assay is predictive of a clinical benefit across a cohort of patients with Cystic Fibrosis. The D23-27 gene edited model suggests that a functional response of complete conversion CFTR mRNA to E22 trunc mRNA achieves ˜25% WT function and up to 50% with VX-770 dose escalation. These levels of function are well within the therapeutic range where one would expect patients to see benefit, potentially with a single responsive allele. Additionally, FRT overexpression of E22 trunc in the presence of modulators results in functional measurements that are in line with other FDA approved modulators CF genotype combinations including Cl— currents that were 1.6× that of F508del overexpression with VX-770/VX-809 (Orkambi) and ˜80% that of F508del VX-770/VX-661 (Symdeko).

Tables. 3A-3B below lists sequences of exemplary ASOs according to some embodiments of the present disclosure.

TABLE 1
List of target CFTR gene and pre-mRNA sequences

| SEQ ID NO. | Sequence Type |
|---|---|
| SEQ ID NO. 1 | Human CFTR gene (Gene ID: 1080, NC_000007.14: |
| | 117480025 . . . 117668665 |
| SEQ ID NO. 2 | Human CFTR pre-mRNA (encoding e.g., Cftr mRNA |
| | NM_000492.4; ENST00000003084.11) |

TABLE 2
Sequences of target intron, exon, or 3′ UTR in human CFTR pre-mRNA
transcripts

| SEQ ID | Sequence | |
|---|---|---|
| NO. | Type | Sequence |
| SEQ ID | Exon 22 gene | ATGCGATCTGTGAGCCGAGTCTTTAAGTTCATTGACA |
| NO. 3 | | TGCCAACAGAAGGTAAACCTACCAAGTCAACCAAACC |
| | | ATACAAGAATGGCCAACTCTCGAAAGTTATGATTATT |
| | | GAGAATTCACACGTGAAGAAAGATGACATCTGGCCC |
| | | TCAGGGGGCCAAATGACTGTCAAAGATCTCACAGCA |
| | | AAATACACAGAAGGTGGAAATGCCATATTAGAGAAC |
| | | ATTTCCTTCTCAATAAGTCCTGGCCAGAGG |
| SEQ ID | Exon 23 gene | GTGGGCCTCTTGGGAAGAACTGGATCAGGGAAGAGT |
| NO. 4 | | ACTTTGTTATCAGCTTTTTTGAGACTACTGAACACTG |
| | | AAGGAGAAATCCAGATCGATGGTGTGTCTTGGGATT |
| | | CAATAACTTTGCAACAGTGGAGGAAAGCCTTTGGAGT |
| | | GATACCACAG |
| SEQ ID | Intron 22 gene | gtgagatttgaacactgcttgctttgttagactgtgttcagtaagtgaatcccagtagcct |
| NO. 5 | | gaagcaatgtgttagcagaatctatttgtaacattattattgtacagtagaatcaatatta |
| | | aacacacatgttttattatatggagtcattatttttaatatgaaatttaatttgcagagtcct |
| | | gaacctatataatgggtttattttaaatgtgattgtacttgcagaatatctaattaattgct |
| | | aggttaataactaaagaagccattaaataaatcaaaattgtaacatgttttagatttccc |
| | | atcttgaaaatgtcttccaaaaatatcttattgctgactccatctattgtcttaaattttatct |
| | | aagttccattctgccaaacaagtgatactttttttctagcttttttcagtttgtttgttttgtttt |
| | | tctttgaagttttaattcagacatagattattttttcccagttatttactatatttattaagcat |
| | | gagtaattgacattattttgaaatccttcttatggatcccagcactgggctgaacacatag |
| | | aaggaacttaatatatactgatttctggaattgattcttggagacagggatggtcattatc |
| | | catatacttcaggctccataaacatatttcttaattgccttcaaatccctattctggactgct |
| | | ctataaatctagacaagagtattatatattttgattgatattttttagataaaataaaagg |
| | | gagctgaaaactgaattgcaaactgaattttaaaactttatctctctgtggttaattgcaa |
| | | acacagatacaaaaatatagagagagatacagttagtaaagatgttaggtcaccgtta |
| | | ctaacactgacatagaaacagttttgctcatgagtttcagaatatatgagtttgattttgcc |
| | | catggattttagaatatttgataaacatttaatgcattgtacaaattctgtgaaaacatat |
| | | atataggatgtgcgaaaagtccctgtgtatcatgtgaaatggcttaaaacagaacacca |
| | | taggtattcatatcagtgaataccataggtagctgaaagtgttttttcctggggtcgccaa |
| | | gatgaatgccaaaagtgatatcattattataaacaatagccagaataggttggtataaa |
| | | cctggtagaaagccttgataaattgactttctctcctcctgacatcctgccacccctttgct |
| | | ttgctgatgctcatttgtccactaaattaaactcaagcaagccctagtaaagtaatagaa |
| | | tttgtggagtcctcattagtataggaagtttccctgatgtgagattagtaattagagatgt |
| | | agcaaaatgagaaagaagtaatatgcttagatatttcattttctctgaacctgtatataca |
| | | aaataggccatgcgtgttcagtaactattcactgcaaggcactctctaggtactttgggg |
| | | gaattggaaattactcacataaggctatggattgtgccatttgtcaaaagacaaaatga |
| | | caacaaatttagtttaaagacctcagtcagctttattttctattctagatttggacagtcctt |
| | | catttcacaaattggagtaagtgttccaataagttgagcaaaggagcttggctttataga |
| | | cccaaaaaaagggccaaaggaagcagaaacaaagaacaataagagaattggtcatt |
| | | tcaaagttacttttcttgaaaggtggggacaaggagacagaataatagaaaagtcactg |
| | | attggttaacattggattaagaattaaaacagaggaaactttaagattgaagtttgaaac |
| | | tgacttgtttgggaaatcaggctgtcttctttcttgatttcttagaaggccggataacaact |
| | | gagttttgctttggtgaacatgggtgactccatttttacttttagtctggtctgttgaggcct |
| | | cgtgagagagcttaatctaaaacaatgacttcctataatttttgtttgacacatccaaaga |
| | | gggactctaatatttattgagagcttatcatatcttaagtactgtttaaacacttttatttgc |
| | | tattacatttgatcttattataactctaaaggcagaaatgattgcttttattttccacaatgg |
| | | aggaaactgaggttcaattaagtgagtaaggaagcagggatcttaaacccagatacca |
| | | ttgctcctctttaaaggtggaagaacagaaaacatggggcaggggaagagagaaagtt |
| | | tctgtcccaggacatgataatctaaaagggaaaacgtaagatccactgaaacctgagg |
| | | cagatttattgtggcaataacaaagcttaagtttcacagaccttcatttgcctgagccaac |
| | | tttgaaggccatgtatctaattttgtttttataattctataatctttattcttgaaaagagccc |
| | | tccctccaaatttacaagctttgggcccccaaaatccttgaaatgcccttgaataagaga |
| | | tatccaggtaaatgctatgggaattcagaggaggaagcagttagtatcagttggcggag |
| | | agttaggctattaagagaaggttttatataggaagtggcatttagaatgaagctttgaga |
| | | actgagctgtgtatttgaacaagtaaaggtggtgttgcagaattttgctccttagttctatt |
| | | aaaaacccgggttcttgtcacatgatccggaaaatttaggcacacagatacattgaagc |
| | | atgagtagagcaggattttattgggcaaaaaggaaaaaaagaaaactcagcaaatcg |
| | | agatggagtcttgctcacagattgaatcccaggccaccacaaaggaactgaagagatc |
| | | gggcttctcccctgcataaggtgcaaattccccatggctccacccacttccccttagtgtg |
| | | catgtggggctccagtccacggtgggcatgcccagacaagccttgggcaggttccctca |
| | | tctgtgcaaaagcatctgatgtaaacacttgaggggtggttcggagattctctgggaccc |
| | | ttttattttcttatctgcctaggcatttggctgtctcagtgggtgggaaagggtgctccagg |
| | | caaagggcataacatgaggcaaagggcatgcacagaaaacagtgactggttcagtca |
| | | ggttgggggatgccaaaggaagtaatgggagacaagattggagcaagatagataaga |
| | | gattgtggattttttttcttttttatctatataaatacagagacagggtctcactatgttgccc |
| | | aggctggtctcaaactcctggcctcaagtgatcctcccacctcatcctcccaaagtgcta |
| | | ggattacaggcatgaggcactgtgcccaacctccaattttggattttgagagctaaagca |
| | | atatagtcgaaaactcagataatccaggtagattttgctattaggtgctatttggttcctg |
| | | gtacagagctaaaacccttggaatttcctaagtgataagagctacaggagcatcttttgt |
| | | tatatgtttccccccctagttcctgaaatagctctagagaaatacaggtgaataacatcct |
| | | ttgttattcatatcaagcccctatcaaccataccccagtttctatttatgaagtggcttttgg |
| | | gaagtccctaaagacaggagtggggaaaggctggttgtcagggggatgggttgaaact |
| | | ttcatcttccccccttgacctccagggagggatgagtggctgaaaattgtgtaaaatcaa |
| | | caatggccagtgatttaatcaaccatgcctatgtaatgaagccacccgataagccttaac |
| | | tggaactttttggagagcctccaggctggtgaagacattgaggtgctcagaaggtggta |
| | | ttccagagagagcacagaatctctgttccccttcccacattcattttgctatgcatctctcc |
| | | catctggctgttcttgagaggtatccgtttataataaactggtaacctagtaagtaaactg |
| | | ttaccctgagttctgtgagccattctagcaaattatcaaacctaaagagttcatggatacg |
| | | tgcaatttacagatgcacagtcagaagcacagatgacaatctgggcttgccattggcatt |
| | | tgaagtgtgttgggaggcagtcttacaggaatgagcccttatcctgtggggtctatgcta |
| | | ataacagacagttgtcagcattgcttggtgtcgaaaacccacattgttggtgtcagaagt |
| | | attgtcagtaggatagggaaaacagtttgttttctttttttagtggtctttggtcatctttaa |
| | | gagcagggcttctcaaagtgtggtccttgaaccagcatcacctgtaccacgtaagaactt |
| | | atgagaaatgttcattcttgggccccaacaaagaattaaaaattctgagggtgtgaacg |
| | | gggtctgagtttcagcacaacttcccgaccatgctgatgcattcttgcccaagcatgaaa |
| | | gccctcccttgtttaagaaggccattagggccgggtgtggtggctcatgcttgtaatcga |
| | | gcactttgagaggacatagtgggaggatcacttgagccctggagttctagacaagcctg |
| | | ggcaacatggcaaaatgctgtctccacaaaaatcacaaaaattaggtgggcgtgtgttg |
| | | tgtgcctataggcccagctacttaggagactgaggcaggaggatcgcttgagcccagg |
| | | agattaaggctgcagcgagctgtgatggcaccactacagcctggatgacagagtgaga |
| | | cactgtctcaaaaaaaaaaaagaaaaagaaaaagaaaaaagaaaggaaaatgaaa |
| | | aagaacgccattaggtataaaggagcaatggtaaaagaccagttgcaaaaggttagg |
| | | gaatgggtggttactgaaataagaagctatgtagaacactagtgttggtggcaggaagt |
| | | agaaagcaagagcactgctctgtgggggatggtcatagcaaatgcaatatggaggcat |
| | | ttgcctctgcactgaggagaaaactatcttttccaagataggaggaaaggagataagtg |
| | | gaattaaagagaacctttgagcacagagttgggaaactgaaggtatttgtgttgtgctcc |
| | | ctcaatcttttaattcaactataagctaaacccatgaaacttgagtagtttcagttatctga |
| | | cttttttcttctcttttgatacagtgttggctattctgggtcttttgcctctctttatgtacttaa |
| | | gaatcagtttgccaatgtatgcaaaataactggctgggattttgattgtgattggcttgaa |
| | | tctatagatggagttgggaaggactgacatcttgacaatgttgaagcttcctattcatcat |
| | | tatgaaatatttctccatttgtttgattctttgatttcttttatcagaatttagttttcctcatat |
| | | agtcttttaaaatattttgttatattttgttcaagtattttgtttttgaggaatgccaatgtaa |
| | | atggtattgtgattttaatttcaaattccaatttttcattgctgttatataggaaaatgatttt |
| | | ttttgcatgttagccttatatctttcaactttgctataatcaattattgatagtttcaaggatt |
| | | ttttggtcaattattttgaatcttctacatagattatcatcatctgaacttagttttatttcttc |
| | | cttcccaatctgtatacctttatctccttttcttatttcattagctaggacttccagtatgatgt |
| | | tgaaagtagtggtgagaggggatatcttggtcttgttcttgatcttagtgggaaaacttca |
| | | agtttcttatcattaagtatgattttagctggagggtttttgtagaagttttttttttttaagtt |
| | | gaagaagtctccttctatttttagtttgctgatttttaaaaagaatcaggaatgggtgttaa |
| | | attttgtgaaatgcttttctgcaactattgatttgagcactttatttttcttctttggcttgttg |
| | | atgtgaagtacattaattgatttttgaatgctgaatcaaccttttgtacctgagattaatcc |
| | | cgtttggttgtggtatataattatttgtatacatgttgagttcgatttgctaatactttttgag |
| | | aatttttgcattggtgttcatgaaaaaatattggtgtgtagttttttgtgacatctttatctgc |
| | | ttatggttttaaggtaatgctggcctcatagcatgagttagggagtatttcctctacttttac |
| | | atttgagaagagattgcagagaattagtaaaattcctactttaaatattttgtggaattca |
| | | ccagtgaacccatctggacctggtgctttctgttttggaaggtcattaattattttaaaata |
| | | gatataggcctattcagattacctattttttctcatgcgagttttagcagattgtctttcaag |
| | | gaattggtctatttcatttaggttatcaaatatgtcaacgtagagttattcatagtattctttt |
| | | attatccttttaatgtgcaagggatctgtagtgatgtccccttttttgttttattgatattagc |
| | | aatttgtgtcacatcttttattttgctttgttagccaggctagagatatctctatttttgatgtt |
| | | tttgatgaaccaactttttgttttattgattttctctgttgatttcgtgatttcaatttcatgatt |
| | | tttaaattatgcttacatttgatttaatttgatcttcttttgctagttatccaaggtggaagct |
| | | tatattgttaagatccttttgcattcttatgcattcaatgatgtaaatttccctctaagcact |
| | | gctttttctgcatctcacaaatattcatgagttgtattttcatgttcatttagtttgaaatattt |
| | | ttaaatttctcttgatatttctcttttgacccatgtgttacttagaagtgtgttgtttaatcacc |
| | | atttttaaaaattttctagctatctttctgttattgatttctagtttaattccattgtggtctga |
| | | gagcatatattgtataattttaatttttataaaatttgttaaggtgtgatttatggcccagaa |
| | | tgtggtctatcttggtgaatgttccatgtaagctttggaagactgtgtattctgctatatttg |
| | | aatgaggtagtctatagacatcaattatgtccagttgattgatggtgctgttgaattcaac |
| | | tatgtccttactgattttccacctgctagatctgtccattctttgcagagggacactgaagt |
| | | ctccaactctagtagtgaatattctatttcttgttacagttttatcaacttctgcttcatgtctt |
| | | ttgatgctttgttgctagaaacatacacatgaagaattggtatgtcttttggagcatgacc |
| | | catttatcctcatataatgcccctcattatttcctcgccctgatgtctgttctctctgaaaga |
| | | aatatagcctctccaggtctcttttggttggtgttaaaatgacttaactttctttatccccctt |
| | | acttttagtttatatgtggttttaaatttaaagtgggtttcttgtagacagcaaatagttcag |
| | | agttgtttttcgatccactttgacaatctttgtcttttaattggtatatttggactattgatatt |
| | | ttaagtgattattgatatagttagataaacatctactatatttattactgttttctgtctgtta |
| | | cactacttgttctttgtttatatttttattgtctactcttttttttccattgtggttttaatcgag |
| | | cattttatatgtttccattttcttttcttagcatagtaattcttctttaaaaaaacattttttagt |
| | | ggttgcccctagagtttgcaatatacatttacaactaatctaagtccattttcaaataatac |
| | | taaataatttcatgtgtagtgcaagtaccttttaataataaaacactcccagttccaccttc |
| | | cagtctcttgtattatagctataatttagttcacttacatatatgggtatacctaagtatata |
| | | cattatcatatttatgattgaatatattgatgaaattattttgaaaaaactgttatcgttaaa |
| | | tcaattaagagtaagaaaaatagttctaattttattataaaatgaaataccttcatttattc |
| | | attctctaatacactttctttctttatgtagatccaagtttctgacctgtataattttccttttct |
| | | ctcttcagcttctttgaacatttcttaccagccagacctactgacaacaattttccccaattt |
| | | ttgtttgtctgatagagactttatttcttcttgacttttgaagaataattccacagggcacag |
| | | aactctagattggtgatttcttcccctcaaacccttaaatatttcattccactgccttcttgct |
| | | tgcattgtttctgagaagttagatataattcttatctttgcctttctataggtaagatgtttttt |
| | | cctctggcttctatcaagattttttctttatgaacatgatatgcctttctttttgaacatgata |
| | | tgcctttctttttgaacatgatatgcctttgtgtcggattttttttggcattattctgcttggttt |
| | | tctctgagtttcttggatatgtggtatggtatctgacactaatttggaaaaattctcagtca |
| | | ttattgcttcaaatatttcttctgttcttttttttcctttattctccttctggtattcccattacat |
| | | gtatgttacagtttttgtagtcatcccgctgttttggatattctgtttttttcagtttttttttcct |
| | | tcgcatttcagtgttggaagtttctattgacatattctcaacctcagagattctttcttcagc |
| | | tgtgttcagtctaccaatgagtccatcaaaggcattttacatttttattacagaatttttgac |
| | | ctatagaatttcttttgattccatctttgaatctccatttctcttctgcttttcatctgttcttgc |
| | | atgttgcctactttttccatgaaaacctttagctttttttttttttctttttgaggtggagtctca |
| | | ctgttgcccaggctggagtgcagtggtgtgatcttggctcactgcaacctctgcctcctgg |
| | | gttcaagtgattctcctcctcagcctcccaagtagctgggattacaggtgcctgccaccat |
| | | gcctgagtaatttttgtatttttagtagagatggggttttatcatgttggccaggcgggtctt |
| | | gaactcctaacctcaagtgatctgcccaccttagcctcccaaattgctgggattataggt |
| | | gtgagccaccatgccctgcctttagcatgttaatcatagttgttttaaattcctgatctgtta |
| | | attccaacatccctgtcatatctgactgtggttctgatgcttgctctgtgttttcaaatggtg |
| | | tttttttttttttgccttttagtaagccttgtaattttttattgaaaggtggacatgatgtgctg |
| | | ggtaaaaggaactgtagtaaataggcctttagtaatgtactggtaggtgtagcagaggg |
| | | tgagggaagtattctgtagtcctatgattaggttttagtcttttagtgagcctgtgcgcctg |
| | | cagcttggaagcacttgtgaagtgttttttcaccccttttggtgggacatagtgactagtgt |
| | | gagcgggagttgagtatttcccttcccctaggtcagttaggctctgaaaaaaccctgata |
| | | ggttaggcatggtaaaatagtctcttttgagggcaggcattgttataagaatagaatgct |
| | | ctggggccaggtgcggtggctcacgcctgtaatccccgcactttgggaggctaaggcag |
| | | gtggatcacctgaggtcaggagttcgagaccagcctggccaacatggtgaaaccccgt |
| | | ctctactaaaaatacaaaaatcagccaggtgtggtggcacacacctataatcccagcta |
| | | ctcaggaggctgaggcaggagaactgcttgaacccagtaagtggaggttacagtgacc |
| | | caagattgtgccactgcagtctagtctgggtgacagagcaagactccgtctcaaaaaaa |
| | | aaagaatgctctggcatatttgaaaatggttacttttccctttttttctctgatcttcactgtg |
| | | agaacctggtaagcatcctataggcaaaattcataaaagtatagaagtcggccagtga |
| | | cttggacccacttggaattttcttgctctcacatcatgcacactgaatctccagcaatttttc |
| | | acttacagtttaggttttcctaccctactactggttctctcagaggtttctgcttattggtttc |
| | | tgttttgtaagttgtgattctctgtacctaactgcctgtctcccattttggggggcagtggtt |
| | | tgccctgtgacctcacttctctgacagatctaagaaaagttgtttatttttcagtgtgctctg |
| | | ctttttacttgttacgatgaagccaaccactttcagaatttctacaaaccagatcagaatc |
| | | tggaagtcctgtttttttattttttttatccctttgtttagcatgttacctatcttaacacatttt |
| | | aaataagtgaatgcatagcttatatctacttctaggttatatgcttccttagaataggaatt |
| | | gattcttaaaatgtcgttctgctcacgcctgtaattccagcactttgggaggccaaggca |
| | | ggcggatcacttggggtcaggagttcaagaccagcctggtcaacatggtaaaaccctgt |
| | | gcctgcaaaaaatacaaaaattagctgggcatggtggtggccatctgtaatcccagcta |
| | | ctagggaagctaaggcatgagaatcacttgaacctgggaggtggaggttgcagtgagc |
| | | tgagatcgcgccactgcactccagcctgggtgacaagagcaaaactccatctcataaat |
| | | aaataaataaataaataaataaataataaaaaaaaaaaataaaataaaacaaaaat |
| | | tttattctgagcagtctctgaagaatataaattctactgccttgcctttagaacttataaca |
| | | gcatctcgcaaactatcacaagatgctccaaacatacttcttatgtgctgaattaagaag |
| | | tcaactcaaatttagtatactagtaatatttttggatatcccaaaacactgccagctcagc |
| | | tttaggctgcccttcttgggggggaaaaaagcagttgaaatttaggacttaagtgggcat |
| | | ctcgtttaatttttaatggatttctatgttgttggttatggtgaagaggtgaaaagaataaa |
| | | tattctgtgcagaaaaattattcagtcttcatgtgaaaacactttgtccatagcaattactt |
| | | tatgaaaaagatgtggtattactttctttgctcttaactgagacctttaatttaaagaacct |
| | | atactttacaagtttttattttcaatgcatgaaaaatgtagcagctatttcacaacctttact |
| | | tttaaaatccatttttctttttaatctcaaatagttttttcttaaaaccttttgactttttatcta |
| | | aattgtaatagccagagcaccttcccacaactagaatatctcatcctttttgtcttttcttttt |
| | | cctctcaaaatgcctactgggaacttaatttggagtcagattcttcatgataaatctggac |
| | | ttaatcaaaattcctcatatggtatattgtatatatcacagtactggatagtcctctgatta |
| | | aatagatatttgatagtactttaaggtctatacttttggatgaacttaactgctttctccatt |
| | | tgtagtctcttgaaaatacagaaatttcagaaataatttataagaatatcaaggattcaa |
| | | atcatatcagcacaaacacctaaatacttgtttgctttgttaaacacatatcccattttcta |
| | | tcttgataaacattggtgtaaagtagttgaatcattcagtgggtataagcagcatattctc |
| | | aatactatgtttcattaataattaatagagatatatgaacacataaaagattcaattataa |
| | | tcaccttgtggatctaaatttcagttgacttgtcatcttgatttctggagaccacaaggtaa |
| | | tgaaaaataattacaagagtcttccatctgttgcagtattaaaatggcgagtaagacacc |
| | | ctgaaaggaaatgttctattcatggtacaatgcaattacagctagcaccaaattcaacac |
| | | tgtttaactttcaacatattattttgatttatcttgatccaacattctcagggaggaggtgca |
| | | ttgaagttattagaaaacactgacttagatttagggtatgtcttaaaagcttatttgcggg |
| | | aagtactctagccttattcaacagatcactgagaagcctggaaaaacaaatcccggaa |
| | | actaattattatgtgccagttatataaacaagaagactttgttgggtacaaaccagtgatt |
| | | ccttgcctttgaaaaatgtgtcagatatcatgcattaccagcagttcaatgatataagga |
| | | aaccagagtaatagctaaaacctttaaagctaaaccaaagatttacaaattgcctcttca |
| | | tccagtctttcccaacctaaaaactgagttctctaaaaattttagtatttttttctgaagaa |
| | | aagggaacatggacatttatctaatcctcattagaaatctgactaatgataacaaggatt |
| | | tagacctcaagcacttcttaccaaaattcttgatatgaccttatagcaaattactttcacct |
| | | gttgaactttcctttcttttattcccctgtacctcacctgcactgggcatattcaagttgctta |
| | | tacaacactttactattgtgttagaaaaatcatgacacatgatgaatgtgtttgtgcaaca |
| | | tgagctgattcataaatgaaaatgtgcattgaaattccacaatattttaaaattaggagtt |
| | | tatctagcaattgaacaaaattgattaaatccattatttgttagatcagctaaattacata |
| | | agttcattcatctgctcataaatccatccattcttccatctggctatcccttagtcaattcaa |
| | | ataaatatttatggggcactttgggtaagccaggtgctaagaattcaatgcaaaacaag |
| | | atagactcccctgtccttgttgaacttatatttttggtacaaacaaaagcaataatcaaga |
| | | aaaaataaaaaaagtactgattgtgattaataatatgaagaaattcaacagagtattgt |
| | | acttaacatttgattgatctgattttctcagttgtctgagaacaaacatttgtgaaaatctc |
| | | attgtagagttcttacgatggatagggggtcaactgtgtcattattgcttatcagcttatcc |
| | | caaagacctagtttattaccagattgcaaatagtgttcaataaattattcttattaagggtt |
| | | gttatgtactctaaaacatttattgtggtcccttcactggttctggtttacaaacttacttttc |
| | | tatgatgacatagtatagaaattgagagtgaatatttagaagttcatttttattatatatttt |
| | | tgaagtattgatatgtagtgaattagaaatttaaaaagaaaacaaaactgtccttcacta |
| | | cagattgaaaagcattatactaaaagaccatttgctcagttatagtatataaaggccaa |
| | | atgacttaaaaacaaattatgtaaggagaaggaaacaaccatttattcagtgccactaa |
| | | ctgtcagccagttttttcagtggtcagttaatgactgcagtagtgttctaccttgctcaaag |
| | | caccctcctcaagttctggcatctaagctgacatcagaacacagagttggggctctctgt |
| | | gggtcacctctagcacttgatctcctcatgcagtgcatggtgctctcacgtctatgctatgt |
| | | tcttatggtctttaggtaacaagaataattttctttcttttccttactatacattttgctttctg |
| | | aaattcccttctcgccaatccaggtgaatgtcagaatgtgatttgacaactgtccaaagt |
| | | actcattcactgaggagtggtaaggccttcgcccaacctgccttctctgggaatatactg |
| | | ctgcctgaacatatcattgtttattgccaggcttgaacttcaccaaattaatttattagggt |
| | | caacatctaaatattagaactatttcagattaatttttaagtcgtatccactttgggtacta |
| | | gatcaaattgcaggtctctgcttctggcttgagcctatgtttagagatgatgtgcatgaag |
| | | acactctttgcttttcctttatgcaaaatgggcattttcaatctttttgtcattagtaaaggtc |
| | | agtgataaaggaagtctgcatcaggggtccaattccttatggccagtttctctattctgttc |
| | | caaggttgtttgtctccatatatcaacattggtcaggattgaaagtgtgcaacaaggtttg |
| | | aatgaataagtgaaaatcttccactggtgacaggataaaatattccaatggtttttattga |
| | | agtacaatactgaattatgtttatggcatggtacctatatgtcacagaagtgatcccatca |
| | | cttttaccttatag |
| SEQ ID | Exon 22 pre- | AUGCGAUCUGUGAGCCGAGUCUUUAAGUUCAUUGAC |
| NO. 6 | mRNA | AUGCCAACAGAAGGUAAACCUACCAAGUCAACCAAA |
| | | CCAUACAAGAAUGGCCAACUCUCGAAAGUUAUGAUU |
| | | AUUGAGAAUUCACACGUGAAGAAAGAUGACAUCUGG |
| | | CCCUCAGGGGGCCAAAUGACUGUCAAAGAUCUCACA |
| | | GCAAAAUACACAGAAGGUGGAAAUGCCAUAUUAGAG |
| | | AACAUUUCCUUCUCAAUAAGUCCUGGCCAGAGG |
| SEQ ID | Exon 23 pre- | GTGGGCCTCTTGGGAAGAACTGGATCAGGGAAGAGT |
| NO. 7 | mRNA | ACTTTGTTATCAGCTTTTTTGAGACTACTGAACACTG |
| | | AAGGAGAAATCCAGATCGATGGTGTGTCTTGGGATT |
| | | CAATAACTTTGCAACAGTGGAGGAAAGCCTTTGGAGT |
| | | GATACCACAG |
| SEQ ID | Intron 22 pre- | gtgagatttgaacactgcttgctttgttagactgtgttcagtaagtgaatcccagtagcct |
| NO. 8 | mRNA | gaagcaatgtgttagcagaatctatttgtaacattattattgtacagtagaatcaatatta |
| | | aacacacatgttttattatatggagtcattatttttaatatgaaatttaatttgcagagtcct |
| | | gaacctatataatgggtttattttaaatgtgattgtacttgcagaatatctaattaattgct |
| | | aggttaataactaaagaagccattaaataaatcaaaattgtaacatgttttagatttccc |
| | | atcttgaaaatgtcttccaaaaatatcttattgctgactccatctattgtcttaaattttatct |
| | | aagttccattctgccaaacaagtgatactttttttctagcttttttcagtttgtttgttttgtttt |
| | | tctttgaagttttaattcagacatagattattttttcccagttatttactatatttattaagcat |
| | | gagtaattgacattattttgaaatccttcttatggatcccagcactgggctgaacacatag |
| | | aaggaacttaatatatactgatttctggaattgattcttggagacagggatggtcattatc |
| | | catatacttcaggctccataaacatatttcttaattgccttcaaatccctattctggactgct |
| | | ctataaatctagacaagagtattatatattttgattgatattttttagataaaataaaagg |
| | | gagctgaaaactgaattgcaaactgaattttaaaactttatctctctgtggttaattgcaa |
| | | acacagatacaaaaatatagagagagatacagttagtaaagatgttaggtcaccgtta |
| | | ctaacactgacatagaaacagttttgctcatgagtttcagaatatatgagtttgattttgcc |
| | | catggattttagaatatttgataaacatttaatgcattgtacaaattctgtgaaaacatat |
| | | atataggatgtgcgaaaagtccctgtgtatcatgtgaaatggcttaaaacagaacacca |
| | | taggtattcatatcagtgaataccataggtagctgaaagtgttttttcctggggtcgccaa |
| | | gatgaatgccaaaagtgatatcattattataaacaatagccagaataggttggtataaa |
| | | cctggtagaaagccttgataaattgactttctctcctcctgacatcctgccacccctttgct |
| | | ttgctgatgctcatttgtccactaaattaaactcaagcaagccctagtaaagtaatagaa |
| | | tttgtggagtcctcattagtataggaagtttccctgatgtgagattagtaattagagatgt |
| | | agcaaaatgagaaagaagtaatatgcttagatatttcattttctctgaacctgtatataca |
| | | aaataggccatgcgtgttcagtaactattcactgcaaggcactctctaggtactttgggg |
| | | gaattggaaattactcacataaggctatggattgtgccatttgtcaaaagacaaaatga |
| | | caacaaatttagtttaaagacctcagtcagctttattttctattctagatttggacagtcctt |
| | | catttcacaaattggagtaagtgttccaataagttgagcaaaggagcttggctttataga |
| | | cccaaaaaaagggccaaaggaagcagaaacaaagaacaataagagaattggtcatt |
| | | tcaaagttacttttcttgaaaggtggggacaaggagacagaataatagaaaagtcactg |
| | | attggttaacattggattaagaattaaaacagaggaaactttaagattgaagtttgaaac |
| | | tgacttgtttgggaaatcaggctgtcttctttcttgatttcttagaaggccggataacaact |
| | | gagttttgctttggtgaacatgggtgactccatttttacttttagtctggtctgttgaggcct |
| | | cgtgagagagcttaatctaaaacaatgacttcctataatttttgtttgacacatccaaaga |
| | | gggactctaatatttattgagagcttatcatatcttaagtactgtttaaacacttttatttgc |
| | | tattacatttgatcttattataactctaaaggcagaaatgattgcttttattttccacaatgg |
| | | aggaaactgaggttcaattaagtgagtaaggaagcagggatcttaaacccagatacca |
| | | ttgctcctctttaaaggtggaagaacagaaaacatggggcaggggaagagagaaagtt |
| | | tctgtcccaggacatgataatctaaaagggaaaacgtaagatccactgaaacctgagg |
| | | cagatttattgtggcaataacaaagcttaagtttcacagaccttcatttgcctgagccaac |
| | | tttgaaggccatgtatctaattttgtttttataattctataatctttattcttgaaaagagccc |
| | | tccctccaaatttacaagctttgggcccccaaaatccttgaaatgcccttgaataagaga |
| | | tatccaggtaaatgctatgggaattcagaggaggaagcagttagtatcagttggcggag |
| | | agttaggctattaagagaaggttttatataggaagtggcatttagaatgaagctttgaga |
| | | actgagctgtgtatttgaacaagtaaaggtggtgttgcagaattttgctccttagttctatt |
| | | aaaaacccgggttcttgtcacatgatccggaaaatttaggcacacagatacattgaagc |
| | | atgagtagagcaggattttattgggcaaaaaggaaaaaaagaaaactcagcaaatcg |
| | | agatggagtcttgctcacagattgaatcccaggccaccacaaaggaactgaagagatc |
| | | gggcttctcccctgcataaggtgcaaattccccatggctccacccacttccccttagtgtg |
| | | catgtggggctccagtccacggtgggcatgcccagacaagccttgggcaggttccctca |
| | | tctgtgcaaaagcatctgatgtaaacacttgaggggtggttcggagattctctgggaccc |
| | | ttttattttcttatctgcctaggcatttggctgtctcagtgggtgggaaagggtgctccagg |
| | | caaagggcataacatgaggcaaagggcatgcacagaaaacagtgactggttcagtca |
| | | ggttgggggatgccaaaggaagtaatgggagacaagattggagcaagatagataaga |
| | | gattgtggattttttttcttttttatctatataaatacagagacagggtctcactatgttgccc |
| | | aggctggtctcaaactcctggcctcaagtgatcctcccacctcatcctcccaaagtgcta |
| | | ggattacaggcatgaggcactgtgcccaacctccaattttggattttgagagctaaagca |
| | | atatagtcgaaaactcagataatccaggtagattttgctattaggtgctatttggttcctg |
| | | gtacagagctaaaacccttggaatttcctaagtgataagagctacaggagcatcttttgt |
| | | tatatgtttccccccctagttcctgaaatagctctagagaaatacaggtgaataacatcct |
| | | ttgttattcatatcaagcccctatcaaccataccccagtttctatttatgaagtggcttttgg |
| | | gaagtccctaaagacaggagtggggaaaggctggttgtcagggggatgggttgaaact |
| | | ttcatcttccccccttgacctccagggagggatgagtggctgaaaattgtgtaaaatcaa |
| | | caatggccagtgatttaatcaaccatgcctatgtaatgaagccacccgataagccttaac |
| | | tggaactttttggagagcctccaggctggtgaagacattgaggtgctcagaaggtggta |
| | | ttccagagagagcacagaatctctgttccccttcccacattcattttgctatgcatctctcc |
| | | catctggctgttcttgagaggtatccgtttataataaactggtaacctagtaagtaaactg |
| | | ttaccctgagttctgtgagccattctagcaaattatcaaacctaaagagttcatggatacg |
| | | tgcaatttacagatgcacagtcagaagcacagatgacaatctgggcttgccattggcatt |
| | | tgaagtgtgttgggaggcagtcttacaggaatgagcccttatcctgtggggtctatgcta |
| | | ataacagacagttgtcagcattgcttggtgtcgaaaacccacattgttggtgtcagaagt |
| | | attgtcagtaggatagggaaaacagtttgttttctttttttagtggtctttggtcatctttaa |
| | | gagcagggcttctcaaagtgtggtccttgaaccagcatcacctgtaccacgtaagaactt |
| | | atgagaaatgttcattcttgggccccaacaaagaattaaaaattctgagggtgtgaacg |
| | | gggtctgagtttcagcacaacttcccgaccatgctgatgcattcttgcccaagcatgaaa |
| | | gccctcccttgtttaagaaggccattagggccgggtgtggtggctcatgcttgtaatcga |
| | | gcactttgagaggacatagtgggaggatcacttgagccctggagttctagacaagcctg |
| | | ggcaacatggcaaaatgctgtctccacaaaaatcacaaaaattaggtgggcgtgtgttg |
| | | tgtgcctataggcccagctacttaggagactgaggcaggaggatcgcttgagcccagg |
| | | agattaaggctgcagcgagctgtgatggcaccactacagcctggatgacagagtgaga |
| | | cactgtctcaaaaaaaaaaaagaaaaagaaaaagaaaaaagaaaggaaaatgaaa |
| | | aagaacgccattaggtataaaggagcaatggtaaaagaccagttgcaaaaggttagg |
| | | gaatgggtggttactgaaataagaagctatgtagaacactagtgttggtggcaggaagt |
| | | agaaagcaagagcactgctctgtgggggatggtcatagcaaatgcaatatggaggcat |
| | | ttgcctctgcactgaggagaaaactatcttttccaagataggaggaaaggagataagtg |
| | | gaattaaagagaacctttgagcacagagttgggaaactgaaggtatttgtgttgtgctcc |
| | | ctcaatcttttaattcaactataagctaaacccatgaaacttgagtagtttcagttatctga |
| | | cttttttcttctcttttgatacagtgttggctattctgggtcttttgcctctctttatgtacttaa |
| | | gaatcagtttgccaatgtatgcaaaataactggctgggattttgattgtgattggcttgaa |
| | | tctatagatggagttgggaaggactgacatcttgacaatgttgaagcttcctattcatcat |
| | | tatgaaatatttctccatttgtttgattctttgatttcttttatcagaatttagttttcctcatat |
| | | agtcttttaaaatattttgttatattttgttcaagtattttgtttttgaggaatgccaatgtaa |
| | | atggtattgtgattttaatttcaaattccaatttttcattgctgttatataggaaaatgatttt |
| | | ttttgcatgttagccttatatctttcaactttgctataatcaattattgatagtttcaaggatt |
| | | ttttggtcaattattttgaatcttctacatagattatcatcatctgaacttagttttatttcttc |
| | | cttcccaatctgtatacctttatctccttttcttatttcattagctaggacttccagtatgatgt |
| | | tgaaagtagtggtgagaggggatatcttggtcttgttcttgatcttagtgggaaaacttca |
| | | agtttcttatcattaagtatgattttagctggagggtttttgtagaagttttttttttttaagtt |
| | | gaagaagtctccttctatttttagtttgctgatttttaaaaagaatcaggaatgggtgttaa |
| | | attttgtgaaatgcttttctgcaactattgatttgagcactttatttttcttctttggcttgttg |
| | | atgtgaagtacattaattgatttttgaatgctgaatcaaccttttgtacctgagattaatcc |
| | | cgtttggttgtggtatataattatttgtatacatgttgagttcgatttgctaatactttttgag |
| | | aatttttgcattggtgttcatgaaaaaatattggtgtgtagttttttgtgacatctttatctgc |
| | | ttatggttttaaggtaatgctggcctcatagcatgagttagggagtatttcctctacttttac |
| | | atttgagaagagattgcagagaattagtaaaattcctactttaaatattttgtggaattca |
| | | ccagtgaacccatctggacctggtgctttctgttttggaaggtcattaattattttaaaata |
| | | gatataggcctattcagattacctattttttctcatgcgagttttagcagattgtctttcaag |
| | | gaattggtctatttcatttaggttatcaaatatgtcaacgtagagttattcatagtattctttt |
| | | attatccttttaatgtgcaagggatctgtagtgatgtccccttttttgttttattgatattagc |
| | | aatttgtgtcacatcttttattttgctttgttagccaggctagagatatctctatttttgatgtt |
| | | tttgatgaaccaactttttgttttattgattttctctgttgatttcgtgatttcaatttcatgatt |
| | | tttaaattatgcttacatttgatttaatttgatcttcttttgctagttatccaaggtggaagct |
| | | tatattgttaagatccttttgcattcttatgcattcaatgatgtaaatttccctctaagcact |
| | | gctttttctgcatctcacaaatattcatgagttgtattttcatgttcatttagtttgaaatattt |
| | | ttaaatttctcttgatatttctcttttgacccatgtgttacttagaagtgtgttgtttaatcacc |
| | | atttttaaaaattttctagctatctttctgttattgatttctagtttaattccattgtggtctga |
| | | gagcatatattgtataattttaatttttataaaatttgttaaggtgtgatttatggcccagaa |
| | | tgtggtctatcttggtgaatgttccatgtaagctttggaagactgtgtattctgctatatttg |
| | | aatgaggtagtctatagacatcaattatgtccagttgattgatggtgctgttgaattcaac |
| | | tatgtccttactgattttccacctgctagatctgtccattctttgcagagggacactgaagt |
| | | ctccaactctagtagtgaatattctatttcttgttacagttttatcaacttctgcttcatgtctt |
| | | ttgatgctttgttgctagaaacatacacatgaagaattggtatgtcttttggagcatgacc |
| | | catttatcctcatataatgcccctcattatttcctcgccctgatgtctgttctctctgaaaga |
| | | aatatagcctctccaggtctcttttggttggtgttaaaatgacttaactttctttatccccctt |
| | | acttttagtttatatgtggttttaaatttaaagtgggtttcttgtagacagcaaatagttcag |
| | | agttgtttttcgatccactttgacaatctttgtcttttaattggtatatttggactattgatatt |
| | | ttaagtgattattgatatagttagataaacatctactatatttattactgttttctgtctgtta |
| | | cactacttgttctttgtttatatttttattgtctactctttttctttccattgtggttttaatcgag |
| | | cattttatatgtttccattttcttttcttagcatagtaattcttctttaaaaaaacattttttagt |
| | | ggttgcccctagagtttgcaatatacatttacaactaatctaagtccattttcaaataatac |
| | | taaataatttcatgtgtagtgcaagtaccttttaataataaaacactcccagttccaccttc |
| | | cagtctcttgtattatagctataatttagttcacttacatatatgggtatacctaagtatata |
| | | cattatcatatttatgattgaatatattgatgaaattattttgaaaaaactgttatcgttaaa |
| | | tcaattaagagtaagaaaaatagttctaattttattataaaatgaaataccttcatttattc |
| | | attctctaatacactttctttctttatgtagatccaagtttctgacctgtataattttccttttct |
| | | ctcttcagcttctttgaacatttcttaccagccagacctactgacaacaattttccccaattt |
| | | ttgtttgtctgatagagactttatttcttcttgacttttgaagaataattccacagggcacag |
| | | aactctagattggtgatttcttcccctcaaacccttaaatatttcattccactgccttcttgct |
| | | tgcattgtttctgagaagttagatataattcttatctttgcctttctataggtaagatgtttttt |
| | | cctctggcttctatcaagattttttctttatgaacatgatatgccttttttttgaacatgata |
| | | tgcctttctttttgaacatgatatgcctttgtgtcggattttttttggcattattctgcttggttt |
| | | tctctgagtttcttggatatgtggtatggtatctgacactaatttggaaaaattctcagtca |
| | | ttattgcttcaaatatttcttctgttcttttttttcctttattctccttctggtattcccattacat |
| | | gtatgttacagtttttgtagtcatcccgctgttttggatattctgtttttttcagtttttttttcct |
| | | tcgcatttcagtgttggaagtttctattgacatattctcaacctcagagattctttcttcagc |
| | | tgtgttcagtctaccaatgagtccatcaaaggcattttacatttttattacagaatttttgac |
| | | ctatagaatttcttttgattccatctttgaatctccatttctcttctgcttttcatctgttcttgc |
| | | atgttgcctactttttccatgaaaacctttagctttttttttttttctttttgaggtggagtctca |
| | | ctgttgcccaggctggagtgcagtggtgtgatcttggctcactgcaacctctgcctcctgg |
| | | gttcaagtgattctcctcctcagcctcccaagtagctgggattacaggtgcctgccaccat |
| | | gcctgagtaatttttgtatttttagtagagatggggttttatcatgttggccaggcgggtctt |
| | | gaactcctaacctcaagtgatctgcccaccttagcctcccaaattgctgggattataggt |
| | | gtgagccaccatgccctgcctttagcatgttaatcatagttgttttaaattcctgatctgtta |
| | | attccaacatccctgtcatatctgactgtggttctgatgcttgctctgtgttttcaaatggtg |
| | | tttttttttttttgccttttagtaagccttgtaattttttattgaaaggtggacatgatgtgctg |
| | | ggtaaaaggaactgtagtaaataggcctttagtaatgtactggtaggtgtagcagaggg |
| | | tgagggaagtattctgtagtcctatgattaggttttagtcttttagtgagcctgtgcgcctg |
| | | cagcttggaagcacttgtgaagtgttttttcaccccttttggtgggacatagtgactagtgt |
| | | gagcgggagttgagtatttcccttcccctaggtcagttaggctctgaaaaaaccctgata |
| | | ggttaggcatggtaaaatagtctcttttgagggcaggcattgttataagaatagaatgct |
| | | ctggggccaggtgcggtggctcacgcctgtaatccccgcactttgggaggctaaggcag |
| | | gtggatcacctgaggtcaggagttcgagaccagcctggccaacatggtgaaaccccgt |
| | | ctctactaaaaatacaaaaatcagccaggtgtggtggcacacacctataatcccagcta |
| | | ctcaggaggctgaggcaggagaactgcttgaacccagtaagtggaggttacagtgacc |
| | | caagattgtgccactgcagtctagtctgggtgacagagcaagactccgtctcaaaaaaa |
| | | aaagaatgctctggcatatttgaaaatggttacttttccctttttttctctgatcttcactgtg |
| | | agaacctggtaagcatcctataggcaaaattcataaaagtatagaagtcggccagtga |
| | | cttggacccacttggaattttcttgctctcacatcatgcacactgaatctccagcaatttttc |
| | | acttacagtttaggttttcctaccctactactggttctctcagaggtttctgcttattggtttc |
| | | tgttttgtaagttgtgattctctgtacctaactgcctgtctcccattttggggggcagtggtt |
| | | tgccctgtgacctcacttctctgacagatctaagaaaagttgtttatttttcagtgtgctctg |
| | | ctttttacttgttacgatgaagccaaccactttcagaatttctacaaaccagatcagaatc |
| | | tggaagtcctgtttttttattttttttatccctttgtttagcatgttacctatcttaacacatttt |
| | | aaataagtgaatgcatagcttatatctacttctaggttatatgcttccttagaataggaatt |
| | | gattcttaaaatgtcgttctgctcacgcctgtaattccagcactttgggaggccaaggca |
| | | ggcggatcacttggggtcaggagttcaagaccagcctggtcaacatggtaaaaccctgt |
| | | gcctgcaaaaaatacaaaaattagctgggcatggtggtggccatctgtaatcccagcta |
| | | ctagggaagctaaggcatgagaatcacttgaacctgggaggtggaggttgcagtgagc |
| | | tgagatcgcgccactgcactccagcctgggtgacaagagcaaaactccatctcataaat |
| | | aaataaataaataaataaataaataataaaaataaaaaaataaaataaaacaaaaat |
| | | tttattctgagcagtctctgaagaatataaattctactgccttgcctttagaacttataaca |
| | | gcatctcgcaaactatcacaagatgctccaaacatacttcttatgtgctgaattaagaag |
| | | tcaactcaaatttagtatactagtaatatttttggatatcccaaaacactgccagctcagc |
| | | tttaggctgcccttcttgggggggaaaaaagcagttgaaatttaggacttaagtgggcat |
| | | ctcgtttaatttttaatggatttctatgttgttggttatggtgaagaggtgaaaagaataaa |
| | | tattctgtgcagaaaaattattcagtcttcatgtgaaaacactttgtccatagcaattactt |
| | | tatgaaaaagatgtggtattactttctttgctcttaactgagacctttaatttaaagaacct |
| | | atactttacaagtttttattttcaatgcatgaaaaatgtagcagctatttcacaacctttact |
| | | tttaaaatccatttttctttttaatctcaaatagttttttcttaaaaccttttgactttttatcta |
| | | aattgtaatagccagagcaccttcccacaactagaatatctcatcctttttgtcttttcttttt |
| | | cctctcaaaatgcctactgggaacttaatttggagtcagattcttcatgataaatctggac |
| | | ttaatcaaaattcctcatatggtatattgtatatatcacagtactggatagtcctctgatta |
| | | aatagatatttgatagtactttaaggtctatacttttggatgaacttaactgctttctccatt |
| | | tgtagtctcttgaaaatacagaaatttcagaaataatttataagaatatcaaggattcaa |
| | | atcatatcagcacaaacacctaaatacttgtttgctttgttaaacacatatcccattttcta |
| | | tcttgataaacattggtgtaaagtagttgaatcattcagtgggtataagcagcatattctc |
| | | aatactatgtttcattaataattaatagagatatatgaacacataaaagattcaattataa |
| | | tcaccttgtggatctaaatttcagttgacttgtcatcttgatttctggagaccacaaggtaa |
| | | tgaaaaataattacaagagtcttccatctgttgcagtattaaaatggcgagtaagacacc |
| | | ctgaaaggaaatgttctattcatggtacaatgcaattacagctagcaccaaattcaacac |
| | | tgtttaactttcaacatattattttgatttatcttgatccaacattctcagggaggaggtgca |
| | | ttgaagttattagaaaacactgacttagatttagggtatgtcttaaaagcttatttgcggg |
| | | aagtactctagccttattcaacagatcactgagaagcctggaaaaacaaatcccggaa |
| | | actaattattatgtgccagttatataaacaagaagactttgttgggtacaaaccagtgatt |
| | | ccttgcctttgaaaaatgtgtcagatatcatgcattaccagcagttcaatgatataagga |
| | | aaccagagtaatagctaaaacctttaaagctaaaccaaagatttacaaattgcctcttca |
| | | tccagtctttcccaacctaaaaactgagttctctaaaaattttagtatttttttctgaagaa |
| | | aagggaacatggacatttatctaatcctcattagaaatctgactaatgataacaaggatt |
| | | tagacctcaagcacttcttaccaaaattcttgatatgaccttatagcaaattactttcacct |
| | | gttgaactttcctttcttttattcccctgtacctcacctgcactgggcatattcaagttgctta |
| | | tacaacactttactattgtgttagaaaaatcatgacacatgatgaatgtgtttgtgcaaca |
| | | tgagctgattcataaatgaaaatgtgcattgaaattccacaatattttaaaattaggagtt |
| | | tatctagcaattgaacaaaattgattaaatccattatttgttagatcagctaaattacata |
| | | agttcattcatctgctcataaatccatccattcttccatctggctatcccttagtcaattcaa |
| | | ataaatatttatggggcactttgggtaagccaggtgctaagaattcaatgcaaaacaag |
| | | atagactcccctgtccttgttgaacttatatttttggtacaaacaaaagcaataatcaaga |
| | | aaaaataaaaaaagtactgattgtgattaataatatgaagaaattcaacagagtattgt |
| | | acttaacatttgattgatctgattttctcagttgtctgagaacaaacatttgtgaaaatctc |
| | | attgtagagttcttacgatggatagggggtcaactgtgtcattattgcttatcagcttatcc |
| | | caaagacctagtttattaccagattgcaaatagtgttcaataaattattcttattaagggtt |
| | | gttatgtactctaaaacatttattgtggtcccttcactggttctggtttacaaacttacttttc |
| | | tatgatgacatagtatagaaattgagagtgaatatttagaagttcatttttattatatatttt |
| | | tgaagtattgatatgtagtgaattagaaatttaaaaagaaaacaaaactgtccttcacta |
| | | cagattgaaaagcattatactaaaagaccatttgctcagttatagtatataaaggccaa |
| | | atgacttaaaaacaaattatgtaaggagaaggaaacaaccatttattcagtgccactaa |
| | | ctgtcagccagttttttcagtggtcagttaatgactgcagtagtgttctaccttgctcaaag |
| | | caccctcctcaagttctggcatctaagctgacatcagaacacagagttggggctctctgt |
| | | gggtcacctctagcacttgatctcctcatgcagtgcatggtgctctcacgtctatgctatgt |
| | | tcttatggtctttaggtaacaagaataattttctttcttttccttactatacattttgctttctg |
| | | aaattcccttctcgccaatccaggtgaatgtcagaatgtgatttgacaactgtccaaagt |
| | | actcattcactgaggagtggtaaggccttcgcccaacctgccttctctgggaatatactg |
| | | ctgcctgaacatatcattgtttattgccaggcttgaacttcaccaaattaatttattagggt |
| | | caacatctaaatattagaactatttcagattaatttttaagtcgtatccactttgggtacta |
| | | gatcaaattgcaggtctctgcttctggcttgagcctatgtttagagatgatgtgcatgaag |
| | | acactctttgcttttcctttatgcaaaatgggcattttcaatctttttgtcattagtaaaggtc |
| | | agtgataaaggaagtctgcatcaggggtccaattccttatggccagtttctctattctgttc |
| | | caaggttgtttgtctccatatatcaacattggtcaggattgaaagtgtgcaacaaggtttg |
| | | aatgaataagtgaaaatcttccactggtgacaggataaaatattccaatggtttttattga |
| | | agtacaatactgaattatgtttatggcatggtacctatatgtcacagaagtgatcccatca |
| | | cttttacttatag |
| SEQ ID | 3′ UTR of the | AGAGCAGCATAAATGTTGACATGGGACATTTGCTCAT |
| NO. 65 | CFTR gene | GGAATTGGAGCTCGTGGGACAGTCACCTCATGGAAT |
| | | TGGAGCTCGTGGAACAGTTACCTCTGCCTCAGAAAAC |
| | | AAGGATGAATTAAGTTTTTTTTTAAAAAAGAAACATT |
| | | TGGTAAGGGGAATTGAGGACACTGATATGGGTCTTG |
| | | ATAAATGGCTTCCTGGCAATAGTCAAATTGTGTGAAA |
| | | GGTACTTCAAATCCTTGAAGATTTACCACTTGTGTTT |
| | | TGCAAGCCAGATTTTCCTGAAAACCCTTGCCATGTGC |
| | | TAGTAATTGGAAAGGCAGCTCTAAATGTCAATCAGCC |
| | | TAGTTGATCAGCTTATTGTCTAGTGAAACTCGTTAAT |
| | | TTGTAGTGTTGGAGAAGAACTGAAATCATACTTCTTA |
| | | GGGTTATGATTAAGTAATGATAACTGGAAACTTCAGC |
| | | GGTTTATATAAGCTTGTATTCCTTTTTCTCTCCTCTCC |
| | | CCATGATGTTTAGAAACACAACTATATTGTTTGCTAA |
| | | GCATTCCAACTATCTCATTTCCAAGCAAGTATTAGAA |
| | | TACCACAGGAACCACAAGACTGCACATCAAAATATGC |
| | | CCCATTCAACATCTAGTGAGCAGTCAGGAAAGAGAA |
| | | CTTCCAGATCCTGGAAATCAGGGTTAGTATTGTCCAG |
| | | GTCTACCAAAAATCTCAATATTTCAGATAATCACAAT |
| | | ACATCCCTTACCTGGGAAAGGGCTGTTATAATCTTTC |
| | | ACAGGGGACAGGATGGTTCCCTTGATGAAGAAGTTG |
| | | ATATGCCTTTTCCCAACTCCAGAAAGTGACAAGCTCA |
| | | CAGACCTTTGAACTAGAGTTTAGCTGGAAAAGTATGT |
| | | TAGTGCAAATTGTCACAGGACAGCCCTTCTTTCCACA |
| | | GAAGCTCCAGGTAGAGGGTGTGTAAGTAGATAGGCC |
| | | ATGGGCACTGTGGGTAGACACACATGAAGTCCAAGC |
| | | ATTTAGATGTATAGGTTGATGGTGGTATGTTTTCAGG |
| | | CTAGATGTATGTACTTCATGCTGTCTACACTAAGAGA |
| | | GAATGAGAGACACACTGAAGAAGCACCAATCATGAA |
| | | TTAGTTTTATATGCTTCTGTTTTATAATTTTGTGAAGC |
| | | AAAATTTTTTCTCTAGGAAATATTTATTTTAATAATGT |
| | | TTCAAACATATATAACAATGCTGTATTTTAAAAGAAT |
| | | GATTATGAATTACATTTGTATAAAATAATTTTTATATT |
| | | TGAAATATTGACTTTTTATGGCACTAGTATTTCTATG |
| | | AAATATTATGTTAAAACTGGGACAGGGGAGAACCTA |
| | | GGGTGATATTAACCAGGGGCCATGAATCACCTTTTGG |
| | | TCTGGAGGGAAGCCTTGGGGCTGATGCAGTTGTTGC |
| | | CCACAGCTGTATGATTCCCAGCCAGCACAGCCTCTTA |
| | | GATGCAGTTCTGAAGAAGATGGTACCACCAGTCTGA |
| | | CTGTTTCCATCAAGGGTACACTGCCTTCTCAACTCCA |
| | | AACTGACTCTTAAGAAGACTGCATTATATTTATTACT |
| | | GTAAGAAAATATCACTTGTCAATAAAATCCATACATT |
| | | TGTGTGAAA |

TABLE 3A
Sequences of exemplary ASOs targeting human CFTR.

| SEQ | | | SEQ ID | |
|---|---|---|---|---|
| ID NO. | | | NO. for | |
| for | ASO | | Target | |
| ASO | Name | ASO sequence | Sequence | Target Sequence |
| 37 | SD1 | agtgttcaaatctcacCCTCTG | 9 | CAGAGGgtgagatttgaacact |
| 38 | SD2 | cagtgttcaaatctcacCCTCT | 10 | AGAGGgtgagatttgaacactg |
| 39 | SD3 | gcagtgttcaaatctcacCCTC | 11 | GAGGgtgagatttgaacactgc |
| 40 | SD4 | agcagtgttcaaatctcacCCT | 12 | AGGgtgagatttgaacactgct |
| 41 | SD5 | aagcagtgttcaaatctcacCC | 13 | GGgtgagatttgaacactgctt |
| 42 | SD6 | caagcagtgttcaaatctcacC | 14 | Ggtgagatttgaacactgcttg |
| 43 | SD7 | gcaagcagtgttcaaatctcac | 15 | gtgagatttgaacactgcttgc |
| 44 | SD8 | agcaagcagtgttcaaatctca | 16 | tgagatttgaacactgcttgct |
| 45 | SD9 | aagcaagcagtgttcaaatctc | 17 | gagatttgaacactgcttgctt |
| 46 | SD10 | aaagcaagcagtgttcaaatct | 18 | agatttgaacactgcttgcttt |
| 47 | SD11 | caaagcaagcagtgttcaaatc | 19 | gatttgaacactgcttgctttg |
| 48 | SD12 | AGGACTTATTGAGAA | 20 | CATTTCCTTCTCAATAAG |
| | | GGAAATG | | TCCT |
| 49 | SD13 | TGGCCAGGACTTATT | 21 | CCTTCTCAATAAGTCCT |
| | | GAGAAGG | | GGCCA |
| 50 | SD14 | cCCTCTGGCCAGGAC | 22 | TCAATAAGTCCTGGCCA |
| | | TTATTGA | | GAGGg |
| 51 | SD15 | tctcacCCTCTGGCCAGG | 23 | AAGTCCTGGCCAGAGGgt |
| | | ACTT | | gaga |
| 52 | SD16 | tcaaatctcacCCTCTGGCC | 24 | CTGGCCAGAGGgtgagatttg |
| | | AG | | a |
| 53 | SD17 | tctaacaaagcaagcagtgttc | 25 | gaacactgcttgctttgttaga |
| 54 | SD18 | cacagtctaacaaagcaagcag | 26 | ctgcttgctttgttagactgtg |
| 55 | SA1 | CCCACctataaggtaaaagtg | 27 | tcacttttaccttatagGTGGG |
| | | a | | |
| 56 | SA2 | CCACctataaggtaaaagtgat | 28 | atcacttttaccttatagGTGG |
| 57 | SA3 | CACctataaggtaaaagtgatg | 29 | catcacttttaccttatagGTG |
| 58 | SA4 | ACctataaggtaaaagtgatgg | 30 | ccatcacttttaccttatagGT |
| 59 | SA5 | Cctataaggtaaaagtgatggg | 31 | cccatcacttttaccttatagG |
| 60 | SA6 | ctataaggtaaaagtgatggga | 32 | tcccatcacttttaccttatag |
| 61 | SA7 | tataaggtaaaagtgatgggat | 33 | atcccatcacttttaccttata |
| 62 | SA8 | ataaggtaaaagtgatgggatc | 34 | gatcccatcacttttaccttat |
| 63 | SD9 | taaggtaaaagtgatgggatca | 35 | tgatcccatcacttttacctta |
| 64 | SD10 | aaggtaaaagtgatgggatcac | 36 | gtgatcccatcacttttacctt |

TABLE 3B
Chemical structures of exemplary ASOs targeting human CFTR.

| SEQ | | |
|---|---|---|
| ID NO. | | |
| for | ASO | |
| ASO | Name | ASO structure |
| 66 | SD1- | /52MOErA/*/i2MOErG/*/i2MOErT/*/i2MOErG/*/i2MOErT/*/ |
| | mod | i2MOErT/*/i2MOErC/*/i2MOErA/*/i2MOErA/*/i2MOErA/*/i2MOErT/ |
| | | */i2MOErC/*/i2MOErT/*/i2MOErC/*/i2MOErA/*/i2MOErC/*/ |
| | | i2MOErC/*/i2MOErC/*/i2MOErT/*/i2MOErC/*/i2MOErT/*/32MOErG |
| | | / |
| 67 | SD2- | /52MOErC/*/i2MOErA/*/i2MOErG/*/i2MOErT/*/i2MOErG/*/ |
| | mod | i2MOErT/*/i2MOErT/*/i2MOErC/*/i2MOErA/*/i2MOErA/*/i2MOErA/ |
| | | */i2MOErT/*/i2MOErC/*/i2MOErT/*/i2MOErC/*/i2MOErA/*/ |
| | | i2MOErC/*/i2MOErC/*/i2MOErC/*/i2MOErT/*/i2MOErC/*/32MOErT |
| | | / |
| 68 | SD3- | /52MOErG/*/i2MOErC/*/i2MOErA/*/i2MOErG/*/i2MOErT/*/ |
| | mod | i2MOErG/*/i2MOErT/*/i2MOErT/*/i2MOErC/*/i2MOErA/*/i2MOErA/ |
| | | */i2MOErA/*/i2MOErT/*/i2MOErC/*/i2MOErT/*/i2MOErC/*/ |
| | | i2MOErA/*/i2MOErC/*/i2MOErC/*/i2MOErC/*/i2MOErT/*/32MOErC |
| | | / |
| 69 | SD4- | /52MOErA/*/i2MOErG/*/i2MOErC/*/i2MOErA/*/i2MOErG/*/ |
| | mod | i2MOErT/*/i2MOErG/*/i2MOErT/*/i2MOErT/*/i2MOErC/*/i2MOErA/ |
| | | */i2MOErA/*/i2MOErA/*/i2MOErT/*/i2MOErC/*/i2MOErT/*/ |
| | | i2MOErC/*/i2MOErA/*/i2MOErC/*/i2MOErC/*/i2MOErC/*/32MOErT |
| | | / |
| 70 | SD5- | /52MOErA/*/i2MOErA/*/i2MOErG/*/i2MOErC/*/i2MOErA/*/ |
| | mod | i2MOErG/*/i2MOErT/*/i2MOErG/*/i2MOErT/*/i2MOErT/*/i2MOErC/ |
| | | */i2MOErA/*/i2MOErA/*/i2MOErA/*/i2MOErT/*/i2MOErC/*/ |
| | | i2MOErT/*/i2MOErC/*/i2MOErA/*/i2MOErC/*/i2MOErC/*/32MOErC |
| | | / |
| 71 | SD6- | /52MOErC/*/i2MOErA/*/i2MOErA/*/i2MOErG/*/i2MOErC/*/ |
| | mod | i2MOErA/*/i2MOErG/*/i2MOErT/*/i2MOErG/*/i2MOErT/*/i2MOErT/ |
| | | */i2MOErC/*/i2MOErA/*/i2MOErA/*/i2MOErA/*/i2MOErT/*/ |
| | | i2MOErC/*/i2MOErT/*/i2MOErC/*/i2MOErA/*/i2MOErC/*/32MOErC |
| | | / |
| 72 | SD7- | /52MOErG/*/i2MOErC/*/i2MOErA/*/i2MOErA/*/i2MOErG/*/ |
| | mod | i2MOErC/*/i2MOErA/*/i2MOErG/*/i2MOErT/*/i2MOErG/*/i2MOErT/ |
| | | */i2MOErT/*/i2MOErC/*/i2MOErA/*/i2MOErA/*/i2MOErA/*/ |
| | | i2MOErT/*/i2MOErC/*/i2MOErT/*/i2MOErC/*/i2MOErA/*/32MOErC |
| | | / |
| 73 | SD8- | /52MOErA/*/i2MOErG/*/i2MOErC/*/i2MOErA/*/i2MOErA/*/ |
| | mod | i2MOErG/*/i2MOErC/*/i2MOErA/*/i2MOErG/*/i2MOErT/*/i2MOErG/ |
| | | */i2MOErT/*/i2MOErT/*/i2MOErC/*/i2MOErA/*/i2MOErA/*/ |
| | | i2MOErA/*/i2MOErT/*/i2MOErC/*/i2MOErT/*/i2MOErC/*/32MOErA |
| | | / |
| 74 | SD9- | /52MOErA/*/i2MOErA/*/i2MOErG/*/i2MOErC/*/i2MOErA/*/ |
| | mod | i2MOErA/*/i2MOErG/*/i2MOErC/*/i2MOErA/*/i2MOErG/*/i2MOErT/ |
| | | */i2MOErG/*/i2MOErT/*/i2MOErT/*/i2MOErC/*/i2MOErA/*/ |
| | | i2MOErA/*/i2MOErA/*/i2MOErT/*/i2MOErC/*/i2MOErT/*/32MOErC |
| | | / |
| 75 | SD10- | /52MOErA/*/i2MOErA/*/i2MOErA/*/i2MOErG/*/i2MOErC/*/ |
| | mod | i2MOErA/*/i2MOErA/*/i2MOErG/*/i2MOErC/*/i2MOErA/*/i2MOErG/ |
| | | */i2MOErT/*/i2MOErG/*/i2MOErT/*/i2MOErT/*/i2MOErC/*/ |
| | | i2MOErA/*/i2MOErA/*/i2MOErA/*/i2MOErT/*/i2MOErC/*/32MOErT |
| | | / |
| 76 | SD11- | /52MOErC/*/i2MOErA/*/i2MOErA/*/i2MOErA/*/i2MOErG/*/ |
| | mod | i2MOErC/*/i2MOErA/*/i2MOErA/*/i2MOErG/*/i2MOErC/*/i2MOErA/ |
| | | */i2MOErG/*/i2MOErT/*/i2MOErG/*/i2MOErT/*/i2MOErT/*/ |
| | | i2MOErC/*/i2MOErA/*/i2MOErA/*/i2MOErA/*/i2MOErT/*/32MOErC |
| | | / |
| 77 | SD12- | /52MOErA/*/i2MOErG/*/i2MOErG/*/i2MOErA/*/i2MOErC/*/ |
| | mod | i2MOErT/*/i2MOErT/*/i2MOErA/*/i2MOErT/*/i2MOErT/*/i2MOErG/ |
| | | */i2MOErA/*/i2MOErG/*/i2MOErA/*/i2MOErA/*/i2MOErG/*/ |
| | | i2MOErG/*/i2MOErA/*/i2MOErA/*/i2MOErA/*/i2MOErT/*/ |
| | | 32MOErG/ |
| 78 | SD13- | /52MOErT/*/i2MOErG/*/i2MOErG/*/i2MOErC/*/i2MOErC/*/ |
| | mod | i2MOErA/*/i2MOErG/*/i2MOErG/*/i2MOErA/*/i2MOErC/*/i2MOErT/ |
| | | */i2MOErT/*/i2MOErA/*/i2MOErT/*/i2MOErT/*/i2MOErG/*/ |
| | | i2MOErA/*/i2MOErG/*/i2MOErA/*/i2MOErA/*/i2MOErG/*/ |
| | | 32MOErG/ |
| 79 | SD14- | /52MOErC/*/i2MOErC/*/i2MOErC/*/i2MOErT/*/i2MOErC/*/ |
| | mod | i2MOErT/*/i2MOErG/*/i2MOErG/*/i2MOErC/*/i2MOErC/*/i2MOErA/ |
| | | */i2MOErG/*/i2MOErG/*/i2MOErA/*/i2MOErC/*/i2MOErT/*/ |
| | | i2MOErT/*/i2MOErA/*/i2MOErT/*/i2MOErT/*/i2MOErG/*/32MOErA |
| | | / |
| 80 | SD15- | /52MOErT/*/i2MOErC/*/i2MOErT/*/i2MOErC/*/i2MOErA/*/ |
| | mod | i2MOErC/*/i2MOErC/*/i2MOErC/*/i2MOErT/*/i2MOErC/*/i2MOErT/ |
| | | */i2MOErG/*/i2MOErG/*/i2MOErC/*/i2MOErC/*/i2MOErA/*/ |
| | | i2MOErG/*/i2MOErG/*/i2MOErA/*/i2MOErC/*/i2MOErT/*/32MOErT |
| | | / |
| 81 | SD16- | /52MOErT/*/i2MOErC/*/i2MOErA/*/i2MOErA/*/i2MOErA/*/ |
| | mod | i2MOErT/*/i2MOErC/*/i2MOErT/*/i2MOErC/*/i2MOErA/*/i2MOErC/ |
| | | */i2MOErC/*/i2MOErC/*/i2MOErT/*/i2MOErC/*/i2MOErT/*/ |
| | | i2MOErG/*/i2MOErG/*/i2MOErC/*/i2MOErC/*/i2MOErA/*/ |
| | | 32MOErG/ |
| 82 | SD17- | /52MOErT/*/i2MOErC/*/i2MOErT/*/i2MOErA/*/i2MOErA/*/ |
| | mod | i2MOErC/*/i2MOErA/*/i2MOErA/*/i2MOErA/*/i2MOErG/*/i2MOErC/ |
| | | */i2MOErA/*/i2MOErA/*/i2MOErG/*/i2MOErC/*/i2MOErA/*/ |
| | | i2MOErG/*/i2MOErT/*/i2MOErG/*/i2MOErT/*/i2MOErT/*/32MOErC |
| | | / |
| 83 | SD18- | /52MOErC/*/i2MOErA/*/i2MOErC/*/i2MOErA/*/i2MOErG/*/ |
| | mod | i2MOErT/*/i2MOErC/*/i2MOErT/*/i2MOErA/*/i2MOErA/*/i2MOErC/ |
| | | */i2MOErA/*/i2MOErA/*/i2MOErA/*/i2MOErG/*/i2MOErC/*/ |
| | | i2MOErA/*/i2MOErA/*/i2MOErG/*/i2MOErC/*/i2MOErA/*/ |
| | | 32MOErG/ |
| 84 | SA1- | /52MOErC/*/i2MOErC/*/i2MOErC/*/i2MOErA/*/i2MOErC/*/ |
| | mod | i2MOErC/*/i2MOErT/*/i2MOErA/*/i2MOErT/*/i2MOErA/*/i2MOErA/ |
| | | */i2MOErG/*/i2MOErG/*/i2MOErT/*/i2MOErA/*/i2MOErA/*/ |
| | | i2MOErA/*/i2MOErA/*/i2MOErG/*/i2MOErT/*/i2MOErG/*/ |
| | | 32MOErA/ |
| 85 | SA2- | /52MOErC/*/i2MOErC/*/i2MOErA/*/i2MOErC/*/i2MOErC/*/ |
| | mod | i2MOErT/*/i2MOErA/*/i2MOErT/*/i2MOErA/*/i2MOErA/*/i2MOErG/ |
| | | */i2MOErG/*/i2MOErT/*/i2MOErA/*/i2MOErA/*/i2MOErA/*/ |
| | | i2MOErA/*/i2MOErG/*/i2MOErT/*/i2MOErG/*/i2MOErA/*/32MOErT |
| | | / |
| 86 | SA3- | /52MOErC/*/i2MOErA/*/i2MOErC/*/i2MOErC/*/i2MOErT/*/ |
| | mod | i2MOErA/*/i2MOErT/*/i2MOErA/*/i2MOErA/*/i2MOErG/*/i2MOErG/ |
| | | */i2MOErT/*/i2MOErA/*/i2MOErA/*/i2MOErA/*/i2MOErA/*/ |
| | | i2MOErG/*/i2MOErT/*/i2MOErG/*/i2MOErA/*/i2MOErT/*/ |
| | | 32MOErG/ |
| 87 | SA4- | /52MOErA/*/i2MOErC/*/i2MOErC/*/i2MOErT/*/i2MOErA/*/ |
| | mod | i2MOErT/*/i2MOErA/*/i2MOErA/*/i2MOErG/*/i2MOErG/*/i2MOErT/ |
| | | */i2MOErA/*/i2MOErA/*/i2MOErA/*/i2MOErA/*/i2MOErG/*/ |
| | | i2MOErT/*/i2MOErG/*/i2MOErA/*/i2MOErT/*/i2MOErG/*/ |
| | | 32MOErG/ |
| 88 | SA5- | /52MOErC/*/i2MOErC/*/i2MOErT/*/i2MOErA/*/i2MOErT/*/ |
| | mod | i2MOErA/*/i2MOErA/*/i2MOErG/*/i2MOErG/*/i2MOErT/*/i2MOErA/ |
| | | */i2MOErA/*/i2MOErA/*/i2MOErA/*/i2MOErG/*/i2MOErT/*/ |
| | | i2MOErG/*/i2MOErA/*/i2MOErT/*/i2MOErG/*/i2MOErG/*/ |
| | | 32MOErG/ |
| 89 | SA6- | /52MOErC/*/i2MOErT/*/i2MOErA/*/i2MOErT/*/i2MOErA/*/ |
| | mod | i2MOErA/*/i2MOErG/*/i2MOErG/*/i2MOErT/*/i2MOErA/*/i2MOErA/ |
| | | */i2MOErA/*/i2MOErA/*/i2MOErG/*/i2MOErT/*/i2MOErG/*/ |
| | | i2MOErA/*/i2MOErT/*/i2MOErG/*/i2MOErG/*/i2MOErG/*/ |
| | | 32MOErA/ |
| 90 | SA7- | /52MOErT/*/i2MOErA/*/i2MOErT/*/i2MOErA/*/i2MOErA/*/ |
| | mod | i2MOErG/*/i2MOErG/*/i2MOErT/*/i2MOErA/*/i2MOErA/*/i2MOErA/ |
| | | */i2MOErA/*/i2MOErG/*/i2MOErT/*/i2MOErG/*/i2MOErA/*/ |
| | | i2MOErT/*/i2MOErG/*/i2MOErG/*/i2MOErG/*/i2MOErA/*/ |
| | | 32MOErT/ |
| 91 | SA8- | /52MOErA/*/i2MOErT/*/i2MOErA/*/i2MOErA/*/i2MOErG/*/ |
| | mod | i2MOErG/*/i2MOErT/*/i2MOErA/*/i2MOErA/*/i2MOErA/*/i2MOErA/ |
| | | */i2MOErG/*/i2MOErT/*/i2MOErG/*/i2MOErA/*/i2MOErT/*/ |
| | | i2MOErG/*/i2MOErG/*/i2MOErG/*/i2MOErA/*/i2MOErT/*/ |
| | | 32MOErC/ |
| 92 | SD9- | /52MOErT/*/i2MOErA/*/i2MOErA/*/i2MOErG/*/i2MOErG/*/ |
| | mod | i2MOErT/*/i2MOErA/*/i2MOErA/*/i2MOErA/*/i2MOErA/*/i2MOErG/ |
| | | */i2MOErT/*/i2MOErG/*/i2MOErA/*/i2MOErT/*/i2MOErG/*/ |
| | | i2MOErG/*/i2MOErG/*/i2MOErA/*/i2MOErT/*/i2MOErC/*/ |
| | | 32MOErA/ |
| 93 | SD10- | /52MOErA/*/i2MOErA/*/i2MOErG/*/i2MOErG/*/i2MOErT/*/ |
| | mod | i2MOErA/*/i2MOErA/*/i2MOErA/*/i2MOErA/*/i2MOErG/*/i2MOErT/ |
| | | */i2MOErG/*/i2MOErA/*/i2MOErT/*/i2MOErG/*/i2MOErG/*/ |
| | | i2MOErG/*/i2MOErA/*/i2MOErT/*/i2MOErC/*/i2MOErA/*/ |
| | | 32MOErC/ |

The ASO structures provided in Table. 3B are chemically modified, as designated by the symbols of chemical modification. 2-Methoxyethoxy RNA bases are shown in Table. 3B as “2MOEr” followed by the name of the RNA bases. For example, “2MOErT” is 2-methoxyethoxy thymine ribonucleotide. Phosphorothioated RNA bases are used and “*” designates phosphorothioate bond. Nucleotides are separated by “/”. The 5′ ends and the 3′ end of the ASO structures are marked by “5” and “3” in front of the nucleotide symbols, respectively. Internal nucleotides are marked by an “i” in front of the nucleotide symbols.

TABLE 4
Sequences of guide RNAs.

| SEQ ID NOs for | |
|---|---|
| guide RNAs | Guide RNAs |
| 94 | TGCTCAGTTATAGTATATAA |
| 95 | TTAGTTATCTGTTTAAACTA |

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

What is claimed is:

1. A method of modulating expression of a CFTR gene in a cell, comprising contacting an agent or a vector encoding the agent to the cell, wherein the cell comprises a pre-mRNA, wherein the pre-mRNA is transcribed from the CFTR gene and comprises a first intron that comprises an alternative polyadenylation site, and wherein the agent modifies the CFTR gene or modulates processing of the pre-mRNA, thereby increasing a level of a processed mRNA that is processed from the pre-mRNA and that lacks nucleic acid sequence of the pre-mRNA downstream of the first intron.

2. The method of claim 1, wherein the cell is a human cell, and the CFTR gene is a human gene.

3. The method of claim 2, wherein the first intron is Intron 22.

4. The method of claim 2, wherein the first intron is located at a region from GRCh38.p14/hg38: chr7: 117,627,771 to GRCh38.p14/hg38: chr7: 117,642,437.

5. The method of any one of claims 1-4, wherein the agent removes from genome of the cell nucleic acid sequence of the CFTR gene that is downstream of the first intron.

6. The method of claim 5, wherein the nucleic acid sequence that is removed from the genome is located from GRCh38.p14/hg38: chr7: 117,642,438 to GRCh38.p14/hg38: chr7: 117,668,665.

7. The method of claim 5 or 6, wherein the agent comprises gene editing agents based on CRISPR/Cas9, TALEN, Zinc Finger, or any combination thereof.

8. The method of claim 5 or 6, wherein the agent comprises a pair of guide RNAs, and wherein the pair of guide RNAs comprise the sequences of SEQ ID NOs: 94 and 95, respectively.

9. The method of any one of claims 1-8, wherein the agent removes from the pre-mRNA the nucleic acid sequence of the pre-mRNA downstream of the first intron.

10. The method of claim 9, wherein the nucleic acid sequence that is removed from the pre-mRNA is located from GRCh38.p14/hg38: chr7: 117,642,438 to GRCh38.p14/hg38: chr7: 117,668,665.

11. The method of any one of claims 1-10, wherein the agent suppresses splicing out of the first intron from the pre-mRNA.

12. A method of modulating expression of a CFTR gene in a cell, comprising contacting an agent or a vector encoding the agent to the cell, wherein the cell comprises a pre-mRNA that is transcribed from the CFTR gene and comprises a first intron that comprises an alternative polyadenylation site, and wherein the agent suppresses splicing out of the first intron from the pre-mRNA during splicing of the pre-mRNA in the cell.

13. The method of claim 12, wherein the cell is a human cell, and the CFTR gene is a human gene.

14. The method of claim 13, wherein the first intron is Intron 22.

15. The method of claim 13, wherein the first intron is located at a region from GRCh38.p14/hg38: chr7: 117,627,771 to GRCh38.p14/hg38: chr7: 117,642,437.

16. The method of any one of claims 12-15, wherein the agent increases a level of a processed mRNA that is processed from the pre-mRNA and that lacks nucleic acid sequence of the pre-mRNA downstream of the first intron.

17. The method of claim 16, wherein the nucleic acid sequence of the pre-mRNA downstream of the first intron is located at a region from GRCh38.p14/hg38: chr7: 117,642,438 to GRCh38.p14/hg38: chr7: 117,668,665.

18. The method of any one of claim 1-11, 16, or 17, wherein the level of the processed mRNA is increased in the cell by at least about 1.5, at least about 2, at least about 3, at least about 4, at least about 5, at least about 6, at least about 7, at least about 8, at least about 9, at least about 10, at least about 11, at least about 12, at least about 13, at least about 14, at least about 15, or at least about 20 times as compared to a corresponding cell that is not contacted with the agent or the vector encoding the agent.

19. The method of any one of claim 1-11, 16, or 17, wherein the level of the processed mRNA is increased in the cell by at least about 10 times as compared to a corresponding cell that is not contacted with the agent or the vector encoding the agent.

20. The method of any one of claim 1-11 or 16-19, wherein the processed mRNA comprises, in a 5′ to 3′ order, 22 exons, an intronic sequence encoding nine amino acids, a stop codon, and an alternative 3′ untranslated region.

21. The method of claim 20, wherein the intronic sequence encoding nine amino acids is transcribed from genomic sequence located from GRCh38.p14/hg38: chr7: 117,627,771 to GRCh38.p14/hg38: chr7: 117,627,797.

22. The method of any one of claim 1-11 or 16-21, wherein the processed mRNA is polyadenylated at an alternative polyadenylation site in a nucleic acid sequence transcribed from genomic sequence located between GRCh38.p14/hg38: chr7: 117,627,771 and GRCh38.p14/hg38: chr7: 117,642,437.

23. The method of any one of claims 1-22, wherein the agent increases a level of a truncated CTFR protein in the cell, which lacks amino acid sequence expressed from exonic sequences of the CFTR gene downstream of the first intron.

24. The method of claim 23, wherein the level of the truncated CFTR protein in the cell is increased by at least about 1.5, at least about 2, at least about 3, at least about 4, at least about 5, at least about 6, at least about 7, at least about 8, at least about 9, at least about 10, at least about 11, at least about 12, at least about 13, at least about 14, at least about 15, or at least about 20 times as compared to a corresponding cell that is not contacted with the agent or the vector encoding the agent.

25. The method of claim 23 or 24, wherein the truncated CFTR protein in the cell has a chloride channel conductivity that is less than 100%, less than 90%, less than 80%, less than 70%, less than 60%, less than 50%, less than 40%, less than 30%, less than 20%, less than 10%, or less than 5% of a chloride channel conductivity of a wildtype CFTR protein.

26. The method of any one of claims 12-25, further comprising contacting the cell with a second agent.

27. The method of claim 26, wherein the second agent comprises a modulator of CFTR protein that potentiates chloride conductance of CFTR in the cells.

28. The method of claim 26 or 27, wherein the second agent comprises ivacaftor, lumacaftor, tezacaftor, elexacaftor, a combination of lumacaftor and ivacaftor, a combination of tezacaftor and ivacaftor, or a combination of elexacaftor, ivacaftor, and tezacaftor.

29. The method of claim 26 or 27, wherein the second agent improves the chloride channel conductivity of the truncated CFTR protein by at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300%, at least 350%, at least 400%, at least 450%, at least 500%, at least 550%, at least 600%, at least 650%, at least 700%, at least 750%, at least 800%, at least at least 850%, at least 900%, at least 950%, at least 1000%.

30. The method of claim 26 or 27, wherein the second agent restores the chloride channel conductivity of the truncated CFTR protein to about at least 5%, at least 10%, at least 15%, at least 20%, at least 25%, at least 30%, at least 35%, at least 40%, least 45%, at least 50%, at least 55%, at least 60%, least 65%, at least 70%, at least 75%, at least 80%, at least 85%, at least 90%, at least 95% or more of the chloride channel conductivity of wildtype CFTR protein.

31. The method of any one of claims 1-30, wherein the agent:
(a) binds to a 5′ splice site of the first intron;
(b) binds to a 3′ splice site of the first intron;
(c) binds to a branch point for the 3′ splice site of the first intron; or
(d) interferes with a splicing factor that is involved in splicing out of the first intron.

32. The method of any one of claims 1-31, wherein the agent comprises a polynucleotide sequence that is at least 80% complementary to at least 8 contiguous nucleic acids of a sequence selected from the group consisting of SEQ ID NOs: 6-36.

33. The method of any one of claims 1-32, wherein the agent comprises a polynucleotide sequence that is at least 80% complementary to at least 10, 12, 14, or 16 contiguous nucleic acids of a sequence selected from the group consisting of SEQ ID NOs: 6-36.

34. The method of any one of claims 1-33, wherein the agent comprises a polynucleotide sequence that is at least 80% complementary to at least 18 contiguous nucleic acids of a sequence selected from the group consisting of SEQ ID NOs: 6-36.

35. The method of any one of claims 1-34, wherein the agent comprises a polynucleotide sequence that is 100% complementary to a sequence selected from the group consisting of SEQ ID NOs: 6-36.

36. The method of any one of claims 1-31, wherein the agent comprises a polynucleotide sequence with at least 80% identity to a sequence selected from the group consisting of SEQ ID NOs: 37-64.

37. The method of claim 36, wherein the agent comprises a polynucleotide sequence that comprises at least 8 contiguous nucleotides of a sequence selected from the group consisting of SEQ ID NOs: 37-64.

38. A method comprising contacting an agent or a vector encoding the agent to the cell, wherein the agent comprises a polynucleotide sequence that is at least 80% complementary to a sequence selected from the group consisting of SEQ ID NOs: 6-36.

39. A method comprising contacting an agent or a vector encoding the agent to the cell, wherein the agent comprises a polynucleotide sequence with at least 80% identity to a sequence selected from the group consisting of SEQ ID NOs: 37-64.

40. A method comprising contacting an agent or a vector encoding the agent to the cell, wherein the agent comprises at least 8 contiguous nucleotides of a sequence selected from the group consisting of SEQ ID NOs: 37-64.

41. The method of any one of claim 36-37 or 39-40, wherein the polynucleotide sequence of the agent has at least 90% identity to a sequence selected from the group consisting of SEQ ID NOs: 37-64.

42. The method of any one of claim 36-37 or 39-40, wherein the polynucleotide sequence of the agent has 100% identity to a sequence selected from the group consisting of SEQ ID NOs: 37-64.

43. The method of any one of claim 36-37 or 39-40, wherein the polynucleotide sequence of the agent comprises at least 10, 12, 14, or 16 contiguous nucleotides of a sequence selected from the group consisting of SEQ ID NOs: 37-64.

44. The method of claim 43, wherein the polynucleotide sequence of the agent comprises at least 18 contiguous nucleotides of a sequence selected from the group consisting of SEQ ID NOs: 37-64.

45. The method of claim 44, wherein the polynucleotide sequence of the agent comprises at least 20 contiguous nucleotides of a sequence selected from the group consisting of SEQ ID NOs: 37-64.

46. The method of any one of claims 1-45, wherein the agent is an antisense oligomer.

47. The method of claim 46, wherein the antisense oligomer comprises a backbone modification, a modified sugar moiety or a combination thereof.

48. The method of claim 46, wherein the antisense oligomer comprises a phosphorothioate linkage or a phosphorodiamidate linkage.

49. The method of any one of claims 46-48, wherein each internucleotide linkage of the antisense oligomer is a phosphorothioate linkage.

50. The method of any one of claims 46-49, wherein the antisense oligomer comprises a phosphorodiamidate morpholino, a locked nucleic acid, a peptide nucleic acid, a 2′-O-methyl moiety, a 2′-fluoro moiety, a 2′-O-methoxyethyl moiety, or a 2′-NMA moiety.

51. The method of any one of claims 46-50, wherein the antisense oligomer comprises at least one modified sugar moiety.

52. The method of claim 51, wherein each nucleotide of the antisense oligomer comprises a modified sugar moiety.

53. The method of claim 51, wherein each nucleotide of the antisense oligomer comprises a 2′-O-methoxyethyl moiety.

54. The method of any one of claims 46-53, wherein the antisense oligomer comprises at least one modified nucleobase.

55. The method of any one of claims 46-53, wherein the antisense oligomer comprises hypoxanthine, xanthine, 7-methylguanine, 5, 6-dihydrouracil, 5-methylcytosine, or 5-hydroxymethoylcytosine.

56. The method of any one of claims 46-55, wherein the antisense oligomer is from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 12 to 50 nucleobases, 12 to 40 nucleobases, 12 to 35 nucleobases, 12 to 30 nucleobases, 12 to 25 nucleobases, 12 to 20 nucleobases, 12 to 15 nucleobases, 15 to 50 nucleobases, 15 to 40 nucleobases, 15 to 35 nucleobases, 15 to 30 nucleobases, 15 to 25 nucleobases, 15 to 20 nucleobases, 15 to 19 nucleobases, 15 to 18 nucleobases, 15 to 16 nucleobases, 16 to 20 nucleobases, 16 to 19 nucleobases, 16 to 18 nucleobases, 17 to 20 nucleobases, 17 to 19 nucleobases, or 18 to 20 nucleobases in length.

57. The method of any one of claims 46-56, wherein the antisense oligomer comprises the sequence set forth in any one of SEQ ID NOs: 66-93.

58. The method of any one of claims 1-57, wherein the method comprises contacting to the cell the vector, and wherein the vector comprises a viral vector encoding the agent.

59. The method of claim 58, wherein the viral vector comprises an adenoviral vector, adeno-associated viral (AAV) vector, lentiviral vector, Herpes Simplex Virus (HSV) viral vector, or retroviral vector.

60. The method of any one of claims 1-59, wherein the CFTR gene comprises a mutation downstream the first intron.

61. The method of claim 60, wherein the mutation downstream of the first intron is a nonsense mutation.

62. The method of any one of claims 1-61, wherein the CFTR gene comprises a mutation that leads to presence of an in-frame premature termination codon that is downstream of the first intron.

63. The method of any one of claims 1-62, wherein at least one allele of the CFTR gene in the cell is a variant selected from the group consisting of: c.3717G>A, c.3717+4A>G, c.3717+5G>A, c.3717+40A>G, c.3718-2477C>T, c.3718-1G>A, c.3718-3T>G, c.3719T>G, c.3731G>A, c.3737C>T, c.3744delA, c.3745G>A, c.3747delG, c.3752G>A, c.3761T>G, c.3763T>C, c.3764C>A, c.3773dupT, c.3806T>A, c.3808delG, c.3808G>A, c.3822G>A, c.3846G>A, c. [3846G>A;3848G>T], c.3848G>T, c.3872A>G, c.3873G>C, c.3873+1G>A, c. (3873+1_3874-1)_(3963+1_3964-1) del, c.3873+2T>C, 3876delA, c.3883_3886delATTT, c.3883delA, c.3889dupT, c.3891dupT, c.3908delA, c.3909C>G, c.3929G>A, c.3937C>T, c. (3963+1_3964-1)_(*1_?) del, c.3964-78_4242+577del, c.3971T>C, c.3988C>T, c.4004T>C, c.4036_4042del, c.4046G>A, c.4077_4080delTGTTinsAA, c.4086dupT, c.4097T>A, c.4111G>T, c.4124A>C, c.4127_4131delTGGAT, c.4144C>T, c.4147dupA, c.4197_4198delCT, c.4231C>T, c.4234C>T, c.4242+1G>T, c.4242+1G>A, c.4251delA, c.4300_4301dup, c.4364C>G, c.4426C>T, and c.4439T>C.

64. A composition comprising an agent or a vector encoding the agent, wherein the agent, when present in a human cell that comprises a pre-mRNA, the pre-mRNA being transcribed from the CFTR gene and comprising a first intron that comprises an alternative polyadenylation site, modifies the CFTR gene or modulates processing of the pre-mRNA, thereby increasing a level of a processed mRNA that is processed from the pre-mRNA and that lacks nucleic acid sequence of the pre-mRNA downstream of the first intron.

65. The composition of claim 64, wherein the cell is a human cell, and the CFTR gene is a human gene.

66. The composition of claim 65, wherein the first intron is Intron 22.

67. The composition of claim 65, wherein the first intron is located at a region from GRCh38.p14/hg38: chr7: 117,627,771 to GRCh38.p14/hg38: chr7: 117,642,437.

68. The composition of any one of claims 64-67, wherein the agent removes from genome of the cell nucleic acid sequence of the CFTR gene that is downstream of the first intron.

69. The composition of claim 68, wherein the removed nucleic acid sequence is located from GRCh38.p14/hg38: chr7: 117,642,438 to GRCh38.p14/hg38: chr7: 117,668,665.

70. The composition of claim 68 or 69, wherein the agent comprises gene editing agents based on CRISPR/Cas9, TALEN, Zinc Finger, or any combination thereof.

71. The composition of claim 68 or 69, wherein the agent comprises a pair of guide RNAs, and wherein the pair of guide RNAs comprise the sequences of SEQ ID NOs: 94 and 95, respectively.

72. The composition of any one of claims 64-71, wherein the agent removes from the pre-mRNA the nucleic acid sequence of the pre-mRNA downstream of the first intron.

73. The composition of claim 72, wherein the removed nucleic acid sequence is located from GRCh38.p14/hg38: chr7: 117,642,438 to GRCh38.p14/hg38: chr7: 117,668,665.

74. The composition of any one of claims 64-73, wherein the agent suppresses splicing out of the first intron.

75. A composition comprising an agent or a vector encoding the agent, wherein the agent, when present in a cell that comprises a pre-mRNA, the pre-mRNA being transcribed from the CFTR gene and comprising a first intron that comprises an alternative polyadenylation site, suppresses splicing out of the first intron.

76. The composition of claim 75, wherein the cell is a human cell, and the CFTR gene is a human gene.

77. The composition of claim 76, wherein the first intron is Intron 22.

78. The composition of claim 76, wherein the first intron is located at a region from GRCh38.p14/hg38: chr7: 117,627,771 to GRCh38.p14/hg38: chr7: 117,642,437.

79. The composition of any one of claims 75-78, wherein the agent increases a level of a processed mRNA that is processed from the pre-mRNA and that lacks nucleic acid sequence of the pre-mRNA downstream of the first intron.

80. The composition of claim 79, wherein the nucleic acid sequence of the pre-mRNA downstream of the first intron is located at a region from GRCh38.p14/hg38: chr7: 117,642,438 to GRCh38.p14/hg38: chr7: 117,668,665.

81. The composition of any one of claim 64-75, 79, or 80, wherein the level of the processed mRNA is increased in the cell by at least about 1.5, at least about 2, at least about 3, at least about 4, at least about 5, at least about 6, at least about 7, at least about 8, at least about 9, at least about 10, at least about 11, at least about 12, at least about 13, at least about 14, at least about 15, or at least about 20 times as compared to a corresponding cell that is not contacted with the agent or the vector encoding the agent.

82. The composition of any one of claim 64-75, 79, or 80, wherein the level of the processed mRNA is increased in the cell by at least about 10 times as compared to a corresponding cell that is not contacted with the agent or the vector encoding the agent.

83. The composition of any one of claim 64-75 or 79-82, wherein the processed mRNA comprises, in a 5′ to 3′ order, 22 exons, an intronic sequence encoding nine amino acids, a stop codon, and an alternative 3′ untranslated region.

84. The composition of claim 83, wherein the intronic sequence encoding nine amino acids is transcribed from genomic sequence located from GRCh38.p14/hg38: chr7: 117,627,771 to GRCh38.p14/hg38: chr7: 117,627,797.

85. The composition of any one of claim 64-75 or 79-84, wherein the processed mRNA is polyadenylated at an alternative polyadenylation site in a nucleic acid sequence transcribed from genomic sequence located between GRCh38.p14/hg38: chr7: 117,627,771 and GRCh38.p14/hg38: chr7: 117,642,437.

86. The composition of any one of claims 64-85, wherein the agent increases a level of a truncated CTFR protein in the cell, which lacks amino acid sequence expressed from exonic sequences of the CFTR gene downstream of the first intron.

87. The composition of claim 86, wherein the level of the truncated CFTR protein in the cell is increased by at least about 1.5, at least about 2, at least about 3, at least about 4, at least about 5, at least about 6, at least about 7, at least about 8, at least about 9, at least about 10, at least about 11, at least about 12, at least about 13, at least about 14, at least about 15, or at least about 20 times as compared to a corresponding cell that is not contacted with the agent or the vector encoding the agent.

88. The composition of claim 87, wherein the truncated CFTR protein in the cell has a chloride channel conductivity that is less than 100%, less than 90%, less than 80%, less than 70%, less than 60%, less than 50%, less than 40%, less than 30%, less than 20%, less than 10%, or less than 5% of a chloride channel conductivity of a wildtype CFTR protein.

89. The composition of any one of claims 64-88, further comprising a second agent.

90. The composition of claim 89, wherein the second agent comprises a modulator of CFTR protein that potentiates chloride conductance of CFTR in the cells.

91. The composition of claim 89 or 90, wherein the second agent comprises ivacaftor, lumacaftor, tezacaftor, elexacaftor, a combination of lumacaftor and ivacaftor, a combination of tezacaftor and ivacaftor, or a combination of elexacaftor, ivacaftor, and tezacaftor.

92. The composition of any one of claims 89-91, wherein the second agent improves the chloride channel conductivity of the truncated CFTR protein by at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300%, at least 350%, at least 400%, at least 450%, at least 500%, at least 550%, at least 600%, at least 650%, at least 700%, at least 750%, at least 800%, at least at least 850%, at least 900%, at least 950%, at least 1000%.

93. The composition of any one of claims 89-92, wherein the second agent restores the chloride channel conductivity of the truncated CFTR protein to about at least 5%, at least 10%, at least 15%, at least 20%, at least 25%, at least 30%, at least 35%, at least 40%, least 45%, at least 50%, at least 55%, at least 60%, least 65%, at least 70%, at least 75%, at least 80%, at least 85%, at least 90%, at least 95% or more of the chloride channel conductivity of wildtype CFTR protein.

94. The composition of any one of claims 64-93, wherein the agent:
(a) binds to a 5′ splice site of the first intron;
(b) binds to a 3′ splice site of the first intron;
(c) binds to a branch point for the 3′ splice site of the first intron; or
(d) interferes with a splicing factor that is involved in splicing out of the first intron.

95. The composition of any one of claims 64-94, wherein the agent comprises a polynucleotide sequence that is at least 80% complementary to at least 8 contiguous nucleic acids of a sequence selected from the group consisting of SEQ ID NOs: 6-36.

96. The composition of any one of claims 64-95, wherein the agent comprises a polynucleotide sequence that is at least 80% complementary to at least 10, 12, 14, or 16 contiguous nucleic acids of a sequence selected from the group consisting of SEQ ID NOs: 6-36.

97. The composition of any one of claims 64-96, wherein the agent comprises a polynucleotide sequence that is at least 80% complementary to at least 18 contiguous nucleic acids of a sequence selected from the group consisting of SEQ ID NOs: 6-36.

98. The composition of any one of claims 64-97, wherein the agent comprises a polynucleotide sequence that is 100% complementary to a sequence selected from the group consisting of SEQ ID NOs: 6-36.

99. The composition of any one of claims 64-94, wherein the agent comprises a polynucleotide sequence with at least 80% identity to a sequence selected from the group consisting of SEQ ID NOs: 37-64.

100. The composition of claim 99, wherein the agent comprises a polynucleotide sequence that comprises at least 8 contiguous nucleotides of a sequence selected from the group consisting of SEQ ID NOs: 37-64.

101. A composition comprising an agent or a vector encoding the agent, wherein the agent comprises a polynucleotide sequence that is at least 80% complementary to a sequence selected from the group consisting of SEQ ID NOs: 6-36.

102. A composition comprising an agent or a vector encoding the agent, wherein the agent comprises a polynucleotide sequence with at least 80% identity to a sequence selected from the group consisting of SEQ ID NOs: 37-64.

103. A composition comprising an agent or a vector encoding the agent, wherein the agent comprises at least 8 contiguous nucleotides of a sequence selected from the group consisting of SEQ ID NOs: 37-64.

104. The composition of any one of claim 99-100 or 102-103, wherein the polynucleotide sequence of the agent has at least 90% identity to a sequence selected from the group consisting of SEQ ID NOs: 37-64.

105. The composition of any one of claim 99-100 or 102-103, wherein the polynucleotide sequence of the agent has 100% identity to a sequence selected from the group consisting of SEQ ID NOs: 37-64.

106. The composition of any one of claim 99-100 or 102-103, wherein the polynucleotide sequence of the agent comprises at least 10, 12, 14, or 16 contiguous nucleotides of a sequence selected from the group consisting of SEQ ID NOs: 37-64.

107. The composition of any one of claim 99-100 or 102-103, wherein the polynucleotide sequence of the agent comprises at least 18 contiguous nucleotides of a sequence selected from the group consisting of SEQ ID NOs: 37-64.

108. The composition of any one of claim 99-100 or 102-103, wherein the polynucleotide sequence of the agent comprises at least 20 contiguous nucleotides of a sequence selected from the group consisting of SEQ ID NOs: 37-64.

109. The composition of any one of claims 101-108, wherein the polynucleotide sequence of the agent is at least 80% complementary to at least 8 contiguous nucleic acids of a sequence selected from the group consisting of SEQ ID NOs: 6-36.

110. The composition of any one of claims 101-108, wherein the polynucleotide sequence of the agent is at least 80% complementary to at least 10, 12, 14, or 16 contiguous nucleic acids of a sequence selected from the group consisting of SEQ ID NOs: 6-36.

111. The composition of any one of claims 101-108, wherein the polynucleotide sequence of the agent is at least 80% complementary to at least 18 contiguous nucleic acids of a sequence selected from the group consisting of SEQ ID NOs: 6-36.

112. The composition of any one of claims 101-108, wherein the polynucleotide sequence of the agent is 100% complementary to a sequence selected from the group consisting of SEQ ID NOs: 6-36.

113. The composition of any one of claims 64-112, wherein the agent is an antisense oligomer.

114. The composition of claim 113, wherein the antisense oligomer comprises a backbone modification, a modified sugar moiety or a combination thereof.

115. The composition of claim 113, wherein the antisense oligomer comprises a phosphorothioate linkage or a phosphorodiamidate linkage.

116. The composition of any one of claims 113-115, wherein each internucleotide linkage of the antisense oligomer is a phosphorothioate linkage.

117. The composition of any one of claims 113-116, wherein the antisense oligomer comprises a phosphorodiamidate morpholino, a locked nucleic acid, a peptide nucleic acid, a 2′-O-methyl moiety, a 2′-fluoro moiety, a 2′-O-methoxyethyl moiety, or a 2′-NMA moiety.

118. The composition of any one of claims 113-117, wherein the antisense oligomer comprises at least one modified sugar moiety.

119. The composition of claim 118, wherein each nucleotide of the antisense oligomer comprises a modified sugar moiety.

120. The composition of claim 119, wherein each nucleotide of the antisense oligomer comprises a 2′-O-methoxyethyl moiety.

121. The composition of any one of claims 113-120, wherein the antisense oligomer comprises at least one modified nucleobase.

122. The composition of any one of claims 113-121, wherein the antisense oligomer comprises hypoxanthine, xanthine, 7-methylguanine, 5, 6-dihydrouracil, 5-methylcytosine, or 5-hydroxymethoylcytosine.

123. The composition of any one of claims 113-122, wherein the antisense oligomer is from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 12 to 50 nucleobases, 12 to 40 nucleobases, 12 to 35 nucleobases, 12 to 30 nucleobases, 12 to 25 nucleobases, 12 to 20 nucleobases, 12 to 15 nucleobases, 15 to 50 nucleobases, 15 to 40 nucleobases, 15 to 35 nucleobases, 15 to 30 nucleobases, 15 to 25 nucleobases, 15 to 20 nucleobases, 15 to 19 nucleobases, 15 to 18 nucleobases, 15 to 16 nucleobases, 16 to 20 nucleobases, 16 to 19 nucleobases, 16 to 18 nucleobases, 17 to 20 nucleobases, 17 to 19 nucleobases, or 18 to 20 nucleobases in length.

124. The composition of any one of claims 113-123, wherein the antisense oligomer comprises the sequence set forth in any one of SEQ ID NOs: 66-93.

125. The composition of any one of claims 64-112, wherein the composition comprises the vector, and wherein the vector comprises a viral vector encoding the agent.

126. The composition of claim 125, wherein the viral vector comprises an adenoviral vector, adeno-associated viral (AAV) vector, lentiviral vector, Herpes Simplex Virus (HSV) viral vector, or retroviral vector.

127. A method of treating a subject in need thereof, comprising contacting cells of the subject with the composition of any one of claims 64-126.

128. The method of claim 127, wherein the cells are ex vivo.

129. The method of claim 127, wherein the cells are in vivo.

130. The method of claim 127, comprising administering the composition to the subject via intranasal administration, intratracheal administration, intravenous injection, intraperitoneal injection, intramuscular injection, intrathecal injection, subcutaneous injection, oral administration, synovial injection, intracerebroventricular injection, intravitreal administration, subretinal injection, topical application, or implantation.

131. The method of claim 127, comprising administering the composition to the subject via respiratory route.

132. The method of any one of claims 127-131, further comprising administering to the subject a second agent.

133. The method of claim 132, wherein the second agent comprises a modulator of CFTR protein that potentiates chloride conductance of CFTR in the cells.

134. The method of claim 132 or 133, wherein the second agent comprises ivacaftor, lumacaftor, tezacaftor, elexacaftor, a combination of lumacaftor and ivacaftor, a combination of tezacaftor and ivacaftor, or a combination of elexacaftor, ivacaftor, and tezacaftor.

135. The method of any one of claims 132-134, wherein the second agent comprises a mucolytic agent for airway clearance, optionally wherein the mucolytic agent for airway clearance is selected from the group consisting of: acetylcysteine, ambroxol, bromhexine, carbocisteine, erdosteine, mecysteine, dornase alfa, hypertonic saline, and mannitol.

136. The method of any one of claims 132-135, wherein the second agent comprises a bronchodilator, optionally wherein the bronchodilator is albuterol.

137. The method of any one of claims 132-136, wherein the second agent comprises an immunosuppressive agent.

138. The method of claim 137, wherein the immunosuppressive agent is a corticosteroid selected from the group consisting of beclomethasone, budesonide, budesonide, formoterol, ciclesonide, fluticasone, salmeterol, umeclidinium, vilanterol, mometasone, or mometasone, prednisone, and methylprednisolone, and pharmaceutically acceptable salts thereof.

139. The method of claim 137, wherein the immunosuppressive agent is a non-steroidal immunosuppressive agent selected from the group consisting of polyclonal anti-lymphocyte antibodies, monoclonal anti-lymphocyte antibodies, interleukin-2 (IL-2) receptor antagonists, calcineurin inhibitors, cell cycle inhibitors, mammalian target of rapamycin (mTOR) inhibitors, methotrexate, cyclophosphamide, an anthracycline, a taxane.

140. The method of any one of claims 127-139, wherein the method treats a disease or condition caused by a mutation in CFTR gene in the subject.

141. The method of any one of claims 127-140, wherein the CFTR gene comprises a mutation downstream the first intron.

142. The method of claim 141, wherein the mutation downstream of the first intron is a nonsense mutation.

143. The method of any one of claims 127-140, wherein the CFTR gene comprises a mutation that leads to presence of an in-frame premature termination codon that is downstream of the first intron.

144. The method of any one of claims 127-140, wherein a least one allele of the CFTR gene in the cells of the subject is a variant selected from the group consisting of: c.3717G>A, c.3717+4A>G, c.3717+5G>A, c.3717+40A>G, c.3718-2477C>T, c.3718-1G>A, c.3718-3T>G, c.3719T>G, c.3731G>A, c.3737C>T, c.3744delA, c.3745G>A, c.3747delG, c.3752G>A, c.3761T>G, c.3763T>C, c.3764C>A, c.3773dupT, c.3806T>A, c.3808delG, c.3808G>A, c.3822G>A, c.3846G>A, c. [3846G>A;3848G>T], c.3848G>T, c.3872A>G, c.3873G>C, c.3873+1G>A, c. (3873+1_3874-1)_(3963+1_3964-1) del, c.3873+2T>C, c.3883_3886delATTT, c.3883delA, c.3889dupT, c.3891dupT, c.3908delA, c.3909C>G, c.3929G>A, c.3937C>T, c. (3963+1_3964-1)_(*1_?) del, c.3964-78_4242+577del, c.3971T>C, c.3988C>T, c.4004T>C, c.4036_4042del, c.4046G>A, c.4077_4080delTGTTinsAA, c.4086dupT, c.4097T>A, c.4111G>T, c.4124A>C, c.4127_4131delTGGAT, c.4144C>T, c.4147dupA, c.4197_4198delCT, c.4231C>T, c.4234C>T, c.4242+1G>T, c.4242+1G>A, c.4251delA, c.4300_4301dup, c.4364C>G, c.4426C>T, and c.4439T>C.

145. The method of any one of claims 127-144, wherein the method ameliorates or prevents one or more symptoms associated with cystic fibrosis.

146. A pharmaceutical composition, comprising:
(a) a pharmaceutically acceptable excipient or carrier; and
(b) the composition of any one of claims 64-126.

147. The pharmaceutical composition of claim 146, wherein the pharmaceutical composition is formulated for intranasal administration, intratracheal administration, intravenous injection, intraperitoneal injection, intramuscular injection, intrathecal injection, subcutaneous injection, oral administration, synovial injection, intracerebroventricular injection, intravitreal administration, subretinal injection, topical application, or implantation.

148. The pharmaceutical composition of claim 146, wherein the pharmaceutical composition is formulated for administration via respiratory route.

149. The pharmaceutical composition of any one of claims 146-148, wherein the pharmaceutical composition further comprises a second therapeutic agent.

150. The pharmaceutical composition of claim 149, wherein the second therapeutic agent comprises a modulator of CFTR protein that potentiates chloride conductance of CFTR in the cells.

151. The pharmaceutical composition of claim 149 or 150, wherein the second agent comprises ivacaftor, lumacaftor, tezacaftor, elexacaftor, a combination of lumacaftor and ivacaftor, a combination of tezacaftor and ivacaftor, or a combination of elexacaftor, ivacaftor, and tezacaftor.

152. The pharmaceutical composition of any one of claims 149-151, wherein the second agent comprises a mucolytic agent for airway clearance, optionally wherein the mucolytic agent for airway clearance is selected from the group consisting of: acetylcysteine, ambroxol, bromhexine, carbocisteine, erdosteine, mecysteine, dornase alfa, hypertonic saline, and mannitol.

153. The pharmaceutical composition of any one of claims 149-152, wherein the second agent comprises a bronchodilator, optionally wherein the bronchodilator is albuterol.

154. The pharmaceutical composition of any one of claims 149-153, wherein the second agent comprises an immunosuppressive agent.

155. The pharmaceutical composition of claim 154, wherein the immunosuppressive agent is a corticosteroid selected from the group consisting of beclomethasone, budesonide, budesonide, formoterol, ciclesonide, fluticasone, salmeterol, umeclidinium, vilanterol, mometasone, or mometasone, prednisone, and methylprednisolone, and pharmaceutically acceptable salts thereof.

156. The pharmaceutical composition of claim 154, wherein the immunosuppressive agent is a non-steroidal immunosuppressive agent selected from the group consisting of polyclonal anti-lymphocyte antibodies, monoclonal anti-lymphocyte antibodies, interleukin-2 (IL-2) receptor antagonists, calcineurin inhibitors, cell cycle inhibitors, mammalian target of rapamycin (mTOR) inhibitors, methotrexate, cyclophosphamide, an anthracycline, a taxane.

157. A kit comprising:
(a) the composition of any one of claims 64-126 or the pharmaceutical composition of any one of claims 146-156; and
(b) instructions for use of the composition or the pharmaceutical composition.

158. A kit comprising:
(a) the composition of any one of claims 64-126 or the pharmaceutical composition of claim 146-156; and
(b) a second therapeutic agent.

159. The kit of claim 158, wherein the second therapeutic agent comprises a modulator of CFTR protein that potentiates chloride conductance of CFTR in the cells.

160. The kit of claim 158 or 159, wherein the second agent comprises ivacaftor, lumacaftor, tezacaftor, elexacaftor, a combination of lumacaftor and ivacaftor, a combination of tezacaftor and ivacaftor, or a combination of elexacaftor, ivacaftor, and tezacaftor.

161. The kit of any one of claims 158-160, wherein the second agent comprises a mucolytic agent for airway clearance, optionally wherein the mucolytic agent for airway clearance is selected from the group consisting of: acetylcysteine, ambroxol, bromhexine, carbocisteine, erdosteine, mecysteine, dornase alfa, hypertonic saline, and mannitol.

162. The kit of any one of claims 158-161, wherein the second agent comprises a bronchodilator, optionally wherein the bronchodilator is albuterol.

163. The kit of any one of claims 158-162, wherein the second agent comprises an immunosuppressive agent.

164. The kit of claim 163, wherein the immunosuppressive agent is a corticosteroid selected from the group consisting of beclomethasone, budesonide, budesonide, formoterol, ciclesonide, fluticasone, salmeterol, umeclidinium, vilanterol, mometasone, or mometasone, prednisone, and methylprednisolone, and pharmaceutically acceptable salts thereof.

165. The kit of claim 163, wherein the immunosuppressive agent is a non-steroidal immunosuppressive agent selected from the group consisting of polyclonal anti-lymphocyte antibodies, monoclonal anti-lymphocyte antibodies, interleukin-2 (IL-2) receptor antagonists, calcineurin inhibitors, cell cycle inhibitors, mammalian target of rapamycin (mTOR) inhibitors, methotrexate, cyclophosphamide, an anthracycline, a taxane.

166. The kit of any one of claims 158-165, further comprising instructions for use of the composition or the pharmaceutical composition, and instructions for use of the second therapeutic agent.
